SEQUENCE LISTING (26 sequences)

SEQ ID NO: 1 - protein, 5 aa, Artificial Sequence
  Synthetic
NGRRT

SEQ ID NO: 2 - protein, 5 aa, Artificial Sequence
  Synthetic
NGRRN

SEQ ID NO: 3 - protein, 8 aa, Artificial Sequence
  Synthetic
NNNNGATT

SEQ ID NO: 4 - protein, 8 aa, Artificial Sequence
  Synthetic
NNNNRYAC

SEQ ID NO: 5 - protein, 7 aa, Artificial Sequence
  Synthetic
NNAGAAW

SEQ ID NO: 6 - protein, 4 aa, Artificial Sequence
  Synthetic
TTTV

SEQ ID NO: 7 - protein, 4 aa, Artificial Sequence
  Synthetic
TTCN

SEQ ID NO: 8 - protein, 288 aa, Artificial Sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 9 - protein, 183 aa, Artificial Sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 10 - protein, 7 aa, Simian virus 40
PKKKRKV

SEQ ID NO: 11 - protein, 10 aa, Simian virus 40
PKKKRKVEAS

SEQ ID NO: 12 - protein, 16 aa, Simian virus 40
KRPAATKKAGQAKKKK

SEQ ID NO: 13 - protein, 9 aa, Homo sapiens
PAAKRVKLD

SEQ ID NO: 14 - protein, 11 aa, Homo sapiens
RQRRNELKRSP

SEQ ID NO: 15 - protein, 38 aa, Homo sapiens
NQSSNFGPMKGGNFGGRSSGPYGGGGQYFAKPRNQGGY

SEQ ID NO: 16 - protein, 42 aa, Homo sapiens
RMRIZFKNKGKDTAELRRRRVEVSVELRKAKKDEQILKRRNV

SEQ ID NO: 17 - protein, 8 aa, Homo sapiens
VSRKRPRP

SEQ ID NO: 18 - protein, 8 aa, Homo sapiens
PPKKARED

SEQ ID NO: 19 - protein, 8 aa, Homo sapiens
PQPKKKPL

SEQ ID NO: 20 - protein, 12 aa, Mus sp.
SALIKKKKKMAP

SEQ ID NO: 21 - protein, 5 aa, Influenza virus
DRLRR

SEQ ID NO: 22 - protein, 7 aa, Influenza virus
PKQKKRK

SEQ ID NO: 23 - protein, 10 aa, Hepatitis virus
RKLKKKIKKL

SEQ ID NO: 24 - protein, 10 aa, Mus sp.
REKKKFLKRR

SEQ ID NO: 25 - protein, 20 aa, Homo sapiens
KRKGDEVDGVDEVAKKKSKK

SEQ ID NO: 26 - protein, 17 aa, Homo sapiens
RKCLQAGMNLEARKTKK

(19) United States
(12) Patent Application Publication
(10) Pub. No.: US 2022/0323406 A1
(43) Pub. Date: Oct. 13, 2022

(54) METHODS AND COMPOSITIONS FOR DIFFERENTIATING STEM CELLS

(71) Applicants: Massachusetts Institute of Technology, Cambridge, MA (US); The Brigham and Women's Hospital, Inc., Boston, MA (US)

(72) Inventors: Benjamin Mead, Cambridge, MA (US); Alexander K. Shalek, Cambridge, MA (US); Jeffrey Karp, Boston, MA (US); Kazuki Hattori, Cambridge, MA (US)

(21) Appl. No.: 17/617,048

(22) PCT Filed: Jun. 5, 2020
(86) PCT No.: PCT/US2020/036446

§ 371 Date: Dec. 7, 2021

Related U.S. Application Data
(60) Provisional application No. 62/858,852, filed on Jun. 7, 2019.
(60) Provisional application No. 62/980,002, filed on Feb. 21, 2020.

(51) Int. Cl. A61K 31/366; A61K 31/506; A61P 1/00; A61K 35/36; A61K 35/38; C12N 5/071

(52) U.S. Cl. CPC A61K 31/366; A61K 31/506; A61P 1/00; A61K 35/36; A61K 35/38; C12N 5/0679; C12N 2533/90

(57) ABSTRACT

The subject matter disclosed herein is generally directed to modulation of genes and pathways that drive differentiation of LGR5+ stem cells. The methods and compositions can be used to treat diseases associated with aberrant epithelial barrier function.

### CROSS-REFERENCE TO RELATED APPLICATIONS

This application claims the benefit of U.S. Provisional Application No. 62/858,852, filed Jun. 7, 2019 and U.S. Provisional Application No. 62/980,002, filed Feb. 21, 2020. The entire contents of the above-identified applications are hereby fully incorporated herein by reference.

### STATEMENT REGARDING FEDERALLY SPONSORED RESEARCH

This invention was made with government support under Grant Nos. GM119419, CA217377 and HL095722 awarded by the National Institutes of Health. The government has certain rights in the invention.

### REFERENCE TO AN ELECTRONIC SEQUENCE LISTING

The contents of the electronic sequence listing (BROD-4380WP_ST25.txt”; Size is 9 kb and it was created on Jun. 2, 2020) is herein incorporated by reference in its entirety.

### TECHNICAL FIELD

The subject matter disclosed herein is generally directed to modulation of pathways that drive differentiation of LGR5+ stem cells.

### BACKGROUND

The intestinal epithelium is a complex tissue that plays a key role in digestion and mediates innate and adaptive immune functions. The small intestinal epithelium is formed by a single layer of cells arranged into villi-primarily composed of enterocytes, absorptive cells, and secretory Goblet cells—and crypts, which contain intestinal stem cells (ISCs) and secretory Paneth cells (PCs). Intestinal stem cells differentiate into mature intestinal cells, but signaling pathways and factors that modulate differentiation to Paneth cells are insufficiently understood. Several inflammatory and disease states are associated with intestinal irregularities, including inflammatory bowel disease, Crohn's disease, necrotizing enterocolitis, and intestinal inflammation. Intestinal stem cell differentiation is related to differentiation of other stem cells (e.g., stem cells found in the inner ear, barrier tissues, respiratory epithelium (lung, nose) and skin). Related diseases associated with irregularities include hearing loss, allergy, asthma, and psoriasis. Thus, there is a need for improved understanding of stem cell differentiation.

### SUMMARY

In one aspect, the present invention provides for a method of differentiating a stem cell or a stem cell enriched population of cells comprising contacting the population of cells with one or more cell cycle inhibitors; and/or contacting the population of cells with one or more agents capable of increasing expression or activity of one or more genes or gene products selected from the group consisting of. Atf3; or Atf3, Klf6, Hbegf, Ubc, Muc13; or Klf6, Atf3, Hbegf, Ubc, Muc13, 2210404007Rik, Pigr, Ndrg1, S1c16a6, Btg2, Thbs1, Cldn4, Edn1, Smim6, Arrdc3, Abcg8, Tmem171, Prr15, Rnase4, Itln1, Mmp7, Gm15284, Defa17, Lyz1, Tff3, Rps26, Rpl13, Hmgn1, Sox4, Malat1, S1c12a2, Atp1a1, Tmprss2, Gls, Mt2, Mt1, mmu-mir-6236 and Gm26924; or upregulated genes in Table 3A; or upregulated pathways in Table 3B, and/or contacting the population of cells with one or more agents capable of decreasing expression or activity of one or more genes or gene products selected from the group consisting of: Topbp1, Rpa1, Mcm7, Atad2, Lig1, Whsc1, Dut1, Urf1, Mcm3, Mcm5, Mcm4, Hells, Mcm6, Mcm2, Pcna, Tgm2, Crip2, Fen1, Ccnd1, Dtl, Cdt1, Ptma, Hook1, Tubb5, Mki67, Ranbp1 and Kpnb1; or Itpr3, Cbr1, Kmt2a, Hsph1, Gm2697, Bex1, Cad, Rpph1, Gclc, Cbr3, Pfkp, Ugdh, Elovl6, Slc25a4, Galk1, Wars, Slc38a1, Parp1, Mrpl12, Gsr, Gnl3, Ybx3, Ckb, Isynal, Nop56, Nolc1, Reep6, Ccnd2, Srm, Gpx2, Add3, Oxct1, Nhp2, Bzw2, Prmt5, Rangap1, H1f0, Sae1, C1qbp, Tpi1, Hmgcs1, Smoc2, Axin2, Cdk4, Reg3 g, Igfbp4, Rgcc, Maged1, Tcof1, Dctpp1, Nasp, Gart, Rif1, Paics, Hjurp, Ezh2, Rrm1, Dnmt1, Ipo5, Fads1, Topbp1, Rpa1, Mcm7, Atad2, Lig1, Whsc1, Dut1, Urf1, Mcm3, Mcm5, Mcm4, Hells, Mcm6, Mcm2, Pcna, Tgm2, Crip2, Fen1, Ccnd1, Dtl, Cdt1, Ptma, Hook1, Tubb5, Mki67, Ranbp1 and Kpnb1; or downregulated genes in Table 3A; or downregulated pathways in Table 3B, and/or contacting the population of cells with one or more agents capable of inducing a quiescent stem cell signature.

In certain embodiments, the one or more agents capable of inducing a quiescent stem cell signature comprise a map kinase inhibitor. In certain embodiments, the map kinase inhibitor is cobimetinib.

In certain embodiments, the one or more agents comprises a small molecule, genetic modifying agent, nucleic acid molecule encoding a gene product, protein, or any combination thereof.

In certain embodiments, the genetic modifying agent comprises a CRISPR system, RNAi system, a zinc finger nuclease system, a TALE system, or a meganuclease. In certain embodiments, the CRISPR system is a Class 1 or Class 2 CRISPR system. In certain embodiments, the Class 2 system comprises a Type II Cas polypeptide. In certain embodiments, the Type II Cas is a Cas9. In certain embodiments, the Class 2 system comprises a Type V Cas polypeptide. In certain embodiments, the Type V Cas is Cas12a, Cas12b, Cas12c, Cas12d (CasY), Cas12e (CasX), or Cas14. In certain embodiments, the Class 2 system comprises a Type VI Cas polypeptide. In certain embodiments, the Type VI Cas is Cas13a, Cas13b, Cas13c or Cas13d. In certain embodiments, the CRISPR system comprises a dCas fused or otherwise linked to a nucleotide deaminase. In certain embodiments, the nucleotide deaminase is a cytidine deaminase or an adenosine deaminase. In certain embodiments, the CRISPR system is a prime editing system.

In certain embodiments, the method further comprises contacting the population of cells with a Wnt agonist. In certain embodiments, the Wnt agonist is CHIR99021. In certain embodiments, the method further comprises contacting the population of cells with a Notch inhibitor. In certain embodiments, the Notch inhibitor is DAPT.

In certain embodiments, the stem cells are leucine-rich repeat-containing G-protein coupled receptor 5-positive (LGR5+) cells. In certain embodiments, the LGR5+ cells are LGR5+ intestinal stem cells (ISC), LGR5+ cochlear progenitors (LCP), LGR5+ stem cells of the respiratory epithelium, or LGR5+ stem cells of the skin.

In certain embodiments, the population of cells is an in vivo population of cells. In certain embodiments, the population of cells is an in vitro organoid model system.

In certain embodiments, the method further comprises obtaining intestinal cells from a subject. In certain embodiments, the method further comprises obtaining inner ear cells from a subject.

In certain embodiments, secretory cells are increased in the population of cells. In certain embodiments, the secretory cells comprise Paneth cells. In certain embodiments, the Paneth cells express one or more genes or gene products selected from the group consisting of human lysozyme (LYZ), a human alpha defensin (DEFA), human matrix metalloproteinase-7 (MMP-7), and cluster of differentiation 24 (CD24). In certain embodiments, the human alpha defensin is human alpha defensin 5 (DEFA5) or human alpha defensin 6 (DEFA6). In certain embodiments, sensory cells are increased in the population of cells. In certain embodiments, the sensory cells comprise inner ear hair cells.

In certain embodiments, the method comprises: a) culturing an LGR5+ enriched population of cells in a hydrogel matrix in the presence of EGF, Noggin, R-spondin 1, CHIR99021 and valproic acid (ENR+CV); b) culturing the ENR+CV cells in the presence of EGF, Noggin, R-spondin 1, CHIR99021 and DAPT (ENR+CD); and c) contacting the cells according to any embodiment herein.

In certain embodiments, the method further comprises introducing differentiated cells to a subject.

In certain embodiments, the method is for treating a condition or disease by increasing Paneth cells in a subject in need thereof. In certain embodiments, the condition or disease is selected from the group consisting of graft-versus-host disease (GVHD), inflammatory bowel disease (IBD), Crohn's disease, necrotizing enterocolitis, microbial dysbiosis, impaired intestinal epithelial barrier function, obesity, intestinal inflammation, allergy, respiratory inflammation, asthma, and psoriasis. In certain embodiments, the method comprises targeted administration of the one or more agents or cells to the intestine of the subject.

In certain embodiments, the method is for treating hearing loss by increasing hair cells in the inner ear of a subject in need thereof. In certain embodiments, the method comprises targeted administration of the one or more agents or cells to the inner ear of the subject.

In another aspect, the present invention provides for a population of cells produced by the method of any embodiment herein.

In another aspect, the present invention provides for a method of treating a condition or disease by increasing Paneth cells in a subject in need thereof comprising systemically administering an inhibitor of nuclear export to the subject in a dosage less than or equal to 0.2 mg/kg. In certain embodiments, the dosage is between 0.01 to 0.2 mg/kg. In certain embodiments, the dosage is less than or equal to 0.01 mg/kg. In certain embodiments, the inhibitor is administered orally. In certain embodiments, the inhibitor is administered by injection. In certain embodiments, the inhibitor of nuclear export inhibits XPO1. In certain embodiments, the inhibitor is KPT-330. In certain embodiments, the inhibitor is KPT-8602. In certain embodiments, the inhibitor is Leptomycin B. In certain embodiments, the condition or disease is selected from the group consisting of inflammatory bowel disease (IBD), Crohn's disease, intestinal inflammation, graft-versus-host disease (GVHD), necrotizing enterocolitis, microbial dysbiosis, impaired intestinal epithelial barrier function, obesity, allergy, respiratory inflammation, asthma, and psoriasis.

In another aspect, the present invention provides for a method of detecting intestinal cell types comprising detecting in a sample comprising intestinal cells the expression of one or markers selected from Table 4 for one or more cell types selected from the group consisting of stem I cells, stem II cells, stem III cells, early enterocytes, enterocytes, early Paneth cells, Paneth cells and enteroendocrine cells. In certain embodiments, the fraction of each cell type is determined.

In another aspect, the present invention provides for a method of identifying an agent capable of differentiating a stem cell or a stem cell enriched population of cells comprising applying a candidate agent to an intestinal organoid model; and detecting one or more intestinal cell types according to claim 44, whereby a shift in cell types indicates that the agent is capable of differentiating a stem cell or a stem cell enriched population of cells.

In another aspect, the present invention provides for a method of differentiating a stem cell or a stem cell enriched population of cells comprising contacting the population of cells with one or more inhibitors of aurora kinase b. In certain embodiments, the inhibitor is ZM447439.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Schematic showing an intestinal organoid model system to study drivers of epithelial composition.

FIG. 2—Schematic for in vitro 6-day PC differentiation screen and multiplexed single-well assays. Also shown is a schematic of the “3-D” organoid culture and “2.5-D” culture, which enables the enhanced multiplexed measurement of secreted supernatant lysozyme and cell pellet adenosine adenosine-5′-triphosphate (ATP). LYZ activity in 1 is referred to as LYZ.NS and LYZ activity in 2 is referred to as LYZ.S.

FIG. 3A-3F—Define hits by significant increases in secretion. A. Replicate UMVUE SSMD for each well and assay in screen, large points are deemed hits above FPL and FNL-determined cutoff, circled points are hits in both LYZ.NS and LYZ.S assays, each point represents the SSMD from 3 replicates of 3 bio. donors relative to whole-plate control. B. SSMD for LYZ.NS, LYZ.S, and ATP assays for 6-day ENR+CD+treatment versus ENR+CD+DMSO (vehicle) control, with an FPL=0.05 determined cutoff (0.89), large dots signify treatment-doses passing cutoffs for both LYZ assays, n=8 well replicates. C. Venn diagram of treatment hits based on replicate SSMD across the 3 assays. D. Biological potency for LYZ.NS versus LYZ.S assays based on mean fold change (based on n=8 well replicates) of treatment relative to control, grey signifies treatments advanced for profiling. E. Mean fold change of assay effect for hits in LYZ.S and LYZ.NS, only points above 1.28 standard deviations of all treatment mean fold changes (corresponding to the top 10% of a normal distribution) are deemed potent hits. F. Table of validated small molecules at their maximal doses (top indicates TGF-beta/Smad pathway, middle indicates Tyrosine kinase pathway).

FIG. 4—Schematic and fold-change (FC) results for early (day 0-3) vs. full (day 0-6) treatment at indicated doses of indicated compounds relative to ENR+CD control in LYZ.NS normalized to ATP, LYZ.S normalized to ATP, and ATP assays.

FIG. 5A-5C—Population RNA-seq suggests KPT-330 drives secretory gene expression. A. Graph showing the number of differentially expressed genes after treatment with the indicated small molecule in the indicated condition (top bar is ENR, middle is ENR+CD, bottom is combined). B. Gene set scores for the indicated cell type after treatment with the small molecules under the ENR and ENR+CD conditions. C. Plots showing differentially expressed genes in response to KPT-330 treatment under the ENR and ENR+CD conditions.

FIG. 6A-6B—Assays in conventional (3D) system confirm KPT-330 differentiation. A. Graph showing measurements of the Paneth cell composition (Lyz+/CD24+) via flow cytometry of ENR+CD organoids treated with the indicated small molecules. B. Graph showing measurements of LYZ secretion compared to ATP in ENR organoids with induced (Cch—carbamyl choline) secretion.

FIG. 7A-7B—Additional Xpo1 inhibitors suggest action through known mode of action. A. Chemical structures of Xpo1 inhibitors. B. Graphs showing measurements of the Paneth cell composition (Lyz+/CD24+) via flow cytometry of ENR+CD organoids treated with the indicated Xpo1 inhibitors.

FIG. 8—(left) Western blotting for LYZ in organoids cultured in ENR+CV or ENR with or without XPO1 inhibitors for six days. (right) Western blotting for LYZ in organoids cultured in ENR+CD with or without XPO1 inhibitors for six days.

FIG. 9—(left) Lysozyme secretion assay normalized by whole-well ATP, conducted on organoids differentiated in ENR+CD for 6 days with multiple XPO1 inhibitors, with both induced (Cch—carbamyl choline) and non-induced secretion. Dunnett's multiple comparison test: ** adj. p<0.01, *** adj. p<0.005, n=5 well replicates. (right) Lysozyme secretion assay normalized by whole-well ATP, conducted on organoids differentiated in ENR for 6 days with multiple XPO1 inhibitors, with both induced (Cch—carbamyl choline) and non-induced secretion. Dunnett's multiple comparison test: ** adj. p<0.01, **** adj. p<0.0001, n=8 well replicates.

FIG. 10A-10B—Population RNA-seq across conditions with organoids (Media: ENR, ENR+C, ENR+D, ENR+CD; Drug: none, KPT-330, KPT-8602; timing: 3 days, 6 days). A. Heatmap showing treatment cell type identity scores for each condition. B. Plot showing the effect of SINEs (Selective Inhibitor of Nuclear Export) on intestinal organoid differentiation.

FIG. 11—Graph showing LYZ secretion and ATP in ENR+CD organoids across the indicated time course with KPT-330 treatment.

FIG. 12—Single cell RNA sequencing. Seq-well results for single cells from each of the indicated organoids (control, treated with KPT-330; and indicated time points).

FIG. 13A-13C—Unsupervised differentiation landscape. A. UMAP clustering of single organoid cells shows cells cluster by cell type. B. (left) UMAP plots with treatment status and time status projected on plots. (right) Expression of the indicated marker genes projected on the plots. C. (left) UMAP clustering of single organoid cells shows cells cluster by cell type. (right) Fraction of indicated cell types across time course in cells treated and untreated with KPT-330.

FIG. 14A-14B—KPT-330 enhances stem conversion to mature cells. A. Organoid cell composition over time in untreated cells. B. Organoid cell composition over time in KPT-330 treated cells.

FIG. 15A-15C—Stem III (cycling)/Stem II (intermediate) express Xpo1+ nuclear export signal (NES) transcripts. A. Violin plots showing Xpo1 expression across intestinal cell types. B. Violin plots showing NES1 expression across intestinal cell types. C. Violin plots showing Xpo1 expression levels and NES-containing gene score within control cells.

FIG. 16—Differentially expressed genes are well-distributed across cell types. Graph showing the number of differentially expressed genes in each cell type in control and KPT-330 treated organoids.

FIG. 17A-17C—KPT-330 induces ‘stress-response’ and cell cycle inhibitory modules in Stem II/III A. Heatmap showing a gene x gene correlation analysis of genes upregulated after KPT-330 treatment (see, Table 1). B. Heatmap showing a gene x gene correlation analysis of genes downregulated after KPT-330 treatment (see, Table 2). C. Violin plots showing expression of Atf3 in response to KPT-330.

FIG. 18A-18B—KPT-330 induces a quiescent ISC signature. A. UMAP clustering of day 0-1 stem cell populations shaded by stem cell type (left), an active ISC signature (middle), and a quiescent ISC signature. B. Violin plots showing changes in stem cell signatures in response to KPT-330 treatment.

FIG. 19—Induction of stem quiescence enhances effect of KPT-330. Graph showing LYZ secretion and ATP from 6 day ENR+CD organoids after treatment with cobimetinib and KPT-330.

FIG. 20—KPT-330 treatment in vivo. Graph showing body weight of C57BL6 wild-type, 10-week-old, male mice from 0 to 8 days after treatment with control or KPT-330 (4 mice/group; 4 times administration per week, 1 week, oral gavage).

FIG. 21A-21C—Analysis of samples collected from the mice of FIG. 20. A. Western blot for lysozyme of samples collected from the small intestine proximal region. B. Graph showing band intensity of the western blot in (A). C. Histological analysis of the distal small intestine from control and treated mice using immunohistochemistry for lysozyme.

FIG. 22—Analysis of samples collected from the mice of FIG. 20. Quantification of histology suggests a pro-differentiation effect. Graphs showing Paneth cell, Goblet cell, and cycling cell numbers in the proximal and distal small intestines under each treatment condition using markers for each cell type.

FIG. 23A-23G—A. Diagram for the stem-enriched to Paneth-enriched organoid differentiation screen, and schematic of the multiplexed functional secretion assays performed on day 6. B. Replicate UMVUE SSMD for each well and assay in screen, colored points are deemed hits above FPL and FNL-determined cutoff in both LYZ.NS and LYZ.S assays, each point represents the SSMD from 3 replicates of 3 bio. donors relative to whole-plate control. C. Mean fold change of assay effect for hits in LYZ.S and LYZ.NS, only points above 1.28 standard deviations of all treatment mean fold changes (corresponding to the top 10% of a normal distribution) are deemed potent hits. D. Biological potency for LYZ.NS versus LYZ.S assays based on mean fold change (based on n=8 well replicates) of treatment relative to control, orange signifies treatments advanced for profiling. E. Flow cytometry analyses of 3D-cultured intestinal organoids, treated with 6 hit compounds during 6 days culture in ENR+CD media. Paneth cells were identified as lysozyme-positive and CD24-positive cells. Means and individual values are shown (N=4), and the dot line represents the average of Paneth cell proportion in control samples. The Dunnett's multiple comparisons test; ****p<0.0001, ***p<0.001. F. ENR+CD media containing increasing concentrations of KPT-330 were treated for 6 days. Organoids were incubated in fresh basal media with or without 10 μM carbamoylcholine chloride (Cch) for 3 h on day 6. All data were normalized to ATP abundance and further standardized to the control in each experiment. Means and individual values are shown (N=5 (D,G,H), N=8 (E)), and the dotted line represents the value 1. G. Flow cytometry analyses of 3D-cultured intestinal organoids, treated with 160 nM KPT-8602 or 2 ng/mL Leptomycin B during 6 days culture in ENR+CD media. Paneth cells were identified as lysozyme-positive and CD24-positive cells. Means and individual values are shown (N=4). Unpaired two-tailed t-test; **p<0.01. Dunnett's multiple comparisons test; ****p<0.0001, ***p<0.001, *p<0.05.

FIG. 24A-24G—A. Diagram for the stem-enriched to Paneth-enriched organoid differentiation. B. UMAP plot with projection of cells at each time point. C. UMAP with projection of cell type clusters. D. Heatmap showing differentially expressed genes between cell types. E. Violin plots showing expression of each module across the cell types. F. Fraction of indicated cell types across time course in cells treated and untreated with KPT-330. G. Odds ratio of indicated cell types across time course in cells treated vs. untreated with KPT-330.

FIG. 25A-25J—A. Relative (log normalized) expression of Xpo1 across stem cells. B. Nuclear export signal module score across stem cells. C. Graph showing experimental conditions for treatment of ENR+CD with KPT-330. D. Graph showing the percentage of Paneth cells after treatment as in E. Plot showing genes upregulated and downregulated after KPT-330 treatment. F. Graph showing GSEA programs upregulated and downregulated after KPT-330 treatment. G. Graphs showing stress module and mitogen signaling module expression across the cell types in treated vs. untreated cells. H. Violin plots showing active ISC module and quiescent ISC module scores+/−KPT-330 treatment. I. Graphs showing the effects on Paneth cells of further treatment with an AP-1 inhibitor or ERK inhibitor. ENR+CD media containing 160 nM KPT-330 and/or 20 nM Cobimetinib were treated for 6 days. Organoids were incubated in fresh basal media with or without 10 μM carbamoylcholine chloride for 3 h on day 6. J. Proposed mechanism for Xpo1 inhibition in rebalancing cycling stem cell fate decisions towards secretory Paneth cells and absorptive enterocytes. All data were normalized to ATP abundance and further standardized to the control in each experiment. Means and individual values are shown (N=5 (D,G,H), N=8 (E)), and the dotted line represents the value 1. One sample t-test compared to 1, followed by the Two-stage linear step-up method of Benjamini, Krieger and Yekutieli for adjusting p-values; **p<0.01, *p<0.05 (D,E,G). Tukey's multiple comparisons test; ****p<0.0001, **p<0.01 (H).

FIG. 26A-26D—A. Design for in vivo oral gavage of KPT-330 in wild-type (WT) C57BL/6 mice. B. Graphs showing mean Paneth cell number in crypts isolated from the small intestine of mice treated with KPT-330. C. Graphs showing mean Olfm4+ stem cell number in crypts isolated from the small intestine of mice treated with KPT-330. D. Graphs showing mean goblet cell number in crypts isolated from the small intestine of mice treated with KPT-330.

FIG. 27A-27J—A. Distribution of all sample data (n=5676 wells) for each assay following data transformation and normalization, dotted line indicates median of distribution for which all fold change calculations are determined. B. Spearman correlation (r) between all sample wells by screen plate and biological replicate. C. ATP, LYZ.NS, LYZ.S assay controls across all plates and replicates, Welch's t test for ATP, one-way ANOVA with Dunnett's multiple comparison test * adj. p<0.05, **** adj. p<0.0001. D. SSMD for LYZ.NS, LYZ.S, and ATP assays for 6-day ENR+CD+treatment versus ENR+CD+DMSO (vehicle) control, with an FPL=0.05 determined cutoff (0.89), circled dots signify treatment-doses passing cutoffs for both LYZ assays, n=8 well replicates. E. Flow cytometry gating strategy to select viable mature Paneth cells. F. Lysozyme secretion assay of 3D-cultured intestinal organoids, treated with 6 hit compounds during 6 days culture in ENR media. 10 μM Carbamoylcholine (Cch) chloride was treated for 3 h on day 6. All data were normalized to ATP abundance and further standardized to the control in each experiment. Means and individual values are shown (N=5), and the dot line represents the value 1. One sample t-test compared to 1, followed by the Two-stage linear step-up method of Benjamini, Krieger and Yekutieli for adjusting p-values; **p<0.01, *p<0.05. G. Lysozyme secretion assay of 3D-cultured intestinal organoids, treated with 160 nM KPT-330, 160 nM KPT-8602 or 2 ng/mL Leptomycin B during 6 days culture in ENR+CD media. Organoids were incubated in fresh basal media with or without 10 μM carbamoylcholine chloride for 3 h on day 6. All data were normalized to ATP abundance and further standardized to the control in each experiment. Means and individual values are shown (N=5), and the dotted line represents the value 1. One sample t-test compared to 1, followed by the Two-stage linear step-up method of Benjamini, Krieger and Yekutieli for adjusting p-values; **p<0.01, *p<0.05. H. Lysozyme secretion assay of 3D-cultured intestinal organoids, treated with 160 nM KPT-330, 160 nM KPT-8602 or 2 ng/mL Leptomycin B during 6 days culture in ENR media. Organoids were incubated in fresh basal media with or without 10 μM carbamoylcholine chloride for 3 h on day 6. All data were normalized to ATP abundance and further standardized to the control in each experiment. Means and individual values are shown (N=5), and the dotted line represents the value 1. One sample t-test compared to 1, followed by the Two-stage linear step-up method of Benjamini, Krieger and Yekutieli for adjusting p-values; **p<0.01, *p<0.05. I. Western blotting of lysozyme in 3D-cultured intestinal organoids, cultured in ENR+CD media for 6 days. J. Western blotting of lysozyme in 3D-cultured intestinal organoids, cultured in ENR media for 6 days.

FIG. 28A-28F—A. Violin plots showing UMI, percent mitochondrial, and detected gene distributions are across samples. B. Violin plots showing UMI, percent mitochondrial, and detected gene distributions are across cell type clusters. C. Projection of lineage-defining gene sets from a murine small intestinal scRNA-seq atlas on UMAP plots. D. Projection of gene sets identified to correspond to known ISC subsets in vivo on UMAP plots of stem cells. E. Violin plots showing module scores for stem cell types. F. UMAP plots showing clusters across all three conditions, day 0 ENR+CV, and day 0.25-6 ENR+CD and ENR+CD+KPT-330.

FIG. 29A-29I—A. Violin plots showing expression of Xpo1 across cell types. B. Violin plots showing expression of genes known to contain a NES across cell types. C. Graph showing the expression of key mediators in the mitogen-activated protein kinase (MAPK) pathway, NFAT, AP-1, and Aurora kinase activity within the stem populations. D. Graph showing a LYZ secretion assay across a time course of KPT-330 treatment. E. Graph showing a LYZ secretion assay across a time course of KPT-8602 treatment. Lysozyme secretion assay of 3D-cultured intestinal organoids, treated with 160 nM KPT-8602 during the indicated time frame in ENRCD media. Organoids were incubated in fresh basal media with 10 μM carbamoylcholine chloride for 3 h on day 6. All data were normalized to ATP abundance and further standardized to the control in each experiment. Means and individual values are shown (N=5), and the dot line represents the value 1. One sample t-test compared to 1, followed by the Two-stage linear step-up method of Benjamini, Krieger and Yekutieli for adjusting p-values; **p<0.01, *p<0.05. F. Graph showing Xpo1, Atf3, Trp53 (p53), Ccnd1, Cdk4/6, and Cdkn1a (p21) expression across all cell types and the fraction of cells in each which express each gene+/− KPT-330 treatment. G. Graph showing the effects on Paneth cells of further treatment with two known P53 inhibitors, pifithrin-α (PFTa) and serdemetan (serd.). H. Graph showing the effects on Paneth cells of treatment with the Cdk4/6 inhibitor palbociclib. I. Graph showing the effects on Paneth cells of treatment with the aurora kinase b inhibitor ZM447439.

FIG. 30A-30E—A. Graph showing body weight of C57BL/6 wild-type mice administered KPT-330 at a dose of 10 mg/kg via oral gavage every other day over a two-week span. B. Graph showing body weight of C57BL/6 wild-type mice administered KPT-330 at a dose of 50-fold (0.2 mg/kg), 200-fold (0.05 mg/kg), and 1,000-fold (0.01 mg/kg) below the 10 mg/kg dose via oral gavage every other day over a two-week span. C. Immunohistochemistry images of Paneth cells within well preserved crypts. D. Immunohistochemistry images of Olfm4+ stem cells within well preserved crypts. E. Immunohistochemistry images of PAS+ goblet cells within well preserved crypts.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to U.S. patent application Ser. No. 16/240,361, filed Jan. 4, 2019 and International Patent Publication No. WO2014159356A1. Reference is also made to the manuscript entitled, “High-throughput organoid screening enables engineering of intestinal epithelial composition,” by Benjamin E. Mead, Kazuki Hattori, Lauren Levy, Marko Vukovic, Daphne Sze, Juan D. Matute, Jinzhi Duan, Robert Langer, Richard S. Blumberg, Jose Ordovas-Montanes, Alex K. Shalek, Jeffrey M. Karp, posted to bioRxiv on Apr. 28, 2020 (bioRxiv 2020.04.27.063727; doi: doi.org/10.1101/2020.04.27.063727).

All publications, published patent documents, and patent applications cited herein are hereby incorporated by reference to the same extent as though each individual publication, published patent document, or patent application was specifically and individually indicated as being incorporated by reference.

### Overview

Stem cells of the small intestine integrate diverse signals to regulate regeneration and differentiation, which in turn set the composition of the intestinal epithelium and supports barrier function. Therapeutically directing stem cell differentiation may therefore provide novel approaches to augment barrier function by altering the abundance or quality of specialized cells of the epithelium, including the secretory Paneth, goblet, and enteroendocrine populations. Utilizing an organoid model of directed intestinal stem cell differentiation into antimicrobial-secreting Paneth cells, Applicants performed a first-of-its-kind high throughput phenotypic screen of over four hundred well-characterized target-specific small molecules for pro-differentiation effects, independent of known cues in Wnt and Notch signaling pathways. Applicants identified and validated three compounds which significantly increase the abundance of mature Paneth cells in the differentiation model though the inhibition of the nuclear exporter Xpo1. With single-cell RNA-sequencing and validating experiments Applicants reveal that enhanced stem-to-Paneth differentiation through Xpo1 inhibition is driven by a pan-epithelial stress response combined with an interruption of mitogen signaling in actively cycling intestinal progenitors. Applicants extended the observation of pro-Paneth cell differentiation in vivo and demonstrated that oral dosing of Xpo1 inhibitor KPT-330 at 1,000-fold lower than used in cancer increases Paneth cell abundance. In totality, Applicants provide a framework to conduct translational studies in organoid models and demonstrate a pathway for discovery of novel molecular targets controlling barrier tissue composition.

Embodiments disclosed herein provide methods and compositions for differentiating stem cells to mature cells. The stem cells may be present in a cell culture, spheroid, organoid, tissue explant, or in vivo (see, e.g., Yin X, Mead B E, Safaee H, Langer R, Karp J M, Levy O. Cell Stem Cell 2016; 18.25-38). The methods may be used to treat diseases requiring an increase in mature cells (e.g., Paneth cells, inner ear hair cells, respiratory cells, stomach cells, kidney cells). “Cell differentiation” refers to the process by which a cell becomes specialized to perform a specific function, such as in the conversion of post-natal stem cells into cells having a more specialized function. In certain embodiments, LGR5⁺ stem cells are differentiated into Paneth cells or cells that express characteristics of Paneth cells.

The intestinal epithelium is a complex tissue that plays a key role in digestion and mediates important innate and adaptive immune functions. The small intestinal epithelium is formed by a single layer of cells arranged into villi-primarily composed of enterocytes, absorptive cells, and secretory Goblet cells—and crypts, which contain intestinal stem cells (ISCs) and secretory Paneth cells (PCs). Cells located in the villi are specialized for nutrient absorption, cells residing in the crypts are integral to regenerating the intestinal epithelium, and specialized cells throughout the epithelium provide for a protective barrier between host and microbe. Goblet cells secrete mucins into the lumen of the intestine to create a physical barrier between the host and the bacteria populating the gut. PCs contribute to the barrier by secreting antimicrobial proteins (AMPs) to form a biochemical barrier. In a healthy small intestinal epithelium, PCs are potent modulators of the gut microflora through the known secretion of multiple antimicrobial protein families including lysozyme (LYZ), angiogenin, ribonuclease A family, regenerating islet-derived 3 gamma (REG3G), and peptides such as cystine-rich (CRS) peptides and alpha-defensins (DEFA). PCs also secrete cytokines including interleukin-17 (IL-17) and are involved in signaling across the innate and adaptive immune system. The gut microbiota participates in a variety of different functions including metabolism, host defense and immune development and has been linked to pathogenesis in gastrointestinal, autoimmune, and other diseases.

Genetic, morphological, and functional alternations in PCs have been shown to drive microbial dysbiosis, impaired intestinal epithelial barrier function, and inflammation. This includes the heterogeneous collection of pathologies that manifest as inflammatory bowel disease (IBD). Genetic associations linked to impaired PC function in IBD populations include abnormalities in NOD2 (innate immune activation), ATG16L1 (granule exocytosis), and XBP1 (ER stress response). The AMPs secreted by PCs also play a crucial role in protection against infection from enteric pathogens. Notably, in in vivo murine models, PC-depleted and AMP deficient mice are more susceptible to bacterial translocation and inflammation. As well, in necrotizing enterocolitis (NEC), AMP secretion and PC number is altered corresponding with intestinal immaturity and dysbiosis. The immature epithelial barrier appears to be more sensitive to bacteria and bacterial translocation, leading to excessive inflammation and systemic infection. Furthermore, PC disruption in mice replicates human NEC pathology, suggesting that PCs may initiate NEC. PCs have also been implicated in Graft versus Host disease (GvHD), which occurs after an allogeneic stem cell transplant in which the donor T cells cause an inflammatory response in the host. Patients with GHVD also exhibit a loss in PC number, reduced expression of AMPs, and dysbiosis. Notably, Gram-negative bacteria become more prevalent and, when paired with impaired barrier function, can lead to severe sepsis.

Additionally, growing evidence implicates the gut microbiota in the development of metabolic syndrome, which precipitates cardiovascular disease, type 2 diabetes, and obesity, affecting nearly a third of Americans. Interestingly, PC abnormalities relating to ER stress response have been correlated with the very obese. Furthermore, increasing the population dynamics of certain ‘protective’ bacteria has been shown to mitigate a pro-obesity effect and metabolic syndrome-associated low-grade inflammation; this microbiota modulation may be done in the future through a PC axis.

The importance of impaired barrier function and dysregulation of the gut microbiota in the etiology of these diseases suggests that PCs present a promising therapeutic axis. This has already been demonstrated in GvHD. Treatment with R-spondin1 (R-Spo1), a potent WNT agonist, can elevate the secretion of alpha-defensins and restore the dysbiosis seen in mice with GvHD by stimulating ISCs to differentiate into PCs. However, while treatment with R-Spo1 illustrates the importance of PC regeneration, it faces many challenges in clinical translation to humans. R-Spo1 is shown to significantly increase crypt size and hyperactive WNT activation is implicated in precancerous hyperplasia and PC metaplasia. While the effects of R-Spo1 are inconclusive with respect to malignancy, WNT signaling must be carefully balanced to ensure homeostasis not priming for cancer. Other signaling pathways known to drive PC differentiation, including Notch signaling, face similar challenges. Activation of Notch signaling amplifies the proliferative progenitor population and promotes an absorptive cell lineage. Conversely, deactivation of Notch signaling amplifies differentiation to all secretory cell types and secretory cell hyperplasia. As these pathways affect multiple cell types in the intestinal epithelium and may lead to hyperplasia, they are not therapeutically viable. Therefore, a more specific PC targeted treatment to accomplish PC regeneration is desirable.

Intestinal stem cell differentiation is related to differentiation of stem cells in other tissues (e.g., stem cells found in the inner ear, barrier tissues, respiratory epithelium (lung, nose) and skin). Related diseases associated with irregularities in other tissues include hearing loss, inflammation, allergy, asthma, and psoriasis. Thus, the methods described herein can be used for regeneration of cells in other organs. The regeneration of cells may result in increased barrier function in other tissues.

As used herein a “barrier cell” or “barrier tissues” refers generally to various epithelial tissues of the body such, but not limited to, those that line the respiratory system, digestive system, urinary system, and reproductive system as well as cutaneous systems. The epithelial barrier may vary in composition between tissues but is composed of basal and apical components, or crypt/villus components in the case of intestine. Reduced epithelial barrier integrity is a characteristic of severe clinical presentations associated with type 2 inflammatory (T2I) responses (see, e.g., International Patent Publication No. WO 2019/018441).

### Stem Cells and Stem Cell Enriched Populations

In certain embodiments, the method of the present invention can be used to differentiate stem cells or a population of cells enriched for stem cells. In certain embodiments, pluripotent cells may be used. In certain embodiments, differentiation of stem cells present in vivo can be used for regeneration of mature epithelial cell types, in particular LGR5+ stem cells present in tissues. Differentiation of stem cells to epithelial cells may increase barrier function. Stem cells differentiated ex vivo may also be used for regeneration of mature epithelial cell types in vivo.

In certain embodiments, organoids enriched for stem cells are differentiated. As used herein, the term “organoid” or “epithelial organoid” refers to a cell cluster or aggregate that resembles an organ, or part of an organ, and possesses cell types relevant to that particular organ. Organoid systems have been described previously, for example, for brain, retinal, stomach, lung, thyroid, small intestine, colon, liver, kidney, pancreas, prostate, mammary gland, fallopian tube, taste buds, salivary glands, and esophagus (see, e.g., Clevers, Modeling Development and Disease with Organoids, Cell. Jun. 16, 2016; 165(7):1586-1597).

Intestinal organoids, derived from intestinal stem cells (ISCs) and composed of ISCs, Paneth cells (PCs), enteroendocrine cells (EECs), goblet cells and absorptive enterocytes, have been invaluable to the study of intestinal biology (Clevers, 2016). Conventional intestinal organoids produced from the spontaneous differentiation of ISCs have been used to study PCs in vitro in multiple contexts (Farin, et al. Paneth cell extrusion and release of antimicrobial products is directly controlled by immune cell-derived IFN-γ. J. Exp. Med. 2014; 211:1393-405; and Wilson, et al., A small intestinal organoid model of non-invasive enteric pathogen-epithelial cell interactions. Mucosal Immunol. Nature; 2014; 8:1-10).

In certain embodiments, the stem cells are LGR5+ stem cells. “LGR5” is an acronym for the leucine-rich repeat-containing G-protein coupled receptor 5, also known as G-protein coupled receptor 49 (GPR49) or G-protein coupled receptor 67 (GPR67). It is a protein that in humans is encoded by the Lgr5 gene. “LGR5+ cell” or “LGR5-positive cell” is a cell that expresses Lgr5. Lgr5 is a member of GPCR class A receptor proteins. R-spondin proteins are the biological ligands of LGR5. LGR5 is a biomarker of adult stem cells in certain tissues. LGR5 is a marker of adult intestinal stem cells. The high turnover rate of the intestinal lining is due to a dedicated population of stem cells found at the base of the intestinal crypt. In vivo lineage tracing showed that LGR5 is expressed in nascent nephron cell cluster within the developing kidney. Specifically, the LGR5+ stem cells contribute into the formation of the thick ascending limb of Henle's loop and the distal convoluted tubule. However, expression is eventually truncated after postnatal day 7, a stark contrast to the facultative expression of LGR5 in actively renewing tissues such as in the intestines (Barker, et al., 2012 “Lgr5+ve stem/progenitor cells contribute to nephron formation during kidney development”. Cell Rep. 2 (3): 540-52). The stomach lining also possesses populations of LGR5+ stem cells, although there are two conflicting theories: one is that LGR5+ stem cells reside in the isthmus, the region between the pit cells and gland cells, where most cellular proliferation takes place. However, lineage tracing had revealed LGR5+ stem cells at the bottom of the gland (Barker, et al., 2010 “Lgr5+ve stem cells drive self-renewal in the stomach and build long-lived gastric units in vitro”. Cell Stem Cell. 6 (1): 25-36), architecture reminiscent to that of the intestinal arrangement. This suggests that LGR5 stem cells give rise to transit-amplifying cells, which migrate towards the isthmus where they proliferate and maintain the stomach epithelium (Barker N, Clevers H, 2010 “Leucine-rich repeat-containing G-protein-coupled receptors as markers of adult stem cells”. Gastroenterology. 138 (5): 1681-96). LGR5+ve stem cells were pinpointed as the precursor for sensory hair cells that line the cochlea (Ruffner, et al. 2012 “R-Spondin potentiates Wnt/0-catenin signaling through orphan receptors LGR4 and LGR5”. PLoS ONE. 7 (7): e40976).

Pluripotent cells may include any mammalian stem cell. As used herein, the term “stem cell” refers to a multipotent cell having the capacity to self-renew and to differentiate into multiple cell lineages. Mammalian stem cells may include, but are not limited to embryonic stem cells of various types, such as murine embryonic stem cells, e.g., as described by Evans & Kaufman 1981 (Nature 292: 154-6) and Martin 1981 (PNAS 78: 7634-8); rat pluripotent stem cells, e.g., as described by lannaccone et al. 1994 (Dev Biol 163: 288-292); hamster embryonic stem cells, e.g., as described by Doetschman et al. 1988 (Dev Biol 127: 224-227); rabbit embryonic stem cells, e.g., as described by Graves et al. 1993 (Mol Reprod Dev 36: 424-433); porcine pluripotent stem cells, e.g., as described by Notarianni et al. 1991 (J Reprod Fertil Suppl 43: 255-60) and Wheeler 1994 (Reprod Fertil Dev 6: 563-8); sheep embryonic stem cells, e.g., as described by Notarianni et al. 1991 (supra); bovine embryonic stem cells, e.g., as described by Roach et al. 2006 (Methods Enzymol 418: 21-37); human embryonic stem (hES) cells, e.g., as described by Thomson et al. 1998 (Science 282: 1 145-1 147); human embryonic germ (hEG) cells, e.g., as described by Shamblott et al. 1998 (PNAS 95: 13726); embryonic stem cells from other primates such as Rhesus stem cells, e.g., as described by Thomson et al. 1995 (PNAS 92:7844-7848) or marmoset stem cells, e.g., as described by Thomson et al. 1996 (Biol Reprod 55: 254-259). In certain embodiments, the pluripotent cells may include, but are not limited to lymphoid stem cells, myeloid stem cells, neural stem cells, skeletal muscle satellite cells, epithelial stem cells, endodermal and neuroectodermal stem cells, germ cells, extraembryonic and embryonic stem cells, mesenchymal stem cells, intestinal stem cells, embryonic stem cells, and induced pluripotent stem cells (iPSCs).

As noted, prototype “human ES cells” are described by Thomson et al. 1998 (supra) and in U.S. Pat. No. 6,200,806. The scope of the term covers pluripotent stem cells that are derived from a human embryo at the blastocyst stage, or before substantial differentiation of the cells into the three germ layers. ES cells, in particular hES cells, are typically derived from the inner cell mass of blastocysts or from whole blastocysts. Derivation of hES cell lines from the morula stage has been documented and ES cells so obtained can also be used in the invention (Strelchenko et al. 2004. Reproductive BioMedicine Online 9: 623-629). As noted, prototype “human EG cells” are described by Shamblott et al. 1998 (supra). Such cells may be derived, e.g., from gonadal ridges and mesenteries containing primordial germ cells from fetuses. In humans, the fetuses may be typically 5-11 weeks post-fertilization.

In certain embodiments, mouse embryonic stem cells are used. In certain embodiments, mouse embryonic stem cells differentiated into a target cell may be transferred to a mouse to perform in vivo functional studies.

Human embryonic stem cells may include, but are not limited to the HUES66, HUES64, HUES3, HUES8, HUES53, HUES28, HUES49, HUES9, HUES48, HUES45, HUES1, HUES44, HUES6, H1, HUES62, HUES65, H7, HUES13 and HUES63 cell lines.

General techniques useful in the practice of this invention in cell culture and media uses are known in the art (e.g., Large Scale Mammalian Cell Culture (Hu et al. 1997. Curr Opin Biotechnol 8: 148); Serum-free Media (K. Kitano. 1991. Biotechnology 17: 73); or Large Scale Mammalian Cell Culture (Curr Opin Biotechnol 2: 375, 1991). The terms “culturing” or “cell culture” are common in the art and broadly refer to maintenance of cells and potentially expansion (proliferation, propagation) of cells in vitro. Typically, animal cells, such as mammalian cells, such as human cells, are cultured by exposing them to (i.e., contacting them with) a suitable cell culture medium in a vessel or container adequate for the purpose (e.g., a 96-, 24-, or 6-well plate, a T-25, T-75, T-150 or T-225 flask, or a cell factory), at art-known conditions conducive to in vitro cell culture, such as temperature of 37° C., 5% v/v CO₂ and >95% humidity.

Methods related to culturing stem cells are also useful in the practice of this invention (see, e.g., “Teratocarcinomas and embryonic stem cells: A practical approach” (E. J. Robertson, ed., IRL Press Ltd. 1987); “Guide to Techniques in Mouse Development” (P. M. Wasserman et al. eds., Academic Press 1993); “Embryonic Stem Cells: Methods and Protocols” (Kursad Turksen, ed., Humana Press, Totowa N.J., 2001); “Embryonic Stem Cell Differentiation in vitro” (M. V. Wiles, Meth. Enzymol. 225: 900, 1993); “Properties and uses of Embryonic Stem Cells: Prospects for Application to Human Biology and Gene Therapy” (P. D. Rathjen et al., al., 1993). Differentiation of stem cells is reviewed, e.g., in Robertson. 1997. Meth Cell Biol 75: 173; Roach and McNeish. 2002. Methods Mol Biol 185: 1-16; and Pedersen. 1998. Reprod Fertil Dev 10: 31). For further elaboration of general techniques useful in the practice of this invention, the practitioner can refer to standard textbooks and reviews in cell biology, tissue culture, and embryology (see, e.g., Culture of Human Stem Cells (R. Ian Freshney, Glyn N. Stacey, Jonathan M. Auerbach—2007); Protocols for Neural Cell Culture (Laurie C. Doering—2009); Neural Stem Cell Assays (Navjot Kaur, Mohan C. Vemuri—2015); Working with Stem Cells (Henning Ulrich, Priscilla Davidson Negraes—2016); and Biomaterials as Stem Cell Niche (Krishnendu Roy—2010)). In certain embodiments, stem cells are spontaneously differentiated or directed to differentiate (see, e.g., Amit and Itskovitz-Eldor, Derivation and spontaneous differentiation of human embryonic stem cells, J Anat. 2002 March; 200(3): 225-232). For further methods of cell culture solutions and systems, see International Patent Publication No. WO2014159356A1.

In certain embodiments, iPSCs or iPSC cell lines are used to identify transcription factors for differentiation of target cells. iPSCs advantageously can be used to generate patient specific models and cell types. iPSCs are a type of pluripotent stem cell that can be generated directly from adult cells. Further, because embryonic stem cells can only be derived from embryos, it has so far not been feasible to create patient-matched embryonic stem cell lines.

Various strategies can be used to induce pluripotency, or increase potency, in cells (Takahashi, K., and Yamanaka, S., Cell 126, 663-676 (2006); Takahashi et al., Cell 131, 861-872 (2007); Yu et al., Science 318, 1917-1920 (2007); Zhou et al., Cell Stem Cell 4, 381-384 (2009); Kim et al., Cell Stem Cell 4, 472-476 (2009); Yamanaka et al., 2009; Saha, K., Jaenisch, R., Cell Stem Cell 5, 584-595 (2009)), and improve the efficiency of reprogramming (Shi et al., Cell Stem Cell 2, 525 20 528 (2008a); Shi et al., Cell Stem Cell 3, 568-574 (2008b); Huangfu et al., Nat Biotechnol 26, 795-797 (2008a); Huangfu et al., Nat Biotechnol 26, 1269-1275 (2008b); Silva et al., Plos Bio 6, e253. doi: 10.1371/journal.pbio. 0060253 (2008); Lyssiotis et al., PNAS 106, 8912-8917 (2009); Ichida et al., Cell Stem Cell 5, 491-503 (2009); Maherali, N., Hochedlinger, K., Curr Biol 19, 1718-1723 (2009b); Esteban et 25 al., Cell Stem Cell 6, 71-79 (2010); and Feng et al., Cell Stem Cell 4, 301-312 (2009)).

Generally, techniques for reprogramming involve modulation of specific cellular pathways, either directly or indirectly, using polynucleotide-, polypeptide and/or small molecule-based approaches (see, e.g., International Patent Publication No. WO 2012/087965A2). The developmental potency of a cell may be increased, for example, by contacting a cell with one or more pluripotency factors. “Contacting”, as used herein, can involve culturing cells in the presence of a pluripotency factor (such as, for example, small molecules, proteins, peptides, etc.) or introducing pluripotency factors into the cell. Pluripotency factors can be introduced into cells by culturing the cells in the presence of the factor, including transcription factors such as proteins, under conditions that allow for introduction of the transcription factor into the cell. See, e.g., Zhou H et al., Cell Stem Cell. May 8, 2009; 4(5):381-4; WO/2009/117439. Introduction into the cell may be facilitated for example, using transient methods, e.g., protein transduction, microinjection, non-integrating gene delivery, mRNA transduction, etc., or any other suitable technique. In some embodiments, the transcription factors are introduced into the cells by expression from a recombinant vector that has been introduced into the cell, or by incubating the cells in the presence of exogenous transcription factor polypeptides such that the polypeptides enter the cell. In particular embodiments, the pluripotency factor is a transcription factor. Exemplary transcription factors that are associated with increasing, establishing, or maintaining the potency of a cell include, but are not limited to Oct-3/4, Cdx-2, 15 Gbx2, Gshl, HesX1, HoxA10, HoxA 11, HoxB1, Irx2, Isl1, Meis1, Meox2, Nanog, Nkx2.2, Onecut, Otx1, Oxt2, Pax5, Pax6, Pdx1, Tcfl, Tcf2, Zfhxlb, Klf-4, Atbfl, Esrrb, Genf, Jarid2, Jmjdla, Jmjd2c, Klf-3, Klf-5, Mel-18, Myst3, Nacl, REST, Rex-i, Rybp, Sa1l4, Sall1, Tifl, YYl, Zeb2, Zfp281, Zfp57, Zic3, Coup-Tfl, Coup-Tf2, Bmil, Rnf2, Mtal, Pias1, Pias2, Pias3, Piasy, Sox2, Lefl, Sox15, Sox6, Tcf-7, Tcf7ll, c-Myc, L-Myc, N-Myc, Handl, Mad1, Mad3, Mad4, Mxil, Myf5, Neurog2, Ngn3, Olig2, Tcf3, Tcf4, Foxc1, Foxd3, BAF155, C/EBPP, mafa, Eomes, Tbx-3; Rfx4, Stat3, Stella, and UTF-1. Exemplary transcription factors include Oct4, Sox2, Klf4, c-Myc, and Nanog.

Small molecule reprogramming agents are also pluripotency factors and may also be employed in the methods of the invention for inducing reprogramming and maintaining or increasing cell potency. In some embodiments of the invention, one or more small molecule reprogramming agents are used to induce pluripotency of a somatic cell, increase or maintain the potency of a cell, or improve the efficiency of reprogramming. In some embodiments, small molecule reprogramming agents are employed in the methods of the invention to improve the efficiency of reprogramming. Improvements in efficiency of reprogramming can be measured by (1) a decrease in the time required for reprogramming and generation of pluripotent cells (e.g., by shortening the time to generate pluripotent cells by at least a day compared to a similar or same process without the small molecule), or alternatively, or in combination, (2) an increase in the number of pluripotent cells generated by a particular process (e.g., increasing the number of cells reprogrammed in a given time period by at least 10%, 30%, 50%, 100%, 200%, 500%, etc. compared to a similar or same process without the small molecule). In some embodiments, a 2-fold to 20-fold improvement in reprogramming efficiency is observed. In some embodiments, reprogramming efficiency is improved by more than 20 fold. In some embodiments, a more than 100 fold improvement in efficiency is observed over the method without the small molecule reprogramming agent (e.g., a more than 100 fold increase in the number of pluripotent cells generated). Several classes of small molecule reprogramming agents may be important to increasing, establishing, and/or maintaining the potency of a cell. Exemplary small molecule reprogramming agents include, but are not limited to: agents that inhibit H3K9 methylation or promote H3K9 demethylation; agents that inhibit H3K4 demethylation or promotes H3K4 methylation; agents that inhibit histone deacetylation or promote histone acetylation; L-type Ca channel agonists; activators of the cAMP pathway; DNA methyltransferase (DNMT) inhibitors; nuclear receptor ligands; GSK3 inhibitors; MEK inhibitors; TGFP receptor/ALK5 inhibitors; HDAC inhibitors; Erk inhibitors; ROCK inhibitors; FGFR inhibitors; and PARP inhibitors. Exemplary small molecule reprogramming agents include GSK3 inhibitors; MEK inhibitors; TGFP receptor/ALK5 inhibitors; HDAC inhibitors; Erk inhibitors; and ROCK inhibitors.

In some embodiments of the invention, small molecule reprogramming agents are used to replace one or more transcription factors in the methods of the invention to induce pluripotency, improve the efficiency of reprogramming, and/or increase or maintain the potency of a cell. For example, in some embodiments, a cell is contacted with one or more small molecule reprogramming agents, wherein the agents are included in an amount sufficient to improve the efficiency of reprogramming. In other embodiments, one or more small molecule reprogramming agents are used in addition to transcription factors in the methods of the invention. In one embodiment, a cell is contacted with at least one pluripotency transcription factor and at least one small molecule reprogramming agent under conditions to increase, establish, and/or maintain the potency of the cell or improve the efficiency of the reprogramming process. In another embodiment, a cell is contacted with at least one pluripotency transcription factor and at least two, at least three, at least four, at least five, at least six, at least seven, at least eight, at least nine, or at least ten small molecule reprogramming agents under conditions and for a time sufficient to increase, establish, and/or maintain the potency of the cell or improve the efficiency of reprogramming. The state of potency or differentiation of cells can be assessed by monitoring the pluripotency characteristics (e.g., expression of markers including, but not limited to SSEA-3, SSEA-4, TRA-1-60, TRA-1-81, TRA-2-49/6E, Oct-3/4, Sox2, Nanog, GDF3, REX1, FGF4, ESG1, DPPA2, DPPA4, and hTERT).

Diseases Associated with Paneth Cells and Stem Cell Regulation

In certain embodiments, diseases associated with Lgr5+ stem cell differentiation (e.g., diseases associated with aberrant barrier function, or diseases of the gut, respiratory system or inner ear) are treated, diagnosed or monitored using the methods and compositions of the present invention. Aberrant barrier function can be associated with increased inflammation and cell death. Epithelial barrier tissues are central to immunological homeostasis, interfacing with stromal and immune cells to coordinate appropriate responses to environmental stimuli. Because of this centrality, therapeutic development for a wide spectrum of disease has sought to identify immune-modifying targets within barrier tissue epithelium. Within the intestinal epithelium, barrier and absorptive function is provided by cellular specialists. Adult intestinal stem cells (ISCs) provide a source of constant regeneration from which an ordered process of differentiation into secretory and absorptive epithelia sets composition, thereby setting function. Compelling evidence suggests that either changes in epithelial composition arising from aberrant cues driving stem cell differentiation or changes in the functional quality of differentiated specialists may be a precipitating factor in certain cancers, infections, and immune-mediated diseases.

The intestinal epithelium is ordered in a single-layer ‘conveyor belt’ originating from ISCs, conventionally identified as Lgr5+(Barker et al., 2007). ISCs are co-localized at the base of intestinal crypts with antimicrobial and niche-supporting Paneth cells. Paneth cells support stem cell function through the secretion of growth signaling molecules that are required for proliferation and maintenance. The epithelial conveyor extends from rapidly-dividing crypt-adjacent progenitors into lumenal protrusions known as villi—primarily composed of nutrient-absorbing enterocytes, along with secretory goblet and enteroendocrine populations. Under homeostatic conditions, Wnt, BMP, and Notch signaling maintain the ISC niche (Kim et al., 2005; Pinto et al., 2003). However, ISCs have a demonstrated capacity to integrate dietary and immune-derived signals to modulate their self-renewal and differentiation into specific secretory lineages (Beyaz et al., 2016; Biton et al., 2018; von Moltke et al., 2016). Further, following major injury to the epithelium, the ISC niche has a remarkable capacity to regenerate from non-stem or quiescent stem pools (Ayyaz et al., 2019; Tetteh et al., 2016; Yan et al., 2017). Cellular identity in the stem cell niche is fluid in response to multiple stimuli and alterations in the barrier which arise from this stem cell population may be directly altered in disease, or potentially controlled via the ‘synthetic’ provision of novel cues.

Compelling evidence in the upper respiratory tract and skin suggest that inflammatory disease may be driven by alterations in the tissue stem cell population (Naik et al., 2017; Ordovas-Montanes et al., 2018). In the upper respiratory tract, alterations in stem cells manifest as shifted differentiation trajectories which alter the quality of mature epithelial specialists. This observation may extend to disease involving the small intestine—for example, the multiple observations of altered Paneth cell quality of unknown origin in inflammatory bowel disease (IBD) (Gassler, 2017; Khor et al., 2011; Liu et al., 2016; McGuckin et al., 2009; Xavier and Podolsky, 2007). Similar Paneth cell aberrations occur in necrotizing enterocolitis (NEC), where Paneth cell number and quality is diminished, corresponding with intestinal immaturity and excessive inflammation and systemic infection (McElroy et al., 2013; Sherman et al., 2005; Tanner et al., 2015; White et al., 2017). Emerging evidence suggest that certain viral pathogens, including a subset of coronavirus, may mediate their profound disruption of the intestinal barrier via a Paneth cell-axis (Wu et al., 2020). Finally, Paneth cells are implicated in Graft versus Host disease (GvHD), which occurs after an allogeneic hematopoietic stem cell transplant in which donor T cells cause an inflammatory response in the host. Patients with GvHD can exhibit a loss in Paneth cell number and quality, and microbial dysbiosis (Eriguchi et al., 2012). In the context of tissue stem cells possessing significant control over barrier composition and function, combined with known connections between stem cell aberrations and human disease, observations of altered Paneth cell quality in disease raises the question of whether targeting ISCs to restore Paneth cells is therapeutically viable.

This has been demonstrated in GvHD. Treatment with R-spondin1 (R), a potent WNT agonist, can elevate the secretion of alpha-defensins and resolve dysbiosis seen in mice with GvHD by stimulating ISCs to differentiate into Paneth cells (Hayase et al., 2017). However, while treatment with R illustrates the importance of stem cell cues driving barrier tissue reconstitution via specialist-specific differentiation, it faces a major challenge in clinical translation because WNT activation is implicated in precancerous hyperplasia (Han et al., 2017; Okubo and Hogan, 2004; Sansom et al., 2004). While the effects of R are inconclusive with respect to malignancy (Kim et al., 2005; Zhou et al., 2017b), WNT signaling must be carefully balanced to ensure homeostasis not priming for cancer. Other signaling pathways known to drive Paneth cell differentiation, including Notch signaling, face similar challenges. Activation of Notch signaling amplifies the proliferative progenitor population and promotes an absorptive cell lineage (Fre et al., 2005; Jensen et al., 2000; VanDussen et al., 2012). Conversely, deactivation of Notch signaling amplifies differentiation to all secretory cell types and secretory cell hyperplasia (VanDussen and Samuelson, 2010). As these pathways affect multiple cell types in the intestinal epithelium and may lead to hyperplasia, they are not therapeutically viable. Therefore, a more specific treatment to accomplish stem to Paneth differentiation is desirable.

A skilled person can readily determine diseases that can be treated by reducing an inflammatory response. Type 2 inflammatory responses have been associated with allergic asthma, therapy resistant-asthma, steroid-resistant severe allergic airway inflammation, systemic steroid-dependent severe eosinophilic asthma, chronic rhino-sinusitis (CRS), atopic dermatitis, food allergies, persistence of chronic airway inflammation, and primary eosinophilic gastrointestinal disorders (EGIDs), including but not limited to eosinophilic esophagitis (EoE), eosinophilic gastritis, eosinophilic gastroenteritis, and eosinophilic colitis (see, e.g., Van Rijt et al., Type 2 innate lymphoid cells: at the cross-roads in allergic asthma, Seminars in Immunopathology July 2016, Volume 38, Issue 4, pp 483-496; Rivas et al., IL-4 production by group 2 innate lymphoid cells promotes food allergy by blocking regulatory T-cell function, J Allergy Clin Immunol. 2016 September; 138(3):801-811.e9; and Morita, Hideaki et al. Innate lymphoid cells in allergic and nonallergic inflammation, Journal of Allergy and Clinical Immunology, Volume 138, Issue 5, 1253-1264). Asthma is characterized by recurrent episodes of wheezing, shortness of breath, chest tightness, and coughing. Sputum may be produced from the lung by coughing but is often hard to bring up. During recovery from an attack, it may appear pus-like due to high levels of eosinophils. Symptoms are usually worse at night and in the early morning or in response to exercise or cold air. Some people with asthma rarely experience symptoms, usually in response to triggers, whereas others may have marked and persistent symptoms. CRS is characterized by inflammation of the mucosal surfaces of the nose and para-nasal sinuses, and it often coexists with allergic asthma. Atopic dermatitis is a chronic inflammatory skin disease that is characterized by eosinophilic infiltration and high serum IgE levels. Similar to allergic asthma and CRS, atopic dermatitis has been associated with increased expression of TSLP, IL-25, and IL-33 in the skin. Primary eosinophilic gastrointestinal disorders (EGIDs), including eosinophilic esophagitis (EoE), eosinophilic gastritis, eosinophilic gastroenteritis, and eosinophilic colitis, are disorders that exhibit eosinophil-rich inflammation in the gastrointestinal tract in the absence of known causes for eosinophilia such as parasite infection and drug reaction.

In certain embodiments, a disease or disorder that can be treated by reducing an inflammatory response or maintaining homeostasis may be any inflammatory disease or disorder such as, but not limited to, asthma, allergy, allergic rhinitis, allergic airway inflammation, atopic dermatitis (AD), chronic obstructive pulmonary disease (COPD), inflammatory bowel disease (IBD), multiple sclerosis, arthritis, psoriasis, eosinophilic esophagitis, eosinophilic pneumonia, eosinophilic psoriasis, hypereosinophilic syndrome, graft-versus-host disease, uveitis, cardiovascular disease, pain, multiple sclerosis, lupus, vasculitis, chronic idiopathic urticaria and Eosinophilic Granulomatosis with Polyangiitis (Churg-Strauss Syndrome).

The asthma may be allergic asthma, non-allergic asthma, severe refractory asthma, asthma exacerbations, viral-induced asthma or viral-induced asthma exacerbations, steroid resistant asthma, steroid sensitive asthma, eosinophilic asthma or non-eosinophilic asthma and other related disorders characterized by airway inflammation or airway hyperresponsiveness (AHR).

The COPD may be a disease or disorder associated in part with, or caused by, cigarette smoke, air pollution, occupational chemicals, allergy or airway hyperresponsiveness.

The allergy may be associated with foods, pollen, mold, dust mites, animals, or animal dander.

The IBD may be ulcerative colitis (UC), Crohn's Disease, collagenous colitis, lymphocytic colitis, ischemic colitis, diversion colitis, Behcet's syndrome, infective colitis, indeterminate colitis, and other disorders characterized by inflammation of the mucosal layer of the large intestine or colon.

The arthritis may be selected from the group consisting of osteoarthritis, rheumatoid arthritis and psoriatic arthritis.

In certain embodiments, hearing loss is treated using the methods of the present invention. In certain embodiments, hearing loss is treated by differentiating stem cells of the inner ear in vivo or by transferring cells differentiated ex vivo. In certain embodiments, the treatment to enrich for Paneth cell differentiation in the intestines by inhibiting nuclear export, as described herein, is used to sustain and/or modulate inner ear stem cells, such that differentiation to hair cells is improved. Deafness can be caused by genetic and environmental factors, mostly affecting the non-regenerating hair cells of the inner ear. Recently work established a protocol for expansion of Lgr5-positive cochlear cells as organoids, to obtain Lgr5-positive cochlear progenitors (LCPs) in large numbers in vitro, using a combination of growth factors and small molecules that are used herein for intestinal stem cells (ISCs) (see, e.g., McLean et al., 2017 Clonal expansion of Lgr5-positive cells from mammalian cochlea and high-purity generation of sensory hair cells. Cell Rep. 18 1917-192; and Lenz et al., Applications of Lgr5-Positive Cochlear Progenitors (LCPs) to the Study of Hair Cell Differentiation Front Cell Dev Biol. 2019; 7: 14). The LCPs could be efficiently differentiated into hair cells. The methods of the present invention provide for an improved targeted differentiation of LGR5+ stem cells.

### Target Genes and Pathways

In certain embodiments, specific pathways or biological programs are modulated to enhance Paneth cell differentiation. In certain embodiments, the specific pathways or biological programs are detected or monitored. As used herein the term “biological program” can be used interchangeably with “expression program” or “transcriptional program” and may refer to a set of genes that share a role in a biological function (e.g., an activation program, cell differentiation program, proliferation program). Biological programs can include a pattern of gene expression that result in a corresponding physiological event or phenotypic trait. Biological programs can include up to several hundred genes that are expressed in a spatially and temporally controlled fashion. Expression of individual genes can be shared between biological programs. Expression of individual genes can be shared among different single cell types; however, expression of a biological program may be cell type specific or temporally specific (e.g., the biological program is expressed in a cell type at a specific time). Multiple biological programs may include the same gene, reflecting the gene's roles in different processes. Applicants have identified that XPO1 inhibitors enhance stem cell conversion to mature cells of the secretory pathway. Applicants performed single sequencing of ex vivo cell-based system treated with XPO1 inhibitors. Applicants identified pathways (e.g., stress response and mitogen signaling) and genes differentially expressed in response to the treatment (see, Tables 1, 2 and 3; and Examples). Applicants identified up and down regulated genes, as well as using correlation analysis to identify genes that are up and down regulated together. In certain embodiments, downstream targets of XPO1 inhibition can be used for any therapeutic, diagnostic or screening methods described herein. In certain embodiments, genes that are up regulated or downregulated in response to nuclear export inhibition are modulated or detected according to the methods described further herein.

TABLE 1
Upregulated KPT-330 Genes

| | | | | |
|---|---|---|---|---|
| Klf6 | Slc16a6 | Tmem171 | Tff3 | Tmprss2 |
| Atf3 | Btg2 | Prr15 | Rps26 | Xpo1 |
| Hbegf | Thbs1 | Rnase4 | Rpl13 | Gls |
| Ubc | Cldn4 | Itln1 | Hmgn1 | Mt2 |
| Muc13 | Edn1 | Mmp7 | Sox4 | Mt1 |
| 2210404007Rik | Smim6 | Gm15284 | Malat1 | mmu-mir-6236 |
| Pigr | Arrdc3 | Defa17 | Slc12a2 | Gm26924 |
| Ndrg1 | Abcg8 | Lyz1 | Atp1a1 | |

| Stress | Secretory |
|---|---|
| response | markers |
| Klf6 | Itln1 |
| Atf3 | Mmp7 |
| Hbegf | Gm15284 |
| Ubc | Defa17 |
| Muc13 | Lyz1 |
| | Tff3 |

TABLE 2
Down regulated KPT-330 Genes

| | | | | |
|---|---|---|---|---|
| Itpr3 | Mrpl12 | H1f0 | Paics | Mcm4 |
| Cbr1 | Gsr | Sae1 | Hjurp | Hells |
| Kmt2a | Gnl3 | C1qbp | Ezh2 | Mcm6 |
| Hsph1 | Ybx3 | Tpi1 | Rrm1 | Mcm2 |
| Gm2697 | Ckb | Hmgcs1 | Dnmt1 | Pcna |
| Bex1 | Isyna1 | Smoc2 | Ipo5 | Tgm2 |
| Cad | Nop56 | Axin2 | Fads1 | Crip2 |
| Rpph1 | Nolc1 | Cdk4 | Topbp1 | Fen1 |
| Gclc | Reep6 | Reg3g | Rpa1 | Ccnd1 |
| Cbr3 | Ccnd2 | Igfbp4 | Mcm7 | Dtl |
| Pfkp | Srm | Rgcc | Atad2 | Cdt1 |
| Ugdh | Gpx2 | Maged1 | Lig1 | Ptma |
| Elovl6 | Add3 | Tcof1 | Whsc1 | Hook1 |
| Slc25a4 | Oxct1 | Dctpp1 | Dut1 | Tubb5 |
| Galk1 | Nhp2 | Nasp | Urf1 | Mki67 |
| Wars | Bzw2 | Gart | Mcm3 | Ranbp1 |
| Slc38a1 | Prmt5 | Rif1 | Mcm5 | Kpnb1 |
| Parp1 | Rangap1 | | | |

Cell Cycle Genes

| | |
|---|---|
| Topbp1 | Pcna |
| Rpa1 | Tgm2 |
| Mcm7 | Crip2 |
| Atad2 | Fen1 |
| Lig1 | Ccnd1 |
| Whsc1 | Dtl |
| Dut1 | Cdt1 |
| Urf1 | Ptma |
| Mcm3 | Hook1 |
| Mcm5 | Tubb5 |
| Mcm4 | Mki67 |
| Hells | Ranbp1 |
| Mcm6 | Kpnb1 |
| Mcm2 | |

Table 3A-B. Differentially expressed genes and gene set enrichment analysis (GSEA) over differentially expressed genes between KPT-330 treated and untreated stem II/III cells over days 0.25, 1, 2 in organoid differentiation time course single-cell RNA-seq.

TABLE 3A
DE results ranked by Log2 fold-change for: stem II/III, 0.25-2 days.
The list of genes was obtained using the following significance
cut-offs: FDR < 0.05, Log2 fold-change > abs(2*st. dev) (0.208).

| gene symbol | avg Log2 FC | FDR | gene symbol | avg Log2 FC | FDR |
|---|---|---|---|---|---|
| Xpo1 | 0.817 | 2.00E−34 | Pa2g4 | −0.243 | 9.10E−05 |
| Kpnb1 | −0.507 | 2.09E−21 | Hjurp | −0.274 | 0.000106543 |
| Malat1 | 0.624 | 1.94E−20 | Smpd4 | −0.215 | 0.000114722 |
| Arrdc3 | 0.686 | 4.68E−17 | E2f8 | −0.218 | 0.000118754 |
| Hells | −0.487 | 9.68E−16 | Gar1 | −0.243 | 0.000142994 |
| Slc16a6 | 0.719 | 3.18E−15 | Strap | −0.248 | 0.00014769 |
| Ptma | −0.403 | 7.07E−14 | Hmgb1 | −0.222 | 0.000149762 |
| Anp32a | −0.376 | 3.31E−13 | D17H6S56E-5 | −0.284 | 0.00015515 |
| Usp1 | −0.424 | 4.50E−13 | Dut | −0.264 | 0.000158272 |
| Mlec | −0.318 | 6.65E−13 | Spc24 | −0.217 | 0.000165679 |
| Sqle | −0.430 | 1.92E−12 | Pfas | −0.220 | 0.000174995 |
| Uhrf1 | −0.514 | 3.14E−12 | Smim6 | 0.452 | 0.000183093 |
| Nasp | −0.359 | 4.77E−12 | Nop56 | −0.235 | 0.000188785 |
| Lmnb1 | −0.390 | 1.45E−11 | Hmgcs1 | −0.273 | 0.00021051 |
| Hnrnpu | −0.302 | 4.46E−11 | Cbr1 | −0.303 | 0.000218288 |
| 2810417H13Rik | −0.410 | 4.55E−11 | Slc25a10 | −0.238 | 0.000246341 |
| Slc12a2 | 0.338 | 5.79E−11 | Wdr36 | −0.228 | 0.000265176 |
| Rrm1 | −0.385 | 6.50E−11 | Mthfd2 | −0.277 | 0.000280871 |
| Anp32b | −0.382 | 1.16E−10 | Rps20 | 0.231 | 0.000286654 |
| Nolc1 | −0.345 | 1.70E−10 | Iqgap3 | −0.269 | 0.000299719 |
| Hook1 | −0.339 | 1.79E−10 | Ipo5 | −0.257 | 0.000305098 |
| Whsc1 | −0.384 | 1.85E−10 | Ugp2 | −0.250 | 0.00030627 |
| Reep6 | −0.376 | 2.08E−10 | Cct6a | −0.241 | 0.000319774 |
| Srm | −0.368 | 3.71E−10 | Hspa4 | −0.239 | 0.000325475 |
| Set | −0.351 | 4.23E−10 | Pcbp1 | −0.222 | 0.000347895 |
| Baz1b | −0.362 | 6.45E−10 | Pbk | −0.268 | 0.000348948 |
| Mt2 | 0.469 | 4.46E−09 | Letm1 | −0.232 | 0.000349742 |
| Ncl | −0.258 | 4.83E−09 | Rgcc | −0.286 | 0.000386031 |
| Mcm5 | −0.379 | 5.36E−09 | Psmd11 | −0.214 | 0.000390385 |
| Tmpo | −0.330 | 6.08E−09 | Parp1 | −0.213 | 0.00040357 |
| Dnmt1 | −0.432 | 7.99E−09 | Mcm6 | −0.292 | 0.000413347 |
| Rrm2 | −0.394 | 1.07E−08 | Mthfd1l | −0.249 | 0.000459775 |
| Mcm4 | −0.384 | 1.09E−08 | Alad | −0.237 | 0.000472444 |
| Lrrc8d | 0.536 | 1.44E−08 | Wars | −0.214 | 0.000560767 |
| Ccnd1 | −0.299 | 1.91E−08 | Orc2 | −0.215 | 0.00057199 |
| Cbr3 | −0.388 | 2.02E−08 | Gins2 | −0.217 | 0.000584392 |
| Atad2 | −0.371 | 4.39E−08 | Psmc2 | −0.209 | 0.000638852 |
| Mcm10 | −0.319 | 4.75E−08 | Pfn1 | −0.215 | 0.000656046 |
| Clspn | −0.316 | 5.20E−08 | Gclc | −0.237 | 0.000718633 |
| Fubp1 | −0.325 | 8.83E−08 | Ncapg2 | −0.232 | 0.000737526 |
| Igfbp4 | −0.358 | 9.82E−08 | Gstm3 | −0.481 | 0.000759259 |
| Nop58 | −0.305 | 9.87E−08 | Noc2l | −0.234 | 0.000883047 |
| Brca2 | −0.309 | 1.27E−07 | Aars | −0.261 | 0.000891501 |
| Hnrnpd | −0.282 | 1.69E−07 | Nadk | −0.208 | 0.000917102 |
| Ddx21 | −0.340 | 1.84E−07 | Gnl3 | −0.249 | 0.000968403 |
| Ezh2 | −0.328 | 1.90E−07 | Dhcr7 | −0.223 | 0.001007814 |
| Mcm7 | −0.318 | 3.11E−07 | Mllt6 | −0.226 | 0.001046223 |
| Grk6 | −0.261 | 3.26E−07 | Foxm1 | −0.214 | 0.001124725 |
| 2700089E24Rik | −0.263 | 5.36E−07 | Gfer | −0.214 | 0.001183443 |
| Abcf2 | −0.257 | 5.50E−07 | Suz12 | −0.220 | 0.001192723 |
| Mcm2 | −0.326 | 5.56E−07 | Smc4 | −0.225 | 0.001221713 |
| Eif4g1 | −0.238 | 6.15E−07 | Rpph1 | −0.253 | 0.001236714 |
| Phf10 | −0.245 | 6.76E−07 | 0610007P14Rik | −0.210 | 0.001318111 |
| Mdc1 | −0.269 | 8.67E−07 | Prkrir | −0.241 | 0.001381649 |
| Tcof1 | −0.277 | 9.56E−07 | Atf3 | 0.637 | 0.00147914 |
| Ecd | −0.252 | 9.58E−07 | Pebp1 | −0.225 | 0.001694394 |
| Dtl | −0.233 | 1.17E−06 | Lig1 | −0.310 | 0.00169927 |
| Atp1a1 | 0.283 | 1.19E−06 | Hunk | −0.272 | 0.001770798 |
| Mki67 | −0.417 | 1.51E−06 | Ccna2 | −0.283 | 0.001904695 |
| Hnrnpul1 | −0.309 | 1.51E−06 | Hmgn1 | 0.293 | 0.001905562 |
| Gsr | −0.329 | 1.87E−06 | Topbp1 | −0.221 | 0.002366154 |
| Mt1 | 0.397 | 2.01E−06 | Spcs3 | −0.224 | 0.002436217 |
| Lyar | −0.264 | 2.19E−06 | Cbx5 | −0.249 | 0.002443027 |
| Rangap1 | −0.300 | 2.37E−06 | Ugdh | −0.217 | 0.00265255 |
| Srpk1 | −0.294 | 2.65E−06 | Elovl6 | −0.215 | 0.002998189 |
| Srrm1 | −0.286 | 3.22E−06 | Rqcd1 | −0.209 | 0.003139837 |
| Mcm3 | −0.317 | 3.64E−06 | Ncapd2 | −0.232 | 0.0033273 |
| Abce1 | −0.260 | 4.62E−06 | Tsc22d1 | 0.404 | 0.003392753 |
| Cnbp | −0.269 | 6.23E−06 | Smc6 | −0.216 | 0.003952449 |
| Kif15 | −0.327 | 1.02E−05 | Prkar2a | −0.218 | 0.004146948 |
| Lrrfip2 | −0.226 | 1.03E−05 | Rpa1 | −0.211 | 0.004226582 |
| Dtymk | −0.319 | 1.08E−05 | Txnrd1 | −0.246 | 0.004396416 |
| Gcn1l1 | −0.220 | 1.18E−05 | Mrpl12 | −0.237 | 0.004669772 |
| Ddx11 | −0.218 | 1.23E−05 | Hnrnph1 | −0.231 | 0.005106349 |
| Ppm1g | −0.315 | 1.27E−05 | Asf1b | −0.209 | 0.005172196 |
| Tbrg1 | 0.442 | 1.35E−05 | Sae1 | −0.241 | 0.005690263 |
| Gmnn | −0.245 | 1.62E−05 | Pdap1 | −0.214 | 0.005841368 |
| Aldh1b1 | −0.272 | 1.70E−05 | Bola3 | −0.246 | 0.006432305 |
| Cdca7l | −0.251 | 1.75E−05 | Pdzd8 | −0.208 | 0.006477462 |
| Pcna | −0.296 | 1.86E−05 | Pigr | 0.250 | 0.007271275 |
| Ndufs6 | −0.247 | 2.03E−05 | Fos | 0.376 | 0.007762989 |
| Pola1 | −0.249 | 2.05E−05 | Rnase4 | 0.376 | 0.009593894 |
| Larp1 | −0.268 | 2.13E−05 | Pole | −0.223 | 0.009855895 |
| Gspt1 | −0.258 | 2.34E−05 | Supt16 | −0.213 | 0.010300849 |
| Mis12 | −0.226 | 2.45E−05 | Nhp2 | −0.240 | 0.012370091 |
| Cad | −0.241 | 2.62E−05 | Hyou1 | −0.208 | 0.013474641 |
| Ticrr | −0.237 | 2.99E−05 | Hp1bp3 | −0.231 | 0.017943001 |
| Psmd4 | −0.245 | 3.12E−05 | Rif1 | −0.242 | 0.020001536 |
| Ranbp1 | −0.270 | 3.27E−05 | Rpl30 | −0.233 | 0.02089545 |
| Ybx3 | −0.253 | 3.53E−05 | Eif5a | −0.224 | 0.02180025 |
| Dag1 | −0.248 | 4.16E−05 | Hnrnpdl | −0.223 | 0.021813346 |
| Heatr1 | −0.233 | 4.70E−05 | Kif20b | −0.211 | 0.024567427 |
| Vars | −0.226 | 4.81E−05 | Dnajc2 | −0.217 | 0.025867776 |
| Utp20 | −0.254 | 5.17E−05 | Dkc1 | −0.215 | 0.027200461 |
| Tars | −0.275 | 5.37E−05 | Tubb4b | −0.235 | 0.028749195 |
| Arpc5l | −0.266 | 6.63E−05 | Kif11 | −0.224 | 0.032695409 |
| Eftud2 | −0.229 | 7.59E−05 | Rfc4 | −0.209 | 0.036527048 |
| Krt23 | 0.389 | 8.20E−05 | Cox8a | −0.208 | 0.039288123 |
| Tagln2 | −0.266 | 8.81E−05 | Ssrp1 | −0.212 | 0.041788924 |

TABLE 3B
GSEA MSigDB Hallmark Genesets v7. The following significance cut-offs were used: FDR < 0.05.

| | Genes | NES | Genes | Genes | FDR |
|---|---|---|---|---|---|
| Name | (tot) | (dist.dir) | (up) | (down) | (both ways) |
| TNFA_SIGNALING_VIA_NFKB | 62 | 0.70018 | 42 | 20 | 0.000 |
| KRAS_SIGNALING_UP | 40 | 0.57951 | 25 | 15 | 0.000 |
| HYPOXIA | 67 | 0.53851 | 44 | 23 | 0.000 |
| INFLAMMATORY_RESPONSE | 36 | 0.50791 | 18 | 18 | 0.002 |
| P53_PATHWAY | 84 | 0.49701 | 51 | 33 | 0.000 |
| IL2_STAT5_SIGNALING | 63 | 0.47062 | 33 | 30 | 0.001 |
| APOPTOSIS | 68 | 0.40492 | 35 | 33 | 0.008 |
| PROTEIN_SECRETION | 65 | 0.36342 | 32 | 33 | 0.030 |
| TGF_BETA_SIGNALING | 31 | 0.36125 | 14 | 17 | 0.125 |
| INTERFERON_GAMMA_RESPONSE | 55 | 0.35762 | 26 | 29 | 0.045 |
| UV_RESPONSE_UP | 79 | 0.32905 | 31 | 48 | 0.044 |
| APICAL_JUNCTION | 59 | 0.30775 | 31 | 28 | 0.120 |
| MYOGENESIS | 38 | 0.30749 | 21 | 17 | 0.233 |
| ESTROGEN_RESPONSE_EARLY | 75 | 0.2674 | 31 | 44 | 0.225 |
| CHOLESTEROL_HOMEOSTASIS | 37 | 0.26035 | 13 | 24 | 0.532 |
| COMPLEMENT | 47 | 0.23354 | 21 | 26 | 0.621 |
| UV_RESPONSE_DN | 52 | −0.12321 | 17 | 35 | 1.000 |
| OXIDATIVE_PHOSPHORYLATION | 143 | −0.16946 | 64 | 79 | 1.000 |
| ADIPOGENESIS | 114 | −0.20577 | 39 | 75 | 0.964 |
| BILE_ACID_METABOLISM | 34 | −0.22354 | 13 | 21 | 1.000 |
| PI3K_AKT_MTOR_SIGNALING | 60 | −0.24339 | 22 | 38 | 0.954 |
| HEME_METABOLISM | 77 | −0.25016 | 27 | 50 | 0.880 |
| ESTROGEN_RESPONSE_LATE | 83 | −0.25633 | 28 | 55 | 0.843 |
| GLYCOLYSIS | 87 | −0.2679 | 32 | 55 | 0.818 |
| FATTY_ACID_METABOLISM | 93 | −0.28206 | 38 | 55 | 0.814 |
| EPITHELIAL_MESENCHYMAL_TRANSITION | 25 | −0.28804 | 10 | 15 | 0.912 |
| XENOBIOTIC_METABOLISM | 76 | −0.28891 | 28 | 48 | 0.775 |
| PEROXISOME | 51 | −0.30147 | 18 | 33 | 0.792 |
| REACTIVE_OXYGEN_SPECIES_PATHWAY | 29 | −0.31117 | 7 | 22 | 0.885 |
| ANDROGEN_RESPONSE | 50 | −0.35605 | 23 | 27 | 0.421 |
| MITOTIC_SPINDLE | 126 | −0.36953 | 41 | 85 | 0.110 |
| DNA_REPAIR | 92 | −0.38213 | 27 | 65 | 0.108 |
| ALLOGRAFT_REJECTION | 37 | −0.39889 | 13 | 24 | 0.307 |
| MTORC1_SIGNALING | 144 | −0.49785 | 41 | 103 | 0.000 |
| UNFOLDED_PROTEIN_RESPONSE | 86 | −0.51466 | 14 | 72 | 0.000 |
| SPERMATOGENESIS | 36 | −0.51987 | 4 | 32 | 0.011 |
| MYC_TARGETS_V1 | 172 | −0.59037 | 20 | 152 | 0.000 |
| G2M_CHECKPOINT | 139 | −0.60605 | 17 | 122 | 0.000 |
| MYC_TARGETS_V2 | 50 | −0.62647 | 4 | 46 | 0.000 |
| E2F_TARGETS | 140 | −0.70507 | 11 | 129 | 0.000 |

The present invention includes the use of gene signatures, biological programs, or pathways. As used herein a “signature” may encompass any gene or genes, protein or proteins, or epigenetic element(s) whose expression profile or whose occurrence is associated with a specific cell type, subtype, or cell state of a specific cell type or subtype within a population of cells. For ease of discussion, when discussing gene expression, any of gene or genes, protein or proteins, or epigenetic element(s) may be substituted. As used herein, the terms “signature”, “expression profile”, or “expression program” may be used interchangeably. It is to be understood that also when referring to proteins (e.g. differentially expressed proteins), such may fall within the definition of “gene” signature. Levels of expression or activity or prevalence may be compared between different cells in order to characterize or identify for instance signatures specific for cell (sub)populations. Increased or decreased expression or activity or prevalence of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and down-regulated genes that are representative of a cell type or subtype. A gene signature as used herein, may also refer to any set of up- and down-regulated genes between different cells or cell (sub)populations derived from a gene-expression profile. For example, a gene signature may comprise a list of genes differentially expressed in a distinction of interest.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single-cells within a population of cells from isolated samples (e.g. tumor samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of cancer cells that are linked to particular pathological condition (e.g. cancer grade), or linked to a particular outcome or progression of the disease (e.g. metastasis), or linked to a particular response to treatment of the disease.

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

In certain embodiments, a signature is characterized as being specific for a particular cell or cell (sub)population if it is upregulated or only present, detected or detectable in that particular cell or cell (sub)population, or alternatively is downregulated or only absent, or undetectable in that particular cell or cell (sub)population. In this context, a signature consists of one or more differentially expressed genes/proteins or differential epigenetic elements when comparing different cells or cell (sub)populations, including comparing different tumor cells or tumor cell (sub)populations, as well as comparing tumor cells or tumor cell (sub)populations with non-tumor cells or non-tumor cell (sub)populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art. In certain embodiments, differential expression may be determined by comparing expression to the mean or median expression of all expressed genes or to a subset of genes.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferable is meant induction or alternatively suppression (or upregulation or downregulation) of at least one gene/protein and/or epigenetic element of the signature, such as for instance at least to, at least three, at least four, at least five, at least six, or all genes/proteins and/or epigenetic elements of the signature.

### In Vitro Models

In certain embodiments, the present invention provides methods of generating target cell types in vitro. In vitro models may be obtained by modulating factors or pathways as described herein.

In certain embodiments, the in vitro models of the present invention may be used to study development, cell biology and disease. In certain embodiments, the in vitro models of the present invention may be used to screen for drugs capable of modulating the target cells or for determining toxicity of drugs (e.g., toxic to Paneth cells). In certain embodiments, the in vitro models of the present invention may be used to identify specific cell states and/or subtypes.

In certain embodiments, the in vitro models of the present invention may be used in perturbation studies. Perturbations may include conditions, substances or agents. Agents may be of physical, chemical, biochemical and/or biological nature. Perturbations may include treatment with a small molecule, protein, RNAi, CRISPR system, TALE system, Zn finger system, meganuclease, pathogen, allergen, biomolecule, or environmental stress. Such methods may be performed in any manner appropriate for the particular application.

In certain embodiments, the in vitro models are configured for performing perturb-seq. Methods and tools for genome-scale screening of perturbations in single cells using CRISPR have been described, herein referred to as perturb-seq (see e.g., Dixit et al., “Perturb-Seq: Dissecting Molecular Circuits with Scalable Single-Cell RNA Profiling of Pooled Genetic Screens” 2016, Cell 167, 1853-1866; Adamson et al., “A Multiplexed Single-Cell CRISPR Screening Platform Enables Systematic Dissection of the Unfolded Protein Response” 2016, Cell 167, 1867-1882; Feldman et al., Lentiviral co-packaging mitigates the effects of intermolecular recombination and multiple integrations in pooled genetic screens, bioRxiv 262121, doi: doi.org/10.1101/262121; Datlinger, et al., 2017, Pooled CRISPR screening with single-cell transcriptome readout. Nature Methods. Vol. 14 No. 3 DOI: 10.1038/nmeth.4177; Hill et al., On the design of CRISPR-based single cell molecular screens, Nat Methods. 2018 April; 15(4): 271-274; Replogle, et al., “Combinatorial single-cell CRISPR screens by direct guide RNA capture and targeted sequencing” Nat Biotechnol (2020). doi.org/10.1038/s41587-020-0470-y; and International Patent Publication No. WO/2017/075294).

### Therapeutic Methods

In certain embodiments, diseases characterized by an inflammatory response are treated by differentiating stem cells in vivo or by transferring cells differentiated ex vivo (e.g., differentiating to Paneth cells). In certain embodiments, a disease may be treated by inducing target cells in vivo. Target cells may be induced in vivo by activating or inhibiting a pathway, modulation of expression or activity of a target gene, such as, expressing transcription factors at a specific site of the disease (e.g., ATF3). Transcription factors may be provided to specific cells at a location of disease. In certain embodiments, mRNA is provided. In certain embodiments, low dose nuclear export inhibitors are administered to a subject.

It will be understood by the skilled person that treating as referred to herein encompasses enhancing treatment, or improving treatment efficacy. Treatment may include inhibition of an inflammatory response, enhancing an immune response, tumor regression as well as inhibition of tumor growth, metastasis or tumor cell proliferation, or inhibition or reduction of otherwise deleterious effects associated with the tumor.

Efficaciousness of treatment is determined in association with any known method for diagnosing or treating the particular disease. The invention comprehends a treatment method comprising any one of the methods or uses herein discussed.

The phrase “therapeutically effective amount” as used herein refers to a sufficient amount of a drug, agent, or compound to provide a desired therapeutic effect.

As used herein “patient” refers to any human being receiving or who may receive medical treatment and is used interchangeably herein with the term “subject”.

Therapy or treatment according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the cancer, and how the patient responds to the treatment. Additionally, a person having a greater risk of developing an inflammatory response (e.g., aberrant barrier function) may receive prophylactic treatment to inhibit or delay symptoms of the disease.

In certain embodiments, a cell-based therapeutic includes engraftment of the cells of the present invention. As used herein, the term “engraft” or “engraftment” refers to the process of cell incorporation into a tissue of interest in vivo through contact with existing cells of the tissue. In certain embodiments, the cell based therapy may comprise adoptive cell transfer (ACT). As used herein adoptive cell transfer and adoptive cell therapy are used interchangeably. In certain embodiments, the target cells differentiated according to the methods described herein may be transferred to a subject in need thereof. If possible, use of autologous cells helps the recipient by minimizing GVHD issues. In certain embodiments, autologous stem cells are harvested from a subject and the cells are modulated to differentiate the stem cells into target cells (e.g., by overexpressing a transcription factor, such as ATF3).

### Pharmaceutical Compositions and Delivery

Target cells of the present invention may be combined with various components to produce compositions of the invention. The compositions may be combined with one or more pharmaceutically acceptable carriers or diluents to produce a pharmaceutical composition (which may be for human or animal use). Suitable carriers and diluents include, but are not limited to, isotonic saline solutions, for example phosphate-buffered saline. The composition of the invention may be administered by direct injection. The composition may be formulated for parenteral, intramuscular, intravenous, subcutaneous, intraocular, oral, transdermal administration, or injection into the spinal fluid.

Compositions comprising target cells may be delivered by injection or implantation. Cells may be delivered in suspension or embedded in a support matrix such as natural and/or synthetic biodegradable matrices. Natural matrices include, but are not limited to, collagen matrices. Synthetic biodegradable matrices include, but are not limited to, polyanhydrides and polylactic acid. These matrices may provide support for fragile cells in vivo.

The compositions may also comprise the target cells of the present invention, and at least one pharmaceutically acceptable excipient, carrier, or vehicle.

Delivery may also be by controlled delivery, i.e., delivered over a period of time which may be from several minutes to several hours or days. Delivery may be systemic (for example by intravenous injection) or directed to a particular site of interest. Cells may be introduced in vivo using liposomal transfer.

Target cells may be administered in doses of from 1×10⁵ to 1×10⁷ cells per kg. For example a 70 kg patient may be administered 1.4×10⁶ cells for reconstitution of tissues. The dosages may be any combination of the target cells listed in this application.

The modifying agents and other modulating agents, or components thereof, or nucleic acid molecules thereof, or nucleic acid molecules encoding or providing components thereof, may be delivered by a delivery system herein described.

Vector delivery, e.g., plasmid, viral delivery: the modulating agents, can be delivered using any suitable vector, e.g., plasmid or viral vectors, such as adeno associated virus (AAV), lentivirus, adenovirus or other viral vector types, or combinations thereof. In some embodiments, the vector, e.g., plasmid or viral vector is delivered to the tissue of interest by, for example, an intramuscular injection, while other times the delivery is via intravenous, transdermal, intranasal, oral, mucosal, or other delivery methods. Such delivery may be either via a single dose, or multiple doses. One skilled in the art understands that the actual dosage to be delivered herein may vary greatly depending upon a variety of factors, such as the vector choice, the target cell, organism, or tissue, the general condition of the subject to be treated, the degree of transformation/modification sought, the administration route, the administration mode, the type of transformation/modification sought, etc.

In certain embodiments, small molecules, proteins, mRNA or cells are administered via targeted injection (e.g., the tissue to be repaired), intravenous, infusion, or other delivery methods. Such delivery may be either via a single dose, or multiple doses. One skilled in the art understands that the actual dosage to be delivered herein may vary greatly depending upon a variety of factors, such as the target cell, or tissue, the general condition of the subject to be treated, the degree of modification sought, the administration route, the administration mode, the type of modification sought, etc.

In certain embodiment, transcription factors are expressed in target tissue cells temporarily. In certain embodiments, the time of transcription factor expression or enhancement is only the time required to differentiate stem cells into target cells. In certain embodiments, transcription factors are expressed or enhanced for 1 to 14 days, preferably, about 2 days. In certain embodiments, the means of delivery does not result in integration of a sequence encoding transcription factors in the genome of target cells.

A “pharmaceutical composition” refers to a composition that usually contains an excipient, such as a pharmaceutically acceptable carrier that is conventional in the art and that is suitable for administration to cells or to a subject.

The pharmaceutical composition according to the present invention can, in one alternative, include a prodrug. When a pharmaceutical composition according to the present invention includes a prodrug, prodrugs and active metabolites of a compound may be identified using routine techniques known in the art. (See, e.g., Bertolini et al., J. Med. Chem., 40, 2011-2016 (1997); Shan et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992); and Prox et al., Xenobiol., 3, 103-112 (1992)).

The term “pharmaceutically acceptable” as used throughout this specification is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilizers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavorings, aromatizers, thickeners, agents for achieving a depot effect, coatings, antifungal agents, preservatives, stabilizers, antioxidants, tonicity controlling agents, absorption delaying agents, and the like. The use of such media and agents for pharmaceutical active components is well known in the art. Such materials should be non-toxic and should not interfere with the activity of the cells or active components.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

The pharmaceutical composition can be applied parenterally, rectally, orally or topically. Preferably, the pharmaceutical composition may be used for intravenous, intramuscular, subcutaneous, peritoneal, peridural, rectal, nasal, pulmonary, mucosal, or oral application. In a preferred embodiment, the pharmaceutical composition according to the invention is intended to be used as an infusion. The skilled person will understand that compositions which are to be administered orally or topically will usually not comprise cells, although it may be envisioned for oral compositions to also comprise cells, for example when gastro-intestinal tract indications are treated. Each of the cells or active components (e.g., immunomodulants) as discussed herein may be administered by the same route or may be administered by a different route. By means of example, and without limitation, cells may be administered parenterally and other active components may be administered orally.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propylene glycol or polyethylene glycol may be included.

The composition may include one or more cell protective molecules, cell regenerative molecules, growth factors, anti-apoptotic factors or factors that regulate gene expression in the cells. Such substances may render the cells independent of their environment.

Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure isosmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further suitably pharmaceutically acceptable carriers or additives are well known to those skilled in the art and for instance may be selected from proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregelatinized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

In certain embodiments, a pharmaceutical cell preparation as taught herein may be administered in a form of liquid composition. In embodiments, the cells or pharmaceutical composition comprising such can be administered systemically, topically, within an organ or at a site of organ dysfunction or lesion.

Preferably, the pharmaceutical compositions may comprise a therapeutically effective amount of the specified immune cells and/or other active components (e.g., immunomodulants). The term “therapeutically effective amount” refers to an amount which can elicit a biological or medicinal response in a tissue, system, animal or human that is being sought by a researcher, veterinarian, medical doctor or other clinician, and in particular can prevent or alleviate one or more of the local or systemic symptoms or features of a disease or condition being treated.

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The medicaments of the invention are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the invention may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Various delivery systems are known and can be used to administer the pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (e.g., a tumor), thus requiring only a fraction of the systemic dose. See, for example, Goodson, In: Medical Applications of Controlled Release, 1984. (CRC Press, Boca Raton, Fla.).

The amount of the agents which will be effective in the treatment of a particular disorder or condition will depend on the nature of the disorder or condition, and may be determined by standard clinical techniques by those of skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of the agent with which to treat each individual patient. In certain embodiments, the attending physician will administer low doses of the agent and observe the patient's response. Larger doses of the agent may be administered until the optimal therapeutic effect is obtained for the patient, and at that point the dosage is not increased further. Effective doses may be extrapolated from dose-response curves derived from in vitro or animal model test systems. Ultimately the attending physician will decide on the appropriate duration of therapy using compositions of the present invention. Dosage will also vary according to the age, weight and response of the individual patient.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection.

### Modulating Agents

Applicants identified up and down regulated genes in response to inhibition of XPO1 (Table 1, 2 and 3). Any of these genes may be targeted to modulate differentiation ex vivo or in vivo. Applicants identified that stress response genes were upregulated and cell cycle genes were downregulated in response to XPO1 inhibition. Applicants also identified that XPO1 inhibition induces a quiescent signature and that differentiation can be enhanced by inducing stem quiescence. Modulating agents targeting these genes and pathways are described further herein.

In certain embodiments, a transcription factor is targeted. In certain embodiments ATF3 is targeted. In certain embodiments, ATF3 activity is enhanced by modulation of post translational modification sites as described further herein. In certain embodiments, ATF3 expression is upregulated as described further herein. In certain embodiments, endogenous ATF3 is expressed in stem cells as described further herein. ATF3 is induced upon physiological stress in various tissues (Chen et al., 1996 “Analysis of ATF3, a transcription factor induced by physiological stresses and modulated by gadd153/Chop10”. Molecular and Cellular Biology. 16 (3): 1157-68). It is also a marker of regeneration following injury of dorsal root ganglion neurons, as injured regenerating neurons activate this transcription factor (Lindi et al., 2011 “Activating transcription factor 3, a useful marker for regenerative response after nerve root injury”. Frontiers in Neurology. 2: 30). Functional validation studies have shown that ATF3 can promote regeneration of peripheral neurons, but is not capable of promoting regeneration of central nervous system neurons (Mahar M, and Cavalli V 2018 “Intrinsic mechanisms of neuronal axon regeneration”. Nature Reviews. Neuroscience. 19 (6): 323-337).

In certain embodiments, the present invention provides for one or more modulating agents against that target signature genes or pathways identified. Targeting the identified signature genes or pathways may provide for enhanced differentiation of stem cells into a target cell. In certain embodiments, the modulating agent is a therapeutic agent used in the treatment of a disease.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse).

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

### Small Molecules

In certain embodiments, the one or more agents is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

One type of small molecule applicable to the present invention is a degrader molecule. Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. Jan. 6, 2017; 57: 107-123; and Lai et al., Modular PROTAC Design for the Degradation of Oncogenic BCR-ABL Angew Chem Int Ed Engl. Jan. 11, 2016; 55(2): 807-810).

### Nuclear Export Inhibitors

In certain embodiments, low dosages of nuclear export inhibitors are administered to a subject in need thereof. In certain embodiments, low dosages include dosages that are below 0.01, 0.05, 0.1, 0.15 or 0.2 mg/kg. In certain embodiments, low dosages administered to the subject may include dosages between 0.001 to 0.02 mg/kg of inhibitor. In certain embodiments, low dosages can be achieved by directly administering the inhibitor to the tissue of interest.

Specific proteins and RNAs are carried into and out of the nucleus by specialized transport molecules, which are classified as importins if they transport molecules into the nucleus, and exportins if they transport molecules out of the nucleus (Terry L J et al. 2007. Crossing the nuclear envelope: hierarchical regulation of nucleocytoplasmic transport. Science 318:1412-1416; and Sorokin A V et al. 2007. Nucleocytoplasmic transport of proteins. Biochemistry 72:1439-1457). Proteins that are transported into or out of the nucleus contain nuclear import/localization (NLS) or export (NES) sequences that allow them to interact with the relevant transporters. Chromosomal Region Maintenance 1 (Crm1), which is also called exportin-1 or Xpo1, is a major exportin.

Crm1 inhibitors have been shown to induce apoptosis in cancer cells even in the presence of activating oncogenic or growth stimulating signals, while sparing normal (untransformed) cells. Most studies of Crm1 inhibition have utilized the natural product Crm1 inhibitor Leptomycin B (LMB). LMB itself is highly toxic to neoplastic cells, but poorly tolerated with marked gastrointestinal toxicity in animals (Roberts et al, 1986) and humans (Newlands et al, 1996). Derivatization of LMB to improve drug-like properties leads to compounds that retain antitumor activity and are better tolerated in animal tumor models (Yang et al, 2007, Yang et al, 2008, Mutka et al, 2009). In certain embodiments, the low dosages of nuclear export inhibitors described herein are not toxic to the subject. In certain embodiments, a dosage is used that is not toxic to a subject. As used herein, “toxic” refers to the ability of a substance or mixture of substances to cause harmful effects over an extended period, usually upon repeated or continuous exposure, sometimes lasting for the entire life of the exposed organism, i.e., capable of causing death or serious debilitation. Non-limiting examples of nuclear export inhibitors applicable to the present invention include KPT-330, KPT-8602, Leptomycin B, Selinexor (Vogl et al., J Clin Oncol. Mar. 20, 2018; 36(9): 859-866) and any of the compounds disclosed in U.S. Pat. No. 9,428,490B2 and U.S. Pat. No. 9,861,614B2.

### Cell Cycle Inhibitors

In certain embodiments, the small molecule or agent is a cell cycle inhibitor (see e.g., Dickson and Schwartz, Development of cell-cycle inhibitors for cancer therapy, Curr Oncol. 2009 March; 16(2): 36-43). In certain embodiments, the cell cycle inhibitor may be, but is not limited to flavopiridol, indisulam, AZD5438, SNS-032, bryostatin-1, seliciclib, PD 0332991, and SCH 727965. In certain embodiments, the cell cycle inhibitor is a CDK inhibitor. In one embodiment, the cell cycle inhibitor is a CDK4/6 inhibitor, such as LEE011, palbociclib (PD-0332991), and Abemaciclib (LY2835219) (see, e.g., U.S. Pat. No. 9,259,399B2; WO2016025650A1; U.S. Patent Publication No. 20140031325; U.S. Patent Publication No. 20140080838; U.S. Patent Publication No. 20130303543; U.S. Patent Publication No. 2007/0027147; U.S. Patent Publication No. 2003/0229026; U.S. patent Publication No 2004/0048915; U.S. Patent Publication No. 2004/0006074; U.S. Patent Publication No. 2007/0179118; each of which is incorporated by reference herein in its entirety). Currently there are three CDK4/6 inhibitors that are either approved or in late-stage development: palbociclib (PD-0332991; Pfizer), ribociclib (LEE011; Novartis), and abemaciclib (LY2835219; Lilly) (see e.g., Hamilton and Infante, Targeting CDK4/6 in patients with cancer, Cancer Treatment Reviews, Volume 45, April 2016, Pages 129-138).

### MEK Inhibitors

In certain embodiments, the small molecule or agent is a MEK inhibitor. A MEK inhibitor is a chemical or drug that inhibits the mitogen-activated protein kinase kinase enzymes MEK1 and/or MEK2. They can be used to affect the MAPK/ERK pathway. Non-limiting examples of MEK inhibitors include Cobimetinib or XL518, Trametinib (GSK1120212), Binimetinib (MEK162), Selumetinib, PD-325901, CI-1040, PD035901, and TAK-733.

In certain embodiments, mRNA encoding a gene product, such as a transcription factor, is delivered to a subject in need thereof. In certain embodiments, the mRNA are modified mRNA (see, e.g., U.S. Pat. No. 9,428,535 B2).

### Vectors

In certain embodiments, vectors are used to overexpress or modulate expression of genes, such as transcription factors. Vectors for introducing CRISPR systems are described further herein.

The term “vector” generally denotes a tool that allows or facilitates the transfer of an entity from one environment to another. More particularly, the term “vector” as used throughout this specification refers to nucleic acid molecules to which nucleic acid fragments (cDNA) may be inserted and cloned, i.e., propagated. Hence, a vector is typically a replicon, into which another nucleic acid segment may be inserted, such as to bring about the replication of the inserted segment in a defined host cell or vehicle organism.

A vector thus typically contains an origin of replication and other entities necessary for replication and/or maintenance in a host cell. A vector may typically contain one or more unique restriction sites allowing for insertion of nucleic acid fragments. A vector may also preferably contain a selection marker, such as, e.g., an antibiotic resistance gene or auxotrophic gene (e.g., URA3, which encodes an enzyme necessary for uracil biosynthesis or TRP1, which encodes an enzyme required for tryptophan biosynthesis), to allow selection of recipient cells that contain the vector. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art.

Expression vectors are generally configured to allow for and/or effect the expression of nucleic acids (e.g., cDNA, CRISPR system) introduced thereto in a desired expression system, e.g., in vitro, in a host cell, host organ and/or host organism. For example, the vector can express nucleic acids functionally or operatively linked to regulatory element(s) and hence the regulatory element(s) drive expression. The promoter(s) can be constitutive promoter(s) and/or conditional promoter(s) and/or inducible promoter(s). In certain embodiments, the vectors comprise regulatory sequences for inducible expression of cDNAs encoding transcription factors. Thus, expression of the transcription factors in cells can induced at particular time points after introducing the vectors. Inducible expression systems are known in the art and may include, for example, Tet on/off systems (see, e.g., Gossen et al., Transcriptional activation by tetracyclines in mammalian cells. Science. Jun. 23, 1995; 268(5218):1766-9).

In certain example embodiments, the vectors disclosed herein may further encode an epitope tag in frame with the gene for use in downstream assessment of protein expression and gene abundance in cell populations respectively. Epitope tags provide high sensitivity and specificity in detection by specific antigen binding molecules (e.g., antibodies, aptamers). Exemplary epitope tags include, but are not limited to, Flag, CBP, GST, HA, HBH, MBP, Myc, polyHis, S-tag, SUMO, TAP, TRX, or V5.

Vectors may include, without limitation, plasmids (which refer to circular double stranded DNA loops which, in their vector form are not bound to the chromosome), episomes, phagemids, bacteriophages, bacteriophage-derived vectors, bacterial artificial chromosomes (BAC), yeast artificial chromosomes (YAC), P1-derived artificial chromosomes (PAC), transposons, cosmids, linear nucleic acids, viral vectors, etc., as appropriate. A vector can be a DNA or RNA vector. A vector can be a self-replicating extrachromosomal vector or a vector which integrates into a host genome, hence, vectors can be autonomous or integrative.

The term “viral vectors” refers to the use as viruses, or virus-associated vectors as carriers of the nucleic acid construct into the cell. Constructs may be integrated and packaged into non-replicating, defective viral genomes like adenovirus, adeno-associated virus (AAV), or herpes simplex virus (HSV) or others, including retroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g., EPV and EBV vectors.

Methods for introducing nucleic acids, including vectors, expression cassettes and expression vectors, into cells (e.g., transfection, transduction or transformation) are known to the person skilled in the art, and may include calcium phosphate co-precipitation, electroporation, micro-injection, protoplast fusion, lipofection, exosome-mediated transfection, transfection employing polyamine transfection reagents, bombardment of cells by nucleic acid-coated tungsten micro projectiles, viral particle delivery, etc.

### Genetic Modulating Agents

In certain embodiments, the one or more modulating agents may be a genetic modifying agent (e.g., gene editing system). The genetic modifying agent may comprise a CRISPR system, a zinc finger nuclease system, a TALEN, a meganuclease, or RNAi.

### CRISPR

In some embodiments, a polynucleotide of the present invention described elsewhere herein (e.g. Table 1, 2 and 3) can be modified using a CRISPR-Cas and/or Cas-based system.

In general, a CRISPR-Cas or CRISPR system as used in herein and in documents, such as International Patent Publication No. WO 2014/093622 (PCT/US2013/074667), refers collectively to transcripts and other elements involved in the expression of or directing the activity of CRISPR-associated (“Cas”) genes, including sequences encoding a Cas gene, a tracr (transactivating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two class are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

In certain embodiments, a CRISPR system is used to enhance expression or activity of transcription factors (e.g., ATF3). In certain embodiments, the transcription factor expression or activity is enhanced temporarily, such that the enhancement is not permanent. In certain embodiments, expression of the transcription from its endogenous gene is enhanced (e.g., by directing an activator to the gene). In certain embodiments, genes are targeted for downregulation. In certain embodiments, genes are targeted for editing.

In certain embodiments, modification of transcription factor mRNA by a Cas13-deaminase system can be used to modulate transcription factor activity in order to generate target cells (see, e.g., International Patent Publication No. WO 2019/084062). In certain embodiments, the modification silences ubiquitination, methylation, acetylation, succinylation, glycosylation, 0-GlcNAc, O-linked glycosylation, iodination, nitrosylation, sulfation, caboxyglutamation, phosphorylation, or a combination thereof. In some embodiments, the modification increases a half-life of a target TF. In certain embodiments, the transcription activity is enhanced by modifying a phosphorylation site on the transcription factor (see, e.g., Hunter and Karin, 1992, The regulation of Transcription by Phosphorylation. Cell, Vol. 70, 375-387; and Whitmarsh and Davis, 2000, Regulation of transcription factor function by phosphorylation. CMLS, Cell. Mol. Life Sci. 57: 1172).

### Class 1 CRISPR-Cas Systems

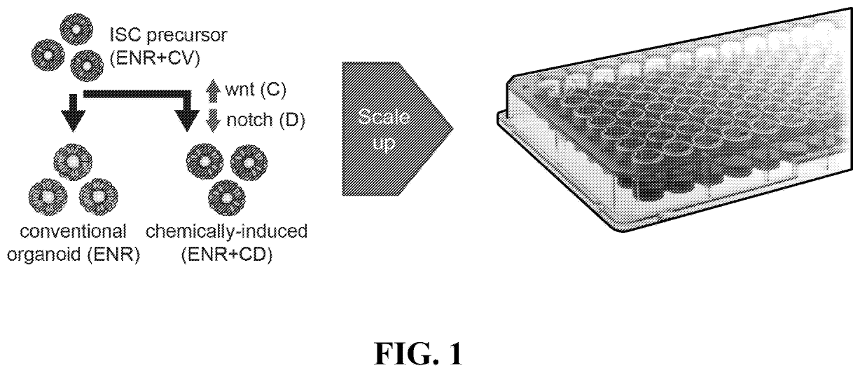

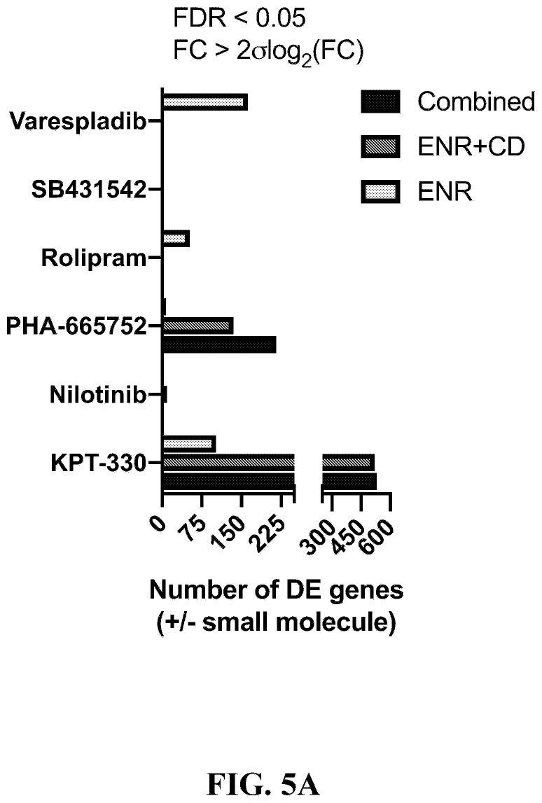

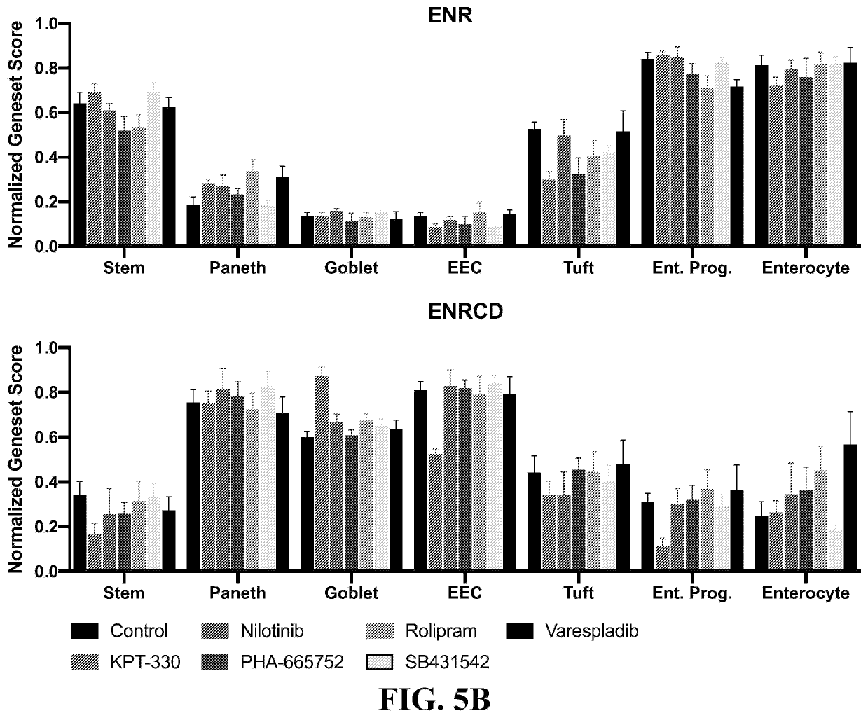

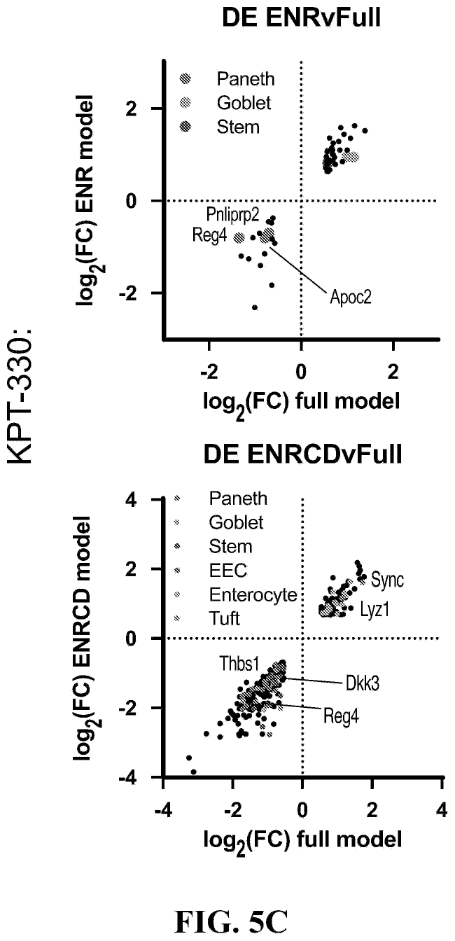

In some embodiments, the CRISPR-Cas system that can be used to modify a polynucleotide of the present invention described herein can be a Class 1 CRISPR-Cas system. Class 1 CRISPR-Cas systems are divided into types I, II, and IV. Makarova et al. 2020. Nat. Rev. 18: 67-83, particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-C, III-D, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI. 10.1073/pnas.1709035114; see also, Makarova et al. 2018. The CRISPR Journal, v. 1, n 5, FIG. 5.

The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g. Cas1, Cas2, RNA nuclease), and/or one or more accessory proteins (e.g. Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits. e.g. Cas 5, Cas6, and/or Cas7. RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

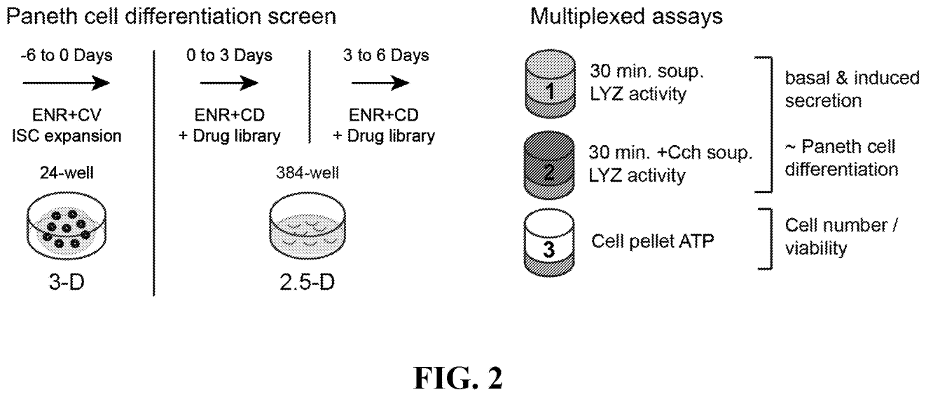

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cas11). See, e.g., FIGS. 1 and 2. Koonin E V, Makarova K S. 2019 Origins and evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas 5, a Cas6, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1 (V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g. Cas9) contain two nuclear domains that are each responsible for the cleavage of one strand of the target DNA, with the HNH nuclease inserted inside the Ruv-C like nuclease domain sequence. The Type V systems (e.g. Cas12) only contain a RuvC-like nuclease domain that cleaves both strands. Type VI (Cas13) are unrelated to the effectors of type II and V systems, contain two HEPN domains and target RNA. Cas13 proteins also display collateral activity that is triggered by target recognition. Some Type V systems have also been found to possess this collateral activity two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), CasX, and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (International Patent Publication No. WO 2014/204725, Ran et al. Cell. Sep. 12, 2013; 154(6):1380-1389), Cas12 (Liu et al. Nature Communications, 8, 2095 (2017), and Cas13 (International Patent Publication Nos. WO 2019/005884, WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g. Zetche et al., 2015. Nat. Biotechnol. 33(2): 139-142, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### Base Editing

In some embodiments, a polynucleotide of the present invention described elsewhere herein (e.g. Table 1, 2 and 3, such as ATF3) can be modified using a base editing system. In some embodiments, a Cas protein is connected or fused to a nucleotide deaminase. Thus, in some embodiments the Cas-based system can be a base editing system. As used herein “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C·G base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471. Base editors may be further engineered to optimize conversion of nucleotides (e.g. A:T to G:C). Richter et al. 2020. Nature Biotechnology. doi.org/10.1038/s41587-020-0453-z.

Other Example Type V base editing systems are described in International Patent Publication Nos. WO 2018/213708 and WO 2018/213726, and International Patent Applications Nos. PCT/US2018/067207, PCT/US2018/067225, and PCT/US2018/067307, which are incorporated by referenced herein.

In certain example embodiments, the base editing system may be a RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA based editor may be used to delete or introduce a post-translation modification site in the expressed mRNA. In contrast to DNA base editors, whose edits are permanent in the modified cell, RNA base editors can provide edits where finer temporal control may be needed, for example in modulating a particular immune response. Example Type VI RNA-base editing systems are described in Cox et al. 2017. Science 358: 1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Prime Editing

In some embodiments, a polynucleotide of the present invention described elsewhere herein (e.g., Table 1, 2 and 3, such as ATF3) can be modified using a prime editing system (See e.g., Anzalone et al. 2019. Nature. 576: 149-157). Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion, and combinations thereof. Generally, a prime editing system, as exemplified by PE1, PE2, and PE3 (Id.), can include a reverse transcriptase fused or otherwise coupled or associated with an RNA-programmable nickase, and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g. sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g. a prime editing guide molecule or peg guide molecule) directly into the target site in the target polynucleotide. See e.g. Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g. is a Cas9 nickase). In some embodiments, the Cas polypeptide is fused to the reverse transcriptase. In some embodiments, the Cas polypeptide is linked to the reverse transcriptase.

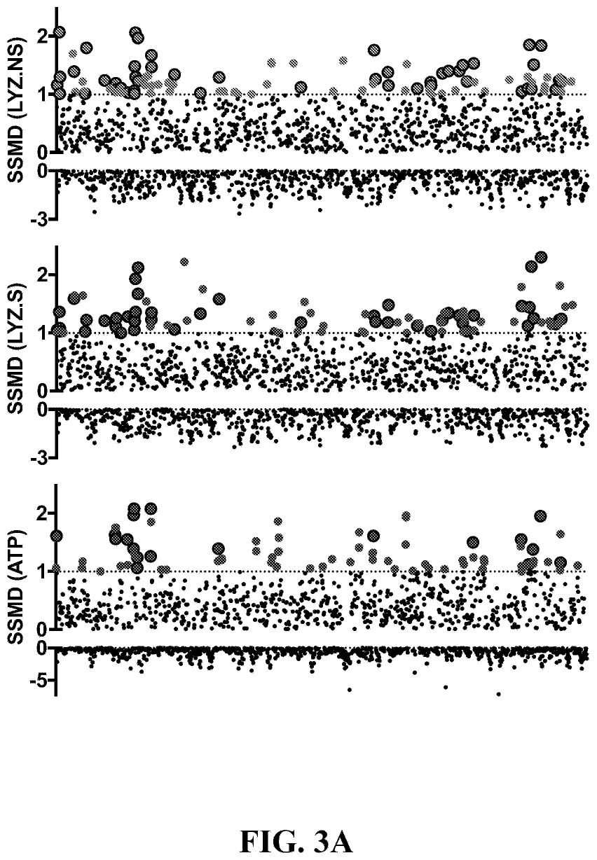

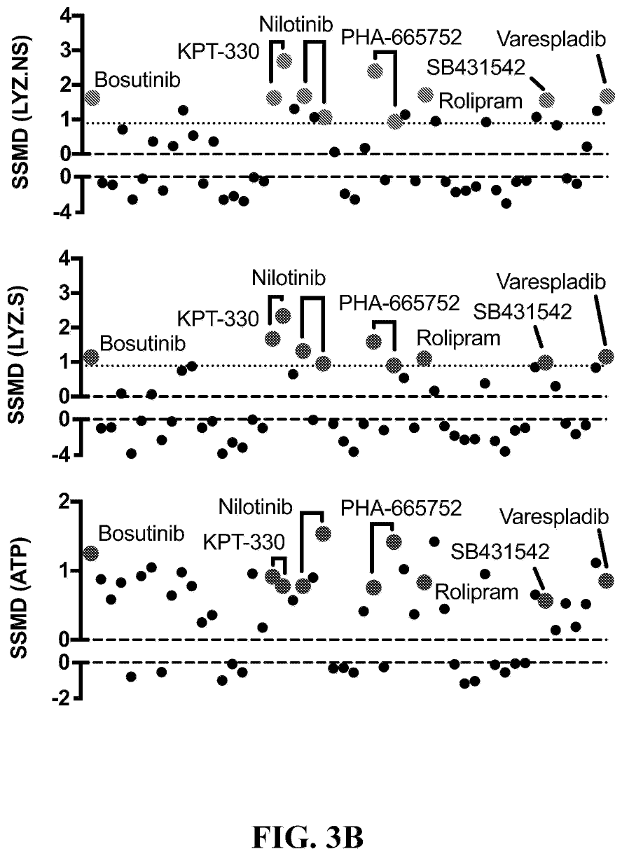

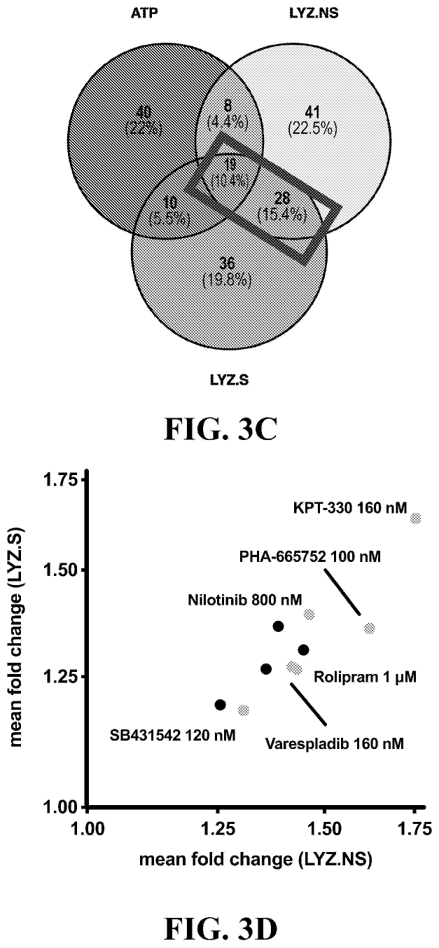

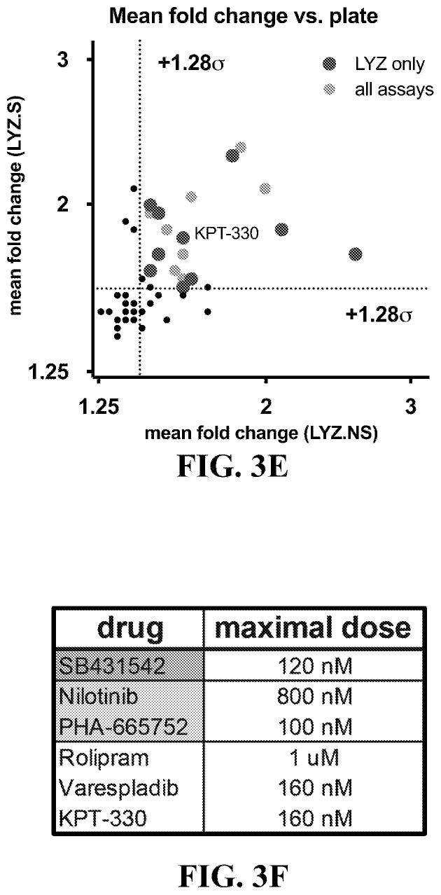

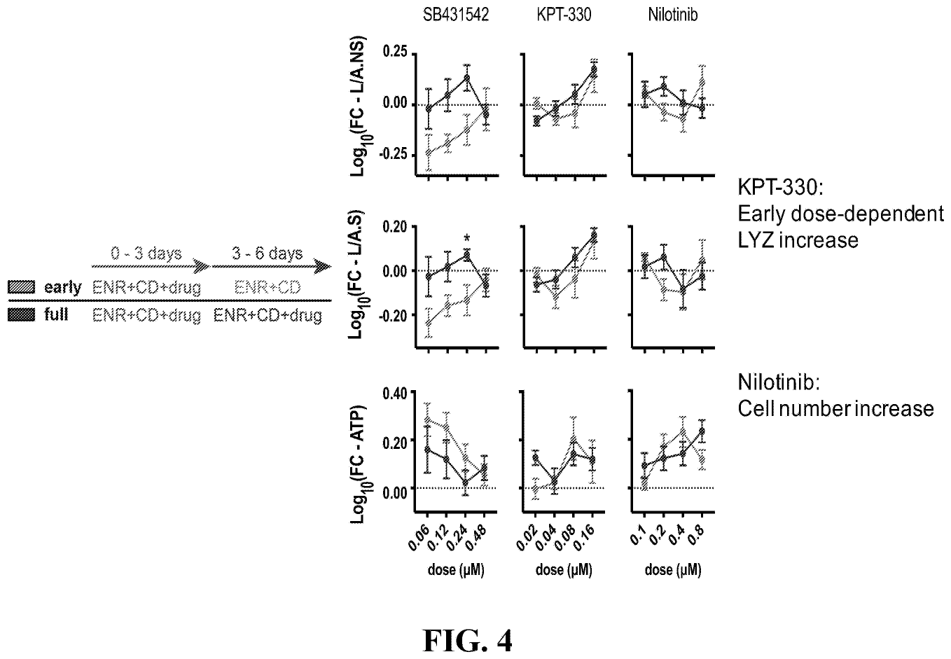

In some embodiments, the prime editing system can be a PE1 system or variant thereof, a PE2 system or variant thereof, or a PE3 (e.g. PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CAST Systems

In some embodiments, a polynucleotide of the present invention described elsewhere herein (e.g. Table 1, 2 and 3, such as ATF3) can be modified using a CRISPR-Associated Transposase (CAST) System, such as any of those described in PCT/US2019/066835. In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science.aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004, BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30-35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e. an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT U.S.2019/045582, specifically paragraphs [0178]-[0333]. which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target polynucleotide can be a polynucleotide or a part of a polynucleotide to which a part of the guide sequence is designed to have complementarity to and to which the effector function mediated by the complex comprising the CRISPR effector protein and a guide molecule is to be directed to. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g. 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead many rely on PFSs, which are discussed elsewhere herein. In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g. Gleditzsch et al. 2019. RNA Biology. 16(4):504-517. Table 4 below shows several Cas polypeptides and the PAM sequence they recognize.

TABLE 4
Example PAM Sequences

| Cas Protein | PAM Sequence |
|---|---|
| SpCas9 | NGG/NRG |
| SaCas9 | NGRRT or NGRRN (SEQ ID |
| | NOs: 1 or 2) |
| NmeCas9 | NNNNGATT (SEQ ID NO: 3) |
| CjCas9 | NNNNRYAC (SEQ ID NO: 4) |
| StCas9 | NNAGAAW (SEQ ID NO: 5) |
| Cas12a (Cpf1) (including | TTTV (SEQ ID NO: 6) |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |
| | (SEQ ID NO: 7) |

In a preferred embodiment, the CRISPR effector protein may recognize a 3′ PAM. In certain embodiments, the CRISPR effector protein may recognize a 3′ PAM which is 5′H, wherein His A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23, 2015; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155 (Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g. LwaCAs13a and PspCas13b) do not seem to have a PFS preference. See e.g. Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g. Gleditzsch et al. 2019. RNA Biology. 16(4):504-517.

Overall Type VI CRISPR-Cas systems appear to have less restrictive rules for substrate (e.g. target sequence) recognition than those that target DNA (e.g. Type V and type II).

### Zinc Finger Nucleases

In some embodiments, the polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments, the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or 34 or 35, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or 34 or 35)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 8)
MDPIRSRTPSPARELLSGPQPDGVQPTADRGVSPPAGGP
LDGLPARRTMSRTRLPSPPAPSPAFSADSFSDLLRQFDPSLFNTS
LFDSLPPFGAHHTEAATGEWDEVQSGLRAADAPPPTMRVAVTA
ARPPRAKPAPRRRAAQPSDASPAAQVDLRTLGYSQQQQEKIKP
KVRSTVAQHHEALVGHGFTHAHIVALSQHPAALGTVAVKYQD
MIAALPEATHEAIVGVGKQWSGARALEALLTVAGELRGPPLQL
DTGQLLKIAKRGGVTAVEAVHAWRNALTGAPLN

An exemplary amino acid sequence of a C-terminal capping region is:

(SEQ ID NO: 9)
RPALESIVAQLSRPDPALAALTNDHLVALACLGGRPAL
DAVKKGLPHAPALIKRTNRRIPERTSHRVADHAQVVRVLGFFQ
CHSHPAQAFDDAMTQFGMSRHGLLQLFRRVGVTELEARSGTLP
PASQRWDRILQASGMKRAKPSPTSTQTPDQASLHAFADSLERD
LDAPSPMHEGDQTRAS

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated by reference.

### Sequences Related to Nucleus Targeting and Transportation

In some embodiments, one or more components (e.g., the Cas protein and/or deaminase, Zn Finger protein, TALE, or meganuclease) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID No. 10) or PKKKRKVEAS (SEQ ID No. 11); the NLS from nucleoplasmin (e.g., the nucleoplasmin bipartite NLS with the sequence KRPAATKKAGQAKKKK (SEQ ID No. 12)); the c-myc NLS having the amino acid sequence PAAKRVKLD (SEQ ID No. 13) or RQRRNELKRSP (SEQ ID No. 14); the hRNPA1 M9 NLS having the sequence NQSSNFGPMKGGNFGGRSSGPYGGGGQYFAKPRNQGGY (SEQ ID No. 15); the sequence RMRIZFKNKGKDTAELRRRRVEVSVELRKAKKDEQILKRRNV (SEQ ID No. 16) of the IBB domain from importin-alpha; the sequences VSRKRPRP (SEQ ID No. 17) and PPKKARED (SEQ ID No. 18) of the myoma T protein; the sequence PQPKKKPL (SEQ ID No. 19) of human p53; the sequence SALIKKKKKMAP (SEQ ID No. 20) of mouse c-abl IV; the sequences DRLRR (SEQ ID No. 21) and PKQKKRK (SEQ ID No. 22) of the influenza virus NS1; the sequence RKLKKKIKKL (SEQ ID No. 23) of the Hepatitis virus delta antigen; the sequence REKKKFLKRR (SEQ ID No. 24) of the mouse M×1 protein; the sequence KRKGDEVDGVDEVAKKKSKK (SEQ ID No. 25) of the human poly(ADP-ribose) polymerase; and the sequence RKCLQAGMNLEARKTKK (SEQ ID No. 26) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g., aptamers) for adapter proteins, which may be linked to or fused to an nucleotide deaminase or catalytic domain thereof. When such a guide forms a CRISPR complex (e.g., CRISPR-Cas protein binding to guide and target) the adapter proteins bind and, the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g., due to steric hindrance within the three dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Templates

In some embodiments, the composition for engineering cells comprise a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include sequence that corresponds to both, a first site on the target sequence that is cleaved in a first Cas protein mediated event, and a second site on the target sequence that is cleaved in a second Cas protein mediated event.

In certain embodiments, the template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include sequence which results in a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 1 10+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may be designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

In certain embodiments, a template nucleic acid for correcting a mutation may be designed for use with a homology-independent targeted integration system. Suzuki et al. describe in vivo genome editing via CRISPR/Cas9 mediated homology-independent targeted integration (2016, Nature 540:144-149). Schmid-Burgk, et al. describe use of the CRISPR-Cas9 system to introduce a double-strand break (DSB) at a user-defined genomic location and insertion of a universal donor DNA (Nat Commun. 2016 Jul. 28; 7:12338). Gao, et al. describe “Plug-and-Play Protein Modification Using Homology-Independent Universal Genome Engineering” (Neuron. Aug. 21, 2019; 103(4):583-597).

### RNAi

In some embodiments, the genetic modulating agents may be interfering RNAs. In certain embodiments, diseases caused by a dominant mutation in a gene is targeted by silencing the mutated gene using RNAi. In some cases, the nucleotide sequence may comprise coding sequence for one or more interfering RNAs. In certain examples, the nucleotide sequence may be interfering RNA (RNAi). As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

In certain embodiments, a modulating agent may comprise silencing one or more endogenous genes. As used herein, “gene silencing” or “gene silenced” in reference to an activity of an RNAi molecule, for example a siRNA or miRNA, refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al., Current Biology, 12, 735-739 (2002), Lagos Quintana et al., Science 294, 853-857 (2001), and Lagos-Quintana et al., RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16.281-297), comprises a dsRNA molecule.

### Antibodies

In certain embodiments, the one or more agents is an antibody. The term “antibody” is used interchangeably with the term “immunoglobulin” herein, and includes intact antibodies, fragments of antibodies, e.g., Fab, F(ab′)₂ fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)₂, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclassess of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, lgM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by p pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 μM or less, 100 μM or less, 50 μM or less or 25 μM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)₂, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and CHI domains; (ii) the Fab′fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and CHI domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., International Patent Publication No. WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptidomimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

### Aptamers

In certain embodiments, the one or more agents is an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH₂), 2′-fluoro (2′-F), and/or 2′-O-methyl (2′-OMe) substituents. Modifications of aptamers may also include, modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, 0-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include, but are not limited to those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colo.). In certain embodiments, the present invention may utilize any aptamer containing any modification as described herein.

### Methods of Detection and Diagnostic Methods

In certain embodiments, target cell types are identified using biomarkers and/or gene signatures. In certain embodiments, biomarkers and/or signatures are identified in a population of cells in response to modulating agents that drive differentiation.

In certain example embodiments, the population of cells is an ex vivo cell-based system that faithfully recapitulates an in vivo phenotype or target system of interest. Source starting materials may include cultured cell lines or cells or tissues isolated directly from an in vivo source, including explants and biopsies. The source materials may be pluripotent cells including stem cells. The cell (sub)type(s) and cell state(s) of the ex vivo system may likewise be determined using known lineage markers. The analysis of cell (sub)type(s) and cell state(s) may be obtained at the time of running the methods described herein. Based on the identified differences, steps to modulate the source material to induce a shift in cell (sub)type(s) and/or cell state(s) may then be selected and applied. In certain embodiments, gene signatures, pathways and/or biomarkers are modulated to shift differentiation of an ex vivo or in vivo system (see modulating agents herein).

The invention provides biomarkers (e.g., phenotype specific or cell type) for the identification, diagnosis, prognosis and manipulation of cell properties, for use in a variety of diagnostic and/or therapeutic indications. Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein.

Biomarkers are useful in methods of diagnosing, prognosing and/or staging an immune response in a subject by detecting a first level of expression, activity and/or function of one or more biomarker and comparing the detected level to a control of level wherein a difference in the detected level and the control level indicates that the presence of an immune response in the subject.

The terms “diagnosis” and “monitoring” are commonplace and well-understood in medical practice. By means of further explanation and without limitation the term “diagnosis” generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The biomarkers of the present invention are useful in methods of identifying patient populations at risk or suffering from an immune response based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments.

The term “monitoring” generally refers to the follow-up of a disease or a condition in a subject for any changes which may occur over time.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-á-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-á-vis a control subject or subject population.

Suitably, an altered quantity or phenotype of the immune cells in the subject compared to a control subject having normal immune status or not having a disease comprising an immune component indicates that the subject has an impaired immune status or has a disease comprising an immune component or would benefit from an immune therapy.

Hence, the methods may rely on comparing the quantity of immune cell populations, biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterised by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value >second value; or decrease: first value <second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, 50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

### Single Cell RNA Sequencing

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science Mar. 15, 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International Patent Application No. PCT/US2016/059239, published as WO2017164936 on Sep. 28, 2017; International Patent Application No. PCT/US2018/060860, published as WO/2019/094984 on May 16, 2019; International Patent Application No. PCT/US2019/055894, published as WO/2020/077236 on Apr. 16, 2020; and Drokhlyansky, et al., “The enteric nervous system of the human and mouse colon at a single-cell resolution,” bioRxiv 746743; doi: doi.org/10.1101/746743, which are herein incorporated by reference in their entirety.

In certain example embodiments, assessing the cell (sub)types and states present in the ex vivo system may comprise analysis of expression matrices from scRNA-seq data, performing dimensionality reduction, graph-based clustering and deriving list of cluster-specific genes in order to identify cell types and/or states present in the system. Further the clustering and gene expression matrix analysis allow for the identification of key genes in the ex vivo system, such as differences in the expression of key transcription factors.

In certain embodiments, dimension reduction is used to cluster nuclei from single cells based on differentially expressed genes. In certain embodiments, the dimension reduction technique may be, but is not limited to, Uniform Manifold Approximation and Projection (UMAP) t-SNE, or PHATE (see, e.g., Becht et al., Evaluation of UMAP as an alternative to t-SNE for single-cell data, bioRxiv 298430; doi.org/10.1101/298430; Becht et al., 2019, Dimensionality reduction for visualizing single-cell data using UMAP, Nature Biotechnology volume 37, pages 38-44; and Moon et al., PHATE: A Dimensionality Reduction Method for Visualizing Trajectory Structures in High-Dimensional Biological Data, bioRxiv 120378; doi: doi.org/10.1101/120378).

### Other Detection Methods

Modulation may be monitored in a number of ways. For example, expression of one or more key marker genes may be measured at regular levels to assess increases in expression levels. Shifting of the ex vivo system may also be measured phenotypically. For example, imaging an immunocytochemistry for key in vivo markers may be assessed at regular intervals to detect increased expression of the key in vivo markers. Likewise, flow cytometry may be used in a similar manner. In addition, to detecting key in vivo markers, imaging modalities may be used to further detect changes in cell morphology of the ex vivo system.

In one embodiment, the signature genes, biomarkers, and/or cells may be detected or isolated by immunofluorescence, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25).

The present invention also may comprise a kit with a detection reagent that binds to one or more biomarkers or can be used to detect one or more biomarkers.

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MvIS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies, etc.) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as International Patent Publication Nos. WO 95/21265, WO 96/31622, WO 97/10365, and WO 97/27317, and European Patent Application Nos. EP 373 203 and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65° C. for 4 hours followed by washes at 25° C. in low stringency wash buffer (1×SSC plus 0.2% SDS) followed by 10 minutes at 25° C. in high stringency wash buffer (0.1 SSC plus 0.2% SDS) (see Shena et al., Proc. Natl. Acad. Sci. U.S.A., Vol. 93, p. 10614 (1996)). Useful hybridization conditions are also provided in, e.g., Tijessen, Hybridization With Nucleic Acid Probes”, Elsevier Science Publishers B. V. (1993) and Kricka, “Nonisotopic DNA Probe Techniques”, Academic Press, San Diego, Calif. (1992).

### ATAC-Seq

In certain embodiments, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. May 22, 2015; 348(6237):910-4. doi: 10.1126/science.aab1601. Epub 2015 May 7; U.S. patent Publication Nos. U.S.20160208323A1 and U.S.20160060691A1; and International Patent Publication No. WO2017156336A1).

### Screening Methods

### Screening for Modulating Agents

A further aspect of the invention relates to a method for identifying an agent capable of modulating one or more phenotypic aspects of a cell or cell population as disclosed herein, comprising: a) applying a candidate agent to the cell or cell population; b) detecting modulation of one or more phenotypic aspects of the cell or cell population by the candidate agent, thereby identifying the agent. The phenotypic aspects of the cell or cell population that is modulated may be a gene signature or biological program specific to a cell type or cell phenotype or phenotype specific to a population of cells. In certain embodiments, steps can include administering candidate modulating agents to cells, detecting identified cell (sub)populations for changes in signatures, or identifying relative changes in cell (sub) populations which may comprise detecting relative abundance of particular gene signatures.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of an immune cell or immune cell population may be modulated without substantially altering other (unintended, undesired) phenotypic aspect(s).

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

The methods of phenotypic analysis can be utilized for evaluating environmental stress and/or state, for screening of chemical libraries, and to screen or identify structural, syntenic, genomic, and/or organism and species variations. For example, a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value. By exposing cells, or fractions thereof, tissues, or even whole animals, to different members of the chemical libraries, and performing the methods described herein, different members of a chemical library can be screened for their effect on immune phenotypes thereof simultaneously in a relatively short amount of time, for example using a high throughput method.

Aspects of the present disclosure relate to the correlation of an agent with the spatial proximity and/or epigenetic profile of the nucleic acids in a sample of cells. In some embodiments, the disclosed methods can be used to screen chemical libraries for agents that modulate chromatin architecture epigenetic profiles, and/or relationships thereof.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures or biological programs of the present invention may be used to screen for drugs that reduce the signature or biological program in cells as described herein. The signature or biological program may be used for GE-HTS. In certain embodiments, pharmacological screens may be used to identify drugs that are selectively toxic to cells having a signature.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a signature or biological program of the present invention in silico.

The invention is further described in the following examples, which do not limit the scope of the invention described in the claims.

### EXAMPLES

### Example 1—Modulation of Paneth Cell Development and Function

### Scalable Platform to Study Paneth Cell Development and Function

Applicants developed a scalable, functional, phenotypic assay for screening ENR+CD-treated cells (FIG. 1). To overcome the limitations to scaling organoid-derived cultures, Applicants first sought to develop a method to preserve the important material and signaling cues supplied by Matrigel scaffolding while enabling automated plating through robotic liquid handlers used in high-throughput screening. Applicants adapted the conventional “3-D” Matrigel droplet culture approach to a 96-well plate pseudo-monolayer “2.5-D” scheme in which organoids are replated partially embedded on the surface of a thick layer of Matrigel (at the Matrigel-media interface) rather than fully encapsulated in the Matrigel structure. This allows for a two-step automated plating procedure, where Matrigel is first deposited and gelled in 96- (or 384-) well plates, and culture media containing suspended cell clusters is then added into each well, and briefly centrifuged at low force to loosely deposit clusters on the surface of the thick Matrigel scaffold. This allows for Matrigel plating, cell seeding, and media additions to be fully automated by a liquid handler and readily scaled. Further, because the deposited cell clusters are now apically-exposed to the culture media, assaying apically-secreted agents (such as LYZ) should be greatly enhanced, while allowing for the multiplexed assaying of underlying embedded cells (FIG. 2).

Applicants seeded 6-day ENR+CD differentiated clusters per well and assayed for cellular ATP as well as basal LYZ secretion over 3 hours. The optimal plating density is between 300-75 clusters/well (10-2.5 clusters/μL media). The 2.5-D platform in a 96-well plate provided an ability to discriminate PC function between conventional, ISC-enriched, and PC-enriched organoids.

Applicants applied the screening platform as a tool to investigate and validate the actions of proposed agents which modulate in vivo PC function. Applicants can determine the approximate standardized effect size of treatment at different doses using replicate-based strictly standardized mean difference (SSMD), an effect size measure based on the statistics of contrast variables, and particularly useful in screening applications as it is a measure which takes into account both mean difference and variance in a single measure. Using the replicate based uniformly minimal variance unbiased estimate (UMVUE) SSMD, Applicants can identify modulators of LYZ secretion. Applicants can distinguish between strong and weak effect agonists of LYZ secretion, and in the case of strong agonists (e.g., cholinergic agonists (CCh)), are able to provide a relatively high-resolution dose-response profile with associated EC50 concentration.

In total, the platform is capable of scaling an organoid-derived culture reproducibly with a simple phenotypic assay for PC function (LYZ secretion). The platform can be used to assess potential short and long-term modulators of differentiated cell function. A primary strength of this PC screening platform is in studying agents that may enhance the pace of PC development and may serve as therapeutic candidates to increase or improve PC quality in diseases where there is a loss of PC number or function, such as Crohn's disease.

### Primary Small Molecule Screen for Molecular Targets to Enhance Paneth Cell Development

While the scaled screening approach enables phenotypic study of differentiated PCs at scale, it also offers an opportunity to examine modulators of the differentiation ‘trajectory’ from ISC precursors. Intervening during the 6-day differentiation of ENR+CD organoids allows for the study of unappreciated molecular pathway activity which influences PC differentiation and may be readily translated beyond the model. Applicants therefore sought to use the scalable “2.5-D” platform to demonstrate a proof-of-concept screen for developmental process of PCs in vitro, and to elucidate molecular pathways and small molecule agents which may afford an axis to enhance PC number therapeutically.

Using a modified version of the 96-well “2.5-D” system and functional assays, Applicants screened for agents which enhance in vitro PC differentiation or survival using a target-selective inhibitor (TSI) library (Selleck Chemicals (Houston, Tex.) L3500) containing 433 small molecules covering 184 well-characterized unique molecular targets with high specificity. This library offers translational advantages as many of the molecules are either presently used in the clinic for a wide variety of conditions or have been used in clinical trials and animal models. Applicants scaled the 96-well 2.5D system to a 384-well plate format with a single-well stimulation protocol and assessed the activity of secreted LYZ (basal—LYZ.NS and Cch-stimulated—LYZ.S) and cell pellet ATP following treatments of each compound. ISC-enriched “small clusters” from 3 biological donors were seeded as 3 replicate screens into differentiation media (ENR+CD), and then screened with each of the 433 different compounds at 4 doses covering the nano-molar to micro-molar range. By screening in the presence of the PC differentiation media Applicants sought to assess how the library compounds may act outside of the known WNT and Notch pathways to influence PC differentiation or function, while simultaneously generating a PC enriched system with which to robustly assay for PC function. Applicants performed the three sequential assays 6 days after initial plating with ENR+CD+library treatment and had an additional media change and drug treatment at day 3 (FIG. 2). Each screen plate was log₁₀ transformed, and LOESS normalized to reduce plate effects, and each well value was reported as fold change (FC), relative to the median assay value of its respective plate (under the assumption that many of the compounds and doses on the plate will not be biologically active and therefore serve as a suitable control).

In the LYZ.NS (first assay—all +drug wells are sampled for basal secretion) non-stimulated ENR+CD controls were significantly greater than that of no cell controls (adj. p<0.0001), and 10 μM CCh-stimulated ENR+CD controls was significantly greater than that of non-stimulated positive controls (adj. p<0.0001). In the LYZ.S (second assay—all+drug wells are sampled for Cch-induced secretion) non-stimulated ENR+CD controls subsequently stimulated with 10 μM CCh versus non-stimulated (adj. p<0.05) and those doubly non-stimulated positive controls versus no cell controls (adj. p<0.0001) showed significant differences. Further, each plate across each replicate was relatively well-correlated for all three assays.

To assess treatment effect size and define primary screen ‘hits’, replicate SSMD was calculated and hits were determined by an SSMD greater than the false-positive and false-negative derived cutoffs (errors equalized to minimum for 3 replicates alpha=0.087) for both LYZ.NS and LYZ.S assays (FIG. 3A). Of these 47 hits, 19 were also hits in the ATP assay, with the remaining 28 as hits in only both LYZ assays. Interestingly, a plurality of hits was assay-specific, suggesting that single-assay hits may either arise from system ‘noise’ or may suggest unique biological effects (FIG. 3C). Applicants further refined the set of 47 double-LYZ assay hits by only including the most biologically ‘potent’ treatments, using a cutoff corresponding to treatments which would fall in the top 10% of a normal distribution (zscored FCs>1.282) (FIG. 3E).

To validate the 13 most potent enhancers of PC differentiation identified in primary screens, Applicants conducted secondary screening using the same assays and screening format as before with an increased number of well replicates per treatment, a lower tolerance for false positives (without consideration for false negatives, High-throughput screening: 384-well secondary screen format and early vs. full treatment analysis), and a more stringent control population (ENR+CD or ENR compared to whole plate in primary screen).

Applicants again assessed treatment effect size with replicate SSMD (using a difference between well replicates and median assay value for all same-plate control wells and hits were considered statistically validated by an SSMD greater than the false-positive-derived cutoffs for both LYZ.NS and LYZ.S assays (FIG. 3B). Per this new cutoff, 10 dose treatment combinations corresponding to 7 small molecules were chosen as hits, with every passing dose-treatment having a greater than 0 ATP effect size. Applicants also profiled the biological potency (mean fold change between treatment and ENR+CD+DMSO for the LYZ.NS and LYZ.S assays), of the 10 validated dose-treatment combinations, showing that the compounds increased basal and stimulated LYZ secretion by 25%-75% relative to the control (FIG. 3D). For each compound with multiple validated doses (KPT-330, PHA-665752, and Nilotinib), the most biologically active dose was advanced, and, because Nilotinib and Bosutinib have similar known mechanisms, only Nilotinib (the more biologically potent) was advanced (FIG. 3F) to additional profiling.

To clarify differences between early and full treatment during differentiation, Applicants looked at the differences between early and late treatment across a range of doses for 3 compounds in the LYZ assays normalized to their matched ATP values (a measure of LYZ/ATP suggests functional capacity per cell) as well as their ATP basis. (FIG. 4). The hits ‘act’ at different points in differentiation.

### Validation of XPO1 Inhibition as a Means to Enhance Paneth Cell Differentiation

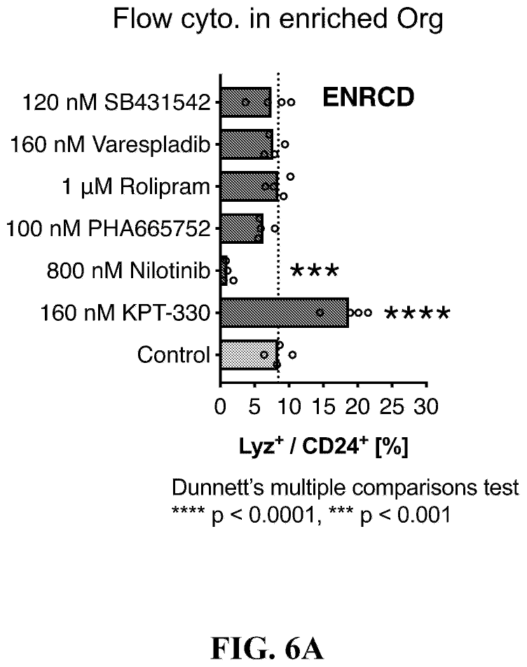

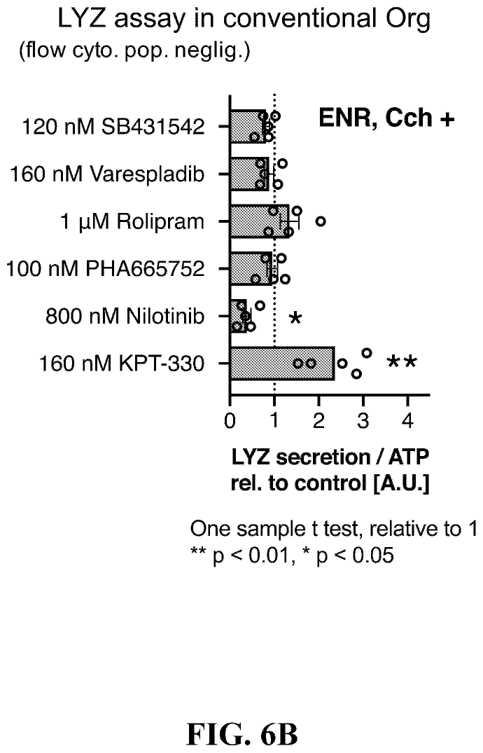

Of the six promising lead small molecules identified, KPT-330 appears to most significantly enhance Paneth cell differentiation, and as such Applicants sought to better understand the mechanism through which KPT-330 may be acting, whether by canonical XPO1 inhibition, or other means. Applicants determined the effect of the hits on gene expression by population RNA sequencing. KPT-330 showed the most differentially expressed genes (FIG. 5A) Gene set scores for the intestinal cell types showed that after treatment with the small molecules secretory cells (Paneth, goblet, and EEC) were increased (FIG. 5B, C). Assays in the conventional (3D) system confirmed KPT-330 differentiation (FIG. 6A, B).

KPT-330 is a chromosome region maintenance-1 (CRM1) inhibitor with antineoplastic activity. CRM1 (also known as XPO1, emb, exp1, exportin 1, CRM-1) is a eukaryotic protein that mediates the nuclear export of proteins, rRNA, snRNA, and some mRNA. KPT-330 acts via the selective inhibition of nuclear export (SINE) approach—by modifying the essential CRM1-cargo binding residue C528, KPT-330 irreversibly inactivates CRM/11-mediated nuclear export of cargo proteins, including growth regulation proteins. CRM1 co-immunoprecipitates with p27kip1, a protein whose constitutive expression causes cell cycle arrest in the G¹ phase that precedes differentiation. Upregulation of CRM1 and decreased levels of p27kip1 are observed in mucosal biopsies of patients with active Crohn's disease. Based on the results showing an increase in PC number and function with ENR+CD+KPT-330 treatment, CRM1 inhibition by KPT-330 may promote p27kip1-mediated cell cycle arrest to allow ISCs to transition first to a secretory cell progenitor, then to terminally differentiated PCs.

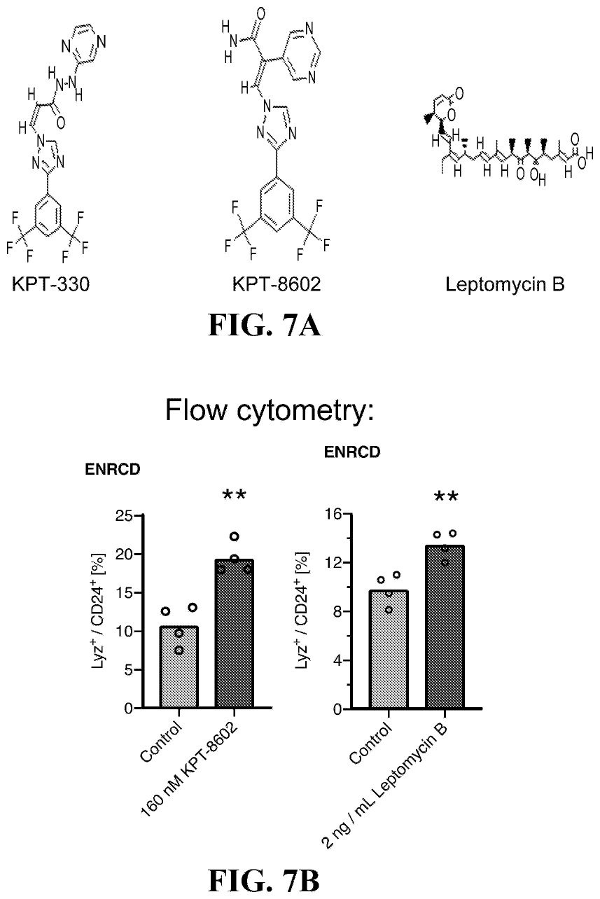

To address whether enhanced Paneth cell differentiation within ENR+CD organoids following KPT-330 treatment is stemming from the known mechanism of XPO1 inhibition, or a potential off-target or non-canonical effect, Applicants repeated organoid differentiation with two additional known XPO1 inhibitors, KPT-8602 and Leptomycin B (FIG. 7A). As measured by flow cytometry, statistically significant increases in Paneth cell representation were observed following treatment with KPT-8602 and Leptomycin B (FIG. 7B).

KPT-330 administration drives Paneth cell differentiation through canonical XPO1 inhibition, as confirmed by parallel assessment with additional known XPO1 inhibitors KPT-8602 and Leptomycin B, which lead to similar, statistically significant increases in the Paneth cell fraction of within ENR+CD differentiated organoids.

Using the high-fidelity PC model in the traditional 3D enteroid culture system, Applicants differentiated ISC-enriched organoids from one biological donor and then sought to assess to what extent XPO1 inhibition is interdependent on Wnt agonism and Notch antagonism to drive secretory (Paneth) cell differentiation. Applicants assessed this through studies of bulk transcripts, protein, and functional assays.

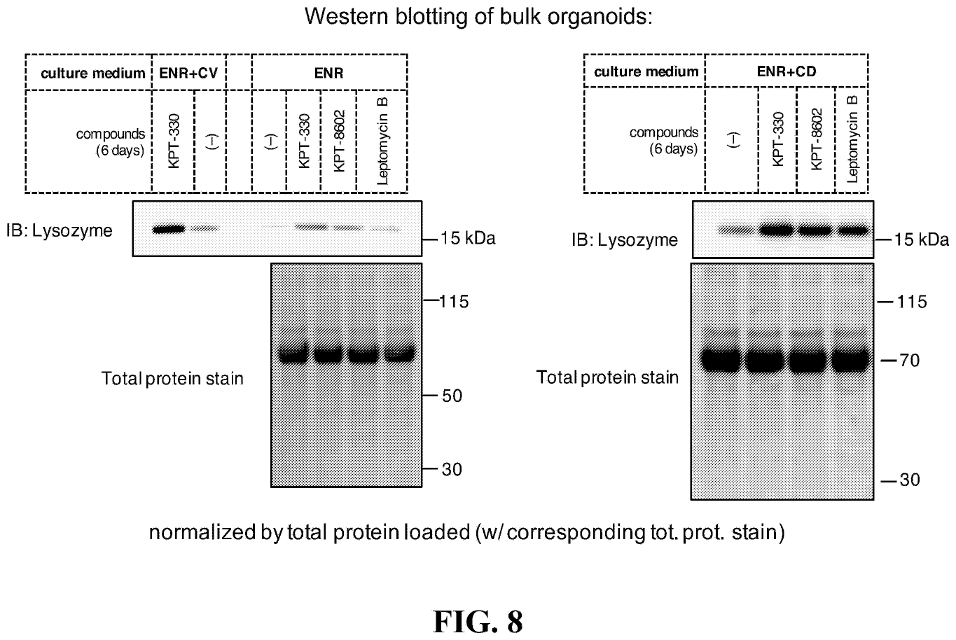

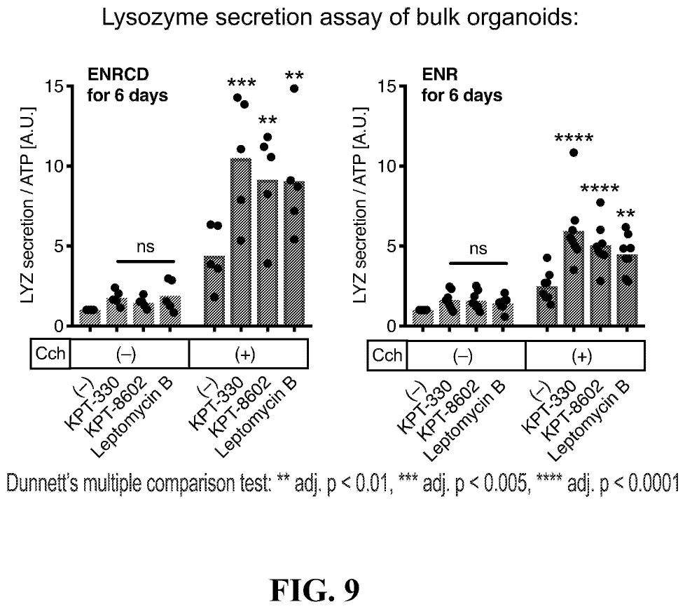

Assaying bulk protein extracted from organoids differentiated under either ENR+CV, ENR+CD, or ENR for LYZ, it is apparent that the addition of XPO1 inhibitors KPT-330, KPT-8602, and Leptomycin B all appreciably increase LYZ abundance on a per-mass protein normalized basis (FIG. 8). Additionally, for both ENR and ENR+CD following 6-day differentiation, functional lysozyme secretion is greatly enhanced when XPO1 inhibitors are added (FIG. 9).

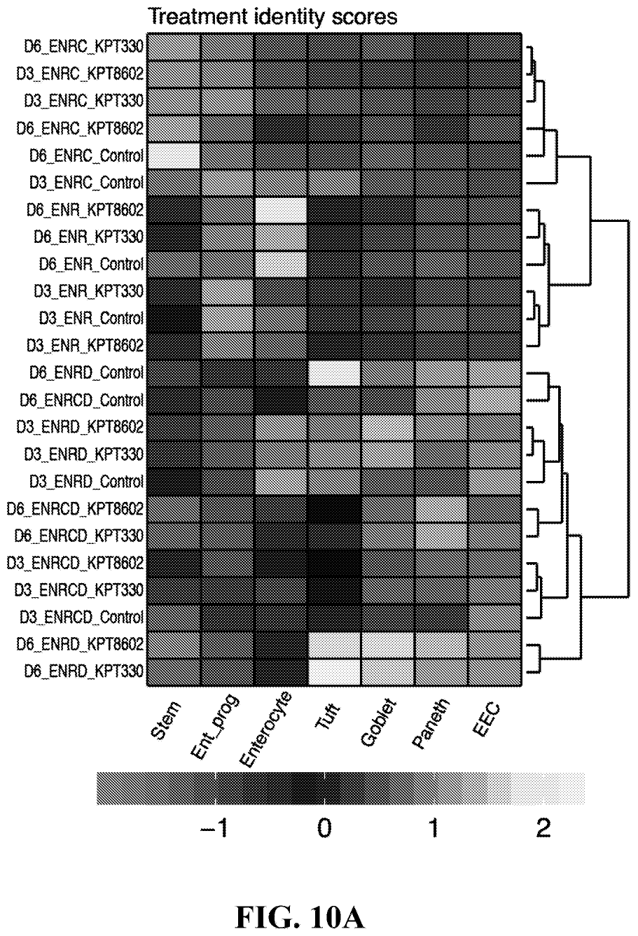

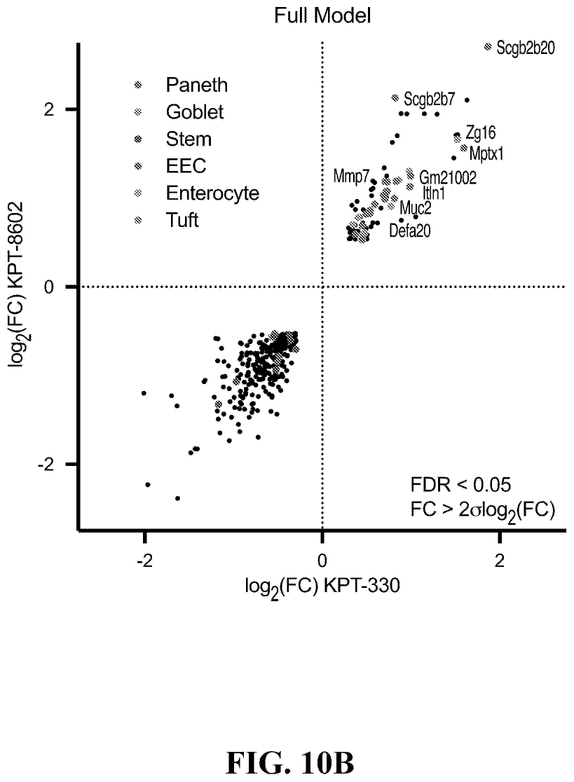

Assaying bulk transcripts from organoids differentiated under either ENR+CV, ENR+CD, or ENR for LYZ, it is apparent that the addition of XPO1 inhibitors KPT-330 and KPT-8602 increase secretory cells (Paneth, goblet, and EEC) (FIG. 10A, B).

### Single Cell RNA Sequencing to Determine Pathways Involved in Differentiation

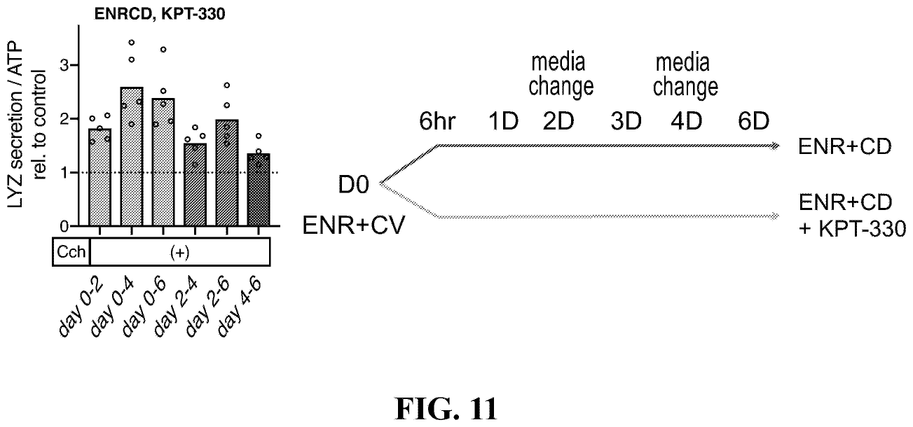

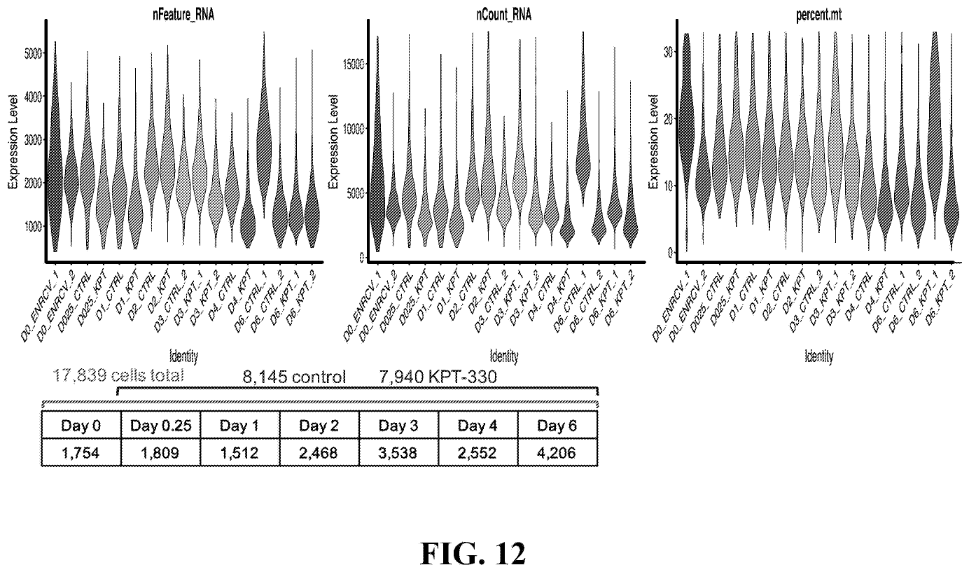

Applicants tested a time course of LYZ/ATP after KPT-330 treatment for use in further analyzing KPT-330 function (FIG. 11). Applicants performed seq-well on cells taken at each time point and treatment and determined quality control factors for each sample (FIG. 12). In total 17,839 cells passed quality control.

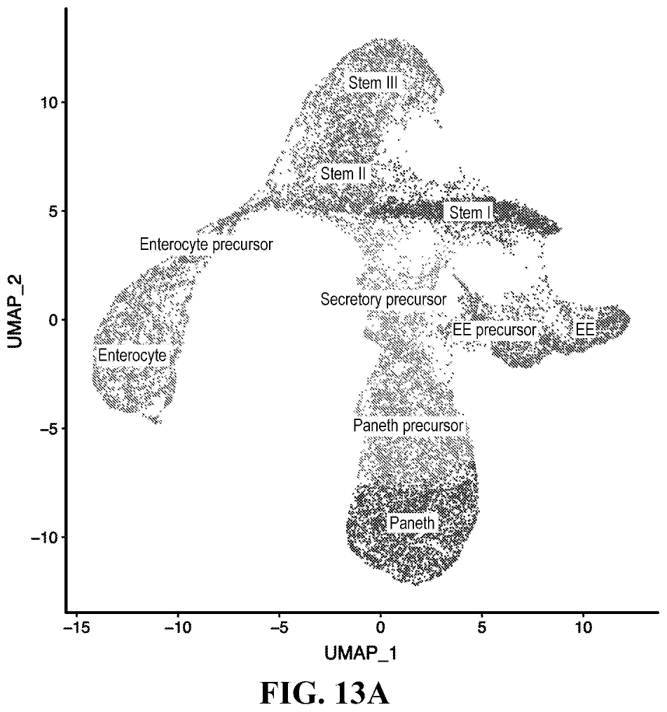

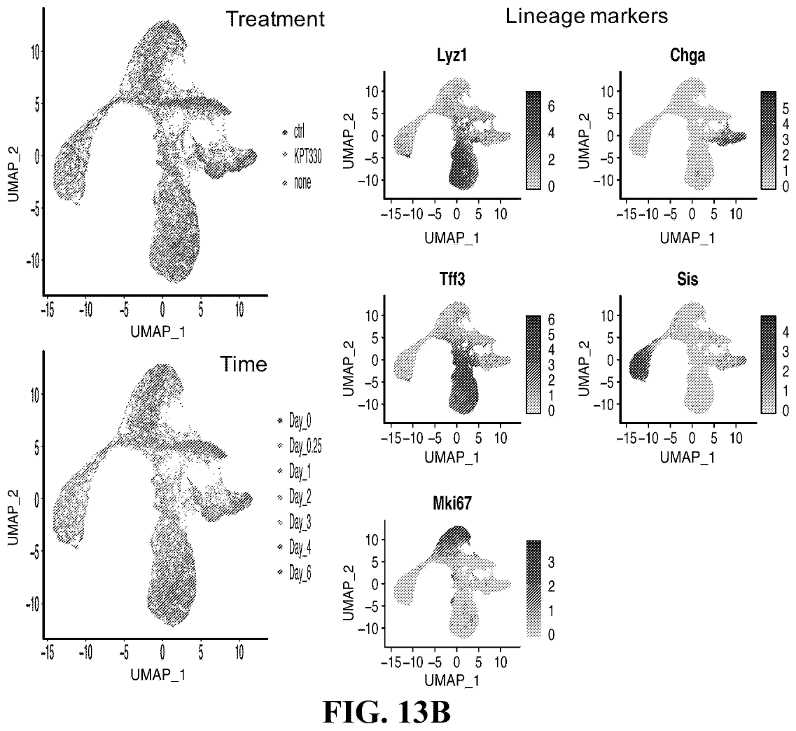

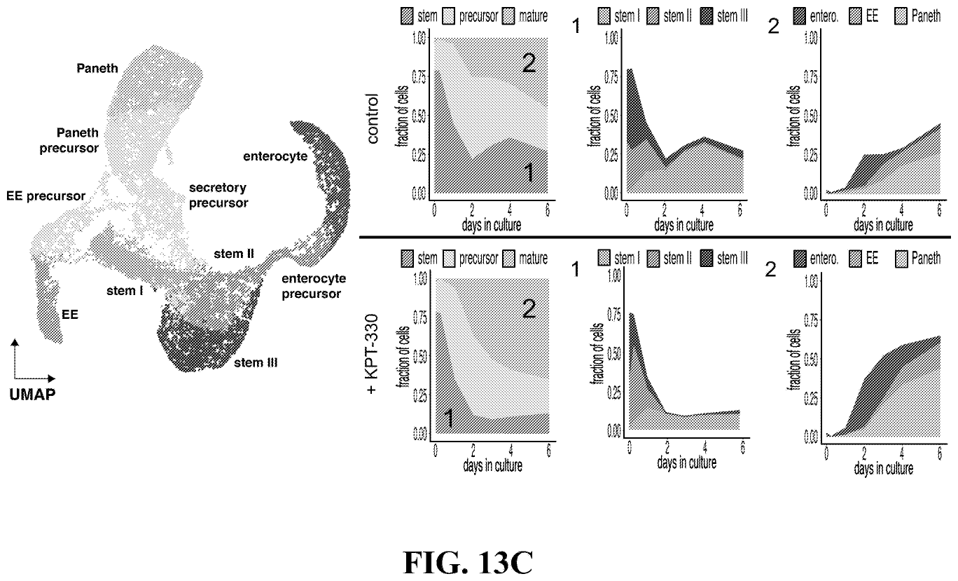

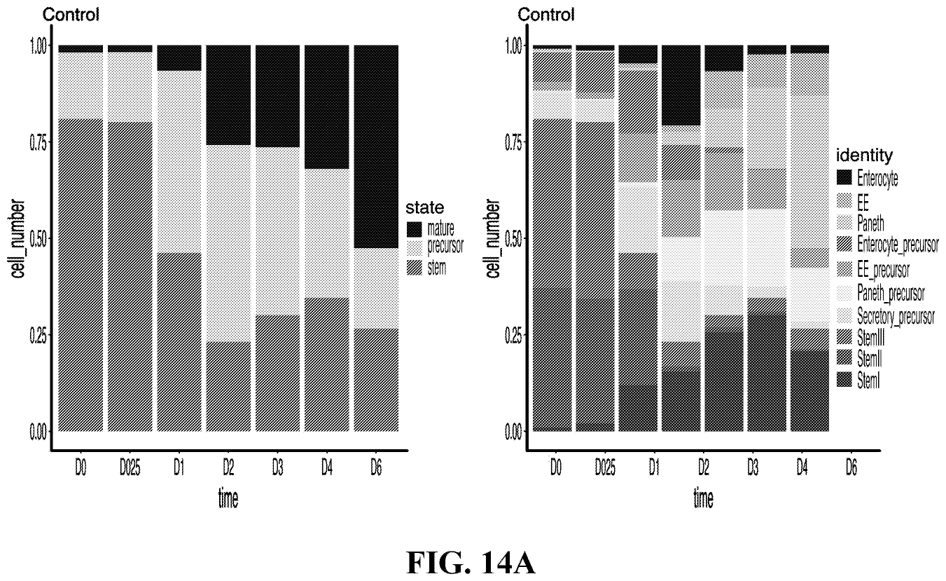

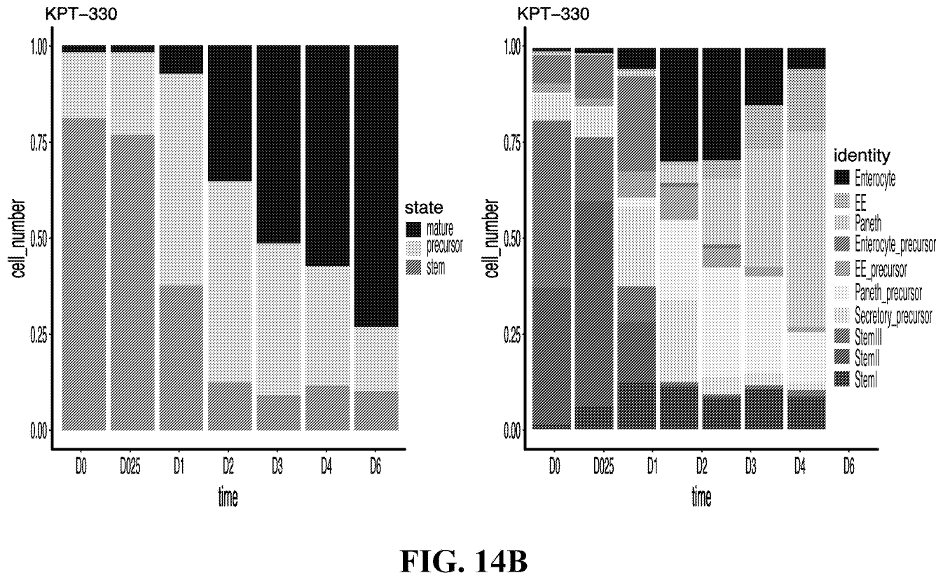

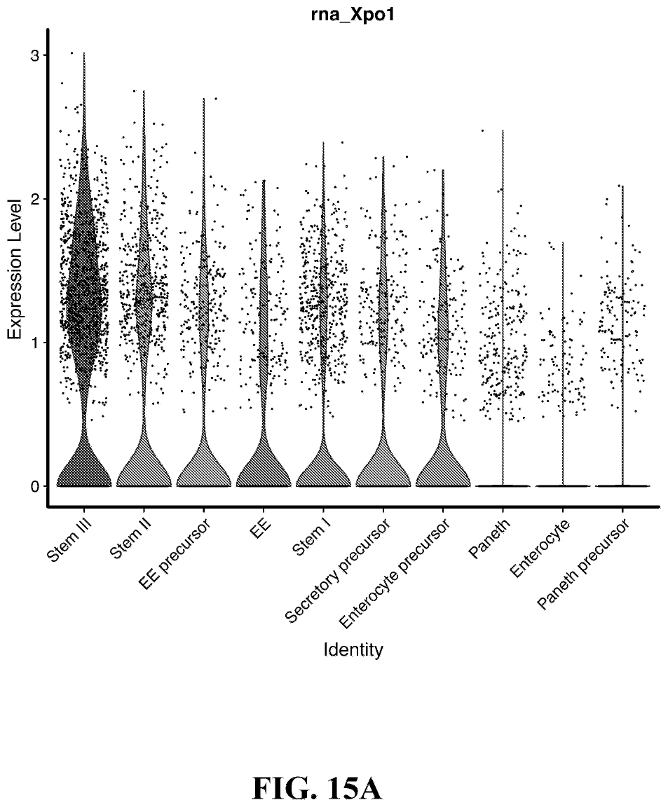

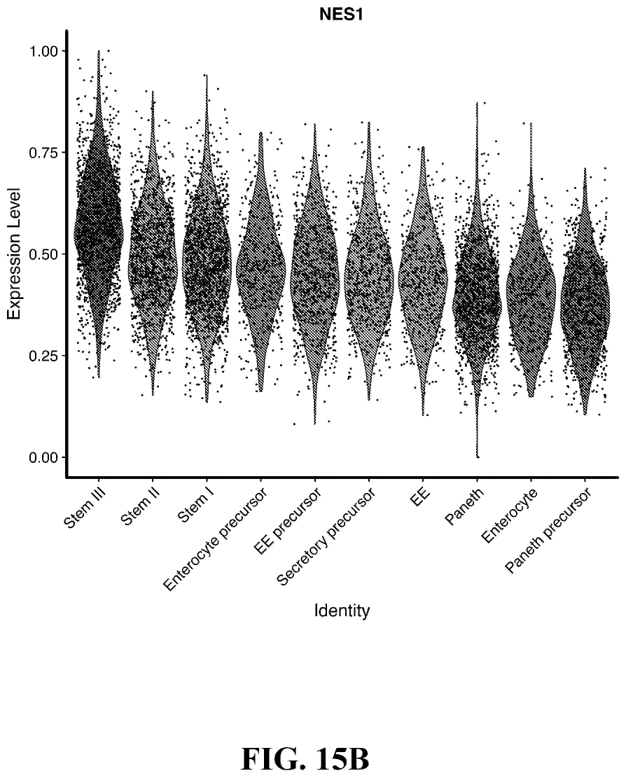

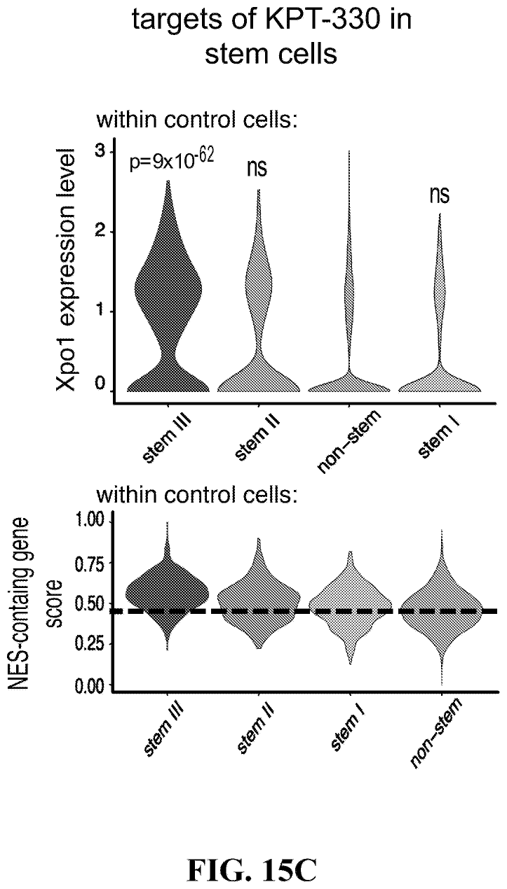

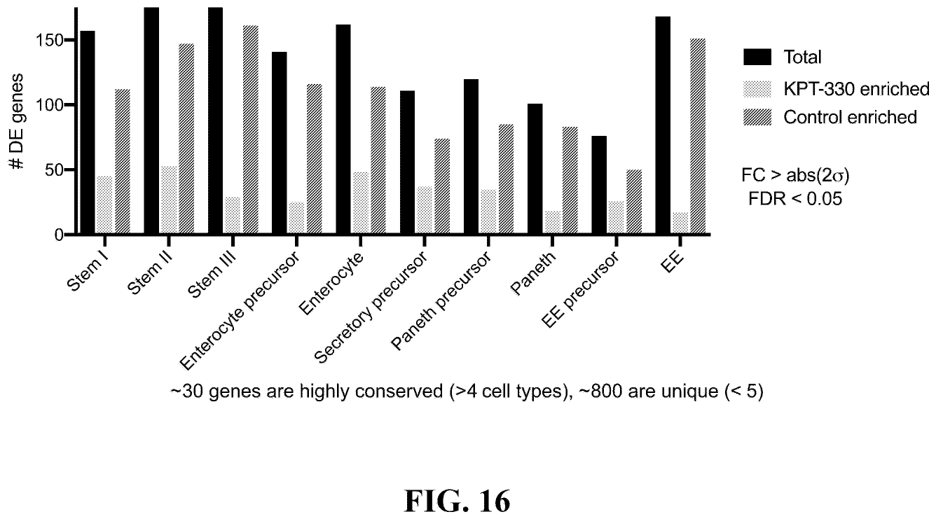

Single cells were analyzed using dimension reduction (UMAP). The single cells clustered by cell type (FIG. 13A, C). The single cells were also analyzed for clustering by treatment and time (FIG. 13B, left). Lineage markers were projected on the clusters and confirm cell lineage and proliferating cells (FIG. 13B, right). The cell numbers and fraction of each cell type over the time course for control and KPT-330 treatment were determined, showing that KPT-330 enhances stem conversion to mature cells (FIG. 14A, B; 13C, right). Applicants analyzed the expression of XPO1 and NES transcripts in the single cells and found the highest expression in stem cells (FIG. 15A, B, C). XPO1 expression was lowest in Paneth cells, enterocytes and Paneth precursor cells (non-stem cells). Applicants determined the number of differentially expressed genes across the single cell types (FIG. 16).

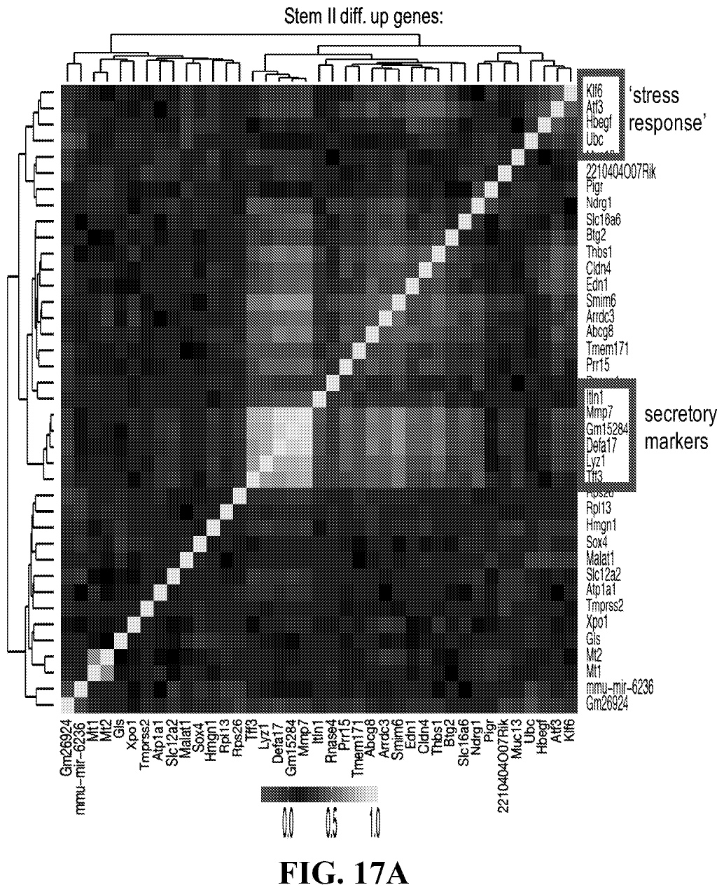

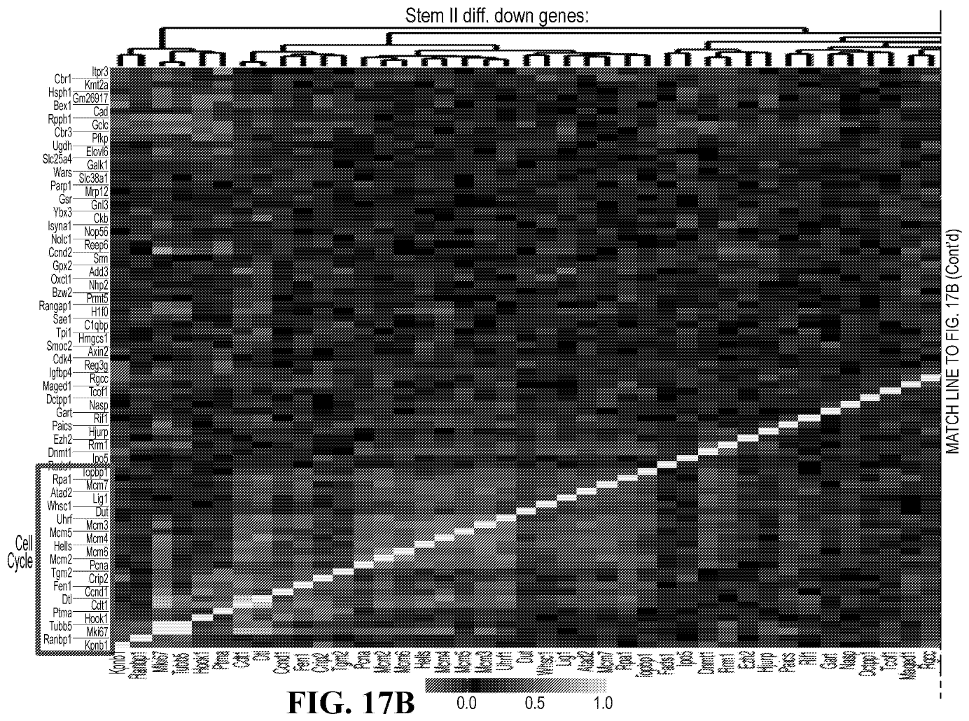

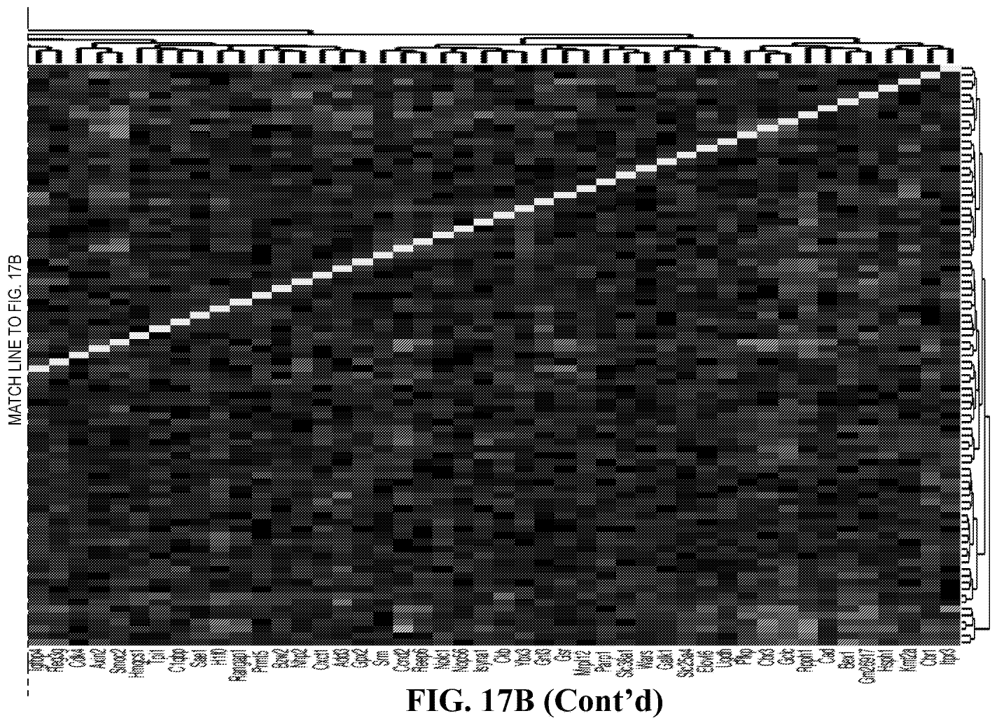

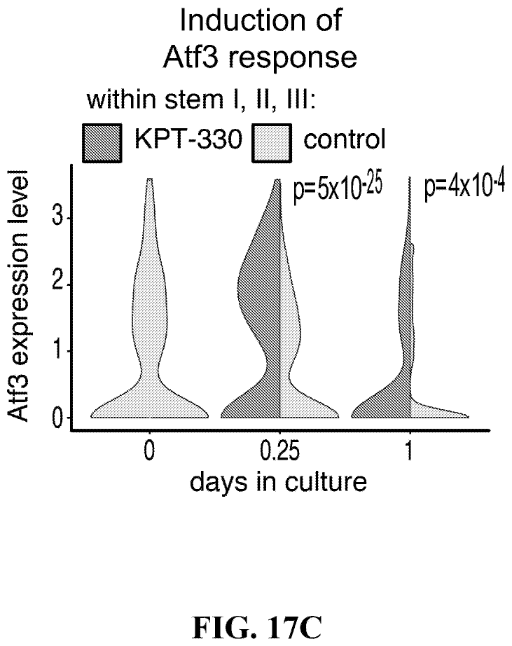

Applicants determined the correlation of genes differentially expressed between control and KPT-330 treated cells. Genes that correlated go up or down in expression together. Applicants found correlation between genes involved in stress response and secretory markers in the upregulated differentially expressed genes (FIG. 17A; Table 1). Applicants found correlation between genes involved in the cell cycle in the downregulated differentially expressed genes (FIG. 17B; Table 2). Thus, Xpo1 inhibition leads to an increase in stress response genes and downregulation of cell cycle genes. Thus, differentiation may be enhanced by treatment with a cell cycle inhibitor. Transcription factors are known to drive differentiation. ATF3 is a transcription factor upregulated by Xpo1 inhibition (KPT-330) and may function to drive differentiation of stem cells to mature secretory cells (FIG. 17C). Thus, enhancing activity of ATF3 (e.g., modifying post-translational modification sites) or increasing expression may be used to drive differentiation in a more focused method than Xpo1 inhibition.

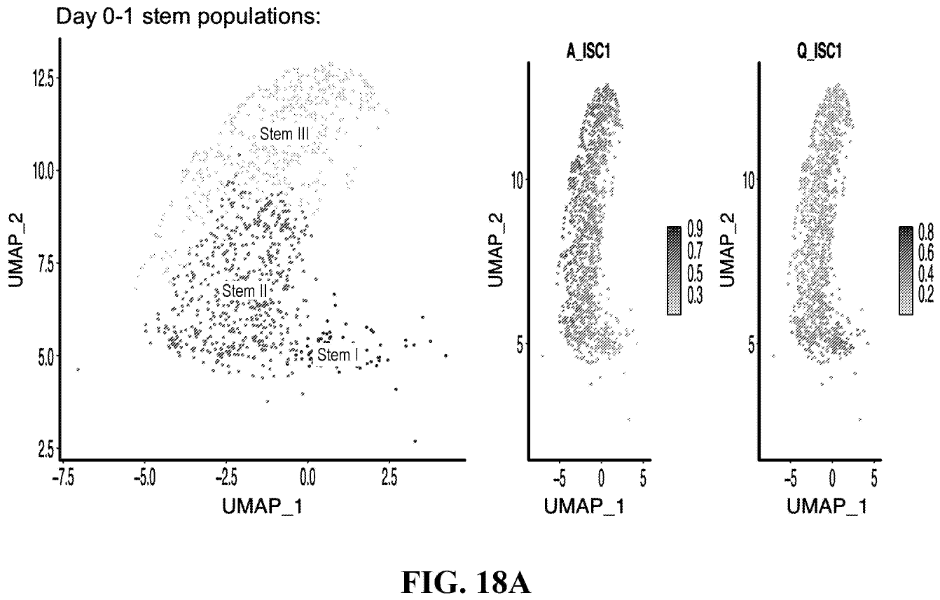

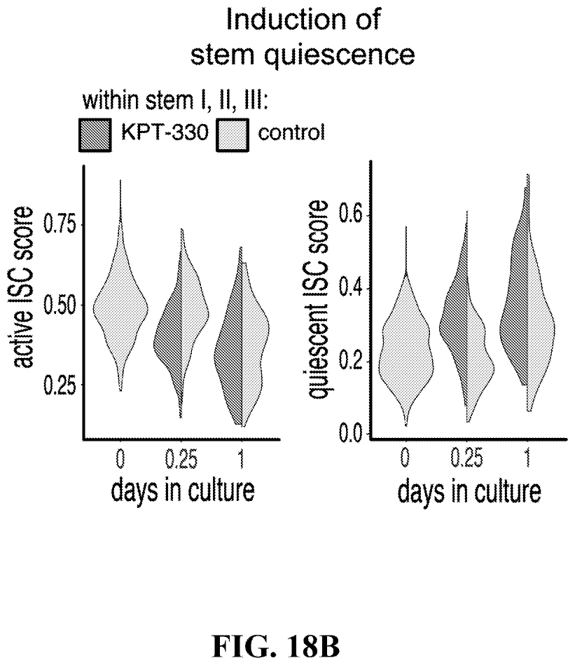

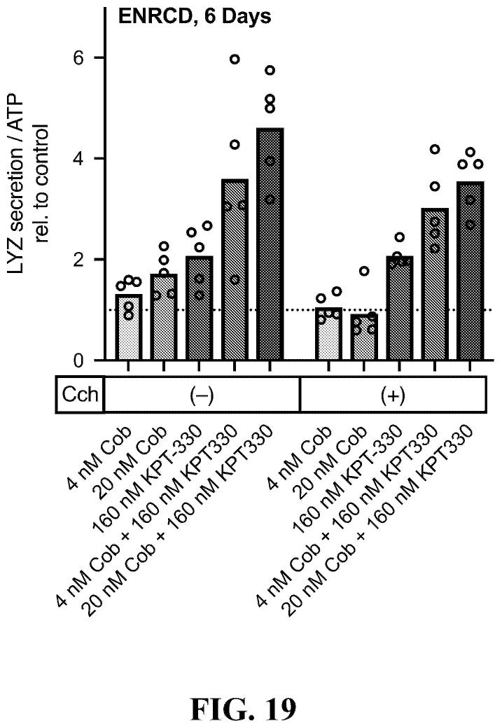

Applicants also determined that KPT-330 induces a quiescent ISC signature and reduces an active ISC signature in Day 0-1 stem cell populations (FIG. 18A, B). Applicants further identified that induction of stem cell quiescence enhances the effect of KPT-330. Applicants determined that differentiation was enhanced using the combination of a map kinase inhibitor, cobimetinib (Cob), and KPT-330 (FIG. 19). Thus, contacting cells with an agent that induces a quiescent ISC signature can be used to drive differentiation.

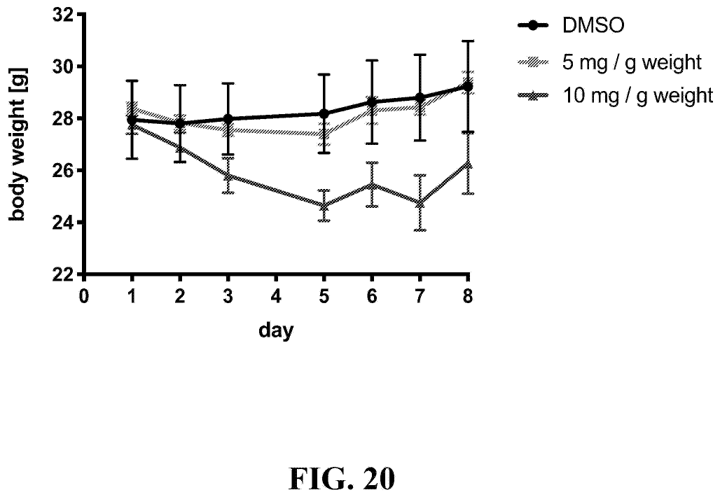

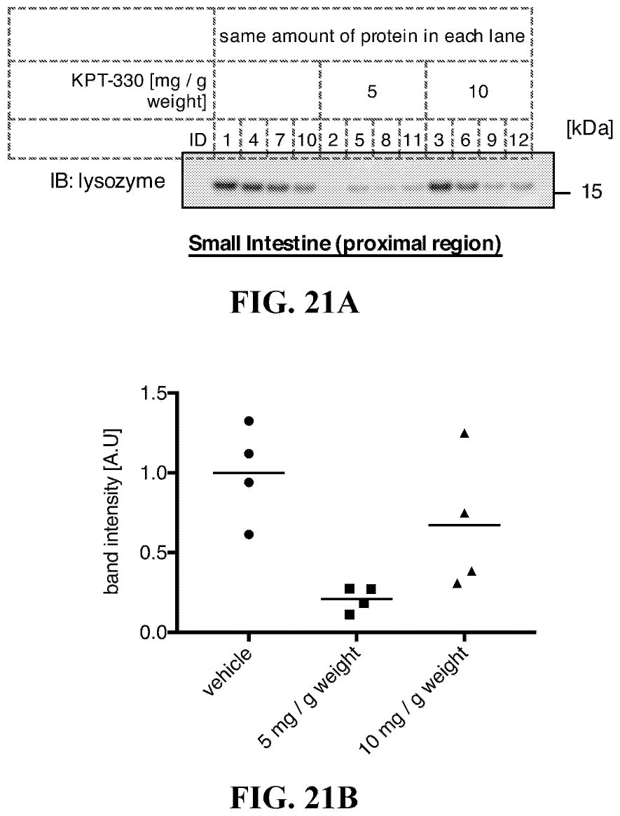

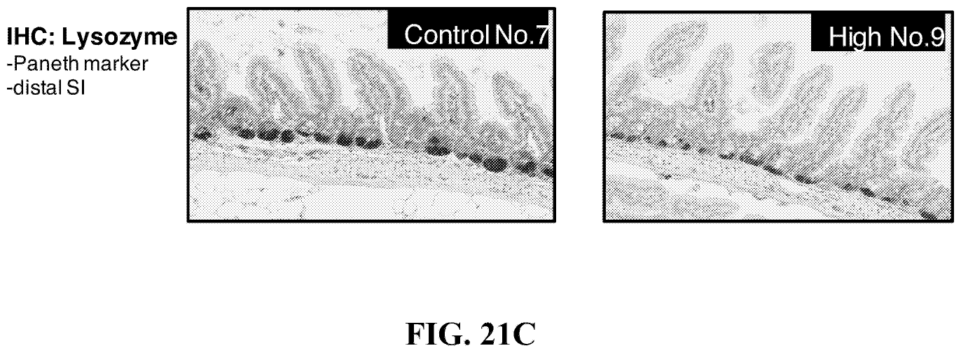

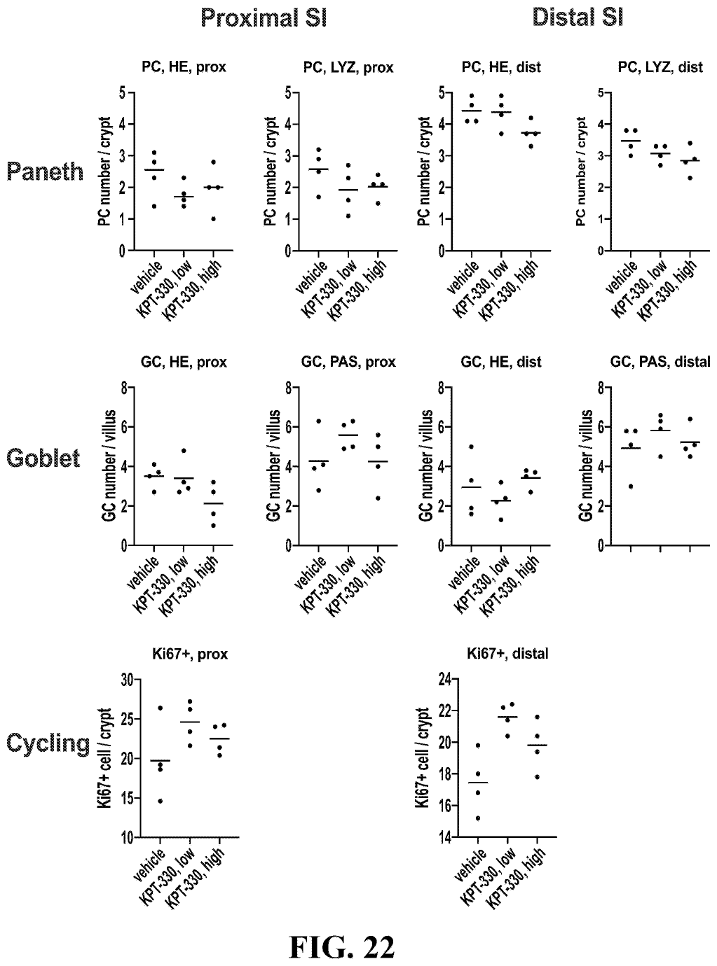

Applicants determined the effect of KPT-330 in vivo (FIGS. 20-22). Applicants observed that high concentration of KPT-330 was toxic to mice (FIG. 20). Thus, a more focused treatment may be beneficial to drive differentiation of stem cells in vivo. Applicants observed using histology that KPT-330 provides a pro-differentiation effect (FIG. 22). In the proximal small intestines, Applicants observed no significant shift in Paneth cell number. In the distal small intestines, Applicants observed that the high dose decreased Paneth cells. In the proximal/distal small intestines Applicants observed no significant shift in goblet cell numbers, but the trend was an increase. In the distal small intestines, Applicants observed a significant increase in cycling cell number at low dose (5 mg/g).

### Materials and Methods

Western blotting. ISC-enriched organoids cultured in 3D Matrigel with ENRCV media were passaged to 24-well plate in 3D Matrigel with ENRCV. At day zero, media were replaced to ENR or ENRCD with or without indicated compounds, and media were replaced every other day. At day six, cells were harvested from Matrigel by mechanical disruption and suspended in basal media. Cell pellets were lysed with Pierce® IP lysis buffer (ThermoFisher, 87787) containing Halt™ Protease Inhibitor Cocktail, EDTA-Free (ThermoFisher, 87785) after 3-minute centrifuge at 300×g at 4° C. Cell extracts were resolved by NuPAGE® SDS-PAGE Gel system (ThermoFisher) and electroblotted onto polyvinylidene difluoride membranes using Criterion™ Blotter (Biorad). The membranes were blocked with 2% Blotting-Grade Blocker (Biorad, 1706404) in TBS-T (50 mM Tris-HCl, 150 mM NaCl, and 0.1% Tween 20, pH 8.0) and then probed with appropriate antibodies. Detection was performed using ECL Select™ Western Blotting Detection Reagent (Amersham, 45-000-999) and ImageQuant LAS4000 (GE Healthcare).

Lysozyme assay (3D culture). ISC-enriched organoids cultured in 3D Matrigel with ENRCV media were passaged to 48-well plate in 3D Matrigel with ENRCV. At day zero, media were replaced to ENR or ENRCD with or without indicated compounds, and media were replaced every other day. At day six, cells were washed twice with basal media and treated with carbamylcholine chloride (Sigma, C4382) for 3 hours. Media were collected and lysozyme activity was measured by EnzChek Lysozyme Assay Kit (ThermoFisher, E22013). Simultaneously, cell viability was measured by CellTiter-Glo®3D Cell Viability Assay (Promega, G9681).

Population RNA-sequencing. Population RNA-seq was performed using a derivative of the Smart-Seq2 protocol for single cells. In brief, organoid media was aspirated and RLT+BME (Qiagen) was added to each well, and plate shaken for 30 minutes to fully lyse. Lysate was aliquoted into 4 identical fractions and stored at −80° C. until reverse transcription. RNA lysate was thawed and cleaned with a 2.2×SPRI ratio using Agencourt RNAClean XP beads (Beckman Coulter, A63987). RNA-seq was performed on a bulk population of sorted basal cells using Smart-Seq2 chemistry, starting with a 2.2×SPRI ratio cleanup. After oligo-dT priming, Maxima H Minus Reverse Transcriptase (ThermoFisher EP0753) was used to synthesize cDNA with an elongation step at 52° C. before PCR amplification (15 cycles for tissue, 18 cycles for sorted basal cells) using KAPA HiFi PCR Mastermix (Kapa Biosystems KK2602). Sequencing libraries were prepared using the Nextera XT DNA tagmentation kit (Illumina FC-131-1096) with 250 pg input for each sample. Libraries were pooled post-Nextera and cleaned using Agencourt AMPure SPRI beads with successive 0.7× and 0.8× ratio SPRIs and sequenced with an Illumina 75 Cycle NextSeq500/550v2 kit (Illumina FC-404-2005) with loading density at 2.2 μM, with paired end 35 cycle read structure. Samples were sequenced at an average read depth of 8.44 million reads per sample and a total of 96 samples.

Organoid samples were aligned to the Mm10 genome and transcriptome using STAR and RSEM. Differential expression analysis was conducted using DESeq2 package for R. Genes regarded as significantly differentially expressed were determined based on an adjusted P value using the Benjamini-Hochberg procedure to correct for multiple comparisons with a false discovery rate <0.01. For module scoring with the in vivo Paneth and enteroendocrine cell-defining genes, Applicants used previously published gene sets and computed per sample module scores through the Seurat package.

Crypt isolation and organoid culture. Small intestinal crypts were isolated from C57BL/6 mice of both sexes, aged between three to six months in all experiments. Crypts were then cultured in a Matrigel culture system. Briefly, crypts were resuspended in basal culture medium (Advanced DMEM/F12 with 2 mM GlutaMAX and 10 mM HEPES; Thermo Fisher Scientific) at a 1:1 ratio with Corning™ Matrigel™ Membrane Matrix—GFR (Fisher Scientific) and plated at the center of each well of 24-well plates. Following Matrigel polymerization, 500 μL of small intestinal crypt culture medium (basal media plus 100× N2 supplement, 50× B27 supplement; Life Technologies, 500× N-acetyl-L-cysteine; Sigma-Aldrich) supplemented with growth factors EGF—E (50 ng/mL, Life Technologies), Noggin—N (100 ng/mL, PeproTech) and R-spondin 1—R (500 ng/mL, PeproTech) and small molecules CHIR99021—C (3 μM, LC Laboratories) and valproic acid—V (1 mM, Sigma-Aldrich) was added to each well. ROCK inhibitor Y-27632—Y (10 μM, R&D Systems) was added for the first 2 days of culture. Cells were cultured at 37° C. with 5% CO₂, and cell culture medium was changed every other day. After 6 days of culture, crypt organoids were isolated from Matrigel by mechanical dissociation. To expand enriched ISCs (ENR+CV/Y) or Paneth Cells (ENR+CD), organoids were cultured in 24-well plates, suspended in 40 uL 3-D gels (50-50 GFR MATRIGEL®, Basal culture media), with 500 uL of crypt media supplemented with necessary growth factors and small molecules. ROCK inhibitor (Y) was added for the first two days of ISC culture following reconstitution from cryopreservation or trypLE passaging to single cells. Cell culture medium was changed every other day. After 4 days of culture in ENR+CV, cell clusters were differentiated to PCs under the ENR+CD condition for 96-well short and longterm screens. For 384-well differentiation screens, 4-day ENR+CV clusters were passaged to single cells using trypLE, replated and expanded another 3 days in 3-D ENR+CVY and then passaged directly into screens. Basal culture medium: Advanced DMEM/F12 with 2 mM GlutaMAX and 10 mM HEPES; Thermo Fisher Scientific.

High-throughput screening: 96-well format. For 96-well plate high-throughput screening, 4-day differentiated (ENR+CD) cell clusters in 3D Matrigel were transferred to a “2.5-D” 96-well plate culture system. Briefly, cell culture gel and medium were homogenized via mechanical disruption and centrifuged at 300 g for 3 min at 4° C. Supernatant was removed, and the pellet resuspended in basal culture medium repeatedly until the cloudy Matrigel was almost gone. On the last repeat, pellet was resuspended in basal culture medium, the number of cell clusters counted, and centrifuged at 300 g for 3 min at 4° C. The cell pellet was resuspended in ENR-CD medium and plated using a Tecan Evo liquid handler at the center of each well of 96-well plates prepared with a 45 uL polymerized 70% Matrigel (30% basal media) coating in each well. Plates were centrifuged at 50 g for 1 min at 4° C. to allow for cells to partially embed in Matrigel coating. At end time points (following 2 days in culture and 3 hours of stimulation), lysozyme secretion and cell viability were assessed using Lysozyme Assay Kit and CellTiter-Glo 3D Cell Viability Assay (Promega), respectively, according to the manufacturers' protocols. Briefly, 2.5D 96-well culture plates are spun at high speed (>2000 g) for 5 min at RT to pellet cell debris, then 25 μl of conditioned supernatant is removed from the top of each well and mixed with 75 μl lysozyme working solution using a black 96-well flat bottom plate (LYZ screen plate). The LYZ screen plate is covered, shaken for 10 min, incubated for 20 min at 37° C., then fluorescence measured (494 nm/518 nm). 25 μl CTG 3D is added to each well of the 2.5D culture plate, which is then shaken for 15 min before reading luminescence (integration time between 0.5 and 1 s). Replicate strictly standardized mean difference (SSMD) was used to determine the statistical effect size of each data point (treatment and dose grouped by replicate) relative to the untreated (basal non-stimulated) control using the formula for the robust uniformly minimal variance unbiased estimate (UMVUE) under the assumption that treatment has the same variance as the control.

Flow cytometry profiling of organoids in “2.5-D”. For flow cytometry profiling of the 6 validate small molecules, ISC-enriched ‘small clusters’ in 3D Matrigel culture were passaged to a “2.5D” 96-well plate culture system for six days of ENR, or ENR+CD+drug culture in the same manner as described previously with the exception of plating in 96-well plates prepared with a polymerized 70% Matrigel coating in each well. Plates were centrifuged at 50 g for 1 min at 4° C. to allow for cells to partially embed in Matrigel coating. Drugs were pinned into their respective wells using the Tecan from a drug stamp plate. Media was changed at day three, including pinning of the drug treatments. At day six, cells were washed 3× with basal media, then harvested from Matrigel by mechanical disruption in TrypLE Express to remove Matrigel and dissociate organoids to single cells. After vigorous pipetting and incubation at 37° C. for 20 mins, dissociated organoids were strained through a 96-well filter plate with a 30-40 m filter (Pall) into an ultra low-bind 96-well plate (Costar) by centrifuging at 300×g for 3 mins at 4° C. The cell filtrate was centrifuged again at 300×g for 3 mins at 4° C. to pellet the cells. Cell pellets were resuspended in FACS buffer (2% FBS in PBS0), then transferred to an ultra low-bind 96-well plate for flow prep. Cells were stained with Zombie-violet viability dye (BioLegend) at 100× for viability staining and/or antibody staining solution. FITC-conjugated antibody for lysozyme and APC-conjugated antibody for CD24 were used at 100× dilution (BioLegend). Flow cytometry was performed using a LSR Fortessa (BD; Koch Institute Flow Cytometry Core at MIT). Flow cytometry data was analyzed using FlowJo X v10.1 software.

Antibodies and Reagents. An antibody against lysozyme was purchased from abcam (Cambridge, Mass., ab108508). KPT-330 (S7252) and KPT-8602 (58397) were purchased from Selleck Chemicals (Houston, Tex.), and leptomycin B was purchased from Cayman Chemical (Ann Arbor, Mich.; 10004976).

Flow cytometry analysis (3D culture). ISC-enriched organoids cultured in 3D Matrigel with ENRCV media were passaged to 24-well plate in 3D Matrigel with ENRCV. At day zero, media were replaced to ENRCD with or without indicated compounds, and media were replaced every other day. At day six, cells were washed with basal media, and then harvested from Matrigel by mechanical disruption in TrypLE Express (ThermoFisher, 12605010) to remove Matrigel and dissociate organoids to single cells. After vigorous pipetting and incubation at 37° C. for 20 min, dissociated organoids were strained through a 35 μm cell strainer into a tube (Falcon, 352235). The cell filtrate was centrifuged again at 300×g for 3 min at 4° C. to pellet the cells. Cell pellets were resuspended in FACS buffer (2% FBS in PBS), and then transferred to an ultra low-bind 96-well plate (Corning, 7007). Cells were stained with Zombie-violet viability dye (BioLegend, 423107) at 100× for viability staining and/or antibody staining solution. FITC-conjugated antibody for lysozyme (Dako, F0372) and APC-conjugated antibody for CD24 (BioLegend, 138505) were used at 100× dilution. Flow cytometry was performed using an LSR Fortessa (BD; Koch Institute Flow Cytometry Core at MIT). The data were analyzed using FlowJo v10 software.

### Example 2—Inhibition of Nuclear Exporter Xpo1 Rebalances Intestinal Stem Cell Differentiation Towards Paneth Cells

Here, Applicants utilize a chemical-induction approach to model Paneth cell differentiation in vitro in an organoid system, and perform a phenotypic screen to identify pharmaceutically-actionable and biologically significant pathways which enhance Paneth cell differentiation independent of Wnt or Notch cues. Organoid models, broadly defined as three-dimensional, stem cell-derived, tissue-like cellular structures, have provided a powerful new tool to understand the adult stem cell niche, and key developmental pathways in stem cell differentiation (Sato et al., 2009; Yin et al., 2014). While high-throughput phenotypic drug discovery provides an efficient platform for the discovery and selection of Paneth cell-specific modulators, no prior knowledge of molecular mechanism of action is required (Ranga et al., 2014); however, high-fidelity models amendable to high-throughput screening are necessary to find effective therapeutics. Cellular models with improved physiological-representation of the intestine include tissue explants (Dionne et al., 2003), organoid models (Clevers, 2016), and organ-on-a-chip approaches (Bhatia and Ingber, 2014); however, these systems are often inflexible, heterogenous and cannot be scaled, limiting their utility in early drug discovery. Building on a protocol to grow ISC-enriched organoids and their apparent progeny, including Paneth cells (Yin et al., 2014) has offered a method to overcome limitations in scalability and high-fidelity lineage-specific representation (Mead et al., 2018). Using a rationally-designed organoid model to conduct a phenotypic screen, Applicants uncover novel targets and clinically-relevant small molecules which enhance ISC differentiation to the Paneth cell lineage. The results identify a series of inhibitors targeting nuclear exporter XPO1. Applicants validate differentiation trajectories by XPO1 inhibition with single-cell RNA-seq of organoids, and orthogonal studies. Overall, Applicants provide a framework to construct organoid models of lineage-specific differentiation that can uncover pathways regulating differentiation and discover compounds controlling barrier tissue composition.

### Small Molecule Screen for Regulators of Paneth Cell Differentiation

To enable small molecule screening in a model of Paneth cell development, Applicants employ a method of chemically-enriching and differentiating intestinal organoids from ISCs to Paneth cells. Murine intestinal organoids are conventionally expanded as heterogeneous structures in a culture media enriched with growth factors and small molecules intended to mimic the ISC niche, namely epidermal growth factor—EGF (E), BMP-antagonist noggin (N), and the aforementioned Wnt-pathway enhancer, R-spondin1 (R). The cellular structures within these cultures contain cycling ISCs and immature absorptive and secretory progeny, including Paneth cells. Compositionally-enriched and functionally-mature Paneth cells can be generated from an expanded ISC-enriched organoid population (cultured indefinitely in the presence of CHIR99021 (C) and valproic acid (V)) using a chemical differentiation with the small molecules (C) and DAPT (D) in a 3-D Matrigel scaffold, as Applicants have previously shown (Mead et al., 2018; Yin et al., 2014). ENR+CD offers a reproducible model from which to screen for new biology along the axis of ISC to Paneth differentiation and is amenable to high-throughput screening by measuring Paneth cell-specific function with a commercially-available assay for secreted lysozyme (LYZ).

However, the original 3-D ENR+CD system required the intricate plating of organoids within temperature-sensitive Matrigel domes, which presented a barrier to the secretion of Paneth cell-derived LYZ, and limited the capacity to automate screening assays. Applicants circumvented these limitations by adapting the existing 3-D system into a 2.5-D pseudo-monolayer platform, as described by others (Langhans, 2018). In brief, Applicants plate ISC-enriched organoids within 384-well plates partially embedded on the surface of a thick layer of Matrigel (at the Matrigel-media interface) rather than fully encapsulated in the Matrigel structure. This technique allows for Matrigel plating, cell seeding, and media additions to be performed in a fully-automated setting making it amenable to high-throughput applications, and allows LYZ secretion directly into cell culture media. Applicants therefore sought use of the scalable 2.5-D platform to demonstrate a proof-of-concept screen for developmental process of Paneth cells in vitro, and to elucidate molecular pathways and small molecule agents which may afford an axis to enhance Paneth cell number therapeutically.

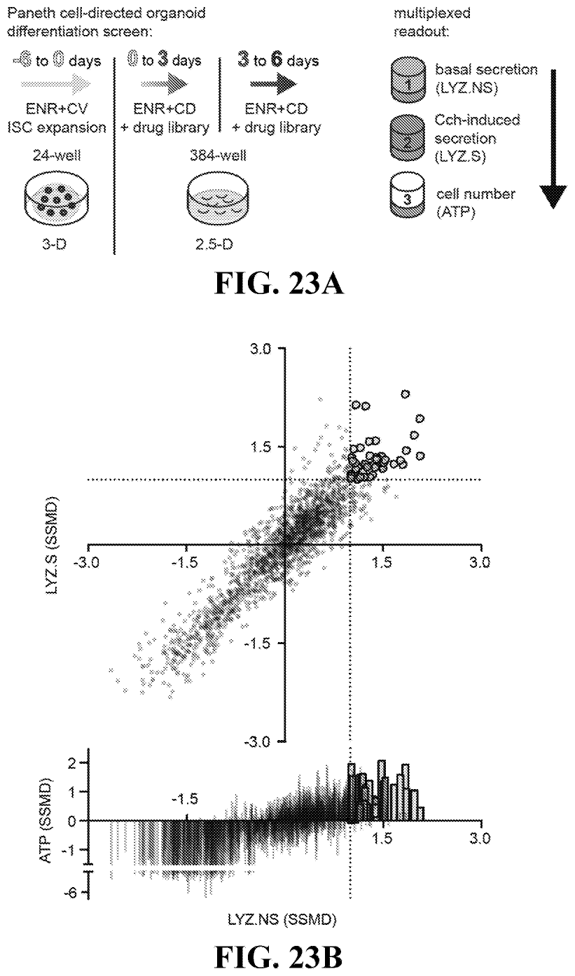

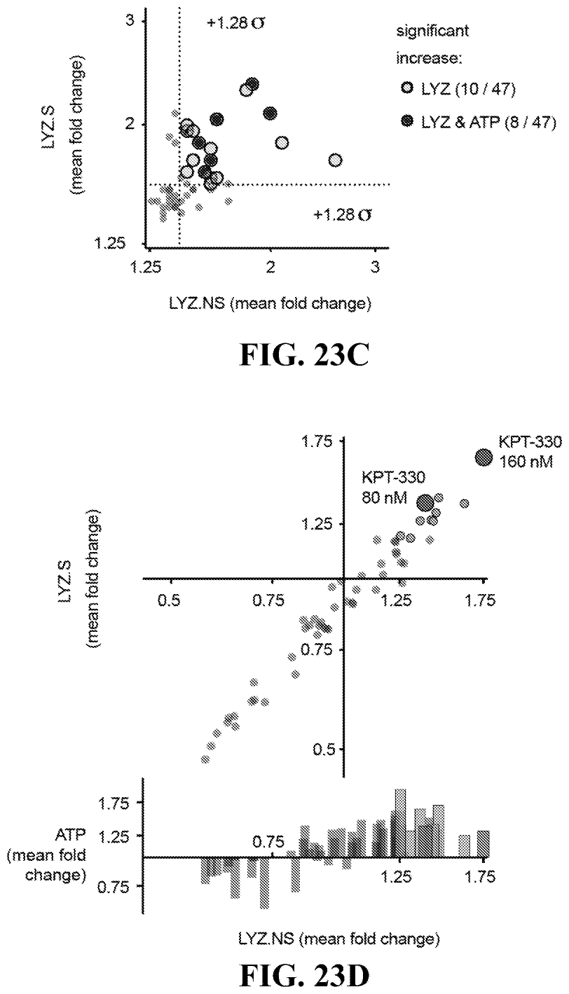

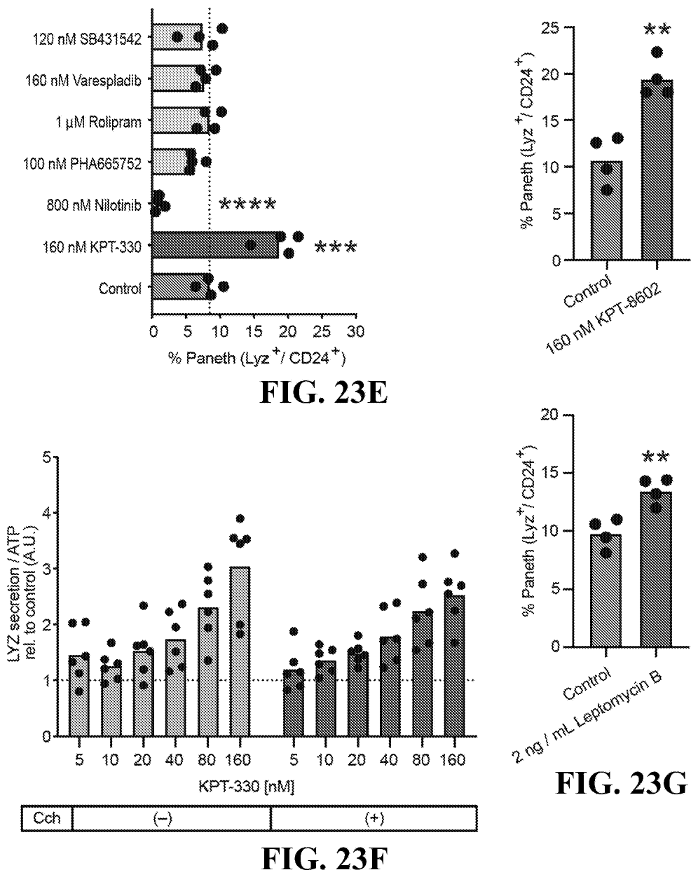

Applicants applied a small molecule library over a 6-day differentiation of ENR+CD organoids from ENR+CV ISC-enriched precursors (n=3 biological replicates) and at day 6, measured functional secretion of lysozyme (LYZ) in media supernatants (FIG. 23A). Small molecules were pinned into distinct wells at four doses per compound (‘quadrant stamp’) at day 0 (day of plating) and day 3 (media change). At day 6, cells were assayed for phenotypic Paneth cell markers—basal LYZ secretion (LYZ.NS) and carbachol (CCh)-induced secretion (LYZ.S)—and ATP as a measure of cell number using a CellTiterGlo (ATP) assay multiplexed within a given well. Applicants used a target-selective inhibitor library (Selleck Chem) with 184 unique molecular targets and 433 compounds with high specificity and many of those targets being implicated in stem cell differentiation (see Methods).

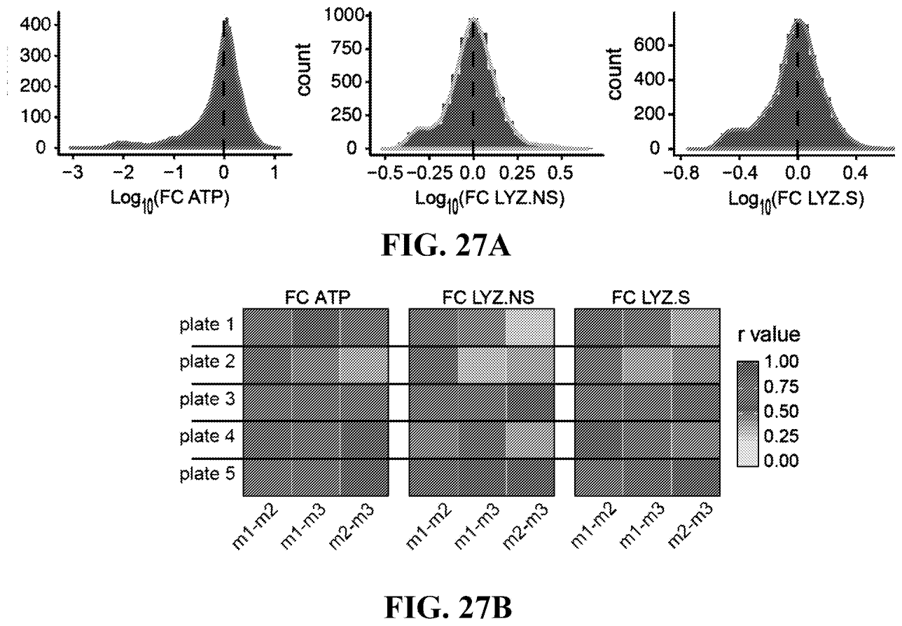

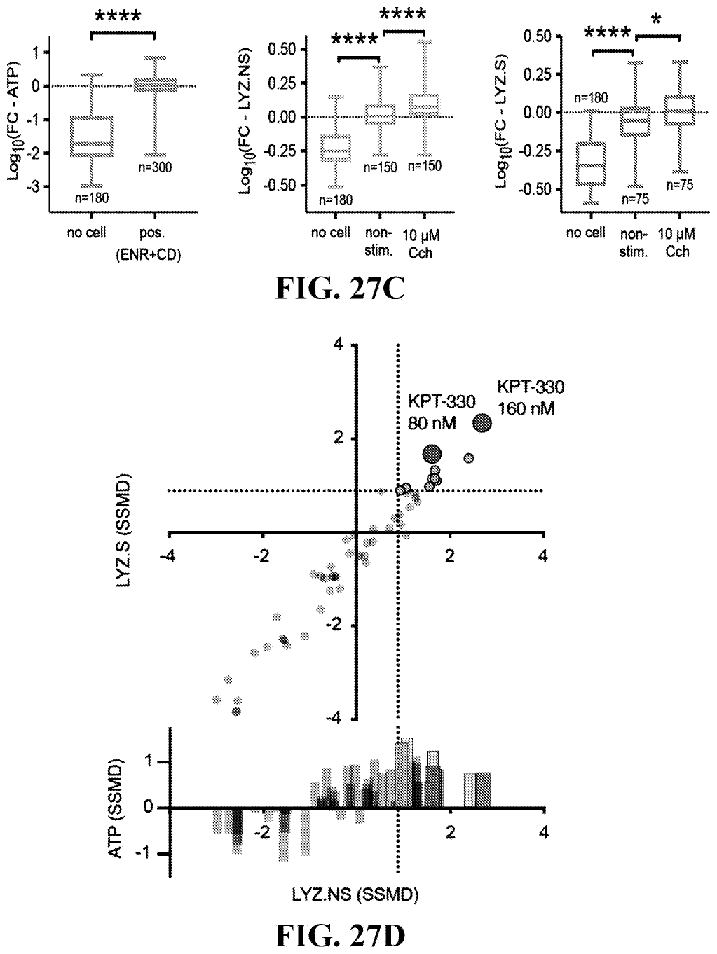

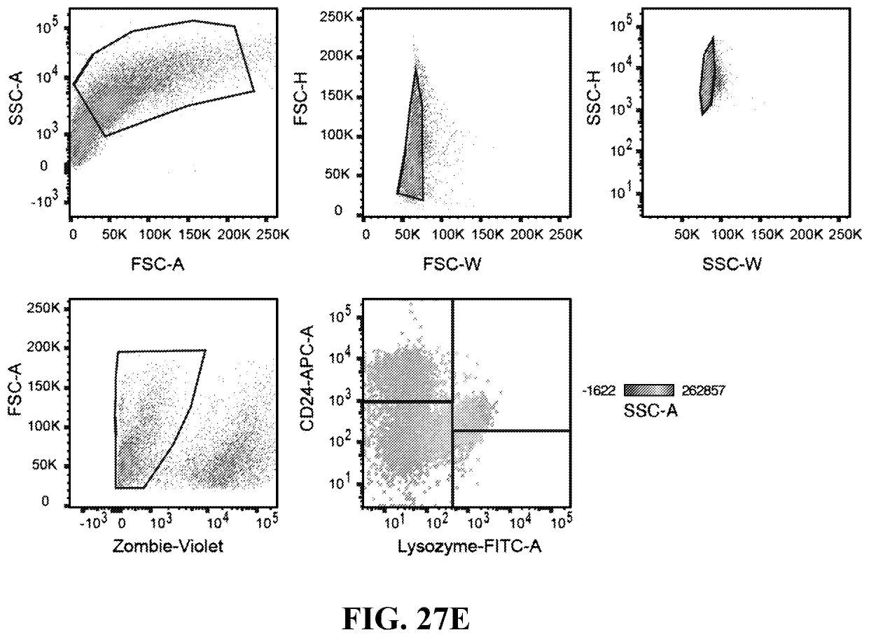

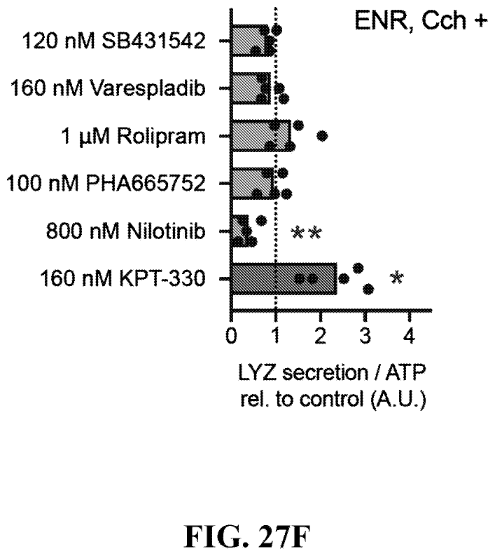

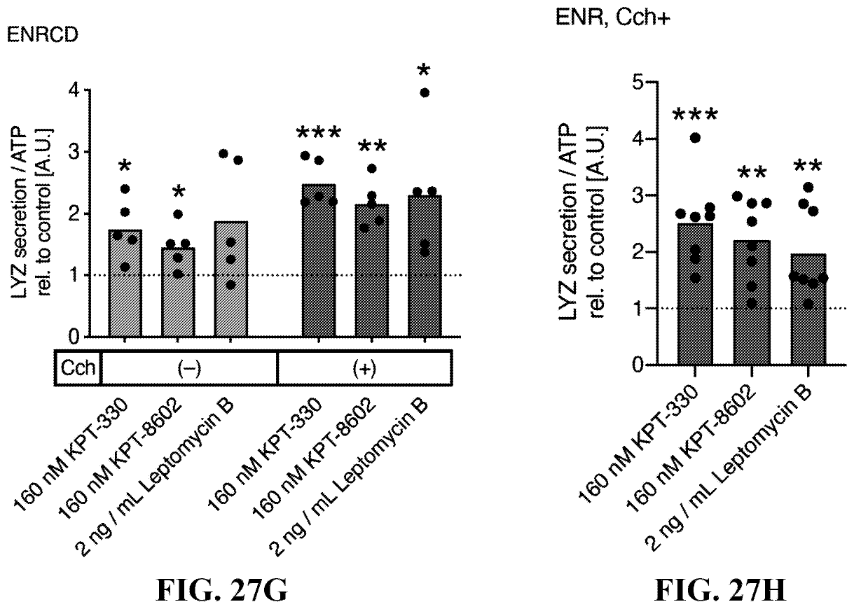

Screen plates were first normalized and assessed for reproducibility and quality. Briefly, raw values were log₁₀ transformed, then a LOESS normalization was applied to each plate to remove systematic error and column/row/edge effects (with wells for controls and compounds being randomly distributed throughout each plate). Following normalization, fold change (FC) was calculated by subtracting the median of the plate (as control) from the LOESS normalized values. For each assay this resulted in approximate-normal distributions, with lower-level tails corresponding to toxic compounds (FIG. 27A). Median FC was also used for each treatment-dose to determine robust z-score. Following data normalization, plates were assessed for correlations between biological replicates for all three assays, indicating an acceptable level of reproducibility across plates and biological replicates (FIG. 27B). As well, FCs of no-cell controls versus cell-containing positive controls (ENR+CD) wells was statistically significant (adj. p<0.0001), indicating positive control wells on average (across plates) contained viable cells (FIG. 27C). Discrimination of biological function was confirmed in the basal LYZ secretion assay (LYZ.NS), where non-stimulated positive controls had significantly greater secreted LYZ than that of no-cell controls (adj. p<0.0001), and 10 μM CCh-stimulated positive controls were significantly greater than that of non-stimulated positive controls (adj. p<0.0001) (FIG. 27C). Biological function was further observed in the stimulated LYZ secretion assay (LYZ.S), where a significant increase in secreted LYZ was observed in non-stimulated positive controls subsequently stimulated with 10 μM CCh versus non-stimulated (adj. p<0.05) and those doubly non-stimulated positive controls versus no cell controls (adj. p<0.0001) (FIG. 27C).

Primary screen ‘hits’ were defined as having replicate strictly standardized mean differences (SSMDs) for both LYZ assays greater than the calculated optimal critical value (#&₁=0.997) (FIG. 23B, data in Table 5A). β_{α}_{₁} was determined as the intersection of false positive and false negative levels (FPL & FNL) for up-regulation SSMD-based decisions (Zhang, 2011). This cutoff identified treatment-dose (grouped by biological replicate) combinations that had a statistical effect size greater than the FNL and FPL levels of the plates, i.e. treatments that had a statistically significant effect on increasing LYZ.NS and LYZ.S secretion without regard to viability (though most hits per this criteria had positive effects on cellular ATP). Following these criteria, 47 selected treatment-dose hits are thus hits in either both LYZ assays or all three assays, meaning hits either improve Paneth cell function and/or survival. The 47 hits were narrowed down to 15 treatment-dose combinations using the z-scored FC to select for combinations that elicited a biological effect in the top 10% of values for both LYZ assays relative to the plate (>1.282). Thus, 15 drugs (covering 18 treatment-dose conditions) from 13 unique molecular targets were identified as primary screen hits (FIG. 23C). For molecular targets with more than one hit treatment-dose-TGF-β inhibitors, PI3K/Akt/mTOR inhibitors, and Tyr kinase inhibitors-only the most robust treatment-dose was selected for further investigation. For TGF-β inhibitors, SB431542 performed almost identically to LY215799 on both LYZ assays but outperformed LY215799 on increasing cell viability and was thus selected as a hit. Both PI3K/Akt/mTOR inhibitors were selected as hits because their targets are substantially different and could mechanistically show a p70-specific effect or a multi-target effect within the whole pathway. For Tyr kinase inhibitors, only Rebastinib was excluded from the hit list due to underperformance on all three assays compared to Bosutinib, for with it shares a primary target. Optimal doses for treatments that were the sole hit in their respective pathway-Safinamide mesylate (catecholamine metabolism), Rolipram (cAMP), Varespladib (phospholipase inhibitor), Dapagliflozin (glucose transporter), KPT-330 (nuclear transporter), and finasteride (5a reductase inhibitor)—were determined by assessing dose response curves for both LYZ assays primarily and the CTG assay secondarily.

To validate primary screen hits against and ENR+CD control, identify narrowed dose-response ranges, and further narrow hits to only the most potent activator(s) of Paneth cell differentiation, Applicants performed secondary screening with the 13 primary screen compounds (Table 5B). Drugs were tested at a narrowed dose range around each treatment's identified optimal dose from the primary screen (4× below, 2× above and below). Hits in the validation screen were chosen by SSMDs for both LYZ assays greater than the calculated optimal critical value (β_{α1}=0.889), with 6 compounds passing this threshold (FIG. 27D). The same treatment-dose conditions passing the SSMD threshold also had the greatest biological effect, and in particular one compound, KPT-330, a known Xpo1 inhibitor, had two doses representing the greatest, and near-greatest biological effect (˜50-75% increases in LYZ.NS and LYZ.S relative to ENR+CD control) (FIG. 23D).

TABLE 5
Results from primary and secondary lysozyme (LYZ) secretion screening grouped by compound and dose,
reported as log2 fold change (FC), standard z score, and strictly standardized mean difference (SSMD).
Table 5A. Primary Screen

| | Dose | | | | | |
|---|---|---|---|---|---|---|
| Treatment | (uM) | Dose.FC.CTG | Dose.FC.LYZ.NS | Dose.FC.LYZ.S | Z.CTG | Z.LYZ.NS |
| (−)-MK 801 Maleate | 0.08 | 0.127 | 0.008 | 0.015 | 0.303 | 0.072 |
| (−)-MK 801 Maleate | 0.40 | −0.377 | −0.099 | −0.179 | −0.899 | −0.864 |
| (−)-MK 801 Maleate | 2.00 | −0.702 | −0.256 | −0.334 | −1.677 | −2.227 |
| (−)-MK 801 Maleate | 10.00 | −0.936 | −0.259 | −0.403 | −2.236 | −2.255 |
| (−)-Parthenolide | 0.08 | 0.135 | 0.058 | 0.124 | 0.322 | 0.506 |
| (−)-Parthenolide | 0.40 | 0.228 | 0.067 | 0.165 | 0.544 | 0.583 |
| (−)-Parthenolide | 2.00 | 0.157 | 0.089 | 0.068 | 0.376 | 0.776 |
| (−)-Parthenolide | 10.00 | 0.157 | 0.133 | 0.082 | 0.374 | 1.159 |
| (+)-Bicuculline | 0.08 | 0.318 | 0.092 | 0.155 | 0.760 | 0.805 |
| (+)-Bicuculline | 0.40 | −0.029 | 0.013 | 0.034 | −0.069 | 0.116 |
| (+)-Bicuculline | 2.00 | −0.166 | 0.031 | −0.026 | −0.396 | 0.270 |
| (+)-Bicuculline | 10.00 | −0.132 | 0.020 | 0.072 | −0.315 | 0.176 |
| (+)-JQ1 | 0.08 | −0.223 | −0.256 | −0.356 | −0.533 | −2.231 |
| (+)-JQ1 | 0.40 | −0.861 | −0.296 | −0.406 | −2.057 | −2.575 |
| (+)-JQ1 | 2.00 | −1.006 | −0.254 | −0.379 | −2.403 | −2.216 |
| (+)-JQ1 | 10.00 | −0.844 | −0.257 | −0.372 | −2.016 | −2.236 |
| (S)-crizotinib | 0.08 | 0.241 | 0.193 | 0.125 | 0.575 | 1.679 |
| (S)-crizotinib | 0.40 | 0.054 | 0.009 | 0.016 | 0.130 | 0.081 |
| (S)-crizotinib | 2.00 | 0.111 | −0.034 | −0.059 | 0.266 | −0.294 |
| (S)-crizotinib | 10.00 | 0.019 | −0.068 | −0.111 | 0.046 | −0.588 |
| 17-AAG (Tanespimycin) | 0.08 | −1.012 | −0.193 | −0.282 | −2.418 | −1.682 |
| 17-AAG (Tanespimycin) | 0.40 | −1.262 | −0.205 | −0.340 | −3.015 | −1.786 |
| 17-AAG (Tanespimycin) | 2.00 | −1.459 | −0.142 | −0.343 | −3.486 | −1.239 |
| 17-AAG (Tanespimycin) | 10.00 | −1.446 | −0.187 | −0.306 | −3.454 | −1.631 |
| 2-Methoxyestradiol (2-MeOE2) | 0.08 | 0.299 | 0.021 | 0.189 | 0.714 | 0.186 |
| 2-Methoxyestradiol (2-MeOE2) | 0.40 | 0.401 | 0.065 | 0.270 | 0.957 | 0.566 |
| 2-Methoxyestradiol (2-MeOE2) | 2.00 | 0.227 | 0.005 | 0.029 | 0.543 | 0.041 |
| 2-Methoxyestradiol (2-MeOE2) | 10.00 | −0.672 | −0.139 | −0.278 | −1.606 | −1.209 |
| 4E1RCat | 0.08 | 0.246 | 0.100 | 0.107 | 0.587 | 0.874 |
| 4E1RCat | 0.40 | 0.362 | −0.018 | 0.038 | 0.864 | −0.154 |
| 4E1RCat | 2.00 | 0.006 | 0.151 | 0.049 | 0.015 | 1.316 |
| 4E1RCat | 10.00 | −0.011 | 0.007 | 0.082 | −0.026 | 0.062 |
| 4EGI-1 | 0.08 | −0.160 | 0.110 | −0.037 | −0.383 | 0.956 |
| 4EGI-1 | 0.40 | −0.131 | 0.036 | 0.054 | −0.314 | 0.315 |
| 4EGI-1 | 2.00 | 0.127 | 0.069 | −0.059 | 0.304 | 0.597 |
| 4EGI-1 | 10.00 | −0.109 | −0.034 | 0.057 | −0.261 | −0.300 |
| 4CE°8C | 0.08 | −0.111 | 0.069 | 0.032 | −0.265 | 0.601 |
| 4CE°8C | 0.40 | 0.007 | 0.047 | 0.011 | 0.018 | 0.413 |
| 4CE°8C | 2.00 | −0.162 | 0.016 | −0.055 | −0.387 | 0.138 |
| 4CE°8C | 10.00 | −0.039 | −0.095 | −0.129 | −0.094 | −0.827 |
| 5-hydroxymethyl Tolterodine | 0.08 | −0.062 | 0.184 | 0.065 | −0.149 | 1.599 |
| (PNU 200577, 5-HMT, 5-HM) | | | | | | |
| 5-hydroxymethyl Tolterodine | 0.40 | 0.087 | 0.076 | 0.036 | 0.207 | 0.665 |
| (PNU 200577, 5-HMT, 5-HM) | | | | | | |
| 5-hydroxymethyl Tolterodine | 2.00 | 0.034 | 0.154 | 0.023 | 0.082 | 1.344 |
| (PNU 200577, 5-HMT, 5-HM) | | | | | | |
| 5-hydroxymethyl Tolterodine | 10.00 | 0.017 | 0.034 | −0.007 | 0.040 | 0.293 |
| (PNU 200577, 5-HMT, 5-HM) | | | | | | |
| A-769662 | 0.08 | −0.074 | 0.045 | 0.066 | −0.177 | 0.394 |
| A-769662 | 0.40 | 0.007 | 0.069 | 0.071 | 0.016 | 0.599 |
| A-769662 | 2.00 | −0.127 | 0.054 | 0.184 | −0.304 | 0.473 |
| A-769662 | 10.00 | −0.015 | 0.009 | 0.019 | −0.036 | 0.075 |
| ABT-199 (GDC-0199) | 0.08 | 0.019 | −0.002 | −0.044 | 0.045 | −0.013 |
| ABT-199 (GDC-0199) | 0.40 | 0.038 | −0.029 | 0.076 | 0.090 | −0.251 |
| ABT-199 (GDC-0199) | 2.00 | −0.278 | −0.016 | −0.063 | −0.665 | −0.136 |
| ABT-199 (GDC-0199) | 10.00 | −0.519 | −0.169 | −0.292 | −1.240 | −1.471 |
| ABT-263 (Navitoclax) | 0.08 | −0.212 | 0.012 | −0.010 | −0.507 | 0.104 |
| ABT-263 (Navitoclax) | 0.40 | −0.264 | 0.007 | −0.020 | −0.631 | 0.062 |
| ABT-263 (Navitoclax) | 2.00 | −0.368 | −0.143 | −0.205 | −0.878 | −1.248 |
| ABT-263 (Navitoclax) | 10.00 | −0.962 | −0.259 | −0.400 | −2.298 | −2.259 |
| Acadesine | 0.08 | 0.094 | 0.023 | 0.004 | 0.225 | 0.198 |
| Acadesine | 0.40 | 0.173 | −0.016 | −0.012 | 0.413 | −0.137 |
| Acadesine | 2.00 | −0.005 | 0.016 | 0.053 | −0.012 | 0.140 |
| Acadesine | 10.00 | 0.076 | −0.070 | −0.102 | 0.182 | −0.607 |
| ADL5859 HCl | 0.08 | 0.270 | 0.053 | 0.079 | 0.645 | 0.458 |
| ADL5859 HCl | 0.40 | 0.163 | 0.019 | 0.104 | 0.389 | 0.163 |
| ADL5859 HCl | 2.00 | 0.291 | 0.066 | 0.070 | 0.695 | 0.571 |
| ADL5859 HCl | 10.00 | 0.194 | −0.056 | −0.040 | 0.465 | −0.488 |
| ADX-47273 | 0.08 | 0.115 | 0.269 | 0.147 | 0.274 | 2.346 |
| ADX-47273 | 0.40 | −0.034 | 0.053 | 0.147 | −0.082 | 0.457 |
| ADX-47273 | 2.00 | −0.210 | −0.033 | −0.039 | −0.501 | −0.286 |
| ADX-47273 | 10.00 | −0.039 | −0.036 | −0.070 | −0.094 | −0.311 |
| AG-14361 | 0.08 | −0.012 | 0.152 | 0.100 | −0.028 | 1.324 |
| AG-14361 | 0.40 | −0.069 | 0.036 | −0.001 | −0.165 | 0.312 |
| AG-14361 | 2.00 | 0.005 | 0.032 | −0.065 | 0.012 | 0.278 |
| AG-14361 | 10.00 | −0.021 | −0.197 | −0.189 | −0.050 | −1.713 |
| AGI-5198 | 0.08 | 0.309 | 0.055 | 0.092 | 0.738 | 0.482 |
| AGI-5198 | 0.40 | 0.154 | 0.042 | 0.185 | 0.368 | 0.362 |
| AGI-5198 | 2.00 | 0.281 | 0.039 | 0.073 | 0.671 | 0.342 |
| AGI-5198 | 10.00 | 0.336 | −0.007 | −0.005 | 0.802 | −0.064 |
| AGI-6780 | 0.08 | 0.281 | 0.069 | 0.095 | 0.671 | 0.597 |
| AGI-6780 | 0.40 | 0.231 | 0.031 | 0.156 | 0.551 | 0.268 |
| AGI-6780 | 2.00 | 0.298 | 0.043 | 0.055 | 0.713 | 0.374 |
| AGI-6780 | 10.00 | 0.107 | −0.090 | −0.054 | 0.256 | −0.785 |
| Agomelatine | 0.08 | 0.106 | 0.026 | 0.090 | 0.252 | 0.227 |
| Agomelatine | 0.40 | 0.081 | 0.080 | 0.132 | 0.193 | 0.695 |
| Agomelatine | 2.00 | −0.198 | 0.069 | −0.037 | −0.472 | 0.597 |
| Agomelatine | 10.00 | 0.024 | −0.033 | 0.008 | 0.056 | −0.285 |
| Allopurinol | 0.08 | 0.212 | 0.019 | 0.026 | 0.506 | 0.168 |
| Allopurinol | 0.40 | −0.098 | −0.036 | −0.086 | −0.234 | −0.314 |
| Allopurinol | 2.00 | −0.162 | −0.004 | −0.064 | −0.387 | −0.036 |
| Allopurinol | 10.00 | −0.454 | −0.051 | −0.119 | −1.085 | −0.442 |
| Aloxistatin | 0.08 | −0.006 | 0.020 | 0.025 | −0.015 | 0.171 |
| Aloxistatin | 0.40 | −0.006 | −0.029 | −0.016 | −0.014 | −0.250 |
| Aloxistatin | 2.00 | −0.129 | −0.037 | −0.091 | −0.308 | −0.324 |
| Aloxistatin | 10.00 | −0.214 | −0.142 | −0.181 | −0.511 | −1.236 |
| AM1241 | 0.08 | 0.159 | 0.052 | 0.046 | 0.379 | 0.454 |
| AM1241 | 0.40 | −0.054 | 0.004 | 0.088 | −0.129 | 0.035 |
| AM1241 | 2.00 | −0.062 | −0.014 | −0.017 | −0.147 | −0.123 |
| AM1241 | 10.00 | −0.001 | −0.003 | −0.080 | −0.002 | −0.028 |
| AMG-517 | 0.08 | 0.161 | 0.020 | 0.084 | 0.385 | 0.178 |
| AMG-517 | 0.40 | 0.113 | 0.060 | 0.060 | 0.269 | 0.522 |
| AMG-517 | 2.00 | 0.112 | −0.006 | 0.002 | 0.268 | −0.051 |
| AMG-517 | 10.00 | 0.056 | 0.031 | 0.266 | 0.135 | 0.267 |
| Amlodipine | 0.08 | −0.351 | 0.026 | −0.042 | −0.837 | 0.228 |
| Amlodipine | 0.40 | −0.363 | 0.024 | −0.015 | −0.866 | 0.210 |
| Amlodipine | 2.00 | −0.372 | −0.065 | −0.122 | −0.888 | −0.568 |
| Amlodipine | 10.00 | −0.421 | −0.135 | −0.155 | −1.006 | −1.172 |
| Anacetrapib (MK-0859) | 0.08 | −0.090 | 0.013 | 0.051 | −0.214 | 0.112 |
| Anacetrapib (MK-0859) | 0.40 | 0.035 | 0.007 | −0.002 | 0.084 | 0.060 |
| Anacetrapib (MK-0859) | 2.00 | −0.094 | −0.015 | −0.046 | −0.225 | −0.129 |
| Anacetrapib (MK-0859) | 10.00 | 0.031 | −0.036 | −0.036 | 0.075 | −0.314 |
| Anastrozole | 0.08 | −0.117 | 0.035 | 0.045 | −0.279 | 0.305 |
| Anastrozole | 0.40 | 0.024 | 0.106 | 0.100 | 0.057 | 0.924 |
| Anastrozole | 2.00 | −0.058 | −0.003 | −0.008 | −0.139 | −0.026 |
| Anastrozole | 10.00 | −0.499 | −0.067 | −0.081 | −1.191 | −0.580 |
| Aniracetam | 0.08 | −0.088 | 0.089 | 0.043 | −0.211 | 0.774 |
| Aniracetam | 0.40 | 0.112 | 0.056 | 0.148 | 0.267 | 0.486 |
| Aniracetam | 2.00 | 0.054 | 0.032 | 0.005 | 0.130 | 0.277 |
| Aniracetam | 10.00 | 0.019 | 0.012 | −0.028 | 0.045 | 0.105 |
| AP26113 | 0.08 | −0.054 | −0.013 | 0.109 | −0.130 | −0.116 |
| AP26113 | 0.40 | 0.061 | −0.062 | −0.038 | 0.145 | −0.541 |
| AP26113 | 2.00 | −0.558 | −0.196 | −0.244 | −1.333 | −1.706 |
| AP26113 | 10.00 | −1.680 | −0.272 | −0.343 | −4.012 | −2.370 |
| Apatinib | 0.08 | 0.223 | 0.103 | 0.173 | 0.532 | 0.897 |
| Apatinib | 0.40 | 0.302 | 0.038 | 0.142 | 0.722 | 0.327 |
| Apatinib | 2.00 | 0.270 | 0.076 | 0.112 | 0.645 | 0.660 |
| Apatinib | 10.00 | 0.218 | 0.012 | 0.075 | 0.520 | 0.101 |
| Apigenin | 0.08 | 0.246 | 0.132 | 0.192 | 0.588 | 1.147 |
| Apigenin | 0.40 | 0.214 | 0.130 | 0.165 | 0.510 | 1.131 |
| Apigenin | 2.00 | 0.315 | 0.232 | 0.196 | 0.753 | 2.021 |
| Apigenin | 10.00 | 0.026 | 0.097 | 0.098 | 0.063 | 0.847 |
| Apixaban | 0.08 | −0.089 | 0.004 | 0.081 | −0.212 | 0.035 |
| Apixaban | 0.40 | 0.111 | −0.030 | 0.029 | 0.266 | −0.260 |
| Apixaban | 2.00 | −0.060 | 0.029 | 0.051 | −0.144 | 0.253 |
| Apixaban | 10.00 | −0.077 | −0.082 | −0.096 | −0.185 | −0.712 |
| Apoptosis Activator 2 | 0.08 | 0.304 | 0.149 | 0.132 | 0.725 | 1.302 |
| Apoptosis Activator 2 | 0.40 | 0.262 | 0.033 | 0.073 | 0.626 | 0.290 |
| Apoptosis Activator 2 | 2.00 | 0.082 | −0.006 | −0.009 | 0.195 | −0.049 |
| Apoptosis Activator 2 | 10.00 | −0.158 | −0.199 | −0.252 | −0.378 | −1.734 |
| Aprepitant | 0.08 | 0.313 | 0.097 | 0.115 | 0.747 | 0.840 |
| Aprepitant | 0.40 | 0.403 | 0.061 | 0.107 | 0.962 | 0.532 |
| Aprepitant | 2.00 | 0.145 | 0.005 | −0.007 | 0.346 | 0.043 |
| Aprepitant | 10.00 | 0.051 | −0.090 | −0.119 | 0.121 | −0.780 |
| AS-252424 | 0.08 | 0.139 | 0.070 | 0.072 | 0.333 | 0.609 |
| AS-252424 | 0.40 | −0.074 | −0.042 | −0.008 | −0.176 | −0.368 |
| AS-252424 | 2.00 | −0.359 | −0.080 | −0.074 | −0.858 | −0.695 |
| AS-252424 | 10.00 | −0.204 | −0.077 | −0.022 | −0.487 | −0.671 |
| AT101 | 0.08 | −0.198 | −0.049 | 0.004 | −0.472 | −0.426 |
| AT101 | 0.40 | 0.019 | −0.068 | −0.013 | 0.044 | −0.592 |
| AT101 | 2.00 | −0.216 | −0.036 | −0.085 | −0.515 | −0.311 |
| AT101 | 10.00 | −0.056 | −0.052 | −0.061 | −0.133 | −0.452 |
| Ataluren (PTC124) | 0.08 | 0.267 | 0.029 | 0.051 | 0.639 | 0.253 |
| Ataluren (PTC124) | 0.40 | −0.340 | 0.042 | 0.034 | −0.813 | 0.362 |
| Ataluren (PTC124) | 2.00 | −0.042 | 0.131 | 0.058 | −0.100 | 1.140 |
| Ataluren (PTC124) | 10.00 | 0.006 | 0.009 | 0.005 | 0.013 | 0.077 |
| Atglistatin | 0.08 | 0.057 | 0.069 | 0.028 | 0.137 | 0.604 |
| Atglistatin | 0.40 | 0.004 | 0.135 | −0.035 | 0.009 | 1.176 |
| Atglistatin | 2.00 | −0.045 | 0.070 | 0.026 | −0.108 | 0.613 |
| Atglistatin | 10.00 | −0.431 | −0.016 | 0.012 | −1.031 | −0.142 |
| Atorvastatin Calcium | 0.08 | −0.129 | −0.009 | 0.022 | −0.309 | −0.077 |
| Atorvastatin Calcium | 0.40 | −0.348 | 0.007 | −0.062 | −0.831 | 0.062 |
| Atorvastatin Calcium | 2.00 | −1.085 | −0.247 | −0.354 | −2.591 | −2.148 |
| Atorvastatin Calcium | 10.00 | −1.583 | −0.235 | −0.305 | −3.780 | −2.045 |
| AUY922 (NVP-AUY922) | 0.08 | −1.185 | −0.144 | −0.209 | −2.830 | −1.251 |
| AUY922 (NVP-AUY922) | 0.40 | −1.343 | −0.154 | −0.184 | −3.208 | −1.343 |
| AUY922 (NVP-AUY922) | 2.00 | −1.137 | −0.138 | −0.216 | −2.715 | −1.200 |
| AUY922 (NVP-AUY922) | 10.00 | −1.270 | −0.153 | −0.214 | −3.032 | −1.330 |
| AVL-292 | 0.08 | 0.217 | 0.095 | 0.235 | 0.518 | 0.831 |
| AVL-292 | 0.40 | 0.057 | 0.052 | 0.106 | 0.135 | 0.451 |
| AVL-292 | 2.00 | −0.058 | −0.112 | −0.206 | −0.138 | −0.979 |
| AVL-292 | 10.00 | −0.778 | −0.213 | −0.376 | −1.859 | −1.858 |
| AZ 3146 | 0.08 | −0.130 | 0.073 | 0.049 | −0.310 | 0.639 |
| AZ 3146 | 0.40 | −0.262 | −0.038 | −0.067 | −0.626 | −0.327 |
| AZ 3146 | 2.00 | −0.369 | −0.079 | −0.130 | −0.881 | −0.687 |
| AZ 3146 | 10.00 | −1.168 | −0.281 | −0.422 | −2.789 | −2.447 |
| AZ191 | 0.08 | 0.292 | 0.128 | 0.143 | 0.697 | 1.111 |
| AZ191 | 0.40 | 0.337 | 0.082 | 0.199 | 0.804 | 0.717 |
| AZ191 | 2.00 | 0.150 | 0.058 | 0.097 | 0.358 | 0.507 |
| AZ191 | 10.00 | −0.533 | −0.148 | −0.200 | −1.272 | −1.289 |
| AZ20 | 0.08 | −0.210 | 0.042 | −0.038 | −0.500 | 0.365 |
| AZ20 | 0.40 | −0.218 | −0.140 | −0.236 | −0.521 | −1.222 |
| AZ20 | 2.00 | −1.054 | −0.215 | −0.373 | −2.519 | −1.868 |
| AZ20 | 10.00 | −0.573 | −0.261 | −0.329 | −1.368 | −2.271 |
| AZD1981 | 0.08 | 0.400 | 0.009 | 0.023 | 0.955 | 0.081 |
| AZD1981 | 0.40 | 0.285 | 0.001 | 0.191 | 0.681 | 0.008 |
| AZD1981 | 2.00 | 0.099 | 0.001 | 0.014 | 0.236 | 0.011 |
| AZD1981 | 10.00 | 0.238 | 0.024 | 0.070 | 0.570 | 0.205 |
| AZD2461 | 0.08 | 0.073 | 0.038 | 0.097 | 0.174 | 0.334 |
| AZD2461 | 0.40 | −0.040 | −0.038 | 0.015 | −0.095 | −0.327 |
| AZD2461 | 2.00 | −0.232 | −0.074 | −0.100 | −0.555 | −0.641 |
| AZD2461 | 10.00 | −0.336 | −0.172 | −0.224 | −0.803 | −1.497 |
| AZD3463 | 0.08 | −0.275 | −0.147 | −0.199 | −0.657 | −1.279 |
| AZD3463 | 0.40 | −0.341 | −0.190 | −0.293 | −0.814 | −1.652 |
| AZD3463 | 2.00 | −0.772 | −0.232 | −0.315 | −1.843 | −2.021 |
| AZD3463 | 10.00 | −0.960 | −0.209 | −0.328 | −2.293 | −1.820 |
| AZD4547 | 0.08 | 0.160 | 0.100 | 0.157 | 0.382 | 0.869 |
| AZD4547 | 0.40 | 0.101 | 0.059 | 0.083 | 0.241 | 0.517 |
| AZD4547 | 2.00 | −0.010 | 0.068 | 0.074 | −0.024 | 0.592 |
| AZD4547 | 10.00 | −0.062 | −0.038 | −0.049 | −0.147 | −0.331 |
| AZD6482 | 0.08 | 0.240 | 0.108 | 0.182 | 0.572 | 0.944 |
| AZD6482 | 0.40 | 0.223 | 0.076 | 0.156 | 0.533 | 0.660 |
| AZD6482 | 2.00 | −0.509 | −0.130 | −0.114 | −1.215 | −1.129 |
| AZD6482 | 10.00 | −1.203 | −0.248 | −0.253 | −2.874 | −2.160 |
| AZD7545 | 0.08 | −0.334 | 0.087 | 0.111 | −0.798 | 0.758 |
| AZD7545 | 0.40 | −0.158 | −0.028 | −0.017 | −0.378 | −0.245 |
| AZD7545 | 2.00 | −0.255 | −0.018 | −0.148 | −0.608 | −0.155 |
| AZD7545 | 10.00 | −0.350 | −0.051 | −0.104 | −0.836 | −0.441 |
| AZD7762 | 0.08 | 0.054 | 0.010 | 0.019 | 0.129 | 0.084 |
| AZD7762 | 0.40 | −0.384 | −0.159 | −0.251 | −0.917 | −1.385 |
| AZD7762 | 2.00 | −0.804 | −0.211 | −0.342 | −1.920 | −1.835 |
| AZD7762 | 10.00 | −1.361 | −0.220 | −0.369 | −3.251 | −1.917 |
| BAM7 | 0.08 | 0.209 | 0.045 | 0.079 | 0.498 | 0.391 |
| BAM7 | 0.40 | −0.010 | −0.009 | 0.039 | −0.024 | −0.082 |
| BAM7 | 2.00 | −0.156 | 0.057 | 0.019 | −0.373 | 0.498 |
| BAM7 | 10.00 | −0.114 | −0.007 | −0.020 | −0.272 | −0.063 |
| Batimastat (BB-94) | 0.08 | 0.035 | −0.003 | 0.053 | 0.084 | −0.025 |
| Batimastat (BB-94) | 0.40 | −0.095 | 0.005 | 0.028 | −0.226 | 0.042 |
| Batimastat (BB-94) | 2.00 | −0.049 | 0.081 | 0.056 | −0.117 | 0.707 |
| Batimastat (BB-94) | 10.00 | −0.283 | −0.075 | −0.107 | −0.676 | −0.649 |
| Bazedoxifene HCl | 0.08 | 0.422 | 0.159 | 0.096 | 1.009 | 1.386 |
| Bazedoxifene HCl | 0.40 | 0.365 | 0.026 | 0.065 | 0.873 | 0.229 |
| Bazedoxifene HCl | 2.00 | −0.780 | −0.146 | −0.189 | −1.862 | −1.267 |
| Bazedoxifene HCl | 10.00 | −1.148 | −0.280 | −0.361 | −2.741 | −2.442 |
| Belinostat (PXD101) | 0.08 | −0.227 | 0.008 | −0.059 | −0.543 | 0.067 |
| Belinostat (PXD101) | 0.40 | −0.055 | 0.129 | 0.042 | −0.133 | 1.125 |
| Belinostat (PXD101) | 2.00 | −0.782 | −0.173 | −0.264 | −1.868 | −1.510 |
| Belinostat (PXD101) | 10.00 | −1.122 | −0.196 | −0.327 | −2.679 | −1.709 |
| Bergenin | 0.08 | 0.288 | 0.164 | 0.198 | 0.687 | 1.432 |
| Bergenin | 0.40 | 0.245 | 0.098 | 0.108 | 0.585 | 0.852 |
| Bergenin | 2.00 | 0.205 | 0.080 | 0.125 | 0.491 | 0.699 |
| Bergenin | 10.00 | 0.344 | 0.103 | 0.144 | 0.821 | 0.898 |
| BGJ398 (NVP-BGJ398) | 0.08 | −0.054 | −0.002 | −0.036 | −0.128 | −0.017 |
| BGJ398 (NVP-BGJ398) | 0.40 | 0.026 | −0.060 | 0.002 | 0.063 | −0.520 |
| BGJ398 (NVP-BGJ398) | 2.00 | −0.185 | −0.052 | −0.062 | −0.442 | −0.450 |
| BGJ398 (NVP-BGJ398) | 10.00 | −1.500 | −0.269 | −0.350 | −3.583 | −2.345 |
| BI 2536 | 0.08 | 0.029 | −0.104 | −0.163 | 0.069 | −0.907 |
| BI 2536 | 0.40 | −0.534 | −0.129 | −0.220 | −1.275 | −1.121 |
| BI 2536 | 2.00 | −0.511 | −0.134 | −0.224 | −1.220 | −1.168 |
| BI 2536 | 10.00 | −1.028 | −0.109 | −0.185 | −2.455 | −0.953 |
| BI-D1870 | 0.08 | 0.186 | 0.141 | 0.162 | 0.444 | 1.225 |
| BI-D1870 | 0.40 | 0.029 | 0.126 | 0.158 | 0.069 | 1.099 |
| BI-D1870 | 2.00 | −0.012 | −0.066 | 0.008 | −0.028 | −0.574 |
| BI-D1870 | 10.00 | −0.700 | −0.285 | −0.378 | −1.673 | −2.483 |
| BIBR 1532 | 0.08 | 0.223 | 0.032 | 0.028 | 0.533 | 0.282 |
| BIBR 1532 | 0.40 | −0.047 | 0.043 | 0.107 | −0.113 | 0.377 |
| BIBR 1532 | 2.00 | −0.034 | −0.013 | −0.081 | −0.080 | −0.117 |
| BIBR 1532 | 10.00 | 0.030 | −0.062 | −0.114 | 0.072 | −0.536 |
| Birinapant | 0.08 | −0.257 | 0.032 | 0.075 | −0.615 | 0.281 |
| Birinapant | 0.40 | 0.095 | −0.012 | 0.103 | 0.226 | −0.102 |
| Birinapant | 2.00 | 0.227 | 0.036 | 0.011 | 0.542 | 0.317 |
| Birinapant | 10.00 | 0.123 | −0.029 | −0.080 | 0.293 | −0.256 |
| Bisoprolol fumarate | 0.08 | −0.104 | 0.085 | 0.086 | −0.249 | 0.739 |
| Bisoprolol fumarate | 0.40 | −0.057 | 0.075 | 0.129 | −0.136 | 0.649 |
| Bisoprolol fumarate | 2.00 | −0.020 | 0.075 | 0.074 | −0.048 | 0.656 |
| Bisoprolol fumarate | 10.00 | 0.058 | 0.003 | 0.015 | 0.139 | 0.027 |
| BML-190 | 0.08 | −0.003 | 0.072 | 0.060 | −0.007 | 0.623 |
| BML-190 | 0.40 | −0.099 | −0.017 | −0.037 | −0.237 | −0.152 |
| BML-190 | 2.00 | −0.106 | 0.116 | 0.017 | −0.254 | 1.011 |
| BML-190 | 10.00 | −0.106 | 0.209 | 0.067 | −0.254 | 1.823 |
| BMS-378806 | 0.08 | 0.233 | 0.141 | 0.159 | 0.557 | 1.231 |
| BM5-378806 | 0.40 | 0.102 | −0.007 | 0.007 | 0.243 | −0.061 |
| BMS-378806 | 2.00 | −0.289 | −0.100 | −0.142 | −0.691 | −0.870 |
| BMS-378806 | 10.00 | −1.155 | −0.227 | −0.322 | −2.758 | −1.977 |
| BMS-707035 | 0.08 | 0.318 | 0.107 | 0.134 | 0.759 | 0.933 |
| BMS-707035 | 0.40 | 0.238 | 0.135 | 0.182 | 0.568 | 1.174 |
| BMS-707035 | 2.00 | 0.279 | 0.110 | 0.048 | 0.667 | 0.955 |
| BMS-707035 | 10.00 | 0.138 | 0.070 | 0.050 | 0.330 | 0.613 |
| Bortezomib (PS-341) | 0.08 | −1.904 | −0.148 | −0.239 | −4.547 | −1.285 |
| Bortezomib (PS-341) | 0.40 | −2.146 | −0.105 | −0.173 | −5.127 | −0.917 |
| Bortezomib (PS-341) | 2.00 | −0.915 | −0.133 | −0.249 | −2.185 | −1.158 |
| Bortezomib (PS-341) | 10.00 | −1.359 | −0.153 | −0.238 | −3.246 | −1.334 |
| Bosentan Hydrate | 0.08 | 0.204 | 0.090 | 0.145 | 0.487 | 0.785 |
| Bosentan Hydrate | 0.40 | 0.207 | 0.068 | 0.100 | 0.495 | 0.589 |
| Bosentan Hydrate | 2.00 | 0.121 | 0.039 | 0.014 | 0.288 | 0.337 |
| Bosentan Hydrate | 10.00 | 0.085 | 0.058 | 0.045 | 0.204 | 0.508 |
| Bosutinib (SKI-606) | 0.08 | 0.628 | 0.213 | 0.308 | 1.499 | 1.858 |
| Bosutinib (SKI-606) | 0.40 | 0.302 | 0.130 | 0.208 | 0.720 | 1.129 |
| Bosutinib (SKI-606) | 2.00 | −0.169 | −0.049 | −0.058 | −0.405 | −0.425 |
| Bosutinib (SKI-606) | 10.00 | −0.668 | −0.115 | −0.223 | −1.595 | −1.005 |
| Brinzolamide | 0.08 | −0.007 | 0.133 | 0.030 | −0.017 | 1.159 |
| Brinzolamide | 0.40 | −0.068 | 0.084 | 0.052 | −0.163 | 0.733 |
| Brinzolamide | 2.00 | 0.118 | 0.059 | 0.064 | 0.282 | 0.511 |
| Brinzolamide | 10.00 | 0.078 | 0.060 | 0.083 | 0.186 | 0.521 |
| BTB06584 | 0.08 | 0.275 | 0.091 | 0.222 | 0.657 | 0.795 |
| BTB06584 | 0.40 | 0.288 | 0.025 | 0.042 | 0.688 | 0.214 |
| BTB06584 | 2.00 | −0.180 | 0.024 | 0.039 | −0.431 | 0.206 |
| BTB06584 | 10.00 | 0.199 | −0.031 | 0.019 | 0.475 | −0.268 |
| BTZ043 Racemate | 0.08 | 0.497 | 0.131 | 0.199 | 1.187 | 1.138 |
| BTZ043 Racemate | 0.40 | 0.545 | 0.186 | 0.252 | 1.303 | 1.619 |
| BTZ043 Racemate | 2.00 | 0.063 | 0.089 | 0.038 | 0.150 | 0.776 |
| BTZ043 Racemate | 10.00 | 0.327 | 0.100 | 0.070 | 0.782 | 0.870 |
| Bupivacaine HCl | 0.08 | −0.003 | 0.002 | 0.082 | −0.008 | 0.016 |
| Bupivacaine HCl | 0.40 | −0.217 | −0.100 | −0.169 | −0.517 | −0.871 |
| Bupivacaine HCl | 2.00 | −0.300 | −0.137 | −0.210 | −0.717 | −1.196 |
| Bupivacaine HCl | 10.00 | −0.364 | −0.195 | −0.295 | −0.870 | −1.700 |
| BX-912 | 0.08 | 0.043 | 0.010 | 0.038 | 0.103 | 0.090 |
| BX-912 | 0.40 | 0.054 | 0.017 | 0.042 | 0.129 | 0.151 |
| BX-912 | 2.00 | −0.246 | −0.089 | −0.161 | −0.587 | −0.771 |
| BX-912 | 10.00 | −0.397 | −0.196 | −0.272 | −0.949 | −1.704 |
| C646 | 0.08 | 0.346 | 0.057 | 0.092 | 0.826 | 0.492 |
| C646 | 0.40 | 0.119 | 0.089 | 0.025 | 0.285 | 0.775 |
| C646 | 2.00 | 0.010 | 0.160 | 0.101 | 0.023 | 1.395 |
| C646 | 10.00 | 0.047 | 0.054 | 0.102 | 0.111 | 0.467 |
| Caffeic Acid Phenethyl Ester | 0.08 | 0.262 | 0.053 | 0.189 | 0.627 | 0.465 |
| Caffeic Acid Phenethyl Ester | 0.40 | −0.011 | 0.167 | 0.070 | −0.026 | 1.453 |
| Caffeic Acid Phenethyl Ester | 2.00 | −0.064 | 0.090 | −0.021 | −0.152 | 0.781 |
| Caffeic Acid Phenethyl Ester | 10.00 | −0.511 | −0.026 | −0.117 | −1.221 | −0.227 |
| Canagliflozin | 0.08 | −0.240 | −0.027 | 0.014 | −0.572 | −0.233 |
| Canagliflozin | 0.40 | −0.098 | −0.046 | −0.032 | −0.235 | −0.398 |
| Canagliflozin | 2.00 | 0.042 | 0.025 | 0.028 | 0.101 | 0.214 |
| Canagliflozin | 10.00 | −0.079 | −0.064 | −0.033 | −0.188 | −0.556 |
| Candesartan | 0.08 | −0.003 | 0.013 | −0.015 | −0.008 | 0.113 |
| Candesartan | 0.40 | −0.125 | −0.015 | −0.032 | −0.299 | −0.128 |
| Candesartan | 2.00 | −0.198 | 0.007 | −0.092 | −0.473 | 0.065 |
| Candesartan | 10.00 | −0.253 | −0.051 | −0.075 | −0.604 | −0.442 |
| Captopril | 0.08 | −0.056 | 0.059 | 0.049 | −0.134 | 0.511 |
| Captopril | 0.40 | 0.108 | 0.035 | 0.109 | 0.257 | 0.307 |
| Captopril | 2.00 | 0.123 | 0.033 | 0.051 | 0.294 | 0.285 |
| Captopril | 10.00 | 0.107 | 0.153 | 0.077 | 0.255 | 1.330 |
| Carvedilol | 0.08 | 0.057 | 0.002 | −0.009 | 0.136 | 0.021 |
| Carvedilol | 0.40 | −0.014 | −0.020 | −0.079 | −0.034 | −0.175 |
| Carvedilol | 2.00 | −0.151 | 0.099 | −0.087 | −0.360 | 0.866 |
| Carvedilol | 10.00 | −0.242 | −0.053 | 0.019 | −0.577 | −0.464 |
| CCT128930 | 0.08 | 0.095 | 0.323 | 0.266 | 0.228 | 2.811 |
| CCT128930 | 0.40 | −0.025 | −0.018 | −0.007 | −0.060 | −0.154 |
| CCT128930 | 2.00 | 0.127 | 0.073 | 0.053 | 0.304 | 0.633 |
| CCT128930 | 10.00 | −0.175 | −0.150 | −0.201 | −0.418 | −1.307 |
| Celecoxib | 0.08 | −0.034 | −0.041 | −0.067 | −0.082 | −0.359 |
| Celecoxib | 0.40 | −0.299 | −0.053 | −0.068 | −0.714 | −0.463 |
| Celecoxib | 2.00 | −0.118 | −0.084 | −0.097 | −0.282 | −0.734 |
| Celecoxib | 10.00 | −0.128 | −0.099 | −0.133 | −0.306 | −0.865 |
| CEP-18770 (Delanzomib) | 0.08 | −0.953 | −0.108 | −0.179 | −2.276 | −0.937 |
| CEP-18770 (Delanzomib) | 0.40 | −1.642 | −0.076 | −0.159 | −3.923 | −0.664 |
| CEP-18770 (Delanzomib) | 2.00 | −0.990 | −0.063 | −0.180 | −2.364 | −0.546 |
| CEP-18770 (Delanzomib) | 10.00 | −1.148 | −0.035 | −0.149 | −2.742 | −0.308 |
| CGK733 | 0.08 | −0.362 | 0.099 | 0.008 | −0.864 | 0.863 |
| CGK733 | 0.40 | −0.093 | −0.026 | −0.018 | −0.221 | −0.222 |
| CGK733 | 2.00 | −0.244 | −0.099 | −0.125 | −0.582 | −0.865 |
| CGK733 | 10.00 | −0.478 | −0.122 | −0.230 | −1.143 | −1.063 |
| CGP57380 | 0.08 | 0.108 | 0.218 | 0.096 | 0.258 | 1.900 |
| CGP57380 | 0.40 | −0.293 | −0.041 | 0.054 | −0.700 | −0.354 |
| CGP57380 | 2.00 | −0.460 | −0.055 | −0.095 | −1.098 | −0.477 |
| CGP57380 | 10.00 | 0.055 | −0.037 | −0.088 | 0.132 | −0.321 |
| CGS 21680 HCl | 0.08 | 0.303 | 0.132 | 0.180 | 0.724 | 1.151 |
| CGS 21680 HCl | 0.40 | 0.439 | 0.167 | 0.191 | 1.049 | 1.452 |
| CGS 21680 HCl | 2.00 | 0.335 | 0.055 | 0.097 | 0.799 | 0.483 |
| CGS 21680 HCl | 10.00 | −1.393 | −0.251 | −0.334 | −3.328 | −2.187 |
| CHIR-124 | 0.08 | −0.256 | −0.155 | −0.267 | −0.612 | −1.352 |
| CHIR-124 | 0.40 | −0.853 | −0.240 | −0.339 | −2.038 | −2.090 |
| CHIR-124 | 2.00 | −0.861 | −0.220 | −0.352 | −2.057 | −1.916 |
| CHIR-124 | 10.00 | −1.555 | −0.140 | −0.332 | −3.714 | −1.223 |
| CHIR-98014 | 0.08 | 0.092 | 0.052 | 0.152 | 0.220 | 0.454 |
| CHIR-98014 | 0.40 | 0.080 | −0.053 | −0.058 | 0.190 | −0.464 |
| CHIR-98014 | 2.00 | 0.040 | −0.128 | −0.118 | 0.094 | −1.113 |
| CHIR-98014 | 10.00 | −0.052 | −0.218 | −0.329 | −0.124 | −1.897 |
| Cilomilast | 0.08 | −0.334 | 0.114 | 0.115 | −0.799 | 0.995 |
| Cilomilast | 0.40 | −0.338 | −0.037 | −0.109 | −0.807 | −0.321 |
| Cilomilast | 2.00 | −0.235 | −0.010 | −0.083 | −0.562 | −0.083 |
| Cilomilast | 10.00 | −0.192 | −0.043 | −0.045 | −0.458 | −0.376 |
| Cinacalcet HCl | 0.08 | −0.444 | −0.062 | −0.084 | −1.061 | −0.536 |
| Cinacalcet HCl | 0.40 | −0.298 | −0.023 | 0.039 | −0.712 | −0.202 |
| Cinacalcet HCl | 2.00 | −0.012 | −0.022 | −0.025 | −0.028 | −0.189 |
| Cinacalcet HCl | 10.00 | −0.451 | −0.091 | −0.130 | −1.076 | −0.790 |
| CK-636 | 0.08 | 0.202 | 0.039 | 0.080 | 0.483 | 0.336 |
| CK-636 | 0.40 | −0.004 | −0.049 | −0.013 | −0.010 | −0.427 |
| CK-636 | 2.00 | −0.201 | −0.066 | 0.072 | −0.479 | −0.573 |
| CK-636 | 10.00 | −0.169 | −0.108 | −0.103 | −0.404 | −0.944 |
| Clemastine Fumarate | 0.08 | 0.242 | 0.024 | 0.096 | 0.578 | 0.210 |
| Clemastine Fumarate | 0.40 | 0.036 | −0.026 | −0.066 | 0.086 | −0.227 |
| Clemastine Fumarate | 2.00 | 0.024 | −0.014 | −0.048 | 0.058 | −0.124 |
| Clemastine Fumarate | 10.00 | −0.424 | −0.061 | 0.001 | −1.013 | −0.534 |
| CNX-774 | 0.08 | 0.259 | 0.075 | 0.014 | 0.619 | 0.655 |
| CNX-774 | 0.40 | 0.177 | −0.008 | −0.027 | 0.422 | −0.067 |
| CNX-774 | 2.00 | −0.109 | 0.158 | 0.082 | −0.260 | 1.378 |
| CNX-774 | 10.00 | −0.081 | −0.043 | −0.089 | −0.194 | −0.376 |
| Costunolide | 0.08 | −0.029 | 0.131 | 0.171 | −0.070 | 1.143 |
| Costunolide | 0.40 | 0.121 | 0.067 | 0.103 | 0.288 | 0.585 |
| Costunolide | 2.00 | 0.050 | −0.018 | −0.006 | 0.120 | −0.157 |
| Costunolide | 10.00 | 0.008 | 0.047 | 0.006 | 0.019 | 0.408 |
| CP-673451 | 0.08 | 0.069 | 0.031 | 0.096 | 0.166 | 0.274 |
| CP-673451 | 0.40 | 0.186 | 0.066 | −0.046 | 0.445 | 0.577 |
| CP-673451 | 2.00 | 0.008 | 0.102 | 0.080 | 0.018 | 0.886 |
| CP-673451 | 10.00 | −0.258 | −0.058 | −0.147 | −0.616 | −0.508 |
| CP-91149 | 0.08 | 0.031 | 0.177 | 0.186 | 0.073 | 1.538 |
| CP-91149 | 0.40 | −0.023 | −0.033 | 0.055 | −0.056 | −0.289 |
| CP-91149 | 2.00 | −0.098 | 0.028 | −0.121 | −0.234 | 0.245 |
| CP-91149 | 10.00 | −0.100 | −0.057 | −0.097 | −0.239 | −0.498 |
| Crenolanib (CP-868596) | 0.08 | −0.069 | 0.121 | 0.094 | −0.165 | 1.051 |
| Crenolanib (CP-868596) | 0.40 | −0.025 | 0.051 | 0.043 | −0.059 | 0.441 |
| Crenolanib (CP-868596) | 2.00 | 0.053 | 0.122 | 0.149 | 0.127 | 1.065 |
| Crenolanib (CP-868596) | 10.00 | −0.338 | −0.224 | −0.292 | −0.808 | −1.951 |
| CRT0044876 | 0.08 | −0.171 | 0.016 | −0.002 | −0.410 | 0.135 |
| CRT0044876 | 0.40 | −0.040 | 0.034 | 0.055 | −0.096 | 0.298 |
| CRT0044876 | 2.00 | −0.081 | 0.047 | 0.014 | −0.192 | 0.408 |
| CRT0044876 | 10.00 | −0.099 | 0.032 | −0.061 | −0.237 | 0.278 |
| Cryptotanshinone | 0.08 | 0.199 | 0.143 | 0.156 | 0.475 | 1.243 |
| Cryptotanshinone | 0.40 | −0.027 | −0.120 | −0.105 | −0.065 | −1.047 |
| Cryptotanshinone | 2.00 | −0.184 | −0.182 | −0.288 | −0.439 | −1.585 |
| Cryptotanshinone | 10.00 | −0.541 | −0.288 | −0.432 | −1.292 | −2.506 |
| Cyproterone Acetate | 0.08 | −0.110 | −0.026 | 0.040 | −0.263 | −0.225 |
| Cyproterone Acetate | 0.40 | 0.051 | −0.074 | −0.019 | 0.122 | −0.642 |
| Cyproterone Acetate | 2.00 | −0.143 | −0.055 | −0.093 | −0.341 | −0.479 |
| Cyproterone Acetate | 10.00 | −0.173 | −0.081 | −0.134 | −0.414 | −0.702 |
| Dabrafenib (GSK2118436) | 0.08 | −0.134 | 0.095 | 0.236 | −0.320 | 0.830 |
| Dabrafenib (GSK2118436) | 0.40 | 0.011 | 0.049 | 0.103 | 0.026 | 0.427 |
| Dabrafenib (GSK2118436) | 2.00 | −0.279 | −0.001 | 0.019 | −0.667 | −0.006 |
| Dabrafenib (GSK2118436) | 10.00 | −0.800 | −0.250 | −0.384 | −1.911 | −2.178 |
| Dalcetrapib (JTT-705, RO4607381) | 0.08 | 0.112 | 0.055 | 0.049 | 0.268 | 0.475 |
| Dalcetrapib (JTT-705, RO4607381) | 0.40 | −0.006 | −0.037 | 0.104 | −0.014 | −0.324 |
| Dalcetrapib (JTT-705, RO4607381) | 2.00 | 0.079 | −0.034 | 0.098 | 0.188 | −0.296 |
| Dalcetrapib (JTT-705, RO4607381) | 10.00 | 0.136 | −0.074 | −0.113 | 0.324 | −0.643 |
| Dapagliflozin | 0.08 | 0.410 | 0.170 | 0.294 | 0.980 | 1.481 |
| Dapagliflozin | 0.40 | 0.254 | 0.097 | 0.135 | 0.607 | 0.842 |
| Dapagliflozin | 2.00 | 0.293 | 0.159 | 0.166 | 0.699 | 1.386 |
| Dapagliflozin | 10.00 | 0.301 | 0.136 | 0.145 | 0.719 | 1.182 |
| Daunorubicin HCl | 0.08 | −0.467 | −0.136 | −0.096 | −1.115 | −1.188 |
| Daunorubicin HCl | 0.40 | −0.601 | −0.241 | −0.329 | −1.436 | −2.099 |
| Daunorubicin HCl | 2.00 | −0.817 | −0.262 | −0.343 | −1.950 | −2.280 |
| Daunorubicin HCl | 10.00 | −1.100 | −0.260 | −0.374 | −2.628 | −2.262 |
| DBeQ | 0.08 | 0.119 | 0.097 | 0.045 | 0.285 | 0.844 |
| DBeQ | 0.40 | −0.013 | 0.064 | 0.210 | −0.031 | 0.558 |
| DBeQ | 2.00 | 0.004 | −0.048 | −0.103 | 0.010 | −0.414 |
| DBeQ | 10.00 | −1.133 | −0.296 | −0.423 | −2.706 | −2.577 |
| DCC-2036 (Rebastinib) | 0.08 | 0.216 | 0.259 | 0.358 | 0.516 | 2.256 |
| DCC-2036 (Rebastinib) | 0.40 | 0.218 | 0.128 | 0.186 | 0.520 | 1.116 |
| DCC-2036 (Rebastinib) | 2.00 | −0.038 | 0.073 | 0.054 | −0.091 | 0.637 |
| DCC-2036 (Rebastinib) | 10.00 | −0.322 | −0.209 | −0.244 | −0.769 | −1.823 |
| DMH1 | 0.08 | −0.031 | 0.018 | 0.078 | −0.075 | 0.155 |
| DMH1 | 0.40 | −0.079 | 0.026 | 0.054 | −0.188 | 0.231 |
| DMH1 | 2.00 | −0.002 | 0.014 | 0.035 | −0.004 | 0.124 |
| DMH1 | 10.00 | −0.001 | −0.075 | −0.092 | −0.003 | −0.657 |
| DMXAA (Vadimezan) | 0.08 | −0.568 | −0.007 | −0.091 | −1.357 | −0.065 |
| DMXAA (Vadimezan) | 0.40 | −0.629 | −0.036 | −0.009 | −1.502 | −0.317 |
| DMXAA (Vadimezan) | 2.00 | −0.477 | −0.038 | −0.094 | −1.139 | −0.334 |
| DMXAA (Vadimezan) | 10.00 | −0.230 | −0.031 | −0.118 | −0.550 | −0.271 |
| Doxazosin Mesylate | 0.08 | 0.072 | 0.073 | 0.093 | 0.171 | 0.633 |
| Doxazosin Mesylate | 0.40 | −0.015 | 0.044 | 0.084 | −0.037 | 0.387 |
| Doxazosin Mesylate | 2.00 | 0.146 | 0.047 | 0.030 | 0.349 | 0.413 |
| Doxazosin Mesylate | 10.00 | 0.075 | 0.039 | 0.015 | 0.179 | 0.340 |
| Dutasteride | 0.08 | 0.057 | 0.065 | 0.001 | 0.137 | 0.568 |
| Dutasteride | 0.40 | −0.266 | 0.006 | −0.054 | −0.636 | 0.052 |
| Dutasteride | 2.00 | −0.098 | −0.105 | −0.074 | −0.234 | −0.915 |
| Dutasteride | 10.00 | −0.057 | −0.092 | −0.151 | −0.137 | −0.799 |
| Dynasore | 0.08 | −0.248 | 0.049 | 0.078 | −0.592 | 0.431 |
| Dynasore | 0.40 | −0.253 | −0.020 | 0.122 | −0.604 | −0.176 |
| Dynasore | 2.00 | −0.152 | 0.036 | −0.024 | −0.364 | 0.314 |
| Dynasore | 10.00 | −0.011 | 0.048 | 0.041 | −0.026 | 0.414 |
| E-64 | 0.08 | 0.355 | 0.150 | 0.150 | 0.847 | 1.308 |
| E-64 | 0.40 | 0.045 | 0.015 | 0.111 | 0.108 | 0.131 |
| E-64 | 2.00 | 0.171 | 0.022 | 0.037 | 0.409 | 0.195 |
| E-64 | 10.00 | 0.225 | 0.037 | 0.094 | 0.537 | 0.325 |
| EHop-016 | 0.08 | 0.081 | 0.177 | 0.091 | 0.193 | 1.537 |
| EHop-016 | 0.40 | 0.062 | 0.035 | 0.012 | 0.147 | 0.302 |
| EHop-016 | 2.00 | 0.088 | 0.029 | −0.011 | 0.211 | 0.252 |
| EHop-016 | 10.00 | −0.757 | −0.203 | −0.299 | −1.808 | −1.769 |
| Elvitegravir (GS-9137, JTK-303) | 0.08 | −0.001 | −0.047 | −0.010 | −0.001 | −0.412 |
| Elvitegravir (GS-9137, JTK-303) | 0.40 | −0.040 | −0.069 | 0.092 | −0.095 | −0.598 |
| Elvitegravir (GS-9137, JTK-303) | 2.00 | −0.344 | −0.094 | −0.126 | −0.823 | −0.821 |
| Elvitegravir (GS-9137, JTK-303) | 10.00 | −0.737 | −0.090 | −0.135 | −1.760 | −0.782 |
| Embelin | 0.08 | −0.323 | −0.015 | −0.034 | −0.770 | −0.129 |
| Embelin | 0.40 | −0.723 | −0.082 | −0.122 | −1.728 | −0.712 |
| Embelin | 2.00 | −0.325 | −0.055 | −0.111 | −0.776 | −0.482 |
| Embelin | 10.00 | −0.720 | −0.094 | −0.119 | −1.720 | −0.819 |
| Empagliflozin (BI 10773) | 0.08 | −0.296 | −0.006 | −0.051 | −0.707 | −0.052 |
| Empagliflozin (BI 10773) | 0.40 | −0.075 | −0.034 | −0.055 | −0.178 | −0.294 |
| Empagliflozin (BI 10773) | 2.00 | −0.192 | −0.009 | −0.103 | −0.458 | −0.081 |
| Empagliflozin (BI 10773) | 10.00 | −0.134 | 0.017 | −0.022 | −0.320 | 0.144 |
| Enalaprilat Dihydrate | 0.08 | −0.190 | 0.003 | −0.001 | −0.454 | 0.027 |
| Enalaprilat Dihydrate | 0.40 | −0.124 | −0.003 | 0.111 | −0.296 | −0.030 |
| Enalaprilat Dihydrate | 2.00 | −0.331 | −0.022 | −0.095 | −0.791 | −0.195 |
| Enalaprilat Dihydrate | 10.00 | −0.979 | −0.157 | −0.281 | −2.338 | −1.364 |
| Entacapone | 0.08 | −0.115 | 0.136 | 0.020 | −0.275 | 1.183 |
| Entacapone | 0.40 | −0.065 | 0.012 | 0.024 | −0.154 | 0.102 |
| Entacapone | 2.00 | 0.206 | 0.085 | 0.072 | 0.493 | 0.744 |
| Entacapone | 10.00 | −0.391 | 0.100 | 0.027 | −0.935 | 0.870 |
| Enzalutamide (MDV3100) | 0.08 | 0.195 | 0.162 | 0.176 | 0.465 | 1.411 |
| Enzalutamide (MDV3100) | 0.40 | 0.256 | 0.166 | 0.276 | 0.611 | 1.446 |
| Enzalutamide (MDV3100) | 2.00 | 0.076 | 0.024 | 0.077 | 0.181 | 0.210 |
| Enzalutamide (MDV3100) | 10.00 | 0.146 | 0.094 | 0.076 | 0.348 | 0.820 |
| Enzastaurin (LY317615) | 0.08 | 0.236 | 0.085 | 0.109 | 0.565 | 0.744 |
| Enzastaurin (LY317615) | 0.40 | 0.475 | 0.131 | 0.251 | 1.135 | 1.143 |
| Enzastaurin (LY317615) | 2.00 | 0.251 | 0.053 | 0.053 | 0.600 | 0.458 |
| Enzastaurin (LY317615) | 10.00 | −0.081 | −0.116 | −0.226 | −0.194 | −1.012 |
| EPZ-6438 | 0.08 | 0.045 | 0.012 | 0.003 | 0.107 | 0.108 |
| EPZ-6438 | 0.40 | 0.075 | −0.023 | −0.023 | 0.178 | −0.203 |
| EPZ-6438 | 2.00 | −0.068 | 0.015 | −0.011 | −0.163 | 0.130 |
| EPZ-6438 | 10.00 | 0.336 | 0.062 | 0.046 | 0.802 | 0.538 |
| Erastin | 0.08 | 0.225 | 0.157 | 0.170 | 0.537 | 1.368 |
| Erastin | 0.40 | 0.285 | 0.158 | 0.160 | 0.681 | 1.375 |
| Erastin | 2.00 | 0.380 | 0.157 | 0.106 | 0.907 | 1.367 |
| Erastin | 10.00 | 0.298 | −0.015 | 0.039 | 0.711 | −0.133 |
| Esomeprazole Sodium | 0.08 | 0.288 | 0.086 | 0.146 | 0.689 | 0.747 |
| Esomeprazole Sodium | 0.40 | 0.199 | 0.233 | 0.174 | 0.475 | 2.030 |
| Esomeprazole Sodium | 2.00 | 0.386 | 0.034 | 0.043 | 0.923 | 0.300 |
| Esomeprazole Sodium | 10.00 | 0.271 | −0.016 | 0.021 | 0.648 | −0.139 |
| Etodolac | 0.08 | −0.061 | 0.046 | 0.034 | −0.147 | 0.400 |
| Etodolac | 0.40 | 0.049 | 0.036 | 0.127 | 0.117 | 0.314 |
| Etodolac | 2.00 | 0.008 | 0.052 | 0.070 | 0.020 | 0.452 |
| Etodolac | 10.00 | 0.023 | −0.009 | −0.011 | 0.054 | −0.078 |
| Etomidate | 0.08 | 0.084 | −0.008 | 0.053 | 0.201 | −0.065 |
| Etomidate | 0.40 | 0.006 | 0.037 | 0.048 | 0.014 | 0.318 |
| Etomidate | 2.00 | 0.163 | 0.015 | 0.054 | 0.389 | 0.131 |
| Etomidate | 10.00 | 0.039 | −0.001 | −0.008 | 0.094 | −0.010 |
| EUK 134 | 0.08 | −0.015 | 0.068 | 0.025 | −0.035 | 0.594 |
| EUK 134 | 0.40 | −0.162 | 0.003 | 0.057 | −0.387 | 0.026 |
| EUK 134 | 2.00 | 0.091 | 0.028 | −0.034 | 0.217 | 0.247 |
| EUK 134 | 10.00 | −0.024 | −0.085 | −0.132 | −0.057 | −0.741 |
| Everolimus (RAD001) | 0.08 | 0.392 | 0.068 | −0.012 | 0.936 | 0.590 |
| Everolimus (RAD001) | 0.40 | 0.127 | 0.110 | −0.007 | 0.304 | 0.956 |
| Everolimus (RAD001) | 2.00 | 0.132 | −0.003 | −0.039 | 0.314 | −0.030 |
| Everolimus (RAD001) | 10.00 | −0.111 | −0.024 | −0.096 | −0.266 | −0.210 |
| EX 527 (Selisistat) | 0.08 | 0.240 | 0.100 | 0.062 | 0.574 | 0.871 |
| EX 527 (Selisistat) | 0.40 | 0.174 | 0.059 | 0.047 | 0.415 | 0.512 |
| EX 527 (Selisistat) | 2.00 | 0.341 | 0.030 | −0.051 | 0.814 | 0.261 |
| EX 527 (Selisistat) | 10.00 | 0.320 | 0.057 | −0.011 | 0.766 | 0.494 |
| Exemestane | 0.08 | −0.046 | 0.051 | 0.062 | −0.110 | 0.447 |
| Exemestane | 0.40 | −0.407 | 0.079 | 0.035 | −0.973 | 0.687 |
| Exemestane | 2.00 | 0.170 | 0.001 | 0.058 | 0.405 | 0.012 |
| Exemestane | 10.00 | −0.167 | 0.007 | −0.005 | −0.398 | 0.060 |
| Felodipine | 0.08 | −0.078 | 0.001 | 0.008 | −0.187 | 0.010 |
| Felodipine | 0.40 | −0.319 | −0.033 | −0.042 | −0.763 | −0.284 |
| Felodipine | 2.00 | 0.011 | 0.002 | −0.003 | 0.025 | 0.017 |
| Felodipine | 10.00 | 0.032 | −0.056 | −0.067 | 0.078 | −0.487 |
| Ferrostatin-1 (Fer-1) | 0.08 | 0.312 | 0.108 | 0.120 | 0.745 | 0.944 |
| Ferrostatin-1 (Fer-1) | 0.40 | 0.453 | 0.116 | 0.207 | 1.082 | 1.012 |
| Ferrostatin-1 (Fer-1) | 2.00 | 0.215 | 0.070 | 0.092 | 0.514 | 0.610 |
| Ferrostatin-1 (Fer-1) | 10.00 | −0.046 | 0.013 | −0.018 | −0.109 | 0.116 |
| FG-4592 | 0.08 | 0.327 | 0.077 | 0.083 | 0.782 | 0.673 |
| FG-4592 | 0.40 | 0.284 | 0.170 | 0.234 | 0.679 | 1.484 |
| FG-4592 | 2.00 | 0.290 | 0.094 | 0.152 | 0.692 | 0.822 |
| FG-4592 | 10.00 | 0.238 | 0.030 | 0.071 | 0.568 | 0.265 |
| Finasteride | 0.08 | 0.151 | 0.205 | 0.199 | 0.360 | 1.781 |
| Finasteride | 0.40 | −0.038 | 0.213 | 0.211 | −0.092 | 1.852 |
| Finasteride | 2.00 | 0.073 | 0.142 | 0.156 | 0.174 | 1.236 |
| Finasteride | 10.00 | −0.041 | 0.067 | 0.056 | −0.097 | 0.587 |
| Fingolimod (FTY720) HCl | 0.08 | −0.080 | −0.054 | −0.046 | −0.191 | −0.471 |
| Fingolimod (FTY720) HCl | 0.40 | 0.103 | 0.071 | 0.077 | 0.245 | 0.616 |
| Fingolimod (FTY720) HCl | 2.00 | −0.468 | 0.021 | −0.060 | −1.119 | 0.186 |
| Fingolimod (FTY720) HCl | 10.00 | −1.304 | −0.225 | −0.362 | −3.116 | −1.961 |
| FLI-06 | 0.08 | 0.089 | 0.050 | 0.079 | 0.213 | 0.439 |
| FLI-06 | 0.40 | 0.095 | −0.026 | 0.010 | 0.226 | −0.230 |
| FLI-06 | 2.00 | 0.076 | −0.054 | −0.077 | 0.182 | −0.466 |
| FLI-06 | 10.00 | −0.343 | −0.140 | −0.170 | −0.819 | −1.216 |
| Fluvastatin Sodium | 0.08 | −0.161 | 0.112 | 0.026 | −0.384 | 0.979 |
| Fluvastatin Sodium | 0.40 | −0.482 | −0.146 | −0.177 | −1.151 | −1.275 |
| Fluvastatin Sodium | 2.00 | −1.111 | −0.203 | −0.293 | −2.653 | −1.764 |
| Fluvastatin Sodium | 10.00 | −1.761 | −0.159 | −0.267 | −4.206 | −1.388 |
| Fluvoxamine maleate | 0.08 | 0.115 | 0.074 | 0.114 | 0.274 | 0.642 |
| Fluvoxamine maleate | 0.40 | 0.254 | 0.087 | 0.172 | 0.607 | 0.756 |
| Fluvoxamine maleate | 2.00 | 0.211 | 0.089 | 0.108 | 0.504 | 0.778 |
| Fluvoxamine maleate | 10.00 | 0.138 | −0.027 | 0.012 | 0.330 | −0.235 |
| Formoterol Hemifumarate | 0.08 | 0.294 | 0.145 | 0.206 | 0.703 | 1.263 |
| Formoterol Hemifumarate | 0.40 | 0.265 | 0.073 | 0.293 | 0.633 | 0.638 |
| Formoterol Hemifumarate | 2.00 | 0.242 | 0.048 | 0.090 | 0.578 | 0.420 |
| Formoterol Hemifumarate | 10.00 | 0.253 | 0.013 | 0.011 | 0.604 | 0.114 |
| Forskolin | 0.08 | −0.213 | −0.007 | −0.004 | −0.508 | −0.058 |
| Forskolin | 0.40 | −0.175 | 0.159 | 0.237 | −0.418 | 1.381 |
| Forskolin | 2.00 | −0.330 | 0.191 | 0.102 | −0.789 | 1.661 |
| Forskolin | 10.00 | −0.119 | 0.219 | 0.128 | −0.284 | 1.905 |
| Fostamatinib (R788) | 0.08 | 0.232 | 0.119 | 0.235 | 0.554 | 1.033 |
| Fostamatinib (R788) | 0.40 | −0.052 | −0.025 | 0.156 | −0.123 | −0.217 |
| Fostamatinib (R788) | 2.00 | −0.231 | −0.099 | −0.015 | −0.552 | −0.860 |
| Fostamatinib (R788) | 10.00 | −0.881 | −0.234 | −0.350 | −2.105 | −2.036 |
| Fulvestrant | 0.08 | 0.119 | 0.057 | 0.051 | 0.285 | 0.500 |
| Fulvestrant | 0.40 | −0.063 | −0.050 | −0.049 | −0.150 | −0.432 |
| Fulvestrant | 2.00 | 0.142 | 0.000 | −0.024 | 0.339 | 0.003 |
| Fulvestrant | 10.00 | −0.204 | −0.043 | −0.150 | −0.488 | −0.376 |
| Ganetespib (STA-9090) | 0.08 | −1.450 | −0.141 | −0.260 | −3.465 | −1.226 |
| Ganetespib (STA-9090) | 0.40 | −2.105 | −0.155 | −0.250 | −5.027 | −1.345 |
| Ganetespib (STA-9090) | 2.00 | −1.721 | −0.150 | −0.260 | −4.110 | −1.309 |
| Ganetespib (STA-9090) | 10.00 | −1.620 | −0.136 | −0.267 | −3.871 | −1.181 |
| GDC-0068 | 0.08 | 0.042 | 0.036 | 0.076 | 0.099 | 0.311 |
| GDC-0068 | 0.40 | 0.118 | 0.054 | 0.087 | 0.283 | 0.469 |
| GDC-0068 | 2.00 | 0.184 | −0.077 | −0.059 | 0.439 | −0.675 |
| GDC-0068 | 10.00 | −0.100 | −0.185 | −0.319 | −0.238 | −1.613 |
| GDC-0152 | 0.08 | 0.151 | −0.002 | −0.020 | 0.361 | −0.013 |
| GDC-0152 | 0.40 | 0.158 | −0.020 | −0.036 | 0.377 | −0.176 |
| GDC-0152 | 2.00 | 0.003 | 0.083 | −0.057 | 0.008 | 0.724 |
| GDC-0152 | 10.00 | 0.241 | 0.103 | −0.009 | 0.575 | 0.900 |
| GDC-0941 | 0.08 | 0.283 | 0.008 | 0.018 | 0.677 | 0.070 |
| GDC-0941 | 0.40 | 0.022 | −0.013 | −0.076 | 0.053 | −0.112 |
| GDC-0941 | 2.00 | −0.197 | −0.128 | −0.248 | −0.472 | −1.113 |
| GDC-0941 | 10.00 | −1.223 | −0.156 | −0.257 | −2.921 | −1.362 |
| Ginkgolide A | 0.08 | 0.005 | 0.045 | 0.124 | 0.011 | 0.395 |
| Ginkgolide A | 0.40 | −0.017 | 0.025 | 0.161 | −0.040 | 0.222 |
| Ginkgolide A | 2.00 | 0.040 | 0.030 | 0.002 | 0.095 | 0.258 |
| Ginkgolide A | 10.00 | −0.012 | 0.052 | −0.028 | −0.028 | 0.450 |
| Ginkgolide B | 0.08 | −0.076 | 0.041 | 0.034 | −0.182 | 0.353 |
| Ginkgolide B | 0.40 | 0.034 | 0.109 | 0.047 | 0.081 | 0.950 |
| Ginkgolide B | 2.00 | −0.154 | 0.061 | 0.033 | −0.369 | 0.528 |
| Ginkgolide B | 10.00 | 0.177 | −0.037 | −0.035 | 0.422 | −0.326 |
| Gliclazide | 0.08 | 0.162 | 0.036 | 0.058 | 0.386 | 0.313 |
| Gliclazide | 0.40 | 0.054 | 0.032 | 0.086 | 0.128 | 0.278 |
| Gliclazide | 2.00 | 0.008 | 0.104 | −0.017 | 0.020 | 0.909 |
| Gliclazide | 10.00 | −0.092 | −0.035 | −0.060 | −0.219 | −0.303 |
| Gliquidone | 0.08 | 0.124 | 0.103 | 0.101 | 0.297 | 0.893 |
| Gliquidone | 0.40 | 0.103 | 0.037 | 0.070 | 0.247 | 0.322 |
| Gliquidone | 2.00 | 0.246 | 0.086 | 0.006 | 0.588 | 0.747 |
| Gliquidone | 10.00 | 0.134 | 0.031 | −0.020 | 0.320 | 0.268 |
| GNF-2 | 0.08 | 0.157 | 0.159 | 0.121 | 0.376 | 1.386 |
| GNF-2 | 0.40 | 0.090 | −0.024 | 0.001 | 0.214 | −0.206 |
| GNF-2 | 2.00 | 0.074 | 0.012 | 0.046 | 0.176 | 0.102 |
| GNF-2 | 10.00 | 0.100 | −0.033 | −0.015 | 0.240 | −0.286 |
| Go 6983 | 0.08 | 0.021 | −0.005 | 0.021 | 0.050 | −0.046 |
| Go 6983 | 0.40 | −0.036 | 0.013 | 0.007 | −0.085 | 0.114 |
| Go 6983 | 2.00 | 0.058 | 0.091 | −0.010 | 0.138 | 0.791 |
| Go 6983 | 10.00 | −0.181 | −0.092 | −0.073 | −0.433 | −0.798 |
| Golgicide A | 0.08 | 0.167 | 0.267 | 0.146 | 0.398 | 2.325 |
| Golgicide A | 0.40 | 0.227 | 0.066 | 0.202 | 0.541 | 0.576 |
| Golgicide A | 2.00 | 0.339 | 0.171 | 0.076 | 0.810 | 1.492 |
| Golgicide A | 10.00 | −0.220 | −0.161 | −0.256 | −0.526 | −1.402 |
| Granisetron HCl | 0.08 | −0.214 | 0.061 | 0.015 | −0.512 | 0.531 |
| Granisetron HCl | 0.40 | −0.086 | −0.002 | 0.131 | −0.205 | −0.021 |
| Granisetron HCl | 2.00 | −0.178 | 0.018 | −0.007 | −0.426 | 0.155 |
| Granisetron HCl | 10.00 | −0.297 | −0.043 | −0.103 | −0.710 | −0.378 |
| GSK J4 HCl | 0.08 | 0.124 | 0.058 | 0.086 | 0.297 | 0.508 |
| GSK J4 HCl | 0.40 | 0.099 | 0.050 | 0.109 | 0.237 | 0.433 |
| GSK J4 HCl | 2.00 | 0.002 | 0.018 | 0.041 | 0.006 | 0.153 |
| GSK J4 HCl | 10.00 | 0.233 | −0.001 | −0.002 | 0.558 | −0.006 |
| GSK1292263 | 0.08 | 0.161 | 0.078 | 0.111 | 0.384 | 0.681 |
| GSK1292263 | 0.40 | 0.192 | 0.050 | 0.101 | 0.458 | 0.434 |
| GSK1292263 | 2.00 | 0.224 | 0.028 | 0.060 | 0.535 | 0.240 |
| GSK1292263 | 10.00 | 0.196 | 0.020 | 0.027 | 0.468 | 0.173 |
| GSK1904529A | 0.08 | 0.202 | 0.102 | 0.133 | 0.484 | 0.890 |
| GSK1904529A | 0.40 | −0.071 | 0.112 | −0.047 | −0.170 | 0.977 |
| GSK1904529A | 2.00 | −0.570 | −0.179 | −0.225 | −1.361 | −1.557 |
| GSK1904529A | 10.00 | −0.691 | −0.163 | −0.276 | −1.649 | −1.419 |
| GSK2656157 | 0.08 | 0.068 | −0.031 | −0.008 | 0.162 | −0.267 |
| GSK2656157 | 0.40 | 0.115 | −0.045 | 0.060 | 0.274 | −0.396 |
| GSK2656157 | 2.00 | 0.083 | −0.033 | −0.020 | 0.197 | −0.291 |
| GSK2656157 | 10.00 | 0.119 | −0.012 | 0.090 | 0.284 | −0.102 |
| GSK429286A | 0.08 | 0.527 | 0.137 | 0.247 | 1.260 | 1.197 |
| GSK429286A | 0.40 | 0.545 | 0.128 | 0.283 | 1.302 | 1.117 |
| GSK429286A | 2.00 | 0.488 | 0.140 | 0.245 | 1.166 | 1.222 |
| GSK429286A | 10.00 | 0.370 | 0.059 | 0.099 | 0.883 | 0.517 |
| GSK461364 | 0.08 | 0.160 | 0.078 | 0.112 | 0.383 | 0.682 |
| GSK461364 | 0.40 | −0.154 | −0.096 | −0.093 | −0.368 | −0.834 |
| GSK461364 | 2.00 | −0.628 | −0.246 | −0.352 | −1.500 | −2.138 |
| GSK461364 | 10.00 | −0.621 | −0.287 | −0.390 | −1.484 | −2.498 |
| GSK690693 | 0.08 | 0.129 | 0.045 | 0.019 | 0.308 | 0.388 |
| GSK690693 | 0.40 | 0.197 | 0.047 | 0.155 | 0.470 | 0.409 |
| GSK690693 | 2.00 | −0.027 | 0.122 | 0.124 | −0.064 | 1.060 |
| GSK690693 | 10.00 | −0.182 | −0.080 | −0.153 | −0.434 | −0.698 |
| GW0742 | 0.08 | −0.751 | 0.052 | 0.048 | −1.794 | 0.454 |
| GW0742 | 0.40 | −0.489 | 0.045 | 0.106 | −1.167 | 0.390 |
| GW0742 | 2.00 | −0.249 | 0.026 | 0.014 | −0.596 | 0.231 |
| GW0742 | 10.00 | −0.451 | −0.152 | −0.183 | −1.078 | −1.322 |
| GW2580 | 0.08 | −0.042 | 0.006 | −0.013 | −0.101 | 0.056 |
| GW2580 | 0.40 | −0.284 | 0.014 | 0.020 | −0.678 | 0.118 |
| GW2580 | 2.00 | 0.029 | −0.060 | −0.060 | 0.068 | −0.525 |
| GW2580 | 10.00 | −0.038 | −0.018 | −0.026 | −0.091 | −0.158 |
| GW3965 HCl | 0.08 | 0.138 | 0.059 | 0.091 | 0.330 | 0.516 |
| GW3965 HCl | 0.40 | 0.201 | 0.002 | 0.074 | 0.481 | 0.013 |
| GW3965 HCl | 2.00 | 0.229 | 0.077 | 0.179 | 0.546 | 0.673 |
| GW3965 HCl | 10.00 | −0.078 | −0.009 | −0.047 | −0.186 | −0.080 |
| GW4064 | 0.08 | −0.134 | 0.170 | 0.028 | −0.320 | 1.476 |
| GW4064 | 0.40 | 0.140 | −0.022 | 0.097 | 0.334 | −0.194 |
| GW4064 | 2.00 | −0.145 | 0.022 | 0.000 | −0.346 | 0.196 |
| GW4064 | 10.00 | −0.157 | −0.059 | −0.071 | −0.375 | −0.516 |
| GW441756 | 0.08 | 0.048 | 0.046 | 0.075 | 0.114 | 0.399 |
| GW441756 | 0.40 | −0.105 | 0.061 | 0.196 | −0.252 | 0.529 |
| GW441756 | 2.00 | 0.152 | 0.117 | 0.125 | 0.364 | 1.023 |
| GW441756 | 10.00 | −0.113 | 0.088 | 0.071 | −0.270 | 0.763 |
| GW9508 | 0.08 | 0.063 | 0.075 | 0.067 | 0.151 | 0.650 |
| GW9508 | 0.40 | 0.174 | 0.019 | 0.049 | 0.417 | 0.169 |
| GW9508 | 2.00 | −0.001 | −0.011 | 0.000 | −0.003 | −0.092 |
| GW9508 | 10.00 | −0.059 | −0.040 | −0.045 | −0.140 | −0.350 |
| GW9662 | 0.08 | 0.115 | 0.035 | 0.036 | 0.274 | 0.305 |
| GW9662 | 0.40 | 0.041 | 0.098 | 0.080 | 0.098 | 0.852 |
| GW9662 | 2.00 | 0.212 | 0.046 | 0.093 | 0.508 | 0.400 |
| GW9662 | 10.00 | 0.133 | 0.030 | 0.020 | 0.318 | 0.264 |
| H 89 2HCl | 0.08 | 0.350 | 0.140 | 0.118 | 0.836 | 1.223 |
| H 89 2HCl | 0.40 | 0.398 | 0.080 | 0.148 | 0.950 | 0.695 |
| H 89 2HCl | 2.00 | 0.421 | 0.086 | 0.087 | 1.005 | 0.747 |
| H 89 2HCl | 10.00 | 0.385 | 0.132 | 0.000 | 0.919 | 1.151 |
| HA14-1 | 0.08 | 0.534 | 0.121 | 0.190 | 1.276 | 1.052 |
| HA14-1 | 0.40 | 0.526 | 0.137 | 0.316 | 1.255 | 1.195 |
| HA14-1 | 2.00 | 0.554 | 0.154 | 0.170 | 1.323 | 1.345 |
| HA14-1 | 10.00 | 0.388 | 0.132 | 0.149 | 0.926 | 1.151 |
| HC-030031 | 0.08 | 0.082 | 0.063 | 0.090 | 0.195 | 0.547 |
| HC-030031 | 0.40 | 0.191 | 0.033 | 0.097 | 0.457 | 0.291 |
| HC-030031 | 2.00 | −0.008 | −0.007 | −0.035 | −0.019 | −0.057 |
| HC-030031 | 10.00 | −0.241 | −0.047 | 0.077 | −0.575 | −0.411 |
| I-BET151 (GSK1210151A) | 0.08 | −0.016 | −0.116 | −0.242 | −0.039 | −1.010 |
| I-BET151 (GSK1210151A) | 0.40 | −0.174 | −0.248 | −0.344 | −0.415 | −2.159 |
| I-BET151 (GSK1210151A) | 2.00 | −0.502 | −0.299 | −0.416 | −1.200 | −2.601 |
| I-BET151 (GSK1210151A) | 10.00 | −0.935 | −0.284 | −0.408 | −2.233 | −2.473 |
| Ibrutinib (PCI-32765) | 0.08 | −0.027 | −0.071 | −0.139 | −0.065 | −0.619 |
| Ibrutinib (PCI-32765) | 0.40 | −0.466 | −0.141 | −0.206 | −1.112 | −1.225 |
| Ibrutinib (PCI-32765) | 2.00 | −0.521 | −0.202 | −0.292 | −1.244 | −1.763 |
| Ibrutinib (PCI-32765) | 10.00 | −0.931 | −0.199 | −0.312 | −2.224 | −1.737 |
| ICG-001 | 0.08 | 0.104 | 0.037 | 0.163 | 0.249 | 0.324 |
| ICG-001 | 0.40 | 0.104 | 0.037 | 0.204 | 0.248 | 0.321 |
| ICG-001 | 2.00 | 0.143 | 0.045 | 0.017 | 0.343 | 0.396 |
| ICG-001 | 10.00 | −0.416 | −0.125 | −0.124 | −0.993 | −1.093 |
| Icotinib | 0.08 | −0.020 | 0.029 | 0.094 | −0.047 | 0.255 |
| Icotinib | 0.40 | −0.061 | −0.073 | −0.090 | −0.146 | −0.632 |
| Icotinib | 2.00 | −0.406 | −0.133 | −0.243 | −0.970 | −1.160 |
| Icotinib | 10.00 | −1.068 | −0.289 | −0.419 | −2.552 | −2.518 |
| Ifenprodil Tartrate | 0.08 | 0.102 | 0.056 | 0.050 | 0.243 | 0.492 |
| Ifenprodil Tartrate | 0.40 | 0.111 | −0.019 | 0.017 | 0.265 | −0.161 |
| Ifenprodil Tartrate | 2.00 | −0.003 | 0.123 | −0.023 | −0.008 | 1.068 |
| Ifenprodil Tartrate | 10.00 | −1.034 | −0.219 | −0.301 | −2.471 | −1.909 |
| IKK-16 (IKK Inhibitor VII) | 0.08 | 0.058 | 0.104 | 0.092 | 0.139 | 0.903 |
| IKK-16 (IKK Inhibitor VII) | 0.40 | −0.051 | 0.020 | 0.028 | −0.122 | 0.176 |
| IKK-16 (IKK Inhibitor VII) | 2.00 | −0.431 | −0.081 | −0.142 | −1.029 | −0.708 |
| IKK-16 (IKK Inhibitor VII) | 10.00 | −0.950 | −0.291 | −0.383 | −2.268 | −2.531 |
| Ilomastat (GM6001, Galardin) | 0.08 | 0.197 | 0.055 | 0.214 | 0.470 | 0.482 |
| Ilomastat (GM6001, Galardin) | 0.40 | 0.163 | −0.011 | 0.117 | 0.389 | −0.092 |
| Ilomastat (GM6001, Galardin) | 2.00 | 0.091 | 0.007 | 0.024 | 0.216 | 0.064 |
| Ilomastat (GM6001, Galardin) | 10.00 | −0.110 | −0.106 | −0.078 | −0.264 | −0.919 |
| Imatinib (STI571) | 0.08 | −0.150 | 0.033 | 0.000 | −0.357 | 0.284 |
| Imatinib (STI571) | 0.40 | 0.079 | 0.012 | 0.038 | 0.188 | 0.104 |
| Imatinib (STI571) | 2.00 | 0.056 | 0.025 | −0.001 | 0.135 | 0.217 |
| Imatinib (STI571) | 10.00 | −0.034 | −0.038 | −0.037 | −0.082 | −0.333 |
| IMD 0354 | 0.08 | −0.007 | 0.004 | −0.047 | −0.017 | 0.035 |
| IMD 0354 | 0.40 | 0.117 | 0.101 | 0.043 | 0.280 | 0.880 |
| IMD 0354 | 2.00 | 0.076 | 0.097 | 0.083 | 0.181 | 0.847 |
| IMD 0354 | 10.00 | −0.480 | −0.089 | −0.128 | −1.146 | −0.777 |
| Imidapril HCl | 0.08 | −0.333 | −0.006 | −0.042 | −0.796 | −0.049 |
| Imidapril HCl | 0.40 | −0.148 | 0.000 | 0.109 | −0.354 | 0.004 |
| Imidapril HCl | 2.00 | −0.236 | 0.014 | −0.035 | −0.564 | 0.121 |
| Imidapril HCl | 10.00 | −0.227 | −0.081 | −0.075 | −0.543 | −0.706 |
| Iniparib (B5I-201) | 0.08 | 0.339 | 0.042 | 0.032 | 0.810 | 0.364 |
| Iniparib (BSI-201) | 0.40 | 0.181 | 0.000 | 0.035 | 0.432 | 0.003 |
| Iniparib (BSI-201) | 2.00 | 0.157 | 0.016 | −0.001 | 0.375 | 0.138 |
| Iniparib (BSI-201) | 10.00 | 0.244 | −0.016 | −0.010 | 0.583 | −0.139 |
| IOX2 | 0.08 | 0.149 | 0.029 | 0.047 | 0.356 | 0.254 |
| IOX2 | 0.40 | 0.078 | 0.028 | 0.071 | 0.185 | 0.246 |
| IOX2 | 2.00 | 0.097 | 0.043 | 0.001 | 0.232 | 0.376 |
| IOX2 | 10.00 | 0.001 | −0.045 | −0.049 | 0.002 | −0.388 |
| IPA-3 | 0.08 | 0.219 | 0.004 | 0.064 | 0.524 | 0.031 |
| IPA-3 | 0.40 | −0.332 | 0.003 | −0.031 | −0.793 | 0.028 |
| IPA-3 | 2.00 | −0.084 | −0.001 | 0.067 | −0.202 | −0.010 |
| IPA-3 | 10.00 | −0.054 | −0.021 | −0.054 | −0.128 | −0.186 |
| Irinotecan | 0.08 | −0.111 | −0.031 | −0.004 | −0.265 | −0.272 |
| Irinotecan | 0.40 | −0.784 | −0.072 | −0.145 | −1.872 | −0.630 |
| Irinotecan | 2.00 | −0.785 | −0.170 | −0.247 | −1.875 | −1.476 |
| Irinotecan | 10.00 | −1.235 | −0.163 | −0.251 | −2.951 | −1.417 |
| Irinotecan HCl Trihydrate | 0.08 | −0.083 | −0.019 | −0.040 | −0.199 | −0.162 |
| Irinotecan HCl Trihydrate | 0.40 | −0.205 | −0.092 | −0.158 | −0.491 | −0.805 |
| Irinotecan HCl Trihydrate | 2.00 | −0.317 | −0.221 | −0.293 | −0.758 | −1.925 |
| Irinotecan HCl Trihydrate | 10.00 | −0.588 | −0.272 | −0.367 | −1.404 | −2.368 |
| Isotretinoin | 0.08 | 0.411 | 0.125 | 0.109 | 0.982 | 1.086 |
| Isotretinoin | 0.40 | 0.404 | 0.047 | 0.047 | 0.965 | 0.407 |
| Isotretinoin | 2.00 | 0.253 | 0.066 | 0.034 | 0.604 | 0.578 |
| Isotretinoin | 10.00 | 0.139 | 0.082 | 0.075 | 0.331 | 0.718 |
| Ispinesib (SB-715992) | 0.08 | −0.323 | −0.222 | −0.271 | −0.772 | −1.931 |
| Ispinesib (SB-715992) | 0.40 | −0.518 | −0.191 | −0.296 | −1.238 | −1.663 |
| Ispinesib (SB-715992) | 2.00 | −0.511 | −0.196 | −0.287 | −1.220 | −1.709 |
| Ispinesib (SB-715992) | 10.00 | −0.713 | −0.201 | −0.286 | −1.704 | −1.752 |
| Istradefylline | 0.08 | −0.045 | −0.015 | −0.029 | −0.107 | −0.131 |
| Istradefylline | 0.40 | 0.066 | −0.035 | −0.087 | 0.158 | −0.304 |
| Istradefylline | 2.00 | −0.157 | −0.003 | −0.096 | −0.375 | −0.022 |
| Istradefylline | 10.00 | −0.038 | −0.041 | −0.057 | −0.091 | −0.353 |
| Ivacaftor (VX-770) | 0.08 | 0.234 | 0.099 | 0.132 | 0.558 | 0.864 |
| Ivacaftor (VX-770) | 0.40 | 0.145 | 0.127 | 0.189 | 0.347 | 1.109 |
| Ivacaftor (VX-770) | 2.00 | −0.148 | 0.082 | 0.019 | −0.354 | 0.717 |
| Ivacaftor (VX-770) | 10.00 | −0.470 | −0.142 | −0.253 | −1.123 | −1.240 |
| JIB-04 | 0.08 | 0.254 | 0.178 | 0.203 | 0.607 | 1.549 |
| JIB-04 | 0.40 | 0.108 | 0.030 | 0.084 | 0.258 | 0.264 |
| JIB-04 | 2.00 | 0.092 | 0.006 | −0.056 | 0.219 | 0.048 |
| JIB-04 | 10.00 | −0.181 | −0.153 | −0.085 | −0.433 | −1.330 |
| JNJ-1661010 | 0.08 | −0.094 | 0.122 | 0.135 | −0.225 | 1.066 |
| JNJ-1661010 | 0.40 | −0.262 | 0.075 | 0.041 | −0.625 | 0.653 |
| JNJ-1661010 | 2.00 | −0.055 | 0.103 | −0.063 | −0.131 | 0.897 |
| JNJ-1661010 | 10.00 | −0.145 | 0.047 | −0.068 | −0.346 | 0.406 |
| JSH-23 | 0.08 | 0.193 | 0.054 | 0.164 | 0.462 | 0.474 |
| JSH-23 | 0.40 | 0.230 | 0.079 | 0.106 | 0.548 | 0.688 |
| JSH-23 | 2.00 | 0.074 | −0.002 | 0.005 | 0.176 | −0.017 |
| JSH-23 | 10.00 | 0.046 | −0.046 | −0.026 | 0.110 | −0.399 |
| Ki16198 | 0.08 | −0.011 | 0.005 | 0.027 | −0.026 | 0.044 |
| Ki16198 | 0.40 | −0.108 | −0.009 | 0.031 | −0.258 | −0.076 |
| Ki16198 | 2.00 | −0.033 | 0.060 | −0.015 | −0.078 | 0.519 |
| Ki16198 | 10.00 | 0.022 | −0.103 | −0.037 | 0.052 | −0.899 |
| Ki16425 | 0.08 | −0.051 | 0.001 | 0.022 | −0.121 | 0.005 |
| Ki16425 | 0.40 | −0.002 | 0.117 | 0.114 | −0.005 | 1.017 |
| Ki16425 | 2.00 | −0.078 | −0.008 | −0.021 | −0.185 | −0.069 |
| Ki16425 | 10.00 | −0.165 | −0.038 | −0.071 | −0.394 | −0.327 |
| KPT-185 | 0.08 | 0.116 | −0.006 | −0.015 | 0.277 | −0.049 |
| KPT-185 | 0.40 | 0.104 | 0.097 | 0.220 | 0.248 | 0.846 |
| KPT-185 | 2.00 | −0.995 | −0.209 | −0.322 | −2.377 | −1.820 |
| KPT-185 | 10.00 | −0.855 | −0.085 | −0.153 | −2.041 | −0.744 |
| KPT-276 | 0.08 | 0.167 | 0.056 | 0.133 | 0.399 | 0.492 |
| KPT-276 | 0.40 | −0.044 | 0.086 | 0.148 | −0.104 | 0.746 |
| KPT-276 | 2.00 | −0.413 | −0.224 | −0.327 | −0.986 | −1.947 |
| KPT-276 | 10.00 | −0.703 | −0.256 | −0.361 | −1.680 | −2.230 |
| KPT-330 | 0.08 | 0.210 | 0.200 | 0.259 | 0.503 | 1.745 |
| KPT-330 | 0.40 | −0.657 | −0.266 | −0.282 | −1.569 | −2.317 |
| KPT-330 | 2.00 | −0.894 | −0.280 | −0.390 | −2.135 | −2.439 |
| KPT-330 | 10.00 | −1.876 | −0.284 | −0.423 | −4.480 | −2.470 |
| KU-55933 (ATM Kinase Inhibitor) | 0.08 | 0.524 | 0.115 | 0.168 | 1.251 | 0.998 |
| KU-55933 (ATM Kinase Inhibitor) | 0.40 | 0.477 | 0.103 | 0.200 | 1.140 | 0.893 |
| KU-55933 (ATM Kinase Inhibitor) | 2.00 | 0.551 | 0.116 | 0.161 | 1.317 | 1.009 |
| KU-55933 (ATM Kinase Inhibitor) | 10.00 | 0.541 | 0.074 | 0.076 | 1.293 | 0.642 |
| KU-60019 | 0.08 | 0.114 | 0.042 | 0.230 | 0.273 | 0.365 |
| KU-60019 | 0.40 | 0.039 | −0.001 | 0.073 | 0.094 | −0.012 |
| KU-60019 | 2.00 | 0.121 | −0.015 | −0.049 | 0.290 | −0.132 |
| KU-60019 | 10.00 | −0.522 | −0.088 | −0.222 | −1.246 | −0.769 |
| KX2-391 | 0.08 | −0.752 | −0.283 | −0.396 | −1.796 | −2.468 |
| KX2-391 | 0.40 | −1.251 | −0.292 | −0.380 | −2.989 | −2.546 |
| KX2-391 | 2.00 | −1.983 | −0.251 | −0.424 | −4.737 | −2.188 |
| KX2-391 | 10.00 | −1.414 | −0.286 | −0.419 | −3.377 | −2.494 |
| Lafutidine | 0.08 | 0.152 | 0.043 | 0.052 | 0.363 | 0.372 |
| Lafutidine | 0.40 | 0.145 | 0.007 | 0.078 | 0.346 | 0.059 |
| Lafutidine | 2.00 | 0.138 | 0.037 | 0.076 | 0.329 | 0.320 |
| Lafutidine | 10.00 | −0.098 | 0.075 | −0.014 | −0.235 | 0.657 |
| LB42708 | 0.08 | −0.072 | −0.116 | −0.089 | −0.172 | −1.010 |
| LB42708 | 0.40 | −0.342 | −0.140 | −0.144 | −0.817 | −1.221 |
| LB42708 | 2.00 | −0.116 | −0.176 | −0.238 | −0.277 | −1.531 |
| LB42708 | 10.00 | −0.706 | −0.182 | −0.233 | −1.685 | −1.587 |
| LDE225 (NVP-LDE225, Erismodegib) | 0.08 | 0.107 | 0.085 | 0.095 | 0.255 | 0.739 |
| LDE225 (NVP-LDE225, Erismodegib) | 0.40 | 0.067 | 0.101 | 0.074 | 0.159 | 0.881 |
| LDE225 (NVP-LDE225, Erismodegib) | 2.00 | 0.229 | 0.011 | 0.002 | 0.546 | 0.094 |
| LDE225 (NVP-LDE225, Erismodegib) | 10.00 | −0.001 | 0.049 | 0.006 | −0.002 | 0.426 |
| LDK378 | 0.08 | 0.135 | 0.061 | 0.118 | 0.322 | 0.534 |
| LDK378 | 0.40 | 0.004 | −0.053 | −0.047 | 0.010 | −0.465 |
| LDK378 | 2.00 | −0.364 | −0.195 | −0.281 | −0.869 | −1.702 |
| LDK378 | 10.00 | −1.292 | −0.338 | −0.487 | −3.087 | −2.939 |
| LDN-212854 | 0.08 | 0.238 | 0.095 | 0.162 | 0.568 | 0.823 |
| LDN-212854 | 0.40 | 0.129 | 0.051 | 0.026 | 0.309 | 0.446 |
| LDN-212854 | 2.00 | −0.475 | −0.217 | −0.292 | −1.134 | −1.885 |
| LDN-212854 | 10.00 | −1.263 | −0.273 | −0.399 | −3.018 | −2.375 |
| Lenalidomide (CC-5013) | 0.08 | 0.046 | 0.147 | 0.099 | 0.110 | 1.283 |
| Lenalidomide (CC-5013) | 0.40 | −0.016 | 0.099 | 0.176 | −0.037 | 0.863 |
| Lenalidomide (CC-5013) | 2.00 | −0.250 | 0.071 | 0.025 | −0.596 | 0.615 |
| Lenalidomide (CC-5013) | 10.00 | −0.316 | −0.041 | −0.045 | −0.754 | −0.360 |
| Letrozole | 0.08 | −0.013 | 0.065 | 0.040 | −0.031 | 0.570 |
| Letrozole | 0.40 | 0.101 | 0.128 | 0.150 | 0.241 | 1.115 |
| Letrozole | 2.00 | 0.099 | 0.009 | 0.006 | 0.236 | 0.082 |
| Letrozole | 10.00 | 0.171 | −0.008 | 0.006 | 0.408 | −0.068 |
| Levosulpiride | 0.08 | −0.043 | 0.013 | −0.017 | −0.102 | 0.114 |
| Levosulpiride | 0.40 | −0.006 | 0.024 | 0.128 | −0.015 | 0.205 |
| Levosulpiride | 2.00 | 0.031 | 0.051 | 0.124 | 0.074 | 0.441 |
| Levosulpiride | 10.00 | 0.090 | −0.028 | −0.026 | 0.215 | −0.240 |
| Lidocaine | 0.08 | −0.079 | 0.106 | 0.083 | −0.188 | 0.927 |
| Lidocaine | 0.40 | −0.153 | 0.017 | 0.026 | −0.365 | 0.144 |
| Lidocaine | 2.00 | 0.034 | 0.001 | −0.038 | 0.081 | 0.008 |
| Lidocaine | 10.00 | −0.256 | 0.019 | −0.009 | −0.611 | 0.167 |
| Linagliptin | 0.08 | 0.163 | 0.208 | 0.140 | 0.389 | 1.809 |
| Linagliptin | 0.40 | 0.209 | 0.055 | 0.047 | 0.499 | 0.478 |
| Linagliptin | 2.00 | 0.023 | 0.021 | −0.032 | 0.054 | 0.182 |
| Linagliptin | 10.00 | −0.011 | 0.080 | 0.050 | −0.027 | 0.695 |
| Lomeguatrib | 0.08 | −0.426 | −0.050 | −0.082 | −1.017 | −0.437 |
| Lomeguatrib | 0.40 | −0.309 | −0.019 | −0.022 | −0.739 | −0.161 |
| Lomeguatrib | 2.00 | 0.075 | −0.022 | −0.055 | 0.178 | −0.193 |
| Lomeguatrib | 10.00 | −0.080 | −0.104 | −0.138 | −0.192 | −0.908 |
| Loratadine | 0.08 | −0.292 | 0.011 | −0.076 | −0.697 | 0.093 |
| Loratadine | 0.40 | 0.145 | −0.001 | 0.011 | 0.347 | −0.005 |
| Loratadine | 2.00 | −0.204 | −0.108 | −0.148 | −0.488 | −0.938 |
| Loratadine | 10.00 | −0.120 | −0.101 | −0.148 | −0.287 | −0.881 |
| Losartan Potassium (DuP 753) | 0.08 | −0.152 | 0.052 | −0.017 | −0.362 | 0.449 |
| Losartan Potassium (DuP 753) | 0.40 | −0.175 | 0.038 | 0.027 | −0.418 | 0.329 |
| Losartan Potassium (DuP 753) | 2.00 | −0.081 | −0.042 | −0.080 | −0.193 | −0.362 |
| Losartan Potassium (DuP 753) | 10.00 | −0.137 | 0.062 | −0.025 | −0.328 | 0.540 |
| Lovastatin | 0.08 | −0.322 | 0.002 | −0.023 | −0.769 | 0.017 |
| Lovastatin | 0.40 | −0.492 | −0.057 | −0.049 | −1.174 | −0.492 |
| Lovastatin | 2.00 | −1.174 | −0.279 | −0.398 | −2.803 | −2.430 |
| Lovastatin | 10.00 | −1.391 | −0.271 | −0.409 | −3.322 | −2.362 |
| Loxistatin Acid (E-64C) | 0.08 | 0.086 | 0.147 | 0.125 | 0.206 | 1.283 |
| Loxistatin Acid (E-64C) | 0.40 | 0.156 | 0.061 | 0.105 | 0.373 | 0.534 |
| Loxistatin Acid (E-64C) | 2.00 | 0.080 | 0.055 | 0.037 | 0.191 | 0.482 |
| Loxistatin Acid (E-64C) | 10.00 | 0.094 | 0.004 | 0.021 | 0.224 | 0.031 |
| LY2157299 | 0.08 | 0.091 | 0.227 | 0.187 | 0.218 | 1.973 |
| LY2157299 | 0.40 | 0.137 | 0.140 | 0.199 | 0.328 | 1.221 |
| LY2157299 | 2.00 | 0.017 | 0.092 | 0.113 | 0.041 | 0.799 |
| LY2157299 | 10.00 | 0.095 | 0.162 | 0.223 | 0.226 | 1.408 |
| LY2228820 | 0.08 | −0.299 | 0.065 | −0.017 | −0.715 | 0.569 |
| LY2228820 | 0.40 | −0.340 | −0.100 | −0.054 | −0.812 | −0.874 |
| LY2228820 | 2.00 | −0.339 | 0.043 | −0.130 | −0.810 | 0.377 |
| LY2228820 | 10.00 | −0.332 | −0.126 | −0.164 | −0.793 | −1.099 |
| LY2603618 | 0.08 | −0.004 | −0.005 | 0.009 | −0.010 | −0.046 |
| LY2603618 | 0.40 | −0.238 | −0.091 | −0.147 | −0.567 | −0.791 |
| LY2603618 | 2.00 | −0.396 | −0.193 | −0.269 | −0.946 | −1.685 |
| LY2603618 | 10.00 | −0.983 | −0.233 | −0.356 | −2.347 | −2.026 |
| LY2784544 | 0.08 | 0.108 | 0.132 | 0.123 | 0.259 | 1.152 |
| LY2784544 | 0.40 | 0.062 | 0.004 | 0.077 | 0.148 | 0.033 |
| LY2784544 | 2.00 | 0.181 | 0.068 | 0.114 | 0.433 | 0.591 |
| LY2784544 | 10.00 | −0.831 | −0.280 | −0.367 | −1.984 | −2.439 |
| LY411575 | 0.08 | 0.035 | 0.040 | 0.024 | 0.083 | 0.347 |
| LY411575 | 0.40 | −0.172 | −0.050 | 0.041 | −0.411 | −0.434 |
| LY411575 | 2.00 | −0.172 | −0.069 | −0.091 | −0.412 | −0.598 |
| LY411575 | 10.00 | −0.259 | −0.109 | −0.157 | −0.618 | −0.949 |
| Maraviroc | 0.08 | −0.084 | 0.047 | 0.054 | −0.201 | 0.411 |
| Maraviroc | 0.40 | 0.079 | 0.041 | 0.195 | 0.188 | 0.358 |
| Maraviroc | 2.00 | −0.009 | 0.031 | 0.005 | −0.022 | 0.269 |
| Maraviroc | 10.00 | 0.139 | 0.087 | 0.077 | 0.331 | 0.759 |
| Mdivi-1 | 0.08 | 0.008 | 0.177 | 0.079 | 0.019 | 1.537 |
| Mdivi-1 | 0.40 | −0.031 | 0.156 | 0.030 | −0.074 | 1.358 |
| Mdivi-1 | 2.00 | −0.112 | −0.022 | −0.048 | −0.267 | −0.194 |
| Mdivi-1 | 10.00 | −0.527 | −0.268 | −0.411 | −1.259 | −2.333 |
| Memantine HCl | 0.08 | 0.083 | 0.069 | 0.091 | 0.199 | 0.598 |
| Memantine HCl | 0.40 | 0.072 | 0.004 | 0.017 | 0.173 | 0.032 |
| Memantine HCl | 2.00 | −0.007 | 0.106 | 0.051 | −0.018 | 0.919 |
| Memantine HCl | 10.00 | 0.077 | −0.047 | −0.021 | 0.184 | −0.411 |
| Methotrexate | 0.08 | −0.615 | −0.097 | −0.159 | −1.469 | −0.843 |
| Methotrexate | 0.40 | −0.722 | −0.038 | −0.129 | −1.724 | −0.332 |
| Methotrexate | 2.00 | −0.701 | −0.130 | −0.177 | −1.674 | −1.132 |
| Methotrexate | 10.00 | −0.642 | −0.076 | −0.156 | −1.534 | −0.658 |
| Mirabegron | 0.08 | 0.029 | 0.138 | 0.148 | 0.069 | 1.201 |
| Mirabegron | 0.40 | −0.335 | −0.030 | −0.016 | −0.801 | −0.259 |
| Mirabegron | 2.00 | 0.019 | −0.035 | −0.049 | 0.046 | −0.301 |
| Mirabegron | 10.00 | −0.051 | 0.022 | −0.010 | −0.123 | 0.193 |
| MK-1775 | 0.08 | 0.165 | 0.038 | 0.066 | 0.393 | 0.334 |
| MK-1775 | 0.40 | −0.450 | −0.153 | −0.217 | −1.074 | −1.331 |
| MK-1775 | 2.00 | −0.804 | −0.215 | −0.208 | −1.919 | −1.869 |
| MK-1775 | 10.00 | −1.026 | −0.181 | −0.254 | −2.451 | −1.574 |
| MK-2206 2HCl | 0.08 | 0.071 | 0.024 | 0.071 | 0.169 | 0.205 |
| MK-2206 2HCl | 0.40 | −0.023 | −0.063 | 0.083 | −0.055 | −0.550 |
| MK-2206 2HCl | 2.00 | −0.178 | −0.182 | −0.239 | −0.425 | −1.587 |
| MK-2206 2HCl | 10.00 | −0.540 | −0.236 | −0.367 | −1.290 | −2.058 |
| MK-2866 (GTx-024) | 0.08 | 0.001 | 0.035 | 0.072 | 0.003 | 0.309 |
| MK-2866 (GTx-024) | 0.40 | −0.198 | 0.028 | 0.050 | −0.474 | 0.241 |
| MK-2866 (GTx-024) | 2.00 | −0.088 | 0.036 | 0.078 | −0.210 | 0.310 |
| MK-2866 (GTx-024) | 10.00 | −0.367 | −0.059 | −0.072 | −0.877 | −0.510 |
| MK-8245 | 0.08 | 0.156 | 0.023 | 0.069 | 0.374 | 0.200 |
| MK-8245 | 0.40 | −0.113 | 0.031 | 0.185 | −0.269 | 0.273 |
| MK-8245 | 2.00 | −0.021 | −0.036 | −0.062 | −0.051 | −0.315 |
| MK-8245 | 10.00 | −0.263 | −0.077 | −0.123 | −0.629 | −0.670 |
| ML130 (Nodinitib-1) | 0.08 | 0.046 | 0.028 | 0.014 | 0.111 | 0.247 |
| ML130 (Nodinitib-1) | 0.40 | 0.051 | 0.007 | 0.014 | 0.123 | 0.061 |
| ML130 (Nodinitib-1) | 2.00 | 0.119 | 0.173 | 0.066 | 0.283 | 1.505 |
| ML130 (Nodinitib-1) | 10.00 | 0.156 | 0.021 | −0.042 | 0.373 | 0.183 |
| ML133 HCl | 0.08 | −0.009 | 0.030 | 0.062 | −0.023 | 0.261 |
| ML133 HCl | 0.40 | −0.151 | −0.013 | −0.018 | −0.360 | −0.116 |
| ML133 HCl | 2.00 | −0.247 | 0.073 | 0.087 | −0.591 | 0.633 |
| ML133 HCl | 10.00 | −0.040 | −0.018 | −0.014 | −0.096 | −0.155 |
| ML161 | 0.08 | 0.086 | 0.018 | 0.017 | 0.205 | 0.158 |
| ML161 | 0.40 | 0.028 | −0.027 | −0.027 | 0.067 | −0.237 |
| ML161 | 2.00 | 0.066 | 0.037 | 0.024 | 0.157 | 0.323 |
| ML161 | 10.00 | −0.035 | −0.057 | −0.066 | −0.084 | −0.494 |
| ML347 | 0.08 | 0.184 | 0.124 | 0.094 | 0.439 | 1.079 |
| ML347 | 0.40 | 0.209 | 0.132 | 0.111 | 0.500 | 1.150 |
| ML347 | 2.00 | 0.137 | 0.069 | 0.072 | 0.328 | 0.600 |
| ML347 | 10.00 | −0.169 | −0.124 | −0.202 | −0.404 | −1.078 |
| MLN2238 | 0.08 | −1.655 | −0.271 | −0.383 | −3.952 | −2.358 |
| MLN2238 | 0.40 | −1.104 | −0.284 | −0.358 | −2.637 | −2.477 |
| MLN2238 | 2.00 | −1.233 | −0.249 | −0.353 | −2.944 | −2.166 |
| MLN2238 | 10.00 | −1.761 | −0.298 | −0.370 | −4.205 | −2.597 |
| MLN8054 | 0.08 | 0.026 | 0.066 | 0.042 | 0.063 | 0.574 |
| MLN8054 | 0.40 | 0.014 | 0.008 | 0.167 | 0.033 | 0.066 |
| MLN8054 | 2.00 | −0.465 | −0.100 | −0.087 | −1.111 | −0.868 |
| MLN8054 | 10.00 | −0.498 | −0.112 | −0.197 | −1.191 | −0.979 |
| MM-102 | 0.08 | −0.205 | −0.025 | −0.027 | −0.489 | −0.220 |
| MM-102 | 0.40 | −0.132 | −0.010 | −0.001 | −0.315 | −0.090 |
| MM-102 | 2.00 | −0.233 | 0.026 | −0.035 | −0.557 | 0.230 |
| MM-102 | 10.00 | −0.403 | −0.164 | −0.194 | −0.964 | −1.424 |
| MNS (3,4-Methylenedioxy- | 0.08 | −0.016 | 0.057 | 0.076 | −0.037 | 0.499 |
| CE≤-nitrostyrene, MDBN) | | | | | | |
| MNS (3,4-Methylenedioxy- | 0.40 | 0.061 | −0.010 | 0.100 | 0.146 | −0.088 |
| CE≤-nitrostyrene, MDBN) | | | | | | |
| MNS (3,4-Methylenedioxy- | 2.00 | 0.062 | 0.040 | 0.019 | 0.148 | 0.347 |
| CE≤-nitrostyrene, MDBN) | | | | | | |
| MNS (3,4-Methylenedioxy- | 10.00 | −0.197 | −0.150 | −0.179 | −0.469 | −1.307 |
| CE≤-nitrostyrene, MDBN) | | | | | | |
| Moclobemide (Ro 111163) | 0.08 | 0.146 | 0.075 | 0.147 | 0.348 | 0.654 |
| Moclobemide (Ro 111163) | 0.40 | 0.106 | 0.121 | 0.094 | 0.252 | 1.055 |
| Moclobemide (Ro 111163) | 2.00 | 0.116 | 0.001 | 0.012 | 0.276 | 0.011 |
| Moclobemide (Ro 111163) | 10.00 | 0.042 | −0.028 | 0.019 | 0.100 | −0.243 |
| Mozavaptan | 0.08 | 0.320 | 0.067 | 0.086 | 0.765 | 0.584 |
| Mozavaptan | 0.40 | 0.235 | −0.027 | 0.165 | 0.562 | −0.233 |
| Mozavaptan | 2.00 | 0.108 | 0.034 | 0.106 | 0.259 | 0.294 |
| Mozavaptan | 10.00 | −0.336 | −0.201 | −0.270 | −0.803 | −1.752 |
| MRS 2578 | 0.08 | −0.142 | 0.116 | 0.095 | −0.339 | 1.011 |
| MRS 2578 | 0.40 | 0.061 | 0.036 | 0.067 | 0.146 | 0.311 |
| MRS 2578 | 2.00 | −0.159 | 0.031 | 0.023 | −0.381 | 0.270 |
| MRS 2578 | 10.00 | −0.228 | 0.032 | 0.013 | −0.544 | 0.276 |
| Mubritinib (TAK 165) | 0.08 | −0.524 | −0.057 | −0.119 | −1.252 | −0.500 |
| Mubritinib (TAK 165) | 0.40 | −0.340 | −0.194 | −0.154 | −0.812 | −1.687 |
| Mubritinib (TAK 165) | 2.00 | −0.530 | −0.224 | −0.299 | −1.265 | −1.950 |
| Mubritinib (TAK 165) | 10.00 | −0.519 | −0.240 | −0.294 | −1.240 | −2.092 |
| Naftopidil | 0.08 | −0.039 | −0.037 | 0.015 | −0.093 | −0.325 |
| Naftopidil | 0.40 | −0.161 | −0.082 | −0.128 | −0.384 | −0.713 |
| Naftopidil | 2.00 | −0.179 | −0.020 | −0.097 | −0.426 | −0.171 |
| Naftopidil | 10.00 | −0.132 | −0.073 | −0.079 | −0.316 | −0.638 |
| Naltrexone HCl | 0.08 | 0.089 | −0.008 | 0.002 | 0.213 | −0.067 |
| Naltrexone HCl | 0.40 | 0.070 | −0.005 | 0.025 | 0.167 | −0.043 |
| Naltrexone HCl | 2.00 | 0.004 | 0.019 | 0.025 | 0.010 | 0.166 |
| Naltrexone HCl | 10.00 | 0.106 | 0.035 | 0.045 | 0.253 | 0.302 |
| Naproxen | 0.08 | −0.098 | 0.141 | 0.067 | −0.233 | 1.226 |
| Naproxen | 0.40 | −0.068 | 0.107 | 0.057 | −0.162 | 0.930 |
| Naproxen | 2.00 | −0.588 | −0.080 | −0.215 | −1.405 | −0.698 |
| Naproxen | 10.00 | −1.282 | −0.122 | −0.215 | −3.063 | −1.064 |
| NC | 0.08 | −1.613 | −0.235 | −0.342 | −3.853 | −2.044 |
| NC | 0.40 | −1.409 | −0.222 | −0.326 | −3.365 | −1.932 |
| NC | 2.00 | −1.502 | −0.226 | −0.318 | −3.587 | −1.965 |
| NC | 10.00 | −1.551 | −0.229 | −0.320 | −3.704 | −1.995 |
| Nebivolol | 0.08 | 0.388 | 0.175 | 0.194 | 0.926 | 1.525 |
| Nebivolol | 0.40 | 0.220 | 0.319 | 0.282 | 0.526 | 2.776 |
| Nebivolol | 2.00 | 0.312 | 0.110 | 0.069 | 0.746 | 0.960 |
| Nebivolol | 10.00 | 0.209 | 0.096 | 0.105 | 0.499 | 0.836 |
| Necrostatin-1 | 0.08 | 0.102 | 0.060 | 0.077 | 0.244 | 0.524 |
| Necrostatin-1 | 0.40 | 0.168 | 0.020 | 0.038 | 0.401 | 0.174 |
| Necrostatin-1 | 2.00 | 0.011 | 0.154 | 0.092 | 0.027 | 1.338 |
| Necrostatin-1 | 10.00 | 0.137 | 0.057 | 0.229 | 0.328 | 0.496 |
| NH125 | 0.08 | −0.347 | −0.035 | −0.114 | −0.829 | −0.301 |
| NH125 | 0.40 | −0.508 | −0.188 | −0.237 | −1.213 | −1.634 |
| NH125 | 2.00 | −1.106 | −0.219 | −0.358 | −2.641 | −1.904 |
| NH125 | 10.00 | −1.787 | −0.263 | −0.374 | −4.268 | −2.293 |
| Nilotinib (AMN-107) | 0.08 | 0.322 | 0.196 | 0.235 | 0.769 | 1.703 |
| Nilotinib (AMN-107) | 0.40 | 0.348 | 0.162 | 0.293 | 0.831 | 1.412 |
| Nilotinib (AMN-107) | 2.00 | 0.242 | 0.085 | 0.033 | 0.577 | 0.742 |
| Nilotinib (AMN-107) | 10.00 | −0.012 | −0.093 | −0.197 | −0.029 | −0.806 |
| Nilvadipine | 0.08 | 0.206 | 0.098 | 0.088 | 0.493 | 0.857 |
| Nilvadipine | 0.40 | 0.135 | 0.004 | 0.013 | 0.324 | 0.034 |
| Nilvadipine | 2.00 | 0.212 | 0.057 | 0.071 | 0.506 | 0.500 |
| Nilvadipine | 10.00 | 0.192 | 0.009 | −0.008 | 0.460 | 0.082 |
| NLG919 | 0.08 | −0.098 | −0.053 | −0.002 | −0.234 | −0.463 |
| NLG919 | 0.40 | −0.168 | −0.115 | −0.085 | −0.400 | −0.998 |
| NLG919 | 2.00 | −0.126 | −0.062 | 0.050 | −0.300 | −0.539 |
| NLG919 | 10.00 | −0.203 | −0.016 | −0.043 | −0.485 | −0.136 |
| NMS-873 | 0.08 | 0.174 | 0.098 | 0.069 | 0.416 | 0.853 |
| NMS-873 | 0.40 | −0.051 | −0.177 | −0.228 | −0.122 | −1.537 |
| NMS-873 | 2.00 | −0.910 | −0.270 | −0.343 | −2.173 | −2.352 |
| NMS-873 | 10.00 | −1.542 | −0.272 | −0.375 | −3.683 | −2.368 |
| NPS-2143 | 0.08 | 0.149 | 0.162 | 0.147 | 0.356 | 1.413 |
| NPS-2143 | 0.40 | −0.195 | −0.048 | −0.062 | −0.466 | −0.416 |
| NPS-2143 | 2.00 | −0.246 | −0.077 | −0.161 | −0.587 | −0.674 |
| NPS-2143 | 10.00 | −0.118 | −0.069 | −0.116 | −0.282 | −0.597 |
| NSC 23766 | 0.08 | 0.043 | 0.081 | 0.080 | 0.103 | 0.701 |
| NSC 23766 | 0.40 | 0.170 | 0.044 | 0.090 | 0.406 | 0.380 |
| NSC 23766 | 2.00 | 0.117 | 0.058 | 0.031 | 0.279 | 0.506 |
| NSC 23766 | 10.00 | 0.324 | 0.009 | 0.030 | 0.774 | 0.080 |
| NSC 319726 | 0.08 | 0.132 | 0.113 | 0.060 | 0.316 | 0.982 |
| NSC 319726 | 0.40 | −0.185 | −0.090 | −0.035 | −0.441 | −0.780 |
| NSC 319726 | 2.00 | −0.447 | −0.152 | −0.248 | −1.068 | −1.323 |
| NSC 319726 | 10.00 | −1.907 | −0.229 | −0.327 | −4.555 | −1.992 |
| NSC 405020 | 0.08 | −0.059 | 0.008 | 0.014 | −0.140 | 0.071 |
| NSC 405020 | 0.40 | −0.071 | −0.028 | 0.093 | −0.170 | −0.246 |
| NSC 405020 | 2.00 | −0.059 | 0.018 | 0.002 | −0.141 | 0.160 |
| NSC 405020 | 10.00 | −0.058 | −0.065 | −0.061 | −0.138 | −0.570 |
| NSC697923 | 0.08 | −0.052 | 0.091 | 0.036 | −0.123 | 0.789 |
| NSC697923 | 0.40 | −0.014 | 0.051 | 0.108 | −0.033 | 0.441 |
| NSC697923 | 2.00 | −0.101 | −0.104 | −0.154 | −0.241 | −0.906 |
| NSC697923 | 10.00 | −0.782 | −0.154 | −0.270 | −1.867 | −1.338 |
| NU7026 | 0.08 | 0.096 | 0.023 | 0.021 | 0.230 | 0.198 |
| NU7026 | 0.40 | 0.104 | 0.023 | 0.034 | 0.248 | 0.198 |
| NU7026 | 2.00 | 0.107 | −0.058 | −0.056 | 0.255 | −0.507 |
| NU7026 | 10.00 | −0.119 | −0.158 | −0.198 | −0.284 | −1.380 |
| NVP-ADW742 | 0.08 | 0.679 | 0.142 | 0.245 | 1.622 | 1.239 |
| NVP-ADW742 | 0.40 | 0.663 | 0.172 | 0.265 | 1.583 | 1.499 |
| NVP-ADW742 | 2.00 | 0.257 | −0.095 | −0.166 | 0.614 | −0.823 |
| NVP-ADW742 | 10.00 | −0.465 | −0.135 | −0.210 | −1.112 | −1.172 |
| OC000459 | 0.08 | 0.077 | 0.048 | 0.090 | 0.184 | 0.416 |
| OC000459 | 0.40 | −0.065 | 0.053 | 0.199 | −0.155 | 0.461 |
| OC000459 | 2.00 | 0.088 | 0.058 | 0.130 | 0.210 | 0.507 |
| OC000459 | 10.00 | 0.088 | 0.033 | 0.051 | 0.211 | 0.287 |
| Odanacatib (MK-0822) | 0.08 | 0.119 | 0.086 | 0.091 | 0.285 | 0.752 |
| Odanacatib (MK-0822) | 0.40 | 0.208 | 0.162 | 0.170 | 0.496 | 1.409 |
| Odanacatib (MK-0822) | 2.00 | −0.008 | 0.024 | 0.036 | −0.020 | 0.212 |
| Odanacatib (MK-0822) | 10.00 | 0.006 | −0.035 | −0.074 | 0.015 | −0.307 |
| OG-L002 | 0.08 | 0.094 | 0.000 | 0.023 | 0.225 | −0.001 |
| OG-L002 | 0.40 | 0.138 | −0.011 | 0.026 | 0.329 | −0.097 |
| OG-L002 | 2.00 | 0.128 | 0.021 | −0.001 | 0.306 | 0.182 |
| OG-L002 | 10.00 | 0.094 | −0.091 | −0.084 | 0.225 | −0.790 |
| Oligomycin A | 0.08 | −0.662 | −0.204 | −0.243 | −1.581 | −1.778 |
| Oligomycin A | 0.40 | −0.706 | −0.231 | −0.274 | −1.685 | −2.013 |
| Oligomycin A | 2.00 | −0.946 | −0.213 | −0.269 | −2.261 | −1.857 |
| Oligomycin A | 10.00 | −1.244 | −0.223 | −0.288 | −2.972 | −1.943 |
| Org 27569 | 0.08 | 0.278 | 0.122 | 0.174 | 0.663 | 1.060 |
| Org 27569 | 0.40 | 0.072 | 0.051 | 0.032 | 0.173 | 0.445 |
| Org 27569 | 2.00 | 0.362 | 0.078 | 0.059 | 0.865 | 0.683 |
| Org 27569 | 10.00 | 0.270 | 0.029 | 0.001 | 0.646 | 0.248 |
| OSI-420 | 0.08 | 0.106 | 0.115 | 0.059 | 0.254 | 1.002 |
| OSI-420 | 0.40 | −0.130 | −0.224 | −0.221 | −0.311 | −1.948 |
| OSI-420 | 2.00 | −1.263 | −0.287 | −0.381 | −3.016 | −2.498 |
| OSI-420 | 10.00 | −1.100 | −0.325 | −0.374 | −2.627 | −2.833 |
| OSI-906 (Linsitinib) | 0.08 | −0.324 | 0.013 | −0.026 | −0.775 | 0.110 |
| OSI-906 (Linsitinib) | 0.40 | −0.158 | −0.093 | −0.119 | −0.377 | −0.806 |
| OSI-906 (Linsitinib) | 2.00 | −0.277 | −0.216 | −0.288 | −0.661 | −1.882 |
| OSI-906 (Linsitinib) | 10.00 | −0.391 | −0.229 | −0.328 | −0.934 | −1.996 |
| OSU-03012 (AR-12) | 0.08 | 0.205 | 0.087 | 0.126 | 0.491 | 0.755 |
| OSU-03012 (AR-12) | 0.40 | 0.114 | 0.033 | 0.113 | 0.272 | 0.285 |
| OSU-03012 (AR-12) | 2.00 | 0.233 | 0.053 | 0.060 | 0.556 | 0.460 |
| OSU-03012 (AR-12) | 10.00 | 0.078 | −0.049 | −0.128 | 0.185 | −0.423 |
| OTX015 | 0.08 | −0.038 | −0.198 | −0.279 | −0.092 | −1.726 |
| OTX015 | 0.40 | −0.398 | −0.266 | −0.350 | −0.952 | −2.317 |
| OTX015 | 2.00 | −0.686 | −0.239 | −0.350 | −1.638 | −2.082 |
| OTX015 | 10.00 | −0.908 | −0.256 | −0.362 | −2.169 | −2.225 |
| Ouabain | 0.08 | 0.010 | −0.003 | 0.040 | 0.023 | −0.025 |
| Ouabain | 0.40 | 0.122 | 0.036 | 0.035 | 0.290 | 0.315 |
| Ouabain | 2.00 | 0.080 | 0.011 | 0.003 | 0.190 | 0.092 |
| Ouabain | 10.00 | −0.288 | 0.165 | 0.050 | −0.688 | 1.433 |
| Oxcarbazepine | 0.08 | −0.021 | 0.004 | 0.043 | −0.051 | 0.035 |
| Oxcarbazepine | 0.40 | −0.103 | 0.000 | 0.154 | −0.247 | −0.001 |
| Oxcarbazepine | 2.00 | −0.082 | 0.040 | 0.042 | −0.197 | 0.349 |
| Oxcarbazepine | 10.00 | −0.150 | −0.061 | −0.057 | −0.359 | −0.527 |
| Oxymetazoline HCl | 0.08 | 0.143 | 0.059 | 0.143 | 0.340 | 0.515 |
| Oxymetazoline HCl | 0.40 | 0.118 | 0.015 | 0.116 | 0.281 | 0.134 |
| Oxymetazoline HCl | 2.00 | −0.007 | 0.023 | −0.012 | −0.016 | 0.199 |
| Oxymetazoline HCl | 10.00 | 0.118 | 0.007 | −0.046 | 0.282 | 0.065 |
| Ozagrel | 0.08 | 0.055 | 0.074 | 0.145 | 0.131 | 0.645 |
| Ozagrel | 0.40 | 0.224 | 0.044 | 0.096 | 0.535 | 0.381 |
| Ozagrel | 2.00 | −0.063 | 0.080 | 0.104 | −0.149 | 0.698 |
| Ozagrel | 10.00 | 0.113 | −0.008 | 0.011 | 0.271 | −0.068 |
| Ozagrel HCl | 0.08 | 0.049 | 0.202 | 0.119 | 0.118 | 1.756 |
| Ozagrel HCl | 0.40 | 0.133 | 0.000 | 0.073 | 0.318 | 0.000 |
| Ozagrel HCl | 2.00 | 0.183 | 0.035 | 0.135 | 0.437 | 0.308 |
| Ozagrel HCl | 10.00 | 0.229 | −0.007 | 0.061 | 0.547 | −0.061 |
| P22077 | 0.08 | −0.216 | 0.039 | −0.182 | −0.516 | 0.338 |
| P22077 | 0.40 | 0.049 | −0.005 | 0.065 | 0.117 | −0.041 |
| P22077 | 2.00 | −0.134 | 0.028 | −0.007 | −0.320 | 0.248 |
| P22077 | 10.00 | −0.130 | −0.056 | −0.063 | −0.311 | −0.487 |
| PAC-1 | 0.08 | 0.095 | 0.157 | 0.128 | 0.228 | 1.365 |
| PAC-1 | 0.40 | 0.113 | 0.053 | 0.059 | 0.270 | 0.457 |
| PAC-1 | 2.00 | −0.241 | 0.126 | −0.006 | −0.576 | 1.094 |
| PAC-1 | 10.00 | 0.002 | 0.068 | 0.092 | 0.004 | 0.596 |
| Pacritinib (SB1518) | 0.08 | −0.006 | 0.091 | 0.066 | −0.014 | 0.795 |
| Pacritinib (SB1518) | 0.40 | 0.099 | −0.034 | −0.032 | 0.236 | −0.299 |
| Pacritinib (SB1518) | 2.00 | −0.235 | −0.219 | −0.304 | −0.560 | −1.908 |
| Pacritinib (SB1518) | 10.00 | −1.276 | −0.275 | −0.419 | −3.047 | −2.391 |
| Palbociclib (PD-0332991) HCl | 0.08 | 0.169 | −0.006 | 0.004 | 0.404 | −0.055 |
| Palbociclib (PD-0332991) HCl | 0.40 | −0.350 | −0.029 | −0.042 | −0.836 | −0.248 |
| Palbociclib (PD-0332991) HCl | 2.00 | −0.083 | −0.051 | −0.122 | −0.199 | −0.440 |
| Palbociclib (PD-0332991) HCl | 10.00 | −0.677 | −0.132 | −0.203 | −1.617 | −1.150 |
| Pancuronium dibromide | 0.08 | 0.217 | 0.128 | 0.213 | 0.519 | 1.118 |
| Pancuronium dibromide | 0.40 | 0.226 | 0.047 | 0.094 | 0.541 | 0.406 |
| Pancuronium dibromide | 2.00 | −0.012 | 0.093 | 0.099 | −0.029 | 0.806 |
| Pancuronium dibromide | 10.00 | 0.062 | 0.035 | 0.004 | 0.148 | 0.302 |
| Panobinostat (LBH589) | 0.08 | 0.295 | 0.002 | −0.034 | 0.706 | 0.014 |
| Panobinostat (LBH589) | 0.40 | 0.212 | −0.027 | −0.042 | 0.506 | −0.232 |
| Panobinostat (LBH589) | 2.00 | −0.537 | −0.153 | −0.285 | −1.283 | −1.329 |
| Panobinostat (LBH589) | 10.00 | −1.003 | −0.217 | −0.320 | −2.395 | −1.891 |
| PD0325901 | 0.08 | −0.502 | −0.120 | −0.212 | −1.200 | −1.046 |
| PD0325901 | 0.40 | −1.155 | −0.122 | −0.219 | −2.760 | −1.064 |
| PD0325901 | 2.00 | −0.867 | −0.139 | −0.227 | −2.072 | −1.215 |
| PD0325901 | 10.00 | −0.963 | −0.187 | −0.269 | −2.301 | −1.625 |
| PD128907 HCl | 0.08 | 0.313 | 0.091 | 0.116 | 0.749 | 0.791 |
| PD128907 HCl | 0.40 | 0.401 | 0.144 | 0.270 | 0.959 | 1.250 |
| PD128907 HCl | 2.00 | 0.273 | 0.089 | 0.142 | 0.651 | 0.776 |
| PD128907 HCl | 10.00 | 0.285 | 0.113 | 0.079 | 0.680 | 0.982 |
| PD184352 (CI-1040) | 0.08 | 0.366 | −0.019 | −0.019 | 0.874 | −0.162 |
| PD184352 (CI-1040) | 0.40 | 0.261 | −0.053 | −0.047 | 0.624 | −0.458 |
| PD184352 (CI-1040) | 2.00 | −0.212 | −0.135 | −0.178 | −0.506 | −1.176 |
| PD184352 (CI-1040) | 10.00 | −0.796 | −0.116 | −0.239 | −1.901 | −1.011 |
| PF-04217903 | 0.08 | 0.136 | 0.085 | 0.110 | 0.326 | 0.742 |
| PF-04217903 | 0.40 | 0.258 | 0.161 | 0.265 | 0.616 | 1.404 |
| PF-04217903 | 2.00 | −0.051 | 0.122 | 0.049 | −0.122 | 1.060 |
| PF-04217903 | 10.00 | 0.116 | 0.071 | 0.127 | 0.276 | 0.617 |
| PF-3845 | 0.08 | −0.047 | 0.061 | 0.027 | −0.112 | 0.532 |
| PF-3845 | 0.40 | 0.052 | −0.077 | −0.061 | 0.125 | −0.668 |
| PF-3845 | 2.00 | −0.103 | −0.015 | −0.032 | −0.246 | −0.131 |
| PF-3845 | 10.00 | −0.286 | −0.131 | −0.195 | −0.684 | −1.137 |
| PF-4708671 | 0.08 | 0.261 | 0.075 | 0.136 | 0.623 | 0.654 |
| PF-4708671 | 0.40 | 0.268 | 0.122 | 0.172 | 0.640 | 1.059 |
| PF-4708671 | 2.00 | 0.302 | 0.198 | 0.209 | 0.721 | 1.721 |
| PF-4708671 | 10.00 | −0.321 | 0.034 | −0.024 | −0.766 | 0.297 |
| PF-5274857 | 0.08 | −0.101 | 0.078 | 0.187 | −0.242 | 0.683 |
| PF-5274857 | 0.40 | −0.040 | −0.029 | −0.034 | −0.096 | −0.256 |
| PF-5274857 | 2.00 | 0.055 | 0.011 | 0.042 | 0.131 | 0.094 |
| PF-5274857 | 10.00 | −0.076 | 0.074 | 0.076 | −0.183 | 0.642 |
| PF-562271 | 0.08 | −0.114 | −0.068 | −0.088 | −0.273 | −0.589 |
| PF-562271 | 0.40 | −0.217 | −0.152 | −0.214 | −0.518 | −1.325 |
| PF-562271 | 2.00 | −0.549 | −0.234 | −0.332 | −1.311 | −2.034 |
| PF-562271 | 10.00 | −0.949 | −0.251 | −0.414 | −2.266 | −2.184 |
| PF-573228 | 0.08 | 0.077 | 0.010 | 0.114 | 0.183 | 0.089 |
| PF-573228 | 0.40 | −0.054 | −0.046 | 0.002 | −0.129 | −0.401 |
| PF-573228 | 2.00 | −0.434 | −0.058 | −0.174 | −1.037 | −0.502 |
| PF-573228 | 10.00 | −0.497 | −0.250 | −0.305 | −1.188 | −2.180 |
| PFI-1 (PF-6405761) | 0.08 | 0.142 | 0.062 | 0.061 | 0.340 | 0.538 |
| PFI-1 (PF-6405761) | 0.40 | 0.390 | 0.079 | 0.104 | 0.931 | 0.691 |
| PFI-1 (PF-6405761) | 2.00 | 0.322 | −0.098 | −0.117 | 0.770 | −0.852 |
| PFI-1 (PF-6405761) | 10.00 | −0.389 | −0.066 | −0.194 | −0.929 | −0.573 |
| PHA-665752 | 0.08 | 0.471 | 0.182 | 0.163 | 1.126 | 1.586 |
| PHA-665752 | 0.40 | 0.620 | 0.272 | 0.373 | 1.482 | 2.368 |
| PHA-665752 | 2.00 | 0.561 | 0.183 | 0.143 | 1.339 | 1.591 |
| PHA-665752 | 10.00 | 0.024 | −0.107 | −0.165 | 0.057 | −0.931 |
| PHA-793887 | 0.08 | 0.113 | 0.032 | 0.046 | 0.271 | 0.275 |
| PHA-793887 | 0.40 | 0.132 | 0.010 | 0.100 | 0.314 | 0.085 |
| PHA-793887 | 2.00 | −0.144 | −0.136 | −0.081 | −0.344 | −1.187 |
| PHA-793887 | 10.00 | −0.856 | −0.267 | −0.292 | −2.045 | −2.324 |
| Piceatannol | 0.08 | 0.232 | 0.080 | 0.094 | 0.554 | 0.698 |
| Piceatannol | 0.40 | −0.090 | 0.043 | 0.045 | −0.216 | 0.375 |
| Piceatannol | 2.00 | 0.074 | 0.025 | 0.016 | 0.177 | 0.221 |
| Piceatannol | 10.00 | −0.071 | −0.021 | −0.104 | −0.170 | −0.184 |
| Pifithrin-CE° | 0.08 | 0.227 | 0.114 | 0.076 | 0.543 | 0.991 |
| Pifithrin-CE° | 0.40 | 0.182 | 0.040 | 0.090 | 0.436 | 0.347 |
| Pifithrin-CE° | 2.00 | 0.219 | 0.135 | 0.182 | 0.524 | 1.173 |
| Pifithrin-CE° | 10.00 | 0.120 | 0.002 | 0.038 | 0.286 | 0.016 |
| Pimobendan | 0.08 | −0.022 | 0.038 | 0.129 | −0.053 | 0.328 |
| Pimobendan | 0.40 | 0.193 | 0.063 | 0.051 | 0.460 | 0.546 |
| Pimobendan | 2.00 | 0.206 | 0.129 | 0.194 | 0.492 | 1.121 |
| Pimobendan | 10.00 | 0.139 | −0.066 | −0.134 | 0.333 | −0.577 |
| PluriSIn #1 (NSC 14613) | 0.08 | −0.146 | 0.083 | 0.057 | −0.349 | 0.720 |
| PluriSIn #1 (NSC 14613) | 0.40 | −0.095 | 0.004 | −0.007 | −0.226 | 0.039 |
| PluriSIn #1 (NSC 14613) | 2.00 | −0.433 | −0.107 | −0.133 | −1.035 | −0.929 |
| PluriSIn #1 (NSC 14613) | 10.00 | −0.853 | −0.284 | −0.372 | −2.038 | −2.475 |
| PNU-120596 | 0.08 | −0.079 | 0.108 | 0.058 | −0.188 | 0.944 |
| PNU-120596 | 0.40 | 0.085 | −0.011 | 0.081 | 0.204 | −0.098 |
| PNU-120596 | 2.00 | 0.114 | 0.068 | 0.065 | 0.271 | 0.594 |
| PNU-120596 | 10.00 | 0.138 | −0.060 | −0.049 | 0.330 | −0.525 |
| Pomalidomide | 0.08 | 0.063 | −0.002 | −0.028 | 0.150 | −0.019 |
| Pomalidomide | 0.40 | 0.224 | −0.001 | −0.082 | 0.536 | −0.013 |
| Pomalidomide | 2.00 | −0.216 | −0.038 | −0.115 | −0.517 | −0.334 |
| Pomalidomide | 10.00 | −0.305 | −0.126 | −0.205 | −0.729 | −1.098 |
| PP2 | 0.08 | −0.693 | 0.033 | 0.058 | −1.655 | 0.285 |
| PP2 | 0.40 | 0.130 | 0.012 | 0.145 | 0.312 | 0.104 |
| PP2 | 2.00 | −0.382 | 0.270 | 0.113 | −0.913 | 2.354 |
| PP2 | 10.00 | −0.246 | −0.027 | 0.027 | −0.587 | −0.235 |
| PR-619 | 0.08 | 0.054 | 0.100 | 0.122 | 0.130 | 0.872 |
| PR-619 | 0.40 | 0.111 | 0.051 | 0.047 | 0.266 | 0.448 |
| PR-619 | 2.00 | 0.022 | 0.057 | 0.081 | 0.053 | 0.495 |
| PR-619 | 10.00 | −0.320 | −0.194 | −0.193 | −0.764 | −1.687 |
| Pralatrexate | 0.08 | −0.649 | −0.135 | −0.183 | −1.551 | −1.173 |
| Pralatrexate | 0.40 | −0.853 | −0.162 | −0.176 | −2.036 | −1.413 |
| Pralatrexate | 2.00 | −0.681 | −0.154 | −0.173 | −1.626 | −1.345 |
| Pralatrexate | 10.00 | −0.922 | −0.148 | −0.215 | −2.201 | −1.292 |
| Pramipexole | 0.08 | 0.109 | 0.057 | 0.050 | 0.261 | 0.495 |
| Pramipexole | 0.40 | 0.182 | 0.027 | 0.123 | 0.436 | 0.234 |
| Pramipexole | 2.00 | 0.026 | 0.027 | 0.041 | 0.062 | 0.239 |
| Pramipexole | 10.00 | 0.231 | 0.005 | 0.071 | 0.551 | 0.043 |
| Propranolol HCl | 0.08 | 0.066 | 0.048 | 0.173 | 0.158 | 0.418 |
| Propranolol HCl | 0.40 | 0.054 | 0.074 | 0.204 | 0.129 | 0.642 |
| Propranolol HCl | 2.00 | 0.117 | 0.164 | 0.091 | 0.279 | 1.427 |
| Propranolol HCl | 10.00 | 0.168 | 0.032 | 0.040 | 0.400 | 0.276 |
| PRT062607 (P505-15, BIIB057) HCl | 0.08 | −0.092 | 0.004 | −0.033 | −0.219 | 0.037 |
| PRT062607 (P505-15, BIIB057) HCl | 0.40 | 0.080 | 0.022 | 0.122 | 0.191 | 0.194 |
| PRT062607 (P505-15, BIIB057) HCl | 2.00 | −0.075 | −0.032 | −0.123 | −0.179 | −0.280 |
| PRT062607 (P505-15, BIIB057) HCl | 10.00 | −0.330 | −0.211 | −0.298 | −0.788 | −1.837 |
| PTC-209 | 0.08 | −0.108 | 0.034 | 0.007 | −0.259 | 0.294 |
| PTC-209 | 0.40 | −0.207 | −0.058 | 0.045 | −0.495 | −0.509 |
| PTC-209 | 2.00 | −1.422 | −0.201 | −0.289 | −3.397 | −1.753 |
| PTC-209 | 10.00 | −1.553 | −0.227 | −0.300 | −3.708 | −1.975 |
| PYR-41 | 0.08 | 0.255 | 0.150 | 0.193 | 0.609 | 1.305 |
| PYR-41 | 0.40 | 0.319 | 0.089 | 0.076 | 0.762 | 0.777 |
| PYR-41 | 2.00 | 0.161 | 0.065 | 0.049 | 0.385 | 0.568 |
| PYR-41 | 10.00 | 0.096 | 0.099 | 0.129 | 0.229 | 0.861 |
| Pyrimethamine | 0.08 | 0.007 | −0.005 | 0.042 | 0.016 | −0.041 |
| Pyrimethamine | 0.40 | −0.259 | −0.018 | −0.026 | −0.618 | −0.160 |
| Pyrimethamine | 2.00 | −0.416 | −0.144 | −0.162 | −0.994 | −1.255 |
| Pyrimethamine | 10.00 | −0.620 | −0.138 | −0.282 | −1.480 | −1.199 |
| Quizartinib (AC220) | 0.08 | 0.351 | 0.096 | 0.154 | 0.838 | 0.836 |
| Quizartinib (AC220) | 0.40 | 0.149 | 0.039 | 0.083 | 0.355 | 0.341 |
| Quizartinib (AC220) | 2.00 | −0.352 | −0.052 | −0.077 | −0.841 | −0.452 |
| Quizartinib (AC220) | 10.00 | −0.644 | −0.173 | −0.200 | −1.539 | −1.505 |
| Raltegravir (MK-0518) | 0.08 | 0.108 | 0.187 | 0.137 | 0.258 | 1.627 |
| Raltegravir (MK-0518) | 0.40 | 0.094 | 0.035 | 0.037 | 0.224 | 0.302 |
| Raltegravir (MK-0518) | 2.00 | 0.197 | 0.029 | 0.122 | 0.471 | 0.254 |
| Raltegravir (MK-0518) | 10.00 | 0.170 | 0.042 | 0.183 | 0.407 | 0.366 |
| Ramelteon | 0.08 | −0.028 | 0.062 | 0.030 | −0.066 | 0.537 |
| Ramelteon | 0.40 | 0.155 | 0.082 | 0.103 | 0.369 | 0.712 |
| Ramelteon | 2.00 | −0.315 | −0.013 | −0.009 | −0.752 | −0.109 |
| Ramelteon | 10.00 | 0.150 | 0.007 | 0.001 | 0.358 | 0.062 |
| Ranitidine | 0.08 | −0.051 | −0.027 | −0.020 | −0.121 | −0.234 |
| Ranitidine | 0.40 | 0.155 | 0.011 | 0.071 | 0.370 | 0.095 |
| Ranitidine | 2.00 | −0.069 | −0.045 | −0.040 | −0.165 | −0.389 |
| Ranitidine | 10.00 | −0.262 | −0.046 | −0.043 | −0.625 | −0.401 |
| Rasagiline Mesylate | 0.08 | 0.154 | 0.072 | 0.140 | 0.367 | 0.631 |
| Rasagiline Mesylate | 0.40 | 0.103 | 0.013 | 0.077 | 0.246 | 0.117 |
| Rasagiline Mesylate | 2.00 | 0.041 | 0.048 | 0.014 | 0.099 | 0.417 |
| Rasagiline Mesylate | 10.00 | 0.104 | −0.034 | 0.026 | 0.249 | −0.294 |
| Rebamipide | 0.08 | −0.256 | 0.096 | −0.014 | −0.612 | 0.832 |
| Rebamipide | 0.40 | −0.510 | −0.091 | 0.014 | −1.217 | −0.793 |
| Rebamipide | 2.00 | −0.203 | −0.090 | −0.106 | −0.484 | −0.780 |
| Rebamipide | 10.00 | −0.281 | −0.089 | −0.063 | −0.672 | −0.773 |
| RepSox | 0.08 | −0.051 | 0.181 | 0.148 | −0.121 | 1.576 |
| RepSox | 0.40 | −0.088 | 0.145 | 0.351 | −0.209 | 1.265 |
| RepSox | 2.00 | −0.216 | 0.080 | 0.171 | −0.517 | 0.701 |
| RepSox | 10.00 | −0.597 | −0.069 | −0.023 | −1.426 | −0.605 |
| Ridaforolimus (Deforolimus, MK-8669) | 0.08 | 0.423 | 0.005 | 0.050 | 1.011 | 0.041 |
| Ridaforolimus (Deforolimus, MK-8669) | 0.40 | 0.066 | 0.116 | 0.069 | 0.157 | 1.012 |
| Ridaforolimus (Deforolimus, MK-8669) | 2.00 | 0.276 | 0.074 | 0.019 | 0.659 | 0.646 |
| Ridaforolimus (Deforolimus, MK-8669) | 10.00 | 0.211 | 0.027 | 0.039 | 0.505 | 0.237 |
| Rigosertib (ON-01910) | 0.08 | −0.905 | −0.242 | −0.309 | −2.162 | −2.105 |
| Rigosertib (ON-01910) | 0.40 | −1.285 | −0.210 | −0.355 | −3.069 | −1.831 |
| Rigosertib (ON-01910) | 2.00 | −1.668 | −0.265 | −0.340 | −3.984 | −2.306 |
| Rigosertib (ON-01910) | 10.00 | −0.847 | −0.242 | −0.330 | −2.022 | −2.107 |
| Rimonabant | 0.08 | 0.256 | 0.108 | 0.089 | 0.611 | 0.936 |
| Rimonabant | 0.40 | 0.206 | −0.063 | −0.003 | 0.492 | −0.545 |
| Rimonabant | 2.00 | 0.235 | −0.054 | −0.053 | 0.561 | −0.467 |
| Rimonabant | 10.00 | 0.063 | −0.044 | −0.098 | 0.152 | −0.385 |
| Rivaroxaban | 0.08 | 0.021 | 0.108 | 0.136 | 0.050 | 0.941 |
| Rivaroxaban | 0.40 | 0.195 | −0.029 | −0.027 | 0.466 | −0.248 |
| Rivaroxaban | 2.00 | 0.052 | 0.153 | 0.063 | 0.123 | 1.336 |
| Rivaroxaban | 10.00 | 0.044 | −0.045 | −0.048 | 0.104 | −0.390 |
| Rizatriptan Benzoate | 0.08 | 0.214 | 0.070 | 0.179 | 0.512 | 0.613 |
| Rizatriptan Benzoate | 0.40 | 0.109 | 0.148 | 0.019 | 0.260 | 1.289 |
| Rizatriptan Benzoate | 2.00 | 0.126 | 0.027 | −0.009 | 0.302 | 0.238 |
| Rizatriptan Benzoate | 10.00 | −0.054 | 0.000 | 0.105 | −0.129 | −0.003 |
| RKI-1447 | 0.08 | −0.397 | −0.006 | −0.056 | −0.948 | −0.055 |
| RKI-1447 | 0.40 | −0.184 | −0.092 | −0.064 | −0.441 | −0.803 |
| RKI-1447 | 2.00 | −0.226 | −0.009 | −0.017 | −0.540 | −0.077 |
| RKI-1447 | 10.00 | −0.342 | −0.183 | −0.297 | −0.816 | −1.594 |
| Rolipram | 0.08 | 0.356 | 0.406 | 0.239 | 0.850 | 3.532 |
| Rolipram | 0.40 | 0.155 | 0.063 | 0.156 | 0.370 | 0.545 |
| Rolipram | 2.00 | 0.396 | 0.169 | 0.337 | 0.945 | 1.470 |
| Rolipram | 10.00 | 0.299 | 0.112 | 0.223 | 0.714 | 0.975 |
| Rotundine | 0.08 | 0.359 | 0.124 | 0.151 | 0.857 | 1.082 |
| Rotundine | 0.40 | 0.343 | 0.032 | 0.049 | 0.818 | 0.282 |
| Rotundine | 2.00 | 0.190 | 0.072 | 0.080 | 0.454 | 0.623 |
| Rotundine | 10.00 | 0.227 | 0.126 | 0.095 | 0.543 | 1.100 |
| Roxatidine Acetate HCl | 0.08 | −0.040 | 0.149 | 0.035 | −0.095 | 1.295 |
| Roxatidine Acetate HCl | 0.40 | 0.021 | −0.029 | 0.099 | 0.050 | −0.256 |
| Roxatidine Acetate HCl | 2.00 | −0.103 | −0.001 | −0.053 | −0.245 | −0.009 |
| Roxatidine Acetate HCl | 10.00 | −0.236 | −0.061 | −0.104 | −0.564 | −0.534 |
| Ruxolitinib (INCB018424) | 0.08 | 0.458 | 0.122 | 0.150 | 1.093 | 1.066 |
| Ruxolitinib (INCB018424) | 0.40 | 0.363 | 0.070 | 0.067 | 0.867 | 0.611 |
| Ruxolitinib (INCB018424) | 2.00 | 0.445 | 0.110 | 0.105 | 1.062 | 0.954 |
| Ruxolitinib (INCB018424) | 10.00 | 0.477 | 0.295 | 0.317 | 1.140 | 2.569 |
| S3I-201 | 0.08 | 0.321 | 0.051 | 0.033 | 0.766 | 0.445 |
| S3I-201 | 0.40 | 0.160 | 0.061 | 0.157 | 0.382 | 0.527 |
| S3I-201 | 2.00 | 0.079 | 0.024 | −0.004 | 0.188 | 0.212 |
| S3I-201 | 10.00 | −0.140 | −0.016 | −0.052 | −0.334 | −0.143 |
| Safinamide Mesylate | 0.08 | 0.265 | 0.160 | 0.302 | 0.633 | 1.394 |
| Safinamide Mesylate | 0.40 | 0.241 | 0.093 | 0.191 | 0.575 | 0.814 |
| Safinamide Mesylate | 2.00 | 0.263 | 0.174 | 0.236 | 0.627 | 1.518 |
| Safinamide Mesylate | 10.00 | 0.369 | 0.057 | 0.143 | 0.883 | 0.499 |
| Sal003 | 0.08 | 0.191 | 0.116 | 0.092 | 0.456 | 1.013 |
| Sal003 | 0.40 | 0.155 | 0.183 | 0.067 | 0.370 | 1.595 |
| Sal003 | 2.00 | 0.196 | 0.077 | 0.040 | 0.468 | 0.670 |
| Sal003 | 10.00 | −0.829 | −0.246 | −0.408 | −1.981 | −2.142 |
| SANT-1 | 0.08 | 0.308 | 0.056 | 0.112 | 0.736 | 0.485 |
| SANT-1 | 0.40 | 0.078 | 0.000 | 0.024 | 0.186 | 0.002 |
| SANT-1 | 2.00 | 0.137 | 0.175 | 0.101 | 0.327 | 1.528 |
| SANT-1 | 10.00 | −0.042 | −0.049 | −0.120 | −0.100 | −0.430 |
| SAR131675 | 0.08 | 0.175 | 0.055 | 0.148 | 0.419 | 0.483 |
| SAR131675 | 0.40 | −0.084 | 0.089 | 0.096 | −0.201 | 0.774 |
| SAR131675 | 2.00 | 0.071 | 0.021 | 0.026 | 0.169 | 0.181 |
| SAR131675 | 10.00 | 0.062 | −0.088 | 0.000 | 0.149 | −0.766 |
| SB203580 | 0.08 | 0.201 | 0.092 | 0.148 | 0.479 | 0.805 |
| SB203580 | 0.40 | 0.149 | 0.127 | 0.098 | 0.356 | 1.109 |
| SB203580 | 2.00 | 0.237 | 0.052 | 0.077 | 0.567 | 0.453 |
| SB203580 | 10.00 | 0.182 | 0.023 | 0.069 | 0.435 | 0.199 |
| SB408124 | 0.08 | 0.410 | 0.172 | 0.198 | 0.978 | 1.494 |
| SB408124 | 0.40 | 0.334 | 0.134 | 0.193 | 0.799 | 1.168 |
| SB408124 | 2.00 | 0.364 | 0.084 | 0.082 | 0.869 | 0.730 |
| SB408124 | 10.00 | 0.091 | −0.070 | −0.148 | 0.216 | −0.607 |
| SB415286 | 0.08 | 0.221 | 0.145 | 0.159 | 0.527 | 1.262 |
| SB415286 | 0.40 | −0.004 | 0.012 | −0.008 | −0.011 | 0.101 |
| SB415286 | 2.00 | −0.097 | 0.118 | −0.053 | −0.231 | 1.024 |
| SB415286 | 10.00 | 0.123 | −0.016 | −0.042 | 0.293 | −0.136 |
| SB431542 | 0.08 | 0.380 | 0.141 | 0.135 | 0.907 | 1.225 |
| SB431542 | 0.40 | 0.473 | 0.174 | 0.309 | 1.130 | 1.514 |
| SB431542 | 2.00 | 0.316 | 0.187 | 0.223 | 0.754 | 1.632 |
| SB431542 | 10.00 | 0.155 | 0.040 | 0.126 | 0.370 | 0.346 |
| SB705498 | 0.08 | 0.133 | 0.074 | 0.094 | 0.318 | 0.642 |
| SB705498 | 0.40 | −0.043 | −0.050 | 0.023 | −0.102 | −0.433 |
| SB705498 | 2.00 | −0.004 | −0.001 | −0.030 | −0.009 | −0.008 |
| SB705498 | 10.00 | −0.037 | −0.018 | −0.023 | −0.089 | −0.154 |
| SB742457 | 0.08 | −0.290 | 0.012 | −0.055 | −0.693 | 0.108 |
| SB742457 | 0.40 | −0.126 | −0.062 | −0.029 | −0.300 | −0.537 |
| SB742457 | 2.00 | −0.177 | 0.069 | −0.003 | −0.423 | 0.598 |
| SB742457 | 10.00 | −0.294 | 0.013 | −0.006 | −0.702 | 0.115 |
| SB743921 | 0.08 | −0.485 | −0.232 | −0.321 | −1.159 | −2.017 |
| SB743921 | 0.40 | −0.777 | −0.247 | −0.329 | −1.855 | −2.148 |
| SB743921 | 2.00 | −0.485 | −0.264 | −0.340 | −1.158 | −2.297 |
| SB743921 | 10.00 | −0.542 | −0.238 | −0.350 | −1.294 | −2.068 |
| SC-514 | 0.08 | 0.051 | 0.174 | 0.064 | 0.123 | 1.512 |
| SC-514 | 0.40 | 0.015 | −0.007 | −0.061 | 0.036 | −0.060 |
| SC-514 | 2.00 | 0.194 | 0.145 | 0.008 | 0.464 | 1.265 |
| SC-514 | 10.00 | 0.212 | 0.079 | 0.025 | 0.506 | 0.688 |
| SC144 | 0.08 | 0.160 | 0.067 | 0.098 | 0.381 | 0.582 |
| SC144 | 0.40 | 0.129 | −0.013 | 0.014 | 0.309 | −0.112 |
| SC144 | 2.00 | −0.257 | −0.025 | −0.131 | −0.613 | −0.220 |
| SC144 | 10.00 | −0.885 | −0.156 | −0.211 | −2.113 | −1.355 |
| Selumetinib (AZD6244) | 0.08 | 0.164 | 0.021 | 0.004 | 0.393 | 0.181 |
| Selumetinib (AZD6244) | 0.40 | −0.165 | −0.022 | −0.047 | −0.395 | −0.188 |
| Selumetinib (AZD6244) | 2.00 | −0.257 | −0.097 | −0.175 | −0.613 | −0.847 |
| Selumetinib (AZD6244) | 10.00 | −0.852 | −0.140 | −0.211 | −2.035 | −1.220 |
| Semagacestat (LY450139) | 0.08 | 0.141 | 0.017 | −0.014 | 0.338 | 0.151 |
| Semagacestat (LY450139) | 0.40 | −0.207 | −0.021 | −0.006 | −0.494 | −0.181 |
| Semagacestat (LY450139) | 2.00 | −0.128 | −0.081 | −0.081 | −0.306 | −0.704 |
| Semagacestat (LY450139) | 10.00 | −0.856 | −0.108 | −0.162 | −2.045 | −0.942 |
| Sertraline HCl | 0.08 | −0.079 | −0.026 | −0.048 | −0.189 | −0.227 |
| Sertraline HCl | 0.40 | 0.087 | −0.082 | −0.076 | 0.208 | −0.714 |
| Sertraline HCl | 2.00 | 0.066 | 0.052 | 0.023 | 0.157 | 0.450 |
| Sertraline HCl | 10.00 | 0.043 | −0.005 | −0.021 | 0.104 | −0.044 |
| SGC0946 | 0.08 | 0.052 | 0.090 | 0.145 | 0.124 | 0.787 |
| SGC0946 | 0.40 | −0.122 | −0.027 | −0.098 | −0.290 | −0.236 |
| SGC0946 | 2.00 | −0.082 | 0.200 | 0.220 | −0.197 | 1.738 |
| SGC0946 | 10.00 | −0.142 | 0.059 | 0.065 | −0.340 | 0.516 |
| SGI-1027 | 0.08 | 0.244 | 0.061 | 0.060 | 0.583 | 0.528 |
| SGI-1027 | 0.40 | 0.298 | 0.084 | 0.145 | 0.712 | 0.735 |
| SGI-1027 | 2.00 | 0.172 | 0.129 | 0.094 | 0.412 | 1.123 |
| SGI-1027 | 10.00 | 0.052 | −0.005 | 0.018 | 0.125 | −0.046 |
| SGI-1776free base | 0.08 | 0.170 | 0.116 | 0.159 | 0.407 | 1.013 |
| SGI-1776free base | 0.40 | 0.278 | 0.042 | 0.088 | 0.665 | 0.362 |
| SGI-1776free base | 2.00 | 0.212 | 0.012 | 0.011 | 0.506 | 0.108 |
| SGI-1776free base | 10.00 | −1.033 | −0.205 | −0.317 | −2.468 | −1.784 |
| Sirtinol | 0.08 | −0.218 | 0.095 | 0.077 | −0.520 | 0.824 |
| Sirtinol | 0.40 | 0.009 | 0.000 | 0.088 | 0.022 | 0.002 |
| Sirtinol | 2.00 | −0.120 | 0.061 | 0.028 | −0.287 | 0.531 |
| Sirtinol | 10.00 | −0.078 | 0.078 | 0.026 | −0.186 | 0.679 |
| Sitaxentan sodium | 0.08 | 0.095 | 0.094 | −0.017 | 0.226 | 0.816 |
| Sitaxentan sodium | 0.40 | −0.200 | −0.052 | −0.028 | −0.478 | −0.455 |
| Sitaxentan sodium | 2.00 | −0.096 | 0.090 | 0.136 | −0.230 | 0.782 |
| Sitaxentan sodium | 10.00 | −0.100 | −0.038 | −0.047 | −0.240 | −0.332 |
| SKI II | 0.08 | 0.293 | 0.078 | 0.090 | 0.699 | 0.683 |
| SKI II | 0.40 | 0.222 | 0.219 | 0.123 | 0.531 | 1.906 |
| SKI II | 2.00 | 0.198 | 0.074 | 0.061 | 0.473 | 0.647 |
| SKI II | 10.00 | −0.336 | −0.156 | −0.191 | −0.802 | −1.362 |
| SMI-4a | 0.08 | −0.153 | 0.022 | 0.036 | −0.366 | 0.188 |
| SMI-4a | 0.40 | −0.317 | −0.060 | −0.063 | −0.758 | −0.524 |
| SMI-4a | 2.00 | −0.332 | 0.032 | −0.042 | −0.793 | 0.283 |
| SMI-4a | 10.00 | −0.327 | −0.074 | −0.134 | −0.781 | −0.643 |
| SN-38 | 0.08 | −0.720 | −0.275 | −0.419 | −1.720 | −2.391 |
| SN-38 | 0.40 | −1.091 | −0.288 | −0.402 | −2.606 | −2.505 |
| SN-38 | 2.00 | −1.226 | −0.305 | −0.391 | −2.929 | −2.655 |
| SN-38 | 10.00 | −1.166 | −0.299 | −0.404 | −2.785 | −2.606 |
| SNS-032 (BMS-387032) | 0.08 | −0.075 | 0.049 | 0.070 | −0.180 | 0.428 |
| SNS-032 (BMS-387032) | 0.40 | −0.083 | −0.143 | −0.206 | −0.198 | −1.241 |
| SNS-032 (BMS-387032) | 2.00 | −0.915 | −0.276 | −0.301 | −2.186 | −2.399 |
| SNS-032 (BMS-387032) | 10.00 | −1.009 | −0.254 | −0.361 | −2.411 | −2.214 |
| SNS-314 Mesylate | 0.08 | −0.666 | −0.071 | −0.115 | −1.591 | −0.620 |
| SNS-314 Mesylate | 0.40 | −0.777 | −0.059 | −0.103 | −1.857 | −0.515 |
| SNS-314 Mesylate | 2.00 | −0.478 | −0.128 | −0.133 | −1.142 | −1.111 |
| SNS-314 Mesylate | 10.00 | −0.620 | −0.127 | −0.198 | −1.480 | −1.109 |
| Sodium 4-Aminosalicylate | 0.08 | 0.119 | 0.080 | 0.162 | 0.283 | 0.695 |
| Sodium 4-Aminosalicylate | 0.40 | 0.204 | 0.041 | 0.050 | 0.488 | 0.354 |
| Sodium 4-Aminosalicylate | 2.00 | 0.038 | 0.156 | 0.099 | 0.092 | 1.354 |
| Sodium 4-Aminosalicylate | 10.00 | 0.247 | 0.091 | 0.143 | 0.590 | 0.796 |
| Sorafenib | 0.08 | 0.032 | 0.052 | 0.124 | 0.077 | 0.457 |
| Sorafenib | 0.40 | 0.165 | 0.060 | 0.164 | 0.395 | 0.520 |
| Sorafenib | 2.00 | −0.206 | 0.095 | 0.014 | −0.493 | 0.830 |
| Sorafenib | 10.00 | 0.114 | −0.021 | −0.062 | 0.273 | −0.180 |
| Sotrastaurin | 0.08 | 0.010 | 0.193 | 0.097 | 0.024 | 1.678 |
| Sotrastaurin | 0.40 | −0.120 | −0.065 | −0.043 | −0.286 | −0.567 |
| Sotrastaurin | 2.00 | −0.272 | 0.014 | −0.085 | −0.650 | 0.125 |
| Sotrastaurin | 10.00 | −0.242 | −0.126 | −0.164 | −0.578 | −1.101 |
| SP600125 | 0.08 | 0.197 | 0.135 | 0.188 | 0.471 | 1.174 |
| SP600125 | 0.40 | 0.145 | 0.027 | 0.092 | 0.347 | 0.232 |
| SP600125 | 2.00 | 0.103 | 0.152 | 0.090 | 0.246 | 1.321 |
| SP600125 | 10.00 | −0.020 | 0.022 | 0.084 | −0.047 | 0.187 |
| SRPIN340 | 0.08 | 0.421 | 0.115 | 0.112 | 1.005 | 1.004 |
| SRPIN340 | 0.40 | 0.445 | 0.040 | 0.098 | 1.063 | 0.348 |
| SRPIN340 | 2.00 | 0.453 | 0.097 | 0.100 | 1.083 | 0.846 |
| SRPIN340 | 10.00 | 0.467 | 0.022 | 0.078 | 1.115 | 0.192 |
| SRT1720 | 0.08 | 0.529 | 0.172 | 0.190 | 1.264 | 1.495 |
| SRT1720 | 0.40 | 0.394 | 0.112 | 0.099 | 0.940 | 0.972 |
| SRT1720 | 2.00 | 0.339 | 0.070 | 0.051 | 0.810 | 0.613 |
| SRT1720 | 10.00 | −1.016 | −0.104 | −0.193 | −2.427 | −0.906 |
| SSR128129E | 0.08 | 0.088 | 0.093 | 0.086 | 0.210 | 0.807 |
| SSR128129E | 0.40 | 0.228 | 0.039 | 0.122 | 0.545 | 0.339 |
| SSR128129E | 2.00 | 0.122 | 0.030 | 0.023 | 0.291 | 0.257 |
| SSR128129E | 10.00 | −0.026 | −0.098 | −0.095 | −0.063 | −0.850 |
| Stattic | 0.08 | −0.080 | 0.130 | 0.150 | −0.191 | 1.131 |
| Stattic | 0.40 | −0.005 | 0.022 | 0.033 | −0.011 | 0.189 |
| Stattic | 2.00 | 0.033 | 0.147 | 0.082 | 0.078 | 1.276 |
| Stattic | 10.00 | −0.025 | −0.021 | −0.019 | −0.060 | −0.186 |
| Stavudine (d4T) | 0.08 | 0.095 | 0.071 | 0.134 | 0.226 | 0.620 |
| Stavudine (d4T) | 0.40 | 0.147 | 0.003 | 0.001 | 0.351 | 0.027 |
| Stavudine (d4T) | 2.00 | 0.075 | 0.009 | −0.003 | 0.179 | 0.074 |
| Stavudine (d4T) | 10.00 | −0.075 | −0.095 | −0.120 | −0.179 | −0.828 |
| STF-118804 | 0.08 | −0.151 | −0.155 | −0.257 | −0.360 | −1.349 |
| STF-118804 | 0.40 | −1.401 | −0.210 | −0.212 | −3.346 | −1.824 |
| STF-118804 | 2.00 | −0.916 | −0.172 | −0.271 | −2.189 | −1.502 |
| STF-118804 | 10.00 | −1.338 | −0.177 | −0.300 | −3.195 | −1.541 |
| SU11274 | 0.08 | 0.357 | 0.084 | 0.115 | 0.852 | 0.735 |
| SU11274 | 0.40 | 0.270 | 0.120 | 0.298 | 0.645 | 1.043 |
| SU11274 | 2.00 | 0.058 | 0.005 | 0.008 | 0.139 | 0.047 |
| SU11274 | 10.00 | −0.818 | −0.207 | −0.300 | −1.954 | −1.804 |
| Suvorexant (MK-4305) | 0.08 | 0.192 | 0.204 | 0.186 | 0.458 | 1.775 |
| Suvorexant (MK-4305) | 0.40 | 0.255 | 0.123 | 0.142 | 0.609 | 1.073 |
| Suvorexant (MK-4305) | 2.00 | 0.375 | 0.123 | 0.101 | 0.895 | 1.073 |
| Suvorexant (MK-4305) | 10.00 | 0.250 | 0.077 | 0.131 | 0.597 | 0.670 |
| T0070907 | 0.08 | 0.112 | 0.010 | −0.005 | 0.267 | 0.084 |
| T0070907 | 0.40 | 0.026 | −0.003 | −0.001 | 0.062 | −0.029 |
| T0070907 | 2.00 | 0.050 | 0.043 | 0.081 | 0.119 | 0.373 |
| T0070907 | 10.00 | −0.756 | −0.218 | −0.312 | −1.806 | −1.897 |
| T0901317 | 0.08 | 0.047 | 0.086 | 0.111 | 0.112 | 0.748 |
| T0901317 | 0.40 | 0.178 | 0.042 | 0.077 | 0.426 | 0.369 |
| T0901317 | 2.00 | 0.096 | 0.074 | 0.070 | 0.228 | 0.644 |
| T0901317 | 10.00 | 0.114 | −0.015 | −0.034 | 0.272 | −0.130 |
| Tadalafil | 0.08 | −0.019 | 0.102 | 0.091 | −0.046 | 0.887 |
| Tadalafil | 0.40 | 0.361 | 0.027 | 0.095 | 0.863 | 0.233 |
| Tadalafil | 2.00 | 0.436 | 0.130 | 0.223 | 1.042 | 1.128 |
| Tadalafil | 10.00 | 0.270 | 0.061 | 0.148 | 0.644 | 0.534 |
| TAE226 (NVP-TAE226) | 0.08 | −0.472 | −0.256 | −0.333 | −1.127 | −2.226 |
| TAE226 (NVP-TAE226) | 0.40 | −1.126 | −0.304 | −0.376 | −2.689 | −2.650 |
| TAE226 (NVP-TAE226) | 2.00 | −1.284 | −0.323 | −0.400 | −3.068 | −2.813 |
| TAE226 (NVP-TAE226) | 10.00 | −0.989 | −0.292 | −0.426 | −2.363 | −2.545 |
| TAK-700 (Orteronel) | 0.08 | −0.276 | 0.176 | 0.113 | −0.659 | 1.535 |
| TAK-700 (Orteronel) | 0.40 | −0.024 | 0.182 | 0.090 | −0.056 | 1.585 |
| TAK-700 (Orteronel) | 2.00 | −0.084 | 0.121 | 0.093 | −0.202 | 1.058 |
| TAK-700 (Orteronel) | 10.00 | 0.092 | −0.002 | 0.032 | 0.219 | −0.018 |
| TAK-875 | 0.08 | 0.239 | 0.096 | 0.212 | 0.572 | 0.839 |
| TAK-875 | 0.40 | −0.164 | 0.023 | 0.014 | −0.392 | 0.199 |
| TAK-875 | 2.00 | −0.157 | −0.043 | −0.031 | −0.376 | −0.378 |
| TAK-875 | 10.00 | −0.291 | −0.138 | −0.060 | −0.694 | −1.199 |
| Tandutinib (MLN518) | 0.08 | 0.585 | 0.095 | 0.145 | 1.397 | 0.828 |
| Tandutinib (MLN518) | 0.40 | 0.354 | 0.122 | 0.178 | 0.845 | 1.066 |
| Tandutinib (MLN518) | 2.00 | 0.358 | 0.116 | 0.093 | 0.856 | 1.012 |
| Tandutinib (MLN518) | 10.00 | 0.586 | 0.047 | 0.100 | 1.401 | 0.407 |
| Tariquidar | 0.08 | −0.117 | 0.053 | 0.040 | −0.280 | 0.465 |
| Tariquidar | 0.40 | 0.054 | 0.037 | 0.073 | 0.128 | 0.326 |
| Tariquidar | 2.00 | 0.376 | 0.031 | 0.059 | 0.899 | 0.270 |
| Tariquidar | 10.00 | 0.227 | 0.034 | 0.019 | 0.543 | 0.292 |
| TCID | 0.08 | −0.136 | 0.049 | 0.062 | −0.325 | 0.431 |
| TCID | 0.40 | −0.007 | −0.018 | 0.017 | −0.017 | −0.155 |
| TCID | 2.00 | −0.217 | 0.066 | −0.066 | −0.517 | 0.578 |
| TCID | 10.00 | −0.228 | −0.018 | −0.054 | −0.545 | −0.159 |
| TCS 359 | 0.08 | 0.150 | 0.085 | 0.098 | 0.358 | 0.742 |
| TCS 359 | 0.40 | 0.250 | 0.058 | 0.069 | 0.598 | 0.502 |
| TCS 359 | 2.00 | 0.050 | 0.018 | −0.006 | 0.119 | 0.156 |
| TCS 359 | 10.00 | −0.089 | −0.008 | 0.026 | −0.212 | −0.072 |
| Telmisartan | 0.08 | 0.134 | 0.139 | 0.140 | 0.320 | 1.213 |
| Telmisartan | 0.40 | 0.244 | 0.129 | 0.106 | 0.584 | 1.124 |
| Telmisartan | 2.00 | 0.064 | 0.057 | 0.063 | 0.153 | 0.499 |
| Telmisartan | 10.00 | 0.230 | −0.007 | −0.003 | 0.549 | −0.060 |
| Temsirolimus (CCI-779, NSC 683864) | 0.08 | 0.514 | 0.091 | 0.082 | 1.228 | 0.790 |
| Temsirolimus (CCI-779, NSC 683864) | 0.40 | 0.583 | 0.088 | 0.152 | 1.393 | 0.763 |
| Temsirolimus (CCI-779, NSC 683864) | 2.00 | 0.578 | 0.172 | 0.132 | 1.382 | 1.494 |
| Temsirolimus (CCI-779, NSC 683864) | 10.00 | 0.200 | 0.119 | 0.153 | 0.478 | 1.035 |
| Tenofovir | 0.08 | 0.019 | 0.077 | 0.045 | 0.045 | 0.673 |
| Tenofovir | 0.40 | −0.084 | −0.076 | −0.080 | −0.200 | −0.662 |
| Tenofovir | 2.00 | −0.129 | −0.132 | −0.171 | −0.308 | −1.153 |
| Tenofovir | 10.00 | −0.337 | −0.218 | −0.271 | −0.804 | −1.899 |
| Tenofovir Disoproxil Fumarate | 0.08 | 0.072 | 0.055 | 0.208 | 0.172 | 0.475 |
| Tenofovir Disoproxil Fumarate | 0.40 | −0.002 | −0.051 | 0.086 | −0.004 | −0.442 |
| Tenofovir Disoproxil Fumarate | 2.00 | −0.158 | −0.079 | −0.065 | −0.378 | −0.689 |
| Tenofovir Disoproxil Fumarate | 10.00 | −0.433 | −0.185 | −0.267 | −1.034 | −1.614 |
| Tenovin-6 | 0.08 | 0.069 | 0.121 | 0.028 | 0.165 | 1.050 |
| Tenovin-6 | 0.40 | −0.506 | −0.053 | −0.109 | −1.209 | −0.463 |
| Tenovin-6 | 2.00 | −1.111 | −0.194 | −0.328 | −2.654 | −1.686 |
| Tenovin-6 | 10.00 | −1.429 | −0.192 | −0.344 | −3.414 | −1.671 |
| TG100-115 | 0.08 | −0.256 | 0.208 | 0.073 | −0.611 | 1.812 |
| TG100-115 | 0.40 | 0.066 | −0.026 | 0.032 | 0.158 | −0.223 |
| TG100-115 | 2.00 | −0.013 | 0.003 | −0.019 | −0.031 | 0.025 |
| TG100-115 | 10.00 | −0.116 | −0.026 | −0.100 | −0.277 | −0.223 |
| Thiazovivin | 0.08 | 0.141 | 0.090 | 0.142 | 0.337 | 0.780 |
| Thiazovivin | 0.40 | 0.113 | 0.070 | 0.060 | 0.271 | 0.611 |
| Thiazovivin | 2.00 | 0.312 | 0.024 | 0.064 | 0.746 | 0.205 |
| Thiazovivin | 10.00 | 0.189 | −0.069 | 0.037 | 0.451 | −0.605 |
| Ticagrelor | 0.08 | −0.080 | 0.115 | 0.042 | −0.190 | 0.998 |
| Ticagrelor | 0.40 | −0.029 | −0.032 | 0.000 | −0.069 | −0.276 |
| Ticagrelor | 2.00 | −0.270 | 0.120 | 0.018 | −0.645 | 1.041 |
| Ticagrelor | 10.00 | −0.241 | 0.020 | 0.022 | −0.576 | 0.170 |
| Ticlopidine HCl | 0.08 | −0.118 | 0.043 | 0.052 | −0.281 | 0.373 |
| Ticlopidine HCl | 0.40 | −0.041 | 0.010 | 0.072 | −0.097 | 0.084 |
| Ticlopidine HCl | 2.00 | −0.234 | −0.002 | −0.022 | −0.559 | −0.018 |
| Ticlopidine HCl | 10.00 | −0.005 | −0.035 | 0.076 | −0.013 | −0.308 |
| Tie2 kinase inhibitor | 0.08 | 0.005 | 0.009 | 0.018 | 0.012 | 0.080 |
| Tie2 kinase inhibitor | 0.40 | 0.074 | 0.002 | −0.021 | 0.177 | 0.019 |
| Tie2 kinase inhibitor | 2.00 | −0.314 | 0.181 | 0.024 | −0.750 | 1.577 |
| Tie2 kinase inhibitor | 10.00 | −0.549 | −0.031 | −0.097 | −1.311 | −0.274 |
| Tioxolone | 0.08 | 0.246 | 0.099 | 0.149 | 0.586 | 0.864 |
| Tioxolone | 0.40 | 0.089 | 0.012 | 0.000 | 0.212 | 0.101 |
| Tioxolone | 2.00 | 0.044 | 0.053 | 0.023 | 0.105 | 0.462 |
| Tioxolone | 10.00 | 0.328 | 0.023 | 0.028 | 0.783 | 0.202 |
| Tofacitinib (CP-690550, Tasocitinib) | 0.08 | 0.130 | −0.017 | 0.022 | 0.312 | −0.146 |
| Tofacitinib (CP-690550, Tasocitinib) | 0.40 | 0.249 | −0.015 | 0.010 | 0.595 | −0.132 |
| Tofacitinib (CP-690550, Tasocitinib) | 2.00 | 0.117 | −0.007 | 0.025 | 0.278 | −0.061 |
| Tofacitinib (CP-690550, Tasocitinib) | 10.00 | 0.236 | −0.004 | 0.010 | 0.563 | −0.036 |
| Tolazoline HCl | 0.08 | 0.245 | 0.144 | 0.169 | 0.585 | 1.255 |
| Tolazoline HCl | 0.40 | 0.222 | 0.084 | 0.128 | 0.530 | 0.728 |
| Tolazoline HCl | 2.00 | −0.051 | 0.088 | 0.069 | −0.122 | 0.765 |
| Tolazoline HCl | 10.00 | −0.208 | −0.049 | −0.063 | −0.496 | −0.424 |
| Tolfenamic Acid | 0.08 | 0.196 | 0.028 | 0.118 | 0.467 | 0.242 |
| Tolfenamic Acid | 0.40 | −0.003 | 0.023 | 0.052 | −0.007 | 0.200 |
| Tolfenamic Acid | 2.00 | −0.159 | 0.107 | −0.023 | −0.379 | 0.927 |
| Tolfenamic Acid | 10.00 | −0.115 | −0.039 | −0.064 | −0.276 | −0.343 |
| Tolvaptan | 0.08 | 0.095 | 0.049 | 0.086 | 0.227 | 0.429 |
| Tolvaptan | 0.40 | −0.008 | −0.041 | 0.031 | −0.019 | −0.355 |
| Tolvaptan | 2.00 | −0.094 | −0.023 | −0.048 | −0.225 | −0.204 |
| Tolvaptan | 10.00 | −0.104 | −0.093 | −0.150 | −0.249 | −0.807 |
| Torcetrapib | 0.08 | 0.283 | 0.114 | 0.320 | 0.677 | 0.991 |
| Torcetrapib | 0.40 | 0.061 | −0.063 | 0.045 | 0.147 | −0.546 |
| Torcetrapib | 2.00 | −0.121 | −0.136 | −0.098 | −0.289 | −1.185 |
| Torcetrapib | 10.00 | −0.151 | −0.012 | −0.056 | −0.360 | −0.108 |
| Toremifene Citrate | 0.08 | 0.344 | 0.051 | 0.078 | 0.822 | 0.446 |
| Toremifene Citrate | 0.40 | 0.119 | 0.060 | 0.229 | 0.285 | 0.522 |
| Toremifene Citrate | 2.00 | −1.012 | 0.030 | −0.034 | −2.417 | 0.258 |
| Toremifene Citrate | 10.00 | −1.396 | −0.198 | −0.283 | −3.335 | −1.721 |
| Tosedostat (CHR2797) | 0.08 | −0.097 | −0.002 | −0.027 | −0.232 | −0.022 |
| Tosedostat (CHR2797) | 0.40 | 0.051 | −0.106 | −0.134 | 0.123 | −0.925 |
| Tosedostat (CHR2797) | 2.00 | −0.201 | −0.050 | −0.128 | −0.480 | −0.432 |
| Tosedostat (CHR2797) | 10.00 | −0.468 | −0.172 | −0.276 | −1.117 | −1.499 |
| TPCA-1 | 0.08 | −0.061 | 0.072 | 0.041 | −0.146 | 0.627 |
| TPCA-1 | 0.40 | −0.004 | −0.047 | −0.029 | −0.010 | −0.413 |
| TPCA-1 | 2.00 | −0.005 | −0.059 | −0.054 | −0.012 | −0.514 |
| TPCA-1 | 10.00 | −0.531 | −0.197 | −0.294 | −1.269 | −1.711 |
| Tranylcypromine (2-PCPA) HCl | 0.08 | −0.064 | 0.033 | 0.050 | −0.153 | 0.285 |
| Tranylcypromine (2-PCPA) HCl | 0.40 | 0.062 | −0.049 | −0.037 | 0.148 | −0.426 |
| Tranylcypromine (2-PCPA) HCl | 2.00 | 0.053 | 0.049 | 0.036 | 0.128 | 0.423 |
| Tranylcypromine (2-PCPA) HCl | 10.00 | −0.533 | −0.045 | −0.096 | −1.274 | −0.389 |
| Trelagliptin | 0.08 | 0.202 | 0.007 | 0.020 | 0.482 | 0.059 |
| Trelagliptin | 0.40 | −0.076 | −0.019 | 0.058 | −0.181 | −0.166 |
| Trelagliptin | 2.00 | 0.050 | −0.033 | −0.022 | 0.120 | −0.292 |
| Trelagliptin | 10.00 | 0.135 | −0.050 | −0.109 | 0.322 | −0.433 |
| Triamterene | 0.08 | −0.029 | 0.103 | 0.048 | −0.070 | 0.894 |
| Triamterene | 0.40 | −0.165 | 0.030 | 0.051 | −0.394 | 0.266 |
| Triamterene | 2.00 | −0.101 | −0.056 | −0.065 | −0.241 | −0.490 |
| Triamterene | 10.00 | −0.462 | −0.187 | −0.290 | −1.103 | −1.630 |
| Trichostatin A (TSA) | 0.08 | 0.191 | −0.064 | −0.124 | 0.457 | −0.554 |
| Trichostatin A (TSA) | 0.40 | −0.801 | −0.195 | −0.254 | −1.913 | −1.697 |
| Trichostatin A (TSA) | 2.00 | −0.904 | −0.153 | −0.210 | −2.159 | −1.334 |
| Trichostatin A (TSA) | 10.00 | −0.826 | −0.149 | −0.202 | −1.974 | −1.298 |
| Trimebutine | 0.08 | 0.007 | −0.014 | 0.000 | 0.017 | −0.121 |
| Trimebutine | 0.40 | −0.202 | −0.019 | 0.002 | −0.483 | −0.166 |
| Trimebutine | 2.00 | −1.008 | −0.202 | −0.277 | −2.409 | −1.763 |
| Trimebutine | 10.00 | −1.066 | −0.214 | −0.332 | −2.545 | −1.863 |
| Tropicamide | 0.08 | 0.116 | −0.042 | −0.037 | 0.276 | −0.364 |
| Tropicamide | 0.40 | −0.063 | 0.041 | −0.028 | −0.151 | 0.361 |
| Tropicamide | 2.00 | 0.249 | 0.010 | 0.072 | 0.594 | 0.090 |
| Tropicamide | 10.00 | 0.055 | 0.003 | 0.004 | 0.132 | 0.026 |
| Trospium chloride | 0.08 | 0.083 | 0.169 | 0.119 | 0.199 | 1.470 |
| Trospium chloride | 0.40 | −0.031 | 0.218 | 0.117 | −0.073 | 1.895 |
| Trospium chloride | 2.00 | 0.200 | −0.002 | −0.008 | 0.477 | −0.017 |
| Trospium chloride | 10.00 | 0.148 | 0.102 | 0.084 | 0.354 | 0.885 |
| TWS119 | 0.08 | 0.328 | 0.094 | 0.207 | 0.784 | 0.820 |
| TWS119 | 0.40 | 0.333 | 0.038 | 0.142 | 0.796 | 0.327 |
| TWS119 | 2.00 | 0.071 | 0.035 | 0.044 | 0.169 | 0.304 |
| TWS119 | 10.00 | −1.200 | −0.233 | −0.283 | −2.866 | −2.027 |
| Tyrphostin AG 879 | 0.08 | −0.347 | 0.010 | −0.083 | −0.828 | 0.088 |
| Tyrphostin AG 879 | 0.40 | −0.238 | −0.078 | −0.084 | −0.569 | −0.680 |
| Tyrphostin AG 879 | 2.00 | −0.232 | −0.080 | −0.051 | −0.554 | −0.698 |
| Tyrphostin AG 879 | 10.00 | −0.368 | −0.110 | −0.123 | −0.879 | −0.960 |
| U-104 | 0.08 | −0.054 | 0.095 | 0.111 | −0.128 | 0.831 |
| U-104 | 0.40 | 0.096 | 0.013 | 0.192 | 0.229 | 0.115 |
| U-104 | 2.00 | −0.165 | 0.056 | −0.017 | −0.394 | 0.490 |
| U-104 | 10.00 | −0.036 | −0.007 | −0.023 | −0.086 | −0.058 |
| U0126-EtOH | 0.08 | −0.350 | 0.099 | 0.114 | −0.835 | 0.859 |
| U0126-EtOH | 0.40 | −0.514 | 0.019 | 0.075 | −1.227 | 0.163 |
| U0126-EtOH | 2.00 | −0.476 | −0.045 | −0.071 | −1.138 | −0.389 |
| U0126-EtOH | 10.00 | −0.790 | −0.117 | −0.165 | −1.887 | −1.021 |
| UNC2250 | 0.08 | 0.265 | 0.115 | 0.142 | 0.633 | 1.005 |
| UNC2250 | 0.40 | 0.279 | 0.067 | 0.113 | 0.667 | 0.587 |
| UNC2250 | 2.00 | 0.105 | 0.085 | 0.134 | 0.250 | 0.744 |
| UNC2250 | 10.00 | −0.064 | −0.153 | −0.205 | −0.153 | −1.336 |
| UNC669 | 0.08 | −0.095 | 0.066 | 0.047 | −0.226 | 0.578 |
| UNC669 | 0.40 | 0.096 | −0.006 | −0.029 | 0.228 | −0.048 |
| UNC669 | 2.00 | −0.235 | 0.070 | 0.025 | −0.561 | 0.606 |
| UNC669 | 10.00 | 0.073 | 0.029 | 0.002 | 0.174 | 0.249 |
| URB597 | 0.08 | 0.314 | 0.087 | 0.141 | 0.750 | 0.754 |
| URB597 | 0.40 | 0.065 | −0.001 | 0.021 | 0.154 | −0.007 |
| URB597 | 2.00 | 0.137 | 0.035 | 0.017 | 0.328 | 0.307 |
| URB597 | 10.00 | −0.021 | −0.014 | −0.044 | −0.051 | −0.123 |
| Vandetanib (ZD6474) | 0.08 | −0.648 | −0.068 | −0.139 | −1.547 | −0.594 |
| Vandetanib (ZD6474) | 0.40 | −0.366 | −0.091 | −0.054 | −0.874 | −0.792 |
| Vandetanib (ZD6474) | 2.00 | −0.874 | −0.271 | −0.384 | −2.089 | −2.356 |
| Vandetanib (ZD6474) | 10.00 | −1.206 | −0.298 | −0.494 | −2.880 | −2.594 |
| Varespladib (LY315920) | 0.08 | 0.428 | 0.176 | 0.274 | 1.022 | 1.533 |
| Varespladib (LY315920) | 0.40 | 0.362 | 0.197 | 0.157 | 0.864 | 1.719 |
| Varespladib (LY315920) | 2.00 | 0.152 | 0.164 | 0.176 | 0.363 | 1.430 |
| Varespladib (LY315920) | 10.00 | 0.241 | 0.074 | 0.051 | 0.575 | 0.643 |
| VE-821 | 0.08 | 0.213 | 0.106 | 0.130 | 0.508 | 0.924 |
| VE-821 | 0.40 | 0.130 | 0.063 | 0.089 | 0.311 | 0.544 |
| VE-821 | 2.00 | −0.137 | 0.103 | −0.004 | −0.328 | 0.901 |
| VE-821 | 10.00 | −0.840 | −0.284 | −0.429 | −2.006 | −2.475 |
| VE-822 | 0.08 | −0.147 | 0.058 | 0.104 | −0.351 | 0.505 |
| VE-822 | 0.40 | −0.287 | −0.040 | −0.064 | −0.685 | −0.351 |
| VE-822 | 2.00 | −0.717 | −0.220 | −0.302 | −1.713 | −1.913 |
| VE-822 | 10.00 | −1.763 | −0.210 | −0.321 | −4.212 | −1.825 |
| Veliparib (ABT-888) | 0.08 | 0.128 | 0.075 | 0.105 | 0.307 | 0.649 |
| Veliparib (ABT-888) | 0.40 | 0.055 | 0.139 | 0.179 | 0.132 | 1.211 |
| Veliparib (ABT-888) | 2.00 | −0.024 | 0.023 | −0.016 | −0.057 | 0.203 |
| Veliparib (ABT-888) | 10.00 | −0.218 | −0.062 | −0.136 | −0.521 | −0.537 |
| Vemurafenib (PLX4032, RG7204) | 0.08 | 0.124 | 0.090 | 0.120 | 0.297 | 0.786 |
| Vemurafenib (PLX4032, RG7204) | 0.40 | −0.082 | 0.053 | 0.150 | −0.195 | 0.462 |
| Vemurafenib (PLX4032, RG7204) | 2.00 | −0.006 | 0.047 | 0.055 | −0.014 | 0.411 |
| Vemurafenib (PLX4032, RG7204) | 10.00 | 0.030 | 0.030 | 0.022 | 0.073 | 0.263 |
| Vildagliptin (LAF-237) | 0.08 | 0.232 | 0.148 | 0.099 | 0.553 | 1.285 |
| Vildagliptin (LAF-237) | 0.40 | 0.229 | 0.230 | 0.113 | 0.547 | 2.004 |
| Vildagliptin (LAF-237) | 2.00 | 0.150 | 0.170 | 0.038 | 0.358 | 1.478 |
| Vildagliptin (LAF-237) | 10.00 | 0.339 | 0.097 | 0.013 | 0.811 | 0.846 |
| Voriconazole | 0.08 | 0.258 | 0.252 | 0.180 | 0.617 | 2.192 |
| Voriconazole | 0.40 | 0.306 | 0.193 | 0.120 | 0.730 | 1.682 |
| Voriconazole | 2.00 | 0.285 | 0.067 | 0.097 | 0.680 | 0.588 |
| Voriconazole | 10.00 | 0.328 | 0.018 | 0.048 | 0.783 | 0.153 |
| VU 0357121 | 0.08 | 0.183 | 0.079 | 0.152 | 0.438 | 0.686 |
| VU 0357121 | 0.40 | 0.148 | −0.010 | 0.034 | 0.352 | −0.090 |
| VU 0357121 | 2.00 | 0.012 | 0.030 | 0.007 | 0.029 | 0.261 |
| VU 0357121 | 10.00 | −0.059 | −0.027 | −0.034 | −0.141 | −0.237 |
| VU 0364439 | 0.08 | 0.024 | 0.082 | 0.108 | 0.058 | 0.715 |
| VU 0364439 | 0.40 | −0.121 | 0.011 | 0.124 | −0.289 | 0.099 |
| VU 0364439 | 2.00 | −0.258 | 0.010 | −0.043 | −0.616 | 0.089 |
| VU 0364439 | 10.00 | 0.009 | −0.055 | −0.038 | 0.022 | −0.476 |
| VU 0364770 | 0.08 | 0.013 | 0.155 | 0.082 | 0.031 | 1.350 |
| VU 0364770 | 0.40 | 0.085 | 0.085 | 0.125 | 0.204 | 0.736 |
| VU 0364770 | 2.00 | 0.083 | 0.117 | 0.139 | 0.199 | 1.020 |
| VU 0364770 | 10.00 | −0.103 | 0.102 | 0.066 | −0.246 | 0.884 |
| VX-680 (Tozasertib, MK-0457) | 0.08 | −0.150 | −0.043 | −0.078 | −0.359 | −0.371 |
| VX-680 (Tozasertib, MK-0457) | 0.40 | −0.596 | −0.151 | −0.245 | −1.425 | −1.317 |
| VX-680 (Tozasertib, MK-0457) | 2.00 | −0.706 | −0.166 | −0.308 | −1.686 | −1.450 |
| VX-680 (Tozasertib, MK-0457) | 10.00 | −0.959 | −0.238 | −0.342 | −2.291 | −2.069 |
| VX-745 | 0.08 | 0.186 | 0.092 | 0.154 | 0.445 | 0.805 |
| VX-745 | 0.40 | 0.146 | 0.004 | 0.054 | 0.349 | 0.031 |
| VX-745 | 2.00 | 0.018 | 0.021 | 0.058 | 0.044 | 0.186 |
| VX-745 | 10.00 | 0.022 | −0.006 | 0.038 | 0.053 | −0.049 |
| VX-765 | 0.08 | 0.154 | 0.043 | 0.091 | 0.368 | 0.371 |
| VX-765 | 0.40 | 0.232 | 0.036 | 0.032 | 0.555 | 0.317 |
| VX-765 | 2.00 | 0.165 | 0.059 | 0.061 | 0.393 | 0.516 |
| VX-765 | 10.00 | 0.254 | 0.022 | 0.174 | 0.608 | 0.190 |
| VX-809 (Lumacaftor) | 0.08 | 0.177 | 0.073 | 0.218 | 0.423 | 0.634 |
| VX-809 (Lumacaftor) | 0.40 | −0.062 | −0.041 | −0.027 | −0.149 | −0.353 |
| VX-809 (Lumacaftor) | 2.00 | −0.145 | −0.032 | −0.073 | −0.346 | −0.279 |
| VX-809 (Lumacaftor) | 10.00 | 0.032 | −0.082 | −0.117 | 0.076 | −0.713 |
| Wnt-C59 (C59) | 0.08 | −0.572 | −0.163 | −0.242 | −1.366 | −1.424 |
| Wnt-C59 (C59) | 0.40 | −0.611 | −0.182 | −0.281 | −1.461 | −1.588 |
| Wnt-C59 (C59) | 2.00 | −0.627 | −0.198 | −0.313 | −1.498 | −1.724 |
| Wnt-C59 (C59) | 10.00 | −0.613 | −0.191 | −0.301 | −1.465 | −1.666 |
| WZ4002 | 0.08 | 0.313 | 0.081 | 0.114 | 0.748 | 0.704 |
| WZ4002 | 0.40 | 0.082 | 0.021 | 0.046 | 0.196 | 0.186 |
| WZ4002 | 2.00 | 0.151 | −0.084 | −0.109 | 0.360 | −0.735 |
| WZ4002 | 10.00 | −0.941 | −0.212 | −0.319 | −2.248 | −1.849 |
| WZ4003 | 0.08 | 0.375 | 0.074 | 0.117 | 0.896 | 0.644 |
| WZ4003 | 0.40 | 0.173 | −0.041 | −0.034 | 0.413 | −0.359 |
| WZ4003 | 2.00 | 0.076 | −0.017 | −0.035 | 0.180 | −0.151 |
| WZ4003 | 10.00 | −0.018 | −0.098 | −0.142 | −0.043 | −0.850 |
| WZ811 | 0.08 | 0.140 | 0.154 | 0.296 | 0.334 | 1.337 |
| WZ811 | 0.40 | −0.034 | 0.019 | 0.007 | −0.082 | 0.162 |
| WZ811 | 2.00 | 0.077 | 0.094 | 0.074 | 0.184 | 0.815 |
| WZ811 | 10.00 | 0.044 | 0.005 | 0.019 | 0.104 | 0.045 |
| XAV-939 | 0.08 | −0.332 | 0.058 | 0.074 | −0.792 | 0.505 |
| XAV-939 | 0.40 | −0.027 | 0.020 | 0.085 | −0.065 | 0.177 |
| XAV-939 | 2.00 | −0.355 | −0.044 | −0.068 | −0.847 | −0.386 |
| XAV-939 | 10.00 | −0.899 | −0.131 | −0.231 | −2.147 | −1.142 |
| XL335 | 0.08 | 0.124 | 0.175 | 0.093 | 0.297 | 1.523 |
| XL335 | 0.40 | −0.258 | −0.082 | −0.109 | −0.615 | −0.711 |
| XL335 | 2.00 | −0.259 | −0.072 | 0.045 | −0.619 | −0.625 |
| XL335 | 10.00 | −0.156 | −0.135 | −0.180 | −0.373 | −1.176 |
| YM155 (Sepantronium Bromide) | 0.08 | 0.222 | 0.114 | 0.172 | 0.531 | 0.995 |
| YM155 (Sepantronium Bromide) | 0.40 | −0.083 | −0.031 | −0.039 | −0.198 | −0.266 |
| YM155 (Sepantronium Bromide) | 2.00 | −0.460 | −0.082 | −0.137 | −1.100 | −0.714 |
| YM155 (Sepantronium Bromide) | 10.00 | −0.970 | −0.096 | −0.210 | −2.316 | −0.839 |
| YO-01027 | 0.08 | 0.221 | 0.051 | 0.105 | 0.528 | 0.443 |
| YO-01027 | 0.40 | −0.475 | 0.085 | −0.058 | −1.135 | 0.739 |
| YO-01027 | 2.00 | −0.571 | −0.080 | −0.020 | −1.363 | −0.699 |
| YO-01027 | 10.00 | −1.032 | −0.157 | −0.184 | −2.464 | −1.369 |
| ZCL278 | 0.08 | −0.964 | −0.084 | −0.107 | −2.303 | −0.729 |
| ZCL278 | 0.40 | 0.018 | −0.037 | 0.077 | 0.044 | −0.325 |
| ZCL278 | 2.00 | −0.228 | 0.025 | −0.079 | −0.545 | 0.218 |
| ZCL278 | 10.00 | −0.104 | 0.015 | −0.080 | −0.247 | 0.130 |
| Zebularine | 0.08 | 0.035 | 0.026 | 0.092 | 0.085 | 0.223 |
| Zebularine | 0.40 | 0.000 | 0.009 | 0.077 | 0.000 | 0.075 |
| Zebularine | 2.00 | −0.089 | 0.095 | 0.051 | −0.213 | 0.828 |
| Zebularine | 10.00 | −0.167 | 0.086 | 0.102 | −0.399 | 0.749 |
| Zibotentan (ZD4054) | 0.08 | 0.104 | 0.094 | 0.168 | 0.249 | 0.820 |
| Zibotentan (ZD4054) | 0.40 | −0.009 | 0.054 | 0.130 | −0.022 | 0.474 |
| Zibotentan (ZD4054) | 2.00 | −0.768 | −0.003 | −0.038 | −1.834 | −0.029 |
| Zibotentan (ZD4054) | 10.00 | −0.001 | 0.101 | 0.050 | −0.003 | 0.877 |
| ZM 306416 | 0.08 | −0.080 | 0.105 | 0.006 | −0.190 | 0.912 |
| ZM 306416 | 0.40 | −0.483 | −0.223 | −0.288 | −1.155 | −1.942 |
| ZM 306416 | 2.00 | −1.312 | −0.291 | −0.403 | −3.135 | −2.531 |
| ZM 306416 | 10.00 | −1.490 | −0.275 | −0.431 | −3.559 | −2.395 |
| ZM 447439 | 0.08 | 0.512 | 0.103 | 0.171 | 1.222 | 0.896 |
| ZM 447439 | 0.40 | 0.304 | 0.107 | 0.108 | 0.725 | 0.932 |
| ZM 447439 | 2.00 | 0.231 | 0.052 | 0.074 | 0.552 | 0.456 |
| ZM 447439 | 10.00 | −0.240 | −0.080 | −0.132 | −0.574 | −0.695 |
| Zosuquidar (LY335979) 3HCl | 0.08 | 0.017 | 0.096 | 0.124 | 0.041 | 0.839 |
| Zosuquidar (LY335979) 3HCl | 0.40 | 0.197 | 0.080 | 0.127 | 0.470 | 0.696 |
| Zosuquidar (LY335979) 3HCl | 2.00 | 0.276 | 0.207 | 0.129 | 0.659 | 1.802 |
| Zosuquidar (LY335979) 3HCl | 10.00 | −0.193 | −0.209 | −0.200 | −0.461 | −1.821 |

| Treatment | Z.LYZ.S | SSMD.CTG.Rep | SSMD.LYZ.NS_Rep | SSMD.LYZ.S_Rep |
|---|---|---|---|---|
| (−)-MK 801 Maleate | 0.099 | 0.442 | 0.070 | 0.116 |
| (−)-MK 801 Maleate | −1.159 | −1.342 | −0.550 | −0.976 |
| (−)-MK 801 Maleate | −2.160 | −1.102 | −1.315 | −1.187 |
| (−)-MK 801 Maleate | −2.604 | −1.696 | −1.708 | −1.768 |
| (−)-Parthenolide | 0.799 | 0.498 | 0.601 | 0.679 |
| (−)-Parthenolide | 1.067 | 0.736 | 0.480 | 0.763 |
| (−)-Parthenolide | 0.441 | 0.482 | 0.920 | 0.506 |
| (−)-Parthenolide | 0.530 | 0.257 | 0.998 | 0.345 |
| (+)-Bicuculline | 1.005 | 0.472 | 0.481 | 0.545 |
| (+)-Bicuculline | 0.221 | −0.078 | 0.106 | 0.216 |
| (+)-Bicuculline | −0.169 | −0.237 | 0.276 | −0.164 |
| (+)-Bicuculline | 0.465 | −0.405 | 0.175 | 0.264 |
| (+)-JQ1 | −2.305 | −0.484 | −1.244 | −1.985 |
| (+)-JQ1 | −2.627 | −1.324 | −2.086 | −1.840 |
| (+)-JQ1 | −2.452 | −3.529 | −1.392 | −1.755 |
| (+)-JQ1 | −2.408 | −1.804 | −1.510 | −1.630 |
| (S)-crizotinib | 0.808 | 0.742 | 0.838 | 0.881 |
| (S)-crizotinib | 0.103 | 0.190 | 0.094 | 0.084 |
| (S)-crizotinib | −0.383 | 0.285 | −0.229 | −0.343 |
| (S)-crizotinib | −0.720 | 0.034 | −0.438 | −0.491 |
| 17-AAG (Tanespimycin) | −1.826 | −0.926 | −1.180 | −1.387 |
| 17-AAG (Tanespimycin) | −2.199 | −1.023 | −1.186 | −1.428 |
| 17-AAG (Tanespimycin) | −2.219 | −1.783 | −1.053 | −1.462 |
| 17-AAG (Tanespimycin) | −1.975 | −1.348 | −1.183 | −1.655 |
| 2-Methoxyestradiol (2-MeOE2) | 1.224 | 0.407 | 0.077 | 1.015 |
| 2-Methoxyestradiol (2-MeOE2) | 1.747 | 0.930 | 0.227 | 0.831 |
| 2-Methoxyestradiol (2-MeOE2) | 0.190 | 0.461 | 0.049 | 0.144 |
| 2-Methoxyestradiol (2-MeOE2) | −1.795 | −0.878 | −0.826 | −1.135 |
| 4E1RCat | 0.690 | 0.790 | 0.883 | 0.712 |
| 4E1RCat | 0.247 | 0.559 | −0.121 | 0.185 |
| 4E1RCat | 0.316 | 0.022 | 0.437 | 0.311 |
| 4E1RCat | 0.530 | −0.026 | 0.053 | 0.321 |
| 4EGI-1 | −0.236 | −0.352 | 0.378 | −0.264 |
| 4EGI-1 | 0.352 | −0.158 | 0.264 | 0.326 |
| 4EGI-1 | −0.380 | 0.407 | 0.252 | −0.328 |
| 4EGI-1 | 0.366 | −0.283 | −0.257 | 0.189 |
| 4CE°8C | 0.205 | −0.402 | 0.497 | 0.194 |
| 4CE°8C | 0.073 | 0.023 | 0.226 | 0.053 |
| 4CE°8C | −0.358 | −0.340 | 0.128 | −0.217 |
| 4CE°8C | −0.836 | −0.109 | −0.861 | −0.707 |
| 5-hydroxymethyl Tolterodine | 0.417 | −0.118 | 0.915 | 0.460 |
| (PNU 200577, 5-HMT, 5-HM) | | | | |
| 5-hydroxymethyl Tolterodine | 0.231 | 0.291 | 0.760 | 0.266 |
| (PNU 200577, 5-HMT, 5-HM) | | | | |
| 5-hydroxymethyl Tolterodine | 0.152 | 0.114 | 0.947 | 0.174 |
| (PNU 200577, 5-HMT, 5-HM) | | | | |
| 5-hydroxymethyl Tolterodine | −0.044 | 0.061 | 0.223 | −0.042 |
| (PNU 200577, 5-HMT, 5-HM) | | | | |
| A-769662 | 0.426 | −0.255 | 0.286 | 0.365 |
| A-769662 | 0.460 | 0.019 | 0.498 | 0.472 |
| A-769662 | 1.191 | −0.312 | 0.554 | 0.488 |
| A-769662 | 0.121 | −0.056 | 0.068 | 0.119 |
| ABT-199 (GDC-0199) | −0.285 | 0.051 | −0.009 | −0.260 |
| ABT-199 (GDC-0199) | 0.494 | 0.111 | −0.192 | 0.443 |
| ABT-199 (GDC-0199) | −0.404 | −0.377 | −0.116 | −0.368 |
| ABT-199 (GDC-0199) | −1.890 | −1.402 | −0.810 | −1.131 |
| ABT-263 (Navitoclax) | −0.064 | −0.462 | 0.062 | −0.041 |
| ABT-263 (Navitoclax) | −0.129 | −0.777 | 0.062 | −0.134 |
| ABT-263 (Navitoclax) | −1.327 | −0.847 | −1.019 | −1.114 |
| ABT-263 (Navitoclax) | −2.586 | −3.196 | −1.699 | −1.631 |
| Acadesine | 0.023 | 0.288 | 0.189 | 0.019 |
| Acadesine | −0.075 | 0.448 | −0.145 | −0.088 |
| Acadesine | 0.341 | −0.017 | 0.141 | 0.254 |
| Acadesine | −0.659 | 0.266 | −0.658 | −0.682 |
| ADL5859 HCl | 0.512 | 1.012 | 0.331 | 0.470 |
| ADL5859 HCl | 0.675 | 0.493 | 0.168 | 0.713 |
| ADL5859 HCl | 0.452 | 0.845 | 0.520 | 0.389 |
| ADL5859 HCl | −0.256 | 0.587 | −0.516 | −0.282 |
| ADX-47273 | 0.947 | 0.314 | 1.525 | 0.815 |
| ADX-47273 | 0.948 | −0.115 | 0.483 | 0.704 |
| ADX-47273 | −0.255 | −0.642 | −0.186 | −0.185 |
| ADX-47273 | −0.452 | −0.123 | −0.201 | −0.349 |
| AG-14361 | 0.644 | −0.020 | 1.070 | 0.577 |
| AG-14361 | −0.008 | −0.240 | 0.296 | −0.007 |
| AG-14361 | −0.419 | 0.013 | 0.260 | −0.255 |
| AG-14361 | −1.222 | −0.042 | −1.369 | −0.890 |
| AGI-5198 | 0.595 | 0.711 | 0.346 | 0.522 |
| AGI-5198 | 1.194 | 0.369 | 0.401 | 0.754 |
| AGI-5198 | 0.473 | 0.814 | 0.321 | 0.434 |
| AGI-5198 | −0.035 | 0.766 | −0.063 | −0.036 |
| AGI-6780 | 0.616 | 0.866 | 0.570 | 0.553 |
| AGI-6780 | 1.007 | 0.611 | 0.272 | 0.968 |
| AGI-6780 | 0.353 | 0.941 | 0.334 | 0.235 |
| AGI-6780 | −0.349 | 0.399 | −0.896 | −0.373 |
| Agomelatine | 0.585 | 0.344 | 0.233 | 0.534 |
| Agomelatine | 0.853 | 0.282 | 0.628 | 0.625 |
| Agomelatine | −0.237 | −0.377 | 0.506 | −0.151 |
| Agomelatine | 0.054 | 0.089 | −0.294 | 0.046 |
| Allopurinol | 0.169 | 0.602 | 0.193 | 0.202 |
| Allopurinol | −0.554 | −0.318 | −0.261 | −0.403 |
| Allopurinol | −0.414 | −0.433 | −0.041 | −0.453 |
| Allopurinol | −0.771 | −0.698 | −0.418 | −0.586 |
| Aloxistatin | 0.163 | −0.023 | 0.142 | 0.124 |
| Aloxistatin | −0.102 | −0.018 | −0.246 | −0.113 |
| Aloxistatin | −0.589 | −0.441 | −0.294 | −0.641 |
| Aloxistatin | −1.169 | −0.531 | −0.985 | −0.769 |
| AM1241 | 0.299 | 0.442 | 0.422 | 0.191 |
| AM1241 | 0.571 | −0.127 | 0.032 | 0.264 |
| AM1241 | −0.110 | −0.223 | −0.134 | −0.087 |
| AM1241 | −0.518 | −0.002 | −0.033 | −0.496 |
| AMG-517 | 0.543 | 0.523 | 0.154 | 0.288 |
| AMG-517 | 0.386 | 0.204 | 0.377 | 0.336 |
| AMG-517 | 0.014 | 0.375 | −0.060 | 0.017 |
| AMG-517 | 1.719 | 0.172 | 0.154 | 1.475 |
| Amlodipine | −0.273 | −0.640 | 0.137 | −0.238 |
| Amlodipine | −0.098 | −0.388 | 0.170 | −0.070 |
| Amlodipine | −0.787 | −0.685 | −0.620 | −0.777 |
| Amlodipine | −1.003 | −0.646 | −1.384 | −1.136 |
| Anacetrapib (MK-0859) | 0.329 | −0.313 | 0.108 | 0.247 |
| Anacetrapib (MK-0859) | −0.013 | 0.128 | 0.058 | −0.013 |
| Anacetrapib (MK-0859) | −0.298 | −0.330 | −0.143 | −0.299 |
| Anacetrapib (MK-0859) | −0.230 | 0.099 | −0.309 | −0.259 |
| Anastrozole | 0.291 | −0.158 | 0.301 | 0.304 |
| Anastrozole | 0.650 | 0.048 | 0.848 | 0.560 |
| Anastrozole | −0.053 | −0.174 | −0.023 | −0.048 |
| Anastrozole | −0.521 | −0.680 | −0.531 | −0.490 |
| Aniracetam | 0.276 | −0.275 | 0.676 | 0.326 |
| Aniracetam | 0.956 | 0.344 | 0.416 | 0.812 |
| Aniracetam | 0.034 | 0.164 | 0.195 | 0.023 |
| Aniracetam | −0.181 | 0.067 | 0.077 | −0.148 |
| AP26113 | 0.706 | −0.124 | −0.130 | 0.563 |
| AP26113 | −0.249 | 0.142 | −0.550 | −0.225 |
| AP26113 | −1.575 | −0.929 | −1.108 | −0.855 |
| AP26113 | −2.218 | −2.814 | −1.072 | −1.119 |
| Apatinib | 1.118 | 0.737 | 0.716 | 0.963 |
| Apatinib | 0.916 | 1.099 | 0.386 | 0.817 |
| Apatinib | 0.721 | 0.890 | 0.522 | 0.575 |
| Apatinib | 0.484 | 0.652 | 0.080 | 0.265 |
| Apigenin | 1.243 | 0.762 | 1.009 | 0.943 |
| Apigenin | 1.065 | 0.524 | 0.807 | 1.024 |
| Apigenin | 1.267 | 0.743 | 1.357 | 1.220 |
| Apigenin | 0.635 | 0.079 | 0.641 | 0.714 |
| Apixaban | 0.526 | −0.279 | 0.035 | 0.528 |
| Apixaban | 0.190 | 0.413 | −0.280 | 0.161 |
| Apixaban | 0.330 | −0.145 | 0.183 | 0.337 |
| Apixaban | −0.620 | −0.285 | −0.701 | −0.684 |
| Apoptosis Activator 2 | 0.851 | 0.620 | 1.201 | 0.551 |
| Apoptosis Activator 2 | 0.473 | 0.610 | 0.234 | 0.383 |
| Apoptosis Activator 2 | −0.058 | 0.303 | −0.051 | −0.068 |
| Apoptosis Activator 2 | −1.627 | −0.327 | −0.915 | −0.891 |
| Aprepitant | 0.740 | 0.907 | 0.603 | 0.444 |
| Aprepitant | 0.692 | 1.161 | 0.586 | 0.812 |
| Aprepitant | −0.045 | 0.444 | 0.049 | −0.048 |
| Aprepitant | −0.770 | 0.131 | −0.838 | −0.716 |
| AS-252424 | 0.465 | 0.498 | 0.437 | 0.340 |
| AS-252424 | −0.051 | −0.183 | −0.407 | −0.060 |
| AS-252424 | −0.476 | −0.508 | −0.583 | −0.286 |
| AS-252424 | −0.141 | −0.654 | −0.801 | −0.091 |
| AT101 | 0.028 | −0.334 | −0.419 | 0.018 |
| AT101 | −0.084 | 0.041 | −0.630 | −0.080 |
| AT101 | −0.552 | −0.610 | −0.269 | −0.489 |
| AT101 | −0.397 | −0.199 | −0.457 | −0.418 |
| Ataluren (PTC124) | 0.333 | 0.842 | 0.213 | 0.266 |
| Ataluren (PTC124) | 0.219 | −0.290 | 0.246 | 0.182 |
| Ataluren (PTC124) | 0.377 | −0.090 | 0.535 | 0.233 |
| Ataluren (PTC124) | 0.033 | 0.015 | 0.069 | 0.034 |
| Atglistatin | 0.184 | 0.214 | 0.551 | 0.192 |
| Atglistatin | −0.229 | 0.014 | 0.390 | −0.183 |
| Atglistatin | 0.167 | −0.091 | 0.537 | 0.178 |
| Atglistatin | 0.078 | −0.679 | −0.081 | 0.026 |
| Atorvastatin Calcium | 0.143 | −0.378 | −0.071 | 0.163 |
| Atorvastatin Calcium | −0.404 | −1.044 | 0.036 | −0.407 |
| Atorvastatin Calcium | −2.288 | −1.528 | −1.386 | −1.655 |
| Atorvastatin Calcium | −1.972 | −2.472 | −1.333 | −1.372 |
| AUY922 (NVP-AUY922) | −1.349 | −1.480 | −1.080 | −1.050 |
| AUY922 (NVP-AUY922) | −1.191 | −1.752 | −1.392 | −1.028 |
| AUY922 (NVP-AUY922) | −1.394 | −1.000 | −1.169 | −1.105 |
| AUY922 (NVP-AUY922) | −1.382 | −2.156 | −1.233 | −1.254 |
| AVL-292 | 1.519 | 0.553 | 0.510 | 1.542 |
| AVL-292 | 0.684 | 0.196 | 0.237 | 0.549 |
| AVL-292 | −1.331 | −0.153 | −0.904 | −0.858 |
| AVL-292 | −2.433 | −1.840 | −1.542 | −1.699 |
| AZ 3146 | 0.318 | −0.293 | 0.768 | 0.278 |
| AZ 3146 | −0.435 | −0.494 | −0.320 | −0.388 |
| AZ 3146 | −0.841 | −0.746 | −0.554 | −0.610 |
| AZ 3146 | −2.730 | −1.900 | −2.015 | −2.054 |
| AZ191 | 0.922 | 0.670 | 0.823 | 0.618 |
| AZ191 | 1.289 | 0.500 | 0.472 | 0.897 |
| AZ191 | 0.628 | 0.498 | 0.481 | 0.718 |
| AZ191 | −1.295 | −0.604 | −0.874 | −0.875 |
| AZ20 | −0.247 | −0.331 | 0.242 | −0.198 |
| AZ20 | −1.524 | −0.424 | −0.845 | −0.870 |
| AZ20 | −2.414 | −1.802 | −1.583 | −1.778 |
| AZ20 | −2.127 | −1.795 | −1.840 | −1.649 |
| AZD1981 | 0.150 | 0.975 | 0.082 | 0.170 |
| AZD1981 | 1.237 | 0.623 | 0.006 | 0.814 |
| AZD1981 | 0.090 | 0.312 | 0.013 | 0.079 |
| AZD1981 | 0.453 | 0.548 | 0.183 | 0.404 |
| AZD2461 | 0.630 | 0.221 | 0.230 | 0.449 |
| AZD2461 | 0.099 | −0.073 | −0.232 | 0.051 |
| AZD2461 | −0.649 | −0.714 | −0.540 | −0.524 |
| AZD2461 | −1.449 | −1.098 | −1.213 | −0.963 |
| AZD3463 | −1.286 | −0.435 | −1.020 | −1.142 |
| AZD3463 | −1.892 | −0.561 | −1.158 | −0.991 |
| AZD3463 | −2.036 | −0.691 | −1.218 | −1.172 |
| AZD3463 | −2.121 | −2.242 | −0.929 | −1.163 |
| AZD4547 | 1.012 | 0.533 | 0.836 | 1.027 |
| AZD4547 | 0.538 | 0.319 | 0.353 | 0.328 |
| AZD4547 | 0.480 | −0.022 | 0.518 | 0.382 |
| AZD4547 | −0.319 | −0.160 | −0.330 | −0.312 |
| AZD6482 | 1.174 | 0.698 | 0.763 | 0.990 |
| AZD6482 | 1.010 | 0.713 | 0.549 | 0.785 |
| AZD6482 | −0.739 | −0.510 | −0.606 | −0.513 |
| AZD6482 | −1.636 | −1.092 | −1.320 | −1.096 |
| AZD7545 | 0.718 | −1.178 | 0.805 | 0.653 |
| AZD7545 | −0.111 | −0.466 | −0.200 | −0.084 |
| AZD7545 | −0.958 | −0.801 | −0.112 | −0.498 |
| AZD7545 | −0.675 | −1.257 | −0.268 | −0.570 |
| AZD7762 | 0.124 | 0.105 | 0.073 | 0.107 |
| AZD7762 | −1.622 | −1.115 | −1.070 | −1.584 |
| AZD7762 | −2.213 | −1.350 | −1.274 | −1.422 |
| AZD7762 | −2.387 | −2.001 | −1.452 | −2.078 |
| BAM7 | 0.509 | 0.724 | 0.226 | 0.338 |
| BAM7 | 0.253 | −0.028 | −0.068 | 0.244 |
| BAM7 | 0.125 | −0.166 | 0.297 | 0.104 |
| BAM7 | −0.128 | −0.177 | −0.046 | −0.124 |
| Batimastat (BB-94) | 0.346 | 0.130 | −0.025 | 0.259 |
| Batimastat (BB-94) | 0.178 | −0.278 | 0.032 | 0.193 |
| Batimastat (BB-94) | 0.363 | −0.153 | 0.596 | 0.432 |
| Batimastat (BB-94) | −0.691 | −0.757 | −0.446 | −0.587 |
| Bazedoxifene HCl | 0.623 | 1.520 | 0.528 | 0.708 |
| Bazedoxifene HCl | 0.420 | 1.349 | 0.217 | 0.354 |
| Bazedoxifene HCl | −1.223 | −0.737 | −1.474 | −1.451 |
| Bazedoxifene HCl | −2.335 | −1.416 | −1.494 | −2.017 |
| Belinostat (PXD101) | −0.382 | −0.708 | 0.061 | −0.320 |
| Belinostat (PXD101) | 0.271 | −0.135 | 0.389 | 0.179 |
| Belinostat (PXD101) | −1.710 | −2.893 | −0.980 | −1.229 |
| Belinostat (PXD101) | −2.113 | −3.679 | −1.486 | −1.383 |
| Bergenin | 1.280 | 0.946 | 1.101 | 1.120 |
| Bergenin | 0.701 | 0.891 | 0.577 | 0.511 |
| Bergenin | 0.810 | 0.382 | 0.676 | 0.670 |
| Bergenin | 0.932 | 0.837 | 0.683 | 0.655 |
| BGJ398 (NVP-BGJ398) | −0.235 | −0.108 | −0.011 | −0.172 |
| BGJ398 (NVP-BGJ398) | 0.011 | 0.059 | −0.402 | 0.005 |
| BGJ398 (NVP-BGJ398) | −0.401 | −0.518 | −0.464 | −0.415 |
| BGJ398 (NVP-BGJ398) | −2.263 | −1.610 | −1.642 | −1.968 |
| BI 2536 | −1.056 | 0.082 | −0.849 | −0.840 |
| BI 2536 | −1.421 | −0.969 | −0.817 | −1.008 |
| BI 2536 | −1.446 | −0.768 | −1.050 | −1.049 |
| BI 2536 | −1.198 | −1.210 | −0.879 | −1.151 |
| BI-D1870 | 1.046 | 0.644 | 1.295 | 0.937 |
| BI-D1870 | 1.025 | 0.082 | 1.096 | 1.123 |
| BI-D1870 | 0.052 | −0.025 | −0.554 | 0.050 |
| BI-D1870 | −2.442 | −1.402 | −2.168 | −1.857 |
| BIBR 1532 | 0.184 | 0.766 | 0.299 | 0.187 |
| BIBR 1532 | 0.689 | −0.082 | 0.363 | 0.585 |
| BIBR 1532 | −0.522 | −0.100 | −0.117 | −0.521 |
| BIBR 1532 | −0.738 | 0.068 | −0.642 | −0.679 |
| Birinapant | 0.487 | −0.787 | 0.334 | 0.210 |
| Birinapant | 0.666 | 0.267 | −0.095 | 0.396 |
| Birinapant | 0.070 | 0.388 | 0.308 | 0.072 |
| Birinapant | −0.520 | 0.450 | −0.280 | −0.527 |
| Bisoprolol fumarate | 0.553 | −0.187 | 0.581 | 0.366 |
| Bisoprolol fumarate | 0.835 | −0.202 | 0.697 | 0.772 |
| Bisoprolol fumarate | 0.478 | −0.063 | 0.564 | 0.495 |
| Bisoprolol fumarate | 0.100 | 0.195 | 0.026 | 0.094 |
| BML-190 | 0.390 | −0.011 | 0.726 | 0.461 |
| BML-190 | −0.238 | −0.269 | −0.124 | −0.172 |
| BML-190 | 0.111 | −0.319 | 0.902 | 0.132 |
| BML-190 | 0.433 | −0.255 | 0.836 | 0.408 |
| BMS-378806 | 1.027 | 0.786 | 1.116 | 1.182 |
| BM5-378806 | 0.045 | 0.347 | −0.033 | 0.037 |
| BMS-378806 | −0.917 | −0.616 | −0.798 | −1.013 |
| BMS-378806 | −2.080 | −1.228 | −1.664 | −1.676 |
| BMS-707035 | 0.867 | 0.970 | 0.827 | 1.023 |
| BMS-707035 | 1.177 | 0.797 | 1.075 | 0.782 |
| BMS-707035 | 0.309 | 0.834 | 0.881 | 0.343 |
| BMS-707035 | 0.320 | 0.457 | 0.448 | 0.289 |
| Bortezomib (PS-341) | −1.548 | −7.160 | −1.205 | −1.277 |
| Bortezomib (PS-341) | −1.117 | −1.311 | −0.842 | −0.897 |
| Bortezomib (PS-341) | −1.612 | −1.144 | −1.125 | −1.426 |
| Bortezomib (PS-341) | −1.539 | −1.076 | −1.380 | −1.229 |
| Bosentan Hydrate | 0.940 | 0.572 | 0.506 | 0.583 |
| Bosentan Hydrate | 0.644 | 0.638 | 0.314 | 0.390 |
| Bosentan Hydrate | 0.089 | 0.416 | 0.236 | 0.080 |
| Bosentan Hydrate | 0.291 | 0.272 | 0.352 | 0.247 |
| Bosutinib (SKI-606) | 1.992 | 1.948 | 1.839 | 2.302 |
| Bosutinib (SKI-606) | 1.343 | 0.783 | 0.790 | 1.181 |
| Bosutinib (SKI-606) | −0.376 | −0.396 | −0.427 | −0.311 |
| Bosutinib (SKI-606) | −1.444 | −1.327 | −0.828 | −1.154 |
| Brinzolamide | 0.196 | −0.017 | 0.871 | 0.093 |
| Brinzolamide | 0.334 | −0.205 | 0.540 | 0.327 |
| Brinzolamide | 0.414 | 0.403 | 0.402 | 0.282 |
| Brinzolamide | 0.536 | 0.204 | 0.519 | 0.477 |
| BTB06584 | 1.436 | 0.606 | 0.690 | 0.951 |
| BTB06584 | 0.274 | 0.861 | 0.162 | 0.179 |
| BTB06584 | 0.254 | −0.205 | 0.138 | 0.184 |
| BTB06584 | 0.120 | 0.652 | −0.219 | 0.090 |
| BTZ043 Racemate | 1.288 | 0.981 | 0.897 | 0.844 |
| BTZ043 Racemate | 1.626 | 0.968 | 0.924 | 1.161 |
| BTZ043 Racemate | 0.247 | 0.168 | 0.757 | 0.264 |
| BTZ043 Racemate | 0.454 | 0.806 | 0.473 | 0.286 |
| Bupivacaine HCl | 0.532 | −0.009 | 0.018 | 0.307 |
| Bupivacaine HCl | −1.094 | −0.614 | −0.862 | −1.059 |
| Bupivacaine HCl | −1.355 | −0.806 | −0.970 | −0.983 |
| Bupivacaine HCl | −1.906 | −1.118 | −1.030 | −1.531 |
| BX-912 | 0.247 | 0.111 | 0.073 | 0.224 |
| BX-912 | 0.269 | 0.186 | 0.121 | 0.186 |
| BX-912 | −1.040 | −0.752 | −0.659 | −0.771 |
| BX-912 | −1.759 | −0.662 | −1.345 | −1.164 |
| C646 | 0.594 | 1.206 | 0.561 | 0.663 |
| C646 | 0.163 | 0.253 | 0.468 | 0.155 |
| C646 | 0.656 | 0.019 | 1.051 | 0.665 |
| C646 | 0.658 | 0.105 | 0.490 | 0.711 |
| Caffeic Acid Phenethyl Ester | 1.219 | 0.691 | 0.553 | 0.630 |
| Caffeic Acid Phenethyl Ester | 0.454 | −0.024 | 0.559 | 0.492 |
| Caffeic Acid Phenethyl Ester | −0.133 | −0.181 | 0.354 | −0.098 |
| Caffeic Acid Phenethyl Ester | −0.756 | −0.707 | −0.155 | −0.532 |
| Canagliflozin | 0.089 | −0.768 | −0.146 | 0.061 |
| Canagliflozin | −0.206 | −0.265 | −0.432 | −0.217 |
| Canagliflozin | 0.178 | 0.156 | 0.232 | 0.194 |
| Canagliflozin | −0.212 | −0.259 | −0.584 | −0.186 |
| Candesartan | −0.098 | −0.008 | 0.058 | −0.067 |
| Candesartan | −0.206 | −0.295 | −0.092 | −0.191 |
| Candesartan | −0.592 | −0.491 | 0.050 | −0.431 |
| Candesartan | −0.482 | −0.598 | −0.474 | −0.476 |
| Captopril | 0.315 | −0.192 | 0.425 | 0.318 |
| Captopril | 0.706 | 0.370 | 0.311 | 0.710 |
| Captopril | 0.328 | 0.416 | 0.200 | 0.260 |
| Captopril | 0.499 | 0.225 | 0.757 | 0.371 |
| Carvedilol | −0.060 | 0.203 | 0.024 | −0.064 |
| Carvedilol | −0.512 | −0.022 | −0.197 | −0.508 |
| Carvedilol | −0.559 | −0.449 | 0.265 | −0.431 |
| Carvedilol | 0.121 | −0.641 | −0.557 | 0.055 |
| CCT128930 | 1.723 | 0.249 | 1.802 | 1.224 |
| CCT128930 | −0.045 | −0.094 | −0.177 | −0.043 |
| CCT128930 | 0.344 | 0.335 | 0.378 | 0.302 |
| CCT128930 | −1.297 | −0.564 | −1.043 | −1.124 |
| Celecoxib | −0.434 | −0.080 | −0.351 | −0.354 |
| Celecoxib | −0.438 | −0.704 | −0.383 | −0.268 |
| Celecoxib | −0.626 | −0.285 | −0.790 | −0.474 |
| Celecoxib | −0.863 | −0.380 | −0.919 | −0.824 |
| CEP-18770 (Delanzomib) | −1.159 | −2.748 | −0.845 | −1.035 |
| CEP-18770 (Delanzomib) | −1.027 | −2.028 | −0.549 | −0.724 |
| CEP-18770 (Delanzomib) | −1.162 | −0.735 | −0.559 | −0.764 |
| CEP-18770 (Delanzomib) | −0.961 | −1.100 | −0.318 | −0.819 |
| CGK733 | 0.050 | −1.055 | 0.490 | 0.057 |
| CGK733 | −0.113 | −0.340 | −0.140 | −0.092 |
| CGK733 | −0.807 | −0.573 | −0.987 | −0.889 |
| CGK733 | −1.487 | −0.987 | −0.346 | −0.797 |
| CGP57380 | 0.621 | 0.234 | 0.707 | 0.636 |
| CGP57380 | 0.348 | −0.328 | −0.394 | 0.213 |
| CGP57380 | −0.616 | −0.810 | −0.440 | −0.431 |
| CGP57380 | −0.571 | 0.109 | −0.194 | −0.329 |
| CGS 21680 HCl | 1.165 | 1.055 | 0.776 | 1.031 |
| CGS 21680 HCl | 1.235 | 1.603 | 1.155 | 1.052 |
| CGS 21680 HCl | 0.629 | 0.967 | 0.428 | 0.525 |
| CGS 21680 HCl | −2.162 | −2.152 | −1.319 | −1.431 |
| CHIR-124 | −1.727 | −0.752 | −0.967 | −1.372 |
| CHIR-124 | −2.191 | −1.552 | −1.664 | −1.637 |
| CHIR-124 | −2.273 | −2.338 | −1.246 | −1.805 |
| CHIR-124 | −2.146 | −1.742 | −1.076 | −1.623 |
| CHIR-98014 | 0.980 | 0.214 | 0.214 | 0.563 |
| CHIR-98014 | −0.377 | 0.236 | −0.427 | −0.308 |
| CHIR-98014 | −0.761 | 0.096 | −1.302 | −0.693 |
| CHIR-98014 | −2.130 | −0.136 | −1.427 | −1.828 |
| Cilomilast | 0.742 | −1.153 | 0.326 | 0.354 |
| Cilomilast | −0.708 | −0.717 | −0.382 | −0.541 |
| Cilomilast | −0.540 | −0.832 | −0.057 | −0.526 |
| Cilomilast | −0.289 | −0.674 | −0.304 | −0.302 |
| Cinacalcet HCl | −0.544 | −0.808 | −0.355 | −0.337 |
| Cinacalcet HCl | 0.251 | −1.018 | −0.119 | 0.212 |
| Cinacalcet HCl | −0.160 | −0.040 | −0.197 | −0.184 |
| Cinacalcet HCl | −0.838 | −0.884 | −0.820 | −0.798 |
| CK-636 | 0.517 | 0.687 | 0.207 | 0.426 |
| CK-636 | −0.082 | −0.011 | −0.424 | −0.071 |
| CK-636 | 0.463 | −0.431 | −0.459 | 0.165 |
| CK-636 | −0.666 | −0.287 | −1.107 | −0.505 |
| Clemastine Fumarate | 0.619 | 0.573 | 0.140 | 0.452 |
| Clemastine Fumarate | −0.424 | 0.102 | −0.226 | −0.402 |
| Clemastine Fumarate | −0.313 | 0.083 | −0.136 | −0.324 |
| Clemastine Fumarate | 0.007 | −0.842 | −0.543 | 0.005 |
| CNX-774 | 0.088 | 0.618 | 0.401 | 0.076 |
| CNX-774 | −0.174 | 0.570 | −0.062 | −0.200 |
| CNX-774 | 0.532 | −0.204 | 1.316 | 0.386 |
| CNX-774 | −0.577 | −0.302 | −0.315 | −0.434 |
| Costunolide | 1.105 | −0.070 | 1.078 | 1.152 |
| Costunolide | 0.666 | 0.408 | 0.537 | 0.670 |
| Costunolide | −0.036 | 0.111 | −0.110 | −0.024 |
| Costunolide | 0.041 | 0.020 | 0.340 | 0.026 |
| CP-673451 | 0.619 | 0.214 | 0.291 | 0.334 |
| CP-673451 | −0.296 | 0.495 | 0.602 | −0.354 |
| CP-673451 | 0.519 | 0.025 | 0.700 | 0.430 |
| CP-673451 | −0.952 | −0.477 | −0.434 | −0.942 |
| CP-91149 | 1.204 | 0.062 | 0.538 | 0.706 |
| CP-91149 | 0.353 | −0.056 | −0.289 | 0.262 |
| CP-91149 | −0.781 | −0.302 | 0.086 | −0.598 |
| CP-91149 | −0.629 | −0.308 | −0.384 | −0.428 |
| Crenolanib (CP-868596) | 0.610 | −0.181 | 1.234 | 0.480 |
| Crenolanib (CP-868596) | 0.279 | −0.056 | 0.446 | 0.315 |
| Crenolanib (CP-868596) | 0.962 | 0.100 | 1.223 | 1.029 |
| Crenolanib (CP-868596) | −1.887 | −0.816 | −1.062 | −1.124 |
| CRT0044876 | −0.012 | −0.303 | 0.107 | −0.009 |
| CRT0044876 | 0.353 | −0.090 | 0.201 | 0.218 |
| CRT0044876 | 0.092 | −0.296 | 0.229 | 0.060 |
| CRT0044876 | −0.392 | −0.290 | 0.099 | −0.347 |
| Cryptotanshinone | 1.007 | 0.615 | 1.141 | 0.997 |
| Cryptotanshinone | −0.677 | −0.040 | −0.371 | −0.251 |
| Cryptotanshinone | −1.861 | −0.472 | −0.524 | −0.675 |
| Cryptotanshinone | −2.795 | −1.992 | −1.715 | −2.245 |
| Cyproterone Acetate | 0.259 | −0.237 | −0.165 | 0.169 |
| Cyproterone Acetate | −0.125 | 0.164 | −0.724 | −0.104 |
| Cyproterone Acetate | −0.599 | −0.260 | −0.398 | −0.446 |
| Cyproterone Acetate | −0.869 | −0.422 | −0.732 | −0.703 |
| Dabrafenib (GSK2118436) | 1.527 | −0.425 | 0.674 | 1.186 |
| Dabrafenib (GSK2118436) | 0.669 | 0.037 | 0.432 | 0.664 |
| Dabrafenib (GSK2118436) | 0.121 | −0.895 | −0.004 | 0.121 |
| Dabrafenib (GSK2118436) | −2.486 | −2.991 | −1.200 | −1.610 |
| Dalcetrapib (JTT-705, RO4607381) | 0.318 | 0.215 | 0.368 | 0.199 |
| Dalcetrapib (JTT-705, RO4607381) | 0.673 | −0.016 | −0.234 | 0.349 |
| Dalcetrapib (JTT-705, RO4607381) | 0.632 | 0.203 | −0.354 | 0.348 |
| Dalcetrapib (JTT-705, RO4607381) | −0.730 | 0.500 | −0.594 | −0.533 |
| Dapagliflozin | 1.901 | 0.982 | 1.086 | 2.137 |
| Dapagliflozin | 0.870 | 0.952 | 0.778 | 0.946 |
| Dapagliflozin | 1.074 | 0.985 | 1.206 | 0.760 |
| Dapagliflozin | 0.936 | 0.854 | 0.921 | 0.570 |
| Daunorubicin HCl | −0.624 | −0.656 | −0.513 | −0.206 |
| Daunorubicin HCl | −2.129 | −2.253 | −1.095 | −0.922 |
| Daunorubicin HCl | −2.218 | −1.023 | −1.073 | −1.039 |
| Daunorubicin HCl | −2.418 | −0.914 | −1.011 | −1.266 |
| DBeQ | 0.292 | 0.282 | 0.519 | 0.242 |
| DBeQ | 1.359 | −0.041 | 0.483 | 0.749 |
| DBeQ | −0.667 | 0.014 | −0.433 | −0.716 |
| DBeQ | −2.733 | −1.177 | −2.168 | −2.062 |
| DCC-2036 (Rebastinib) | 2.318 | 0.461 | 2.060 | 1.927 |
| DCC-2036 (Rebastinib) | 1.204 | 0.612 | 1.297 | 1.360 |
| DCC-2036 (Rebastinib) | 0.347 | −0.068 | 0.498 | 0.296 |
| DCC-2036 (Rebastinib) | −1.578 | −0.748 | −1.114 | −1.482 |
| DMH1 | 0.505 | −0.115 | 0.165 | 0.453 |
| DMH1 | 0.348 | −0.230 | 0.128 | 0.212 |
| DMH1 | 0.229 | −0.004 | 0.083 | 0.163 |
| DMH1 | −0.595 | −0.004 | −0.684 | −0.610 |
| DMXAA (Vadimezan) | −0.587 | −0.691 | −0.040 | −0.465 |
| DMXAA (Vadimezan) | −0.059 | −0.772 | −0.278 | −0.033 |
| DMXAA (Vadimezan) | −0.610 | −0.524 | −0.325 | −0.677 |
| DMXAA (Vadimezan) | −0.765 | −0.571 | −0.209 | −0.809 |
| Doxazosin Mesylate | 0.603 | 0.226 | 0.631 | 0.560 |
| Doxazosin Mesylate | 0.545 | −0.039 | 0.317 | 0.345 |
| Doxazosin Mesylate | 0.197 | 0.488 | 0.437 | 0.235 |
| Doxazosin Mesylate | 0.100 | 0.250 | 0.222 | 0.081 |
| Dutasteride | 0.009 | 0.191 | 0.336 | 0.008 |
| Dutasteride | −0.349 | −0.541 | 0.039 | −0.273 |
| Dutasteride | −0.480 | −0.200 | −0.590 | −0.328 |
| Dutasteride | −0.974 | −0.186 | −0.903 | −0.895 |
| Dynasore | 0.501 | −0.493 | 0.360 | 0.308 |
| Dynasore | 0.787 | −0.247 | −0.171 | 0.382 |
| Dynasore | −0.158 | −0.232 | 0.346 | −0.160 |
| Dynasore | 0.265 | −0.031 | 0.305 | 0.242 |
| E-64 | 0.968 | 0.854 | 1.336 | 1.064 |
| E-64 | 0.719 | 0.162 | 0.116 | 0.578 |
| E-64 | 0.241 | 0.379 | 0.125 | 0.249 |
| E-64 | 0.610 | 0.389 | 0.336 | 0.612 |
| EHop-016 | 0.586 | 0.187 | 0.573 | 0.346 |
| EHop-016 | 0.076 | 0.201 | 0.284 | 0.079 |
| EHop-016 | −0.070 | 0.219 | 0.210 | −0.071 |
| EHop-016 | −1.932 | −1.119 | −0.785 | −0.988 |
| Elvitegravir (GS-9137, JTK-303) | −0.066 | −0.001 | −0.315 | −0.055 |
| Elvitegravir (GS-9137, JTK-303) | 0.597 | −0.078 | −0.527 | 0.215 |
| Elvitegravir (GS-9137, JTK-303) | −0.813 | −1.265 | −0.706 | −0.799 |
| Elvitegravir (GS-9137, JTK-303) | −0.873 | −0.829 | −0.533 | −0.483 |
| Embelin | −0.217 | −0.433 | −0.055 | −0.104 |
| Embelin | −0.791 | −0.463 | −0.460 | −0.401 |
| Embelin | −0.716 | −0.539 | −0.257 | −0.496 |
| Embelin | −0.769 | −0.451 | −0.698 | −0.730 |
| Empagliflozin (BI 10773) | −0.331 | −0.732 | −0.046 | −0.302 |
| Empagliflozin (BI 10773) | −0.353 | −0.217 | −0.332 | −0.398 |
| Empagliflozin (BI 10773) | −0.667 | −0.696 | −0.077 | −0.529 |
| Empagliflozin (BI 10773) | −0.139 | −0.344 | 0.157 | −0.151 |
| Enalaprilat Dihydrate | −0.004 | −0.542 | 0.026 | −0.004 |
| Enalaprilat Dihydrate | 0.718 | −0.330 | −0.028 | 0.429 |
| Enalaprilat Dihydrate | −0.617 | −0.735 | −0.170 | −0.515 |
| Enalaprilat Dihydrate | −1.816 | −0.922 | −0.787 | −1.042 |
| Entacapone | 0.130 | −0.272 | 0.851 | 0.083 |
| Entacapone | 0.153 | −0.095 | 0.071 | 0.109 |
| Entacapone | 0.468 | 0.736 | 0.763 | 0.471 |
| Entacapone | 0.173 | −1.336 | 0.477 | 0.137 |
| Enzalutamide (MDV3100) | 1.140 | 0.429 | 1.243 | 1.206 |
| Enzalutamide (MDV3100) | 1.783 | 0.618 | 0.720 | 0.640 |
| Enzalutamide (MDV3100) | 0.496 | 0.181 | 0.226 | 0.566 |
| Enzalutamide (MDV3100) | 0.493 | 0.225 | 0.366 | 0.233 |
| Enzastaurin (LY317615) | 0.702 | 0.758 | 0.888 | 0.756 |
| Enzastaurin (LY317615) | 1.626 | 1.160 | 1.046 | 0.813 |
| Enzastaurin (LY317615) | 0.340 | 0.875 | 0.390 | 0.347 |
| Enzastaurin (LY317615) | −1.462 | −0.296 | −1.127 | −1.024 |
| EPZ-6438 | 0.022 | 0.113 | 0.085 | 0.017 |
| EPZ-6438 | −0.151 | 0.258 | −0.223 | −0.160 |
| EPZ-6438 | −0.069 | −0.194 | 0.096 | −0.060 |
| EPZ-6438 | 0.297 | 0.392 | 0.156 | 0.095 |
| Erastin | 1.099 | 0.529 | 1.553 | 0.995 |
| Erastin | 1.038 | 0.767 | 1.532 | 0.863 |
| Erastin | 0.686 | 1.161 | 0.908 | 0.588 |
| Erastin | 0.252 | 0.475 | −0.080 | 0.187 |
| Esomeprazole Sodium | 0.942 | 0.840 | 0.679 | 0.644 |
| Esomeprazole Sodium | 1.127 | 0.526 | 1.114 | 1.002 |
| Esomeprazole Sodium | 0.279 | 1.090 | 0.282 | 0.288 |
| Esomeprazole Sodium | 0.135 | 0.797 | −0.116 | 0.127 |
| Etodolac | 0.221 | −0.229 | 0.330 | 0.224 |
| Etodolac | 0.819 | 0.177 | 0.239 | 0.536 |
| Etodolac | 0.450 | 0.027 | 0.330 | 0.391 |
| Etodolac | −0.068 | 0.083 | −0.076 | −0.062 |
| Etomidate | 0.345 | 0.312 | −0.075 | 0.213 |
| Etomidate | 0.309 | 0.012 | 0.196 | 0.204 |
| Etomidate | 0.351 | 0.458 | 0.154 | 0.359 |
| Etomidate | −0.053 | 0.101 | −0.006 | −0.028 |
| EUK 134 | 0.163 | −0.051 | 0.466 | 0.182 |
| EUK 134 | 0.368 | −0.355 | 0.031 | 0.228 |
| EUK 134 | −0.219 | 0.196 | 0.219 | −0.245 |
| EUK 134 | −0.856 | −0.037 | −0.747 | −0.671 |
| Everolimus (RAD001) | −0.078 | 0.911 | 0.401 | −0.069 |
| Everolimus (RAD001) | −0.045 | 0.218 | 0.872 | −0.040 |
| Everolimus (RAD001) | −0.252 | 0.492 | −0.035 | −0.273 |
| Everolimus (RAD001) | −0.622 | −0.199 | −0.238 | −0.736 |
| EX 527 (Selisistat) | 0.404 | 0.863 | 0.671 | 0.278 |
| EX 527 (Selisistat) | 0.306 | 0.585 | 0.520 | 0.356 |
| EX 527 (Selisistat) | −0.329 | 0.985 | 0.235 | −0.211 |
| EX 527 (Selisistat) | −0.068 | 0.833 | 0.438 | −0.050 |
| Exemestane | 0.403 | −0.079 | 0.201 | 0.272 |
| Exemestane | 0.228 | −0.388 | 0.307 | 0.115 |
| Exemestane | 0.377 | 0.416 | 0.008 | 0.355 |
| Exemestane | −0.030 | −0.267 | 0.043 | −0.022 |
| Felodipine | 0.049 | −0.247 | 0.008 | 0.040 |
| Felodipine | −0.273 | −0.574 | −0.285 | −0.248 |
| Felodipine | −0.022 | 0.040 | 0.016 | −0.023 |
| Felodipine | −0.436 | 0.113 | −0.356 | −0.355 |
| Ferrostatin-1 (Fer-1) | 0.778 | 0.785 | 0.825 | 0.805 |
| Ferrostatin-1 (Fer-1) | 1.341 | 1.244 | 0.939 | 1.312 |
| Ferrostatin-1 (Fer-1) | 0.597 | 0.595 | 0.509 | 0.577 |
| Ferrostatin-1 (Fer-1) | −0.119 | −0.132 | 0.088 | −0.097 |
| FG-4592 | 0.538 | 0.795 | 0.391 | 0.396 |
| FG-4592 | 1.516 | 0.834 | 0.676 | 0.661 |
| FG-4592 | 0.982 | 1.082 | 0.910 | 1.122 |
| FG-4592 | 0.456 | 0.711 | 0.296 | 0.510 |
| Finasteride | 1.284 | 0.464 | 2.067 | 1.356 |
| Finasteride | 1.361 | −0.086 | 1.007 | 1.080 |
| Finasteride | 1.008 | 0.176 | 1.296 | 1.034 |
| Finasteride | 0.359 | −0.090 | 0.395 | 0.238 |
| Fingolimod (FTY720) HCl | −0.295 | −0.219 | −0.453 | −0.259 |
| Fingolimod (FTY720) HCl | 0.498 | 0.354 | 0.206 | 0.337 |
| Fingolimod (FTY720) HCl | −0.388 | −0.564 | 0.118 | −0.340 |
| Fingolimod (FTY720) HCl | −2.342 | −2.003 | −1.347 | −1.554 |
| FLI-06 | 0.512 | 0.225 | 0.302 | 0.414 |
| FLI-06 | 0.063 | 0.233 | −0.274 | 0.074 |
| FLI-06 | −0.495 | 0.205 | −0.485 | −0.400 |
| FLI-06 | −1.100 | −0.412 | −1.122 | −0.803 |
| Fluvastatin Sodium | 0.169 | −0.470 | 0.568 | 0.192 |
| Fluvastatin Sodium | −1.141 | −1.446 | −1.022 | −1.024 |
| Fluvastatin Sodium | −1.898 | −3.149 | −1.178 | −1.529 |
| Fluvastatin Sodium | −1.727 | −1.779 | −1.057 | −1.271 |
| Fluvoxamine maleate | 0.737 | 0.285 | 0.651 | 0.709 |
| Fluvoxamine maleate | 1.115 | 0.613 | 0.742 | 1.004 |
| Fluvoxamine maleate | 0.697 | 0.602 | 0.504 | 0.564 |
| Fluvoxamine maleate | 0.075 | 0.384 | −0.163 | 0.068 |
| Formoterol Hemifumarate | 1.331 | 0.816 | 1.386 | 1.588 |
| Formoterol Hemifumarate | 1.894 | 0.846 | 0.551 | 1.639 |
| Formoterol Hemifumarate | 0.582 | 0.460 | 0.454 | 0.651 |
| Formoterol Hemifumarate | 0.069 | 0.700 | 0.075 | 0.056 |
| Forskolin | −0.026 | −0.680 | −0.039 | −0.019 |
| Forskolin | 1.531 | −0.617 | 0.913 | 0.682 |
| Forskolin | 0.658 | −0.652 | 0.666 | 0.315 |
| Forskolin | 0.827 | −0.311 | 1.168 | 0.522 |
| Fostamatinib (R788) | 1.521 | 0.587 | 0.666 | 1.124 |
| Fostamatinib (R788) | 1.007 | −0.134 | −0.199 | 0.548 |
| Fostamatinib (R788) | −0.098 | −0.366 | −0.826 | −0.073 |
| Fostamatinib (R788) | −2.262 | −1.331 | −1.552 | −1.705 |
| Fulvestrant | 0.332 | 0.321 | 0.442 | 0.239 |
| Fulvestrant | −0.319 | −0.079 | −0.282 | −0.181 |
| Fulvestrant | −0.155 | 0.395 | 0.003 | −0.150 |
| Fulvestrant | −0.968 | −0.705 | −0.392 | −0.704 |
| Ganetespib (STA-9090) | −1.680 | −2.231 | −1.046 | −1.137 |
| Ganetespib (STA-9090) | −1.619 | −6.452 | −1.132 | −1.136 |
| Ganetespib (STA-9090) | −1.683 | −2.356 | −1.281 | −1.261 |
| Ganetespib (STA-9090) | −1.727 | −2.162 | −1.025 | −1.201 |
| GDC-0068 | 0.489 | 0.152 | 0.274 | 0.401 |
| GDC-0068 | 0.565 | 0.435 | 0.324 | 0.421 |
| GDC-0068 | −0.380 | 0.589 | −0.507 | −0.340 |
| GDC-0068 | −2.062 | −0.301 | −1.107 | −1.784 |
| GDC-0152 | −0.132 | 0.407 | −0.015 | −0.135 |
| GDC-0152 | −0.231 | 0.509 | −0.183 | −0.244 |
| GDC-0152 | −0.366 | 0.009 | 0.355 | −0.421 |
| GDC-0152 | −0.055 | 0.641 | 0.721 | −0.061 |
| GDC-0941 | 0.117 | 0.949 | 0.075 | 0.119 |
| GDC-0941 | −0.492 | 0.068 | −0.124 | −0.532 |
| GDC-0941 | −1.601 | −0.466 | −1.079 | −1.529 |
| GDC-0941 | −1.661 | −1.686 | −1.108 | −1.395 |
| Ginkgolide A | 0.803 | 0.012 | 0.456 | 0.405 |
| Ginkgolide A | 1.041 | −0.038 | 0.177 | 0.678 |
| Ginkgolide A | 0.014 | 0.125 | 0.228 | 0.015 |
| Ginkgolide A | −0.179 | −0.041 | 0.204 | −0.213 |
| Ginkgolide B | 0.220 | −0.202 | 0.223 | 0.138 |
| Ginkgolide B | 0.302 | 0.106 | 0.496 | 0.348 |
| Ginkgolide B | 0.212 | −0.244 | 0.604 | 0.201 |
| Ginkgolide B | −0.225 | 0.414 | −0.271 | −0.203 |
| Gliclazide | 0.376 | 0.493 | 0.368 | 0.449 |
| Gliclazide | 0.559 | 0.177 | 0.199 | 0.419 |
| Gliclazide | −0.112 | 0.021 | 0.265 | −0.087 |
| Gliclazide | −0.386 | −0.271 | −0.250 | −0.286 |
| Gliquidone | 0.654 | 0.251 | 0.738 | 0.406 |
| Gliquidone | 0.455 | 0.160 | 0.271 | 0.443 |
| Gliquidone | 0.038 | 0.327 | 0.240 | 0.018 |
| Gliquidone | −0.129 | 0.210 | 0.315 | −0.141 |
| GNF-2 | 0.784 | 0.388 | 0.737 | 0.832 |
| GNF-2 | 0.008 | 0.159 | −0.098 | 0.003 |
| GNF-2 | 0.295 | 0.241 | 0.091 | 0.264 |
| GNF-2 | −0.096 | 0.261 | −0.313 | −0.094 |
| Go 6983 | 0.133 | 0.045 | −0.038 | 0.121 |
| Go 6983 | 0.045 | −0.109 | 0.098 | 0.045 |
| Go 6983 | −0.063 | 0.215 | 0.462 | −0.068 |
| Go 6983 | −0.474 | −0.274 | −0.615 | −0.340 |
| Golgicide A | 0.944 | 0.396 | 1.029 | 0.521 |
| Golgicide A | 1.305 | 0.656 | 0.397 | 0.793 |
| Golgicide A | 0.489 | 0.801 | 0.489 | 0.552 |
| Golgicide A | −1.655 | −0.321 | −0.651 | −0.836 |
| Granisetron HCl | 0.099 | −0.762 | 0.320 | 0.107 |
| Granisetron HCl | 0.848 | −0.309 | −0.021 | 0.720 |
| Granisetron HCl | −0.048 | −0.617 | 0.175 | −0.052 |
| Granisetron HCl | −0.669 | −0.882 | −0.197 | −0.401 |
| GSK J4 HCl | 0.556 | 0.300 | 0.459 | 0.533 |
| GSK J4 HCl | 0.704 | 0.232 | 0.266 | 0.418 |
| GSK J4 HCl | 0.265 | 0.005 | 0.107 | 0.245 |
| GSK J4 HCl | −0.013 | 0.801 | −0.005 | −0.009 |
| GSK1292263 | 0.715 | 0.515 | 0.604 | 0.538 |
| GSK1292263 | 0.655 | 0.498 | 0.419 | 0.608 |
| GSK1292263 | 0.390 | 0.816 | 0.196 | 0.352 |
| GSK1292263 | 0.173 | 0.604 | 0.114 | 0.112 |
| GSK1904529A | 0.860 | 0.539 | 0.959 | 0.963 |
| GSK1904529A | −0.301 | −0.153 | 0.347 | −0.265 |
| GSK1904529A | −1.453 | −0.946 | −1.610 | −1.057 |
| GSK1904529A | −1.788 | −2.130 | −1.142 | −1.539 |
| GSK2656157 | −0.055 | 0.224 | −0.254 | −0.053 |
| GSK2656157 | 0.390 | 0.408 | −0.448 | 0.207 |
| GSK2656157 | −0.130 | 0.253 | −0.222 | −0.107 |
| GSK2656157 | 0.581 | 0.405 | −0.080 | 0.228 |
| GSK429286A | 1.599 | 1.428 | 0.833 | 1.795 |
| GSK429286A | 1.832 | 1.544 | 1.053 | 1.463 |
| GSK429286A | 1.586 | 1.092 | 0.948 | 1.439 |
| GSK429286A | 0.643 | 1.002 | 0.374 | 0.473 |
| GSK461364 | 0.724 | 0.555 | 0.557 | 0.733 |
| GSK461364 | −0.601 | −0.571 | −0.831 | −0.579 |
| GSK461364 | −2.273 | −1.748 | −1.614 | −1.667 |
| GSK461364 | −2.520 | −2.083 | −2.291 | −1.693 |
| GSK690693 | 0.123 | 0.338 | 0.444 | 0.116 |
| GSK690693 | 1.002 | 0.503 | 0.360 | 0.820 |
| GSK690693 | 0.800 | −0.092 | 0.987 | 0.756 |
| GSK690693 | −0.991 | −0.200 | −0.552 | −0.552 |
| GW0742 | 0.307 | −0.456 | 0.252 | 0.279 |
| GW0742 | 0.689 | −0.426 | 0.406 | 0.508 |
| GW0742 | 0.092 | −0.721 | 0.267 | 0.077 |
| GW0742 | −1.183 | −1.392 | −0.740 | −0.927 |
| GW2580 | −0.082 | −0.099 | 0.045 | −0.071 |
| GW2580 | 0.128 | −0.465 | 0.111 | 0.098 |
| GW2580 | −0.389 | 0.056 | −0.615 | −0.441 |
| GW2580 | −0.168 | −0.097 | −0.161 | −0.175 |
| GW3965 HCl | 0.590 | 0.501 | 0.291 | 0.364 |
| GW3965 HCl | 0.480 | 0.634 | 0.011 | 0.326 |
| GW3965 HCl | 1.156 | 0.650 | 0.590 | 0.967 |
| GW3965 HCl | −0.301 | −0.214 | −0.065 | −0.280 |
| GW4064 | 0.184 | −0.460 | 0.703 | 0.171 |
| GW4064 | 0.627 | 0.491 | −0.227 | 0.540 |
| GW4064 | −0.003 | −0.373 | 0.166 | −0.003 |
| GW4064 | −0.460 | −0.540 | −0.544 | −0.488 |
| GW441756 | 0.486 | 0.120 | 0.478 | 0.466 |
| GW441756 | 1.268 | −0.329 | 0.568 | 0.602 |
| GW441756 | 0.806 | 0.468 | 1.215 | 0.911 |
| GW441756 | 0.458 | −0.350 | 0.357 | 0.449 |
| GW9508 | 0.434 | 0.128 | 0.536 | 0.329 |
| GW9508 | 0.316 | 0.384 | 0.122 | 0.225 |
| GW9508 | 0.003 | −0.003 | −0.064 | 0.002 |
| GW9508 | −0.289 | −0.134 | −0.358 | −0.176 |
| GW9662 | 0.235 | 0.301 | 0.272 | 0.218 |
| GW9662 | 0.520 | 0.117 | 0.722 | 0.534 |
| GW9662 | 0.599 | 0.715 | 0.336 | 0.571 |
| GW9662 | 0.131 | 0.378 | 0.282 | 0.149 |
| H 89 2HCl | 0.764 | 1.039 | 0.995 | 0.529 |
| H 89 2HCl | 0.955 | 0.952 | 0.816 | 0.703 |
| H 89 2HCl | 0.565 | 0.830 | 0.521 | 0.329 |
| H 89 2HCl | −0.001 | 1.211 | 0.374 | −0.001 |
| HA14-1 | 1.231 | 1.389 | 1.056 | 1.264 |
| HA14-1 | 2.041 | 1.961 | 1.013 | 1.052 |
| HA14-1 | 1.099 | 2.075 | 1.483 | 1.202 |
| HA14-1 | 0.964 | 1.289 | 0.640 | 0.449 |
| HC-030031 | 0.584 | 0.200 | 0.506 | 0.499 |
| HC-030031 | 0.627 | 0.606 | 0.276 | 0.539 |
| HC-030031 | −0.225 | −0.025 | −0.048 | −0.200 |
| HC-030031 | 0.499 | −0.715 | −0.383 | 0.322 |
| I-BET151 (GSK1210151A) | −1.563 | −0.045 | −0.856 | −1.087 |
| I-BET151 (GSK1210151A) | −2.225 | −0.433 | −1.677 | −1.784 |
| I-BET151 (GSK1210151A) | −2.687 | −1.788 | −1.629 | −1.815 |
| I-BET151 (GSK1210151A) | −2.641 | −2.304 | −1.342 | −1.694 |
| Ibrutinib (PCI-32765) | −0.902 | −0.062 | −0.704 | −0.995 |
| Ibrutinib (PCI-32765) | −1.335 | −0.574 | −1.031 | −1.410 |
| Ibrutinib (PCI-32765) | −1.888 | −1.005 | −1.175 | −1.339 |
| Ibrutinib (PCI-32765) | −2.016 | −0.875 | −1.325 | −1.361 |
| ICG-001 | 1.054 | 0.298 | 0.280 | 0.711 |
| ICG-001 | 1.318 | 0.360 | 0.339 | 0.796 |
| ICG-001 | 0.109 | 0.530 | 0.413 | 0.122 |
| ICG-001 | −0.802 | −0.731 | −1.162 | −0.824 |
| Icotinib | 0.609 | −0.065 | 0.227 | 0.689 |
| Icotinib | −0.583 | −0.162 | −0.710 | −0.544 |
| Icotinib | −1.572 | −0.865 | −1.321 | −1.726 |
| Icotinib | −2.708 | −3.189 | −2.654 | −1.917 |
| Ifenprodil Tartrate | 0.325 | 0.218 | 0.372 | 0.255 |
| Ifenprodil Tartrate | 0.107 | 0.279 | −0.116 | 0.091 |
| Ifenprodil Tartrate | −0.151 | −0.009 | 0.504 | −0.103 |
| Ifenprodil Tartrate | −1.947 | −1.277 | −1.176 | −1.030 |
| IKK-16 (IKK Inhibitor VII) | 0.593 | 0.131 | 0.817 | 0.496 |
| IKK-16 (IKK Inhibitor VII) | 0.179 | −0.190 | 0.207 | 0.203 |
| IKK-16 (IKK Inhibitor VII) | −0.918 | −0.877 | −0.481 | −0.698 |
| IKK-16 (IKK Inhibitor VII) | −2.475 | −1.581 | −2.119 | −1.351 |
| Ilomastat (GM6001, Galardin) | 1.385 | 0.482 | 0.305 | 1.175 |
| Ilomastat (GM6001, Galardin) | 0.754 | 0.406 | −0.056 | 0.580 |
| Ilomastat (GM6001, Galardin) | 0.153 | 0.321 | 0.066 | 0.164 |
| Ilomastat (GM6001, Galardin) | −0.502 | −0.348 | −0.635 | −0.329 |
| Imatinib (STI571) | 0.001 | −0.258 | 0.326 | 0.001 |
| Imatinib (STI571) | 0.246 | 0.254 | 0.108 | 0.236 |
| Imatinib (STI571) | −0.005 | 0.194 | 0.152 | −0.004 |
| Imatinib (STI571) | −0.237 | −0.097 | −0.330 | −0.241 |
| IMD 0354 | −0.303 | −0.019 | 0.021 | −0.335 |
| IMD 0354 | 0.281 | 0.258 | 0.439 | 0.203 |
| IMD 0354 | 0.534 | 0.274 | 0.612 | 0.537 |
| IMD 0354 | −0.826 | −0.748 | −0.729 | −0.736 |
| Imidapril HCl | −0.274 | −1.054 | −0.047 | −0.269 |
| Imidapril HCl | 0.702 | −0.525 | 0.004 | 0.315 |
| Imidapril HCl | −0.226 | −0.548 | 0.101 | −0.262 |
| Imidapril HCl | −0.487 | −0.699 | −0.541 | −0.404 |
| Iniparib (B5I-201) | 0.205 | 0.883 | 0.375 | 0.148 |
| Iniparib (BSI-201) | 0.229 | 0.347 | 0.003 | 0.247 |
| Iniparib (BSI-201) | −0.006 | 0.475 | 0.109 | −0.005 |
| Iniparib (BSI-201) | −0.062 | 0.721 | −0.163 | −0.070 |
| IOX2 | 0.305 | 0.501 | 0.296 | 0.359 |
| IOX2 | 0.456 | 0.264 | 0.182 | 0.428 |
| IOX2 | 0.005 | 0.287 | 0.408 | 0.006 |
| IOX2 | −0.314 | 0.003 | −0.420 | −0.298 |
| IPA-3 | 0.413 | 0.818 | 0.027 | 0.356 |
| IPA-3 | −0.202 | −0.408 | 0.026 | −0.184 |
| IPA-3 | 0.431 | −0.316 | −0.008 | 0.324 |
| IPA-3 | −0.347 | −0.172 | −0.175 | −0.305 |
| Irinotecan | −0.023 | −0.378 | −0.217 | −0.018 |
| Irinotecan | −0.939 | −1.558 | −0.556 | −0.721 |
| Irinotecan | −1.596 | −1.491 | −1.415 | −1.400 |
| Irinotecan | −1.624 | −2.716 | −1.102 | −1.217 |
| Irinotecan HCl Trihydrate | −0.261 | −0.305 | −0.182 | −0.311 |
| Irinotecan HCl Trihydrate | −1.020 | −0.694 | −0.831 | −0.747 |
| Irinotecan HCl Trihydrate | −1.895 | −1.060 | −1.832 | −1.550 |
| Irinotecan HCl Trihydrate | −2.375 | −1.809 | −2.032 | −1.941 |
| Isotretinoin | 0.708 | 1.156 | 0.753 | 0.726 |
| Isotretinoin | 0.306 | 0.966 | 0.312 | 0.275 |
| Isotretinoin | 0.223 | 0.779 | 0.410 | 0.210 |
| Isotretinoin | 0.484 | 0.304 | 0.680 | 0.499 |
| Ispinesib (SB-715992) | −1.753 | −0.942 | −1.681 | −1.495 |
| Ispinesib (SB-715992) | −1.914 | −0.971 | −1.354 | −1.509 |
| Ispinesib (SB-715992) | −1.859 | −1.118 | −1.617 | −1.657 |
| Ispinesib (SB-715992) | −1.850 | −1.236 | −1.093 | −1.525 |
| Istradefylline | −0.190 | −0.113 | −0.084 | −0.139 |
| Istradefylline | −0.560 | 0.225 | −0.362 | −0.661 |
| Istradefylline | −0.621 | −0.412 | −0.012 | −0.578 |
| Istradefylline | −0.367 | −0.106 | −0.336 | −0.289 |
| Ivacaftor (VX-770) | 0.853 | 0.431 | 0.882 | 0.705 |
| Ivacaftor (VX-770) | 1.220 | 0.280 | 0.825 | 1.209 |
| Ivacaftor (VX-770) | 0.124 | −0.331 | 0.582 | 0.148 |
| Ivacaftor (VX-770) | −1.636 | −1.317 | −1.167 | −1.350 |
| JIB-04 | 1.312 | 0.552 | 0.941 | 0.895 |
| JIB-04 | 0.545 | 0.331 | 0.239 | 0.407 |
| JIB-04 | −0.365 | 0.282 | 0.042 | −0.339 |
| JIB-04 | −0.552 | −0.531 | −0.845 | −0.195 |
| JNJ-1661010 | 0.873 | −0.169 | 0.805 | 0.612 |
| JNJ-1661010 | 0.263 | −0.659 | 0.332 | 0.250 |
| JNJ-1661010 | −0.408 | −0.113 | 0.332 | −0.454 |
| JNJ-1661010 | −0.441 | −0.309 | 0.237 | −0.449 |
| JSH-23 | 1.064 | 0.462 | 0.371 | 1.259 |
| JSH-23 | 0.686 | 0.536 | 0.452 | 0.611 |
| JSH-23 | 0.032 | 0.179 | −0.012 | 0.030 |
| JSH-23 | −0.171 | 0.139 | −0.250 | −0.147 |
| Ki16198 | 0.176 | −0.028 | 0.052 | 0.136 |
| Ki16198 | 0.204 | −0.359 | −0.083 | 0.231 |
| Ki16198 | −0.095 | −0.118 | 0.211 | −0.105 |
| Ki16198 | −0.239 | 0.073 | −0.914 | −0.262 |
| Ki16425 | 0.142 | −0.145 | 0.006 | 0.130 |
| Ki16425 | 0.739 | −0.008 | 0.483 | 0.421 |
| Ki16425 | −0.135 | −0.226 | −0.072 | −0.133 |
| Ki16425 | −0.459 | −0.527 | −0.246 | −0.394 |
| KPT-185 | −0.096 | 0.296 | −0.049 | −0.092 |
| KPT-185 | 1.420 | 0.270 | 0.872 | 0.978 |
| KPT-185 | −2.085 | −1.033 | −1.718 | −1.455 |
| KPT-185 | −0.989 | −0.723 | −0.298 | −0.431 |
| KPT-276 | 0.860 | 0.494 | 0.486 | 0.806 |
| KPT-276 | 0.958 | −0.146 | 0.742 | 0.771 |
| KPT-276 | −2.111 | −0.772 | −1.426 | −1.399 |
| KPT-276 | −2.335 | −2.520 | −1.384 | −1.417 |
| KPT-330 | 1.677 | 0.686 | 1.021 | 1.329 |
| KPT-330 | −1.821 | −0.942 | −1.822 | −0.894 |
| KPT-330 | −2.523 | −2.044 | −1.556 | −1.575 |
| KPT-330 | −2.733 | −2.988 | −1.714 | −1.599 |
| KU-55933 (ATM Kinase Inhibitor) | 1.088 | 1.626 | 1.188 | 1.113 |
| KU-55933 (ATM Kinase Inhibitor) | 1.291 | 1.743 | 1.035 | 0.994 |
| KU-55933 (ATM Kinase Inhibitor) | 1.043 | 1.554 | 1.076 | 1.246 |
| KU-55933 (ATM Kinase Inhibitor) | 0.494 | 1.744 | 0.504 | 0.400 |
| KU-60019 | 1.490 | 0.371 | 0.305 | 0.938 |
| KU-60019 | 0.474 | 0.118 | −0.014 | 0.312 |
| KU-60019 | −0.317 | 0.175 | −0.103 | −0.261 |
| KU-60019 | −1.436 | −0.916 | −0.570 | −0.923 |
| KX2-391 | −2.561 | −2.473 | −2.177 | −2.252 |
| KX2-391 | −2.455 | −2.845 | −1.855 | −1.641 |
| KX2-391 | −2.741 | −2.822 | −1.701 | −1.898 |
| KX2-391 | −2.709 | −1.255 | −2.061 | −2.140 |
| Lafutidine | 0.339 | 0.452 | 0.285 | 0.331 |
| Lafutidine | 0.506 | 0.476 | 0.064 | 0.460 |
| Lafutidine | 0.491 | 0.505 | 0.359 | 0.503 |
| Lafutidine | −0.093 | −0.249 | 0.581 | −0.111 |
| LB42708 | −0.576 | −0.254 | −1.118 | −0.519 |
| LB42708 | −0.934 | −0.531 | −1.157 | −0.630 |
| LB42708 | −1.539 | −0.186 | −1.034 | −0.965 |
| LB42708 | −1.509 | −1.059 | −0.939 | −0.864 |
| LDE225 (NVP-LDE225, Erismodegib) | 0.613 | 0.313 | 0.869 | 0.704 |
| LDE225 (NVP-LDE225, Erismodegib) | 0.481 | 0.163 | 0.731 | 0.415 |
| LDE225 (NVP-LDE225, Erismodegib) | 0.013 | 0.681 | 0.074 | 0.013 |
| LDE225 (NVP-LDE225, Erismodegib) | 0.038 | −0.001 | 0.268 | 0.025 |
| LDK378 | 0.763 | 0.320 | 0.246 | 0.574 |
| LDK378 | −0.306 | 0.013 | −0.527 | −0.290 |
| LDK378 | −1.817 | −0.596 | −1.818 | −1.400 |
| LDK378 | −3.149 | −1.329 | −2.550 | −2.075 |
| LDN-212854 | 1.050 | 0.686 | 0.567 | 0.799 |
| LDN-212854 | 0.167 | 0.242 | 0.395 | 0.136 |
| LDN-212854 | −1.888 | −1.244 | −1.438 | −1.525 |
| LDN-212854 | −2.582 | −1.589 | −1.649 | −1.672 |
| Lenalidomide (CC-5013) | 0.639 | 0.169 | 1.215 | 0.593 |
| Lenalidomide (CC-5013) | 1.137 | −0.049 | 0.674 | 0.873 |
| Lenalidomide (CC-5013) | 0.162 | −0.710 | 0.472 | 0.193 |
| Lenalidomide (CC-5013) | −0.290 | −0.802 | −0.318 | −0.298 |
| Letrozole | 0.258 | −0.043 | 0.554 | 0.212 |
| Letrozole | 0.970 | 0.336 | 1.104 | 0.764 |
| Letrozole | 0.038 | 0.288 | 0.063 | 0.041 |
| Letrozole | 0.040 | 0.606 | −0.042 | 0.028 |
| Levosulpiride | −0.108 | −0.129 | 0.092 | −0.076 |
| Levosulpiride | 0.830 | −0.019 | 0.226 | 0.774 |
| Levosulpiride | 0.800 | 0.087 | 0.501 | 0.531 |
| Levosulpiride | −0.168 | 0.297 | −0.195 | −0.154 |
| Lidocaine | 0.537 | −0.237 | 0.904 | 0.448 |
| Lidocaine | 0.167 | −0.371 | 0.132 | 0.197 |
| Lidocaine | −0.244 | 0.116 | 0.008 | −0.268 |
| Lidocaine | −0.061 | −0.889 | 0.183 | −0.070 |
| Linagliptin | 0.905 | 0.299 | 1.046 | 0.797 |
| Linagliptin | 0.305 | 0.612 | 0.306 | 0.215 |
| Linagliptin | −0.204 | 0.047 | 0.118 | −0.126 |
| Linagliptin | 0.322 | −0.025 | 0.671 | 0.374 |
| Lomeguatrib | −0.528 | −0.489 | −0.474 | −0.435 |
| Lomeguatrib | −0.139 | −0.422 | −0.137 | −0.121 |
| Lomeguatrib | −0.356 | 0.193 | −0.189 | −0.346 |
| Lomeguatrib | −0.893 | −0.190 | −1.038 | −0.820 |
| Loratadine | −0.490 | −0.683 | 0.098 | −0.535 |
| Loratadine | 0.069 | 0.535 | −0.004 | 0.082 |
| Loratadine | −0.959 | −0.609 | −0.783 | −0.811 |
| Loratadine | −0.956 | −0.360 | −0.977 | −1.029 |
| Losartan Potassium (DuP 753) | −0.112 | −0.338 | 0.273 | −0.104 |
| Losartan Potassium (DuP 753) | 0.173 | −0.304 | 0.263 | 0.194 |
| Losartan Potassium (DuP 753) | −0.519 | −0.274 | −0.341 | −0.616 |
| Losartan Potassium (DuP 753) | −0.164 | −0.390 | 0.423 | −0.185 |
| Lovastatin | −0.147 | −0.609 | 0.014 | −0.128 |
| Lovastatin | −0.320 | −1.511 | −0.516 | −0.236 |
| Lovastatin | −2.572 | −2.978 | −1.430 | −1.424 |
| Lovastatin | −2.645 | −1.911 | −1.590 | −1.288 |
| Loxistatin Acid (E-64C) | 0.808 | 0.218 | 0.757 | 0.833 |
| Loxistatin Acid (E-64C) | 0.677 | 0.402 | 0.330 | 0.449 |
| Loxistatin Acid (E-64C) | 0.238 | 0.176 | 0.411 | 0.159 |
| Loxistatin Acid (E-64C) | 0.138 | 0.270 | 0.027 | 0.141 |
| LY2157299 | 1.209 | 0.343 | 0.656 | 1.145 |
| LY2157299 | 1.287 | 0.369 | 0.737 | 1.157 |
| LY2157299 | 0.731 | 0.048 | 0.827 | 0.808 |
| LY2157299 | 1.441 | 0.314 | 1.243 | 1.219 |
| LY2228820 | −0.107 | −0.908 | 0.186 | −0.087 |
| LY2228820 | −0.348 | −0.614 | −0.798 | −0.344 |
| LY2228820 | −0.844 | −0.794 | 0.123 | −0.692 |
| LY2228820 | −1.062 | −0.937 | −1.170 | −1.268 |
| LY2603618 | 0.056 | −0.013 | −0.044 | 0.065 |
| LY2603618 | −0.953 | −0.880 | −0.697 | −0.885 |
| LY2603618 | −1.737 | −1.281 | −1.609 | −1.421 |
| LY2603618 | −2.300 | −2.490 | −1.826 | −1.890 |
| LY2784544 | 0.794 | 0.378 | 0.395 | 0.911 |
| LY2784544 | 0.498 | 0.135 | 0.037 | 0.595 |
| LY2784544 | 0.737 | 0.500 | 0.582 | 0.492 |
| LY2784544 | −2.373 | −1.283 | −2.015 | −2.074 |
| LY411575 | 0.155 | 0.086 | 0.217 | 0.094 |
| LY411575 | 0.266 | −0.312 | −0.432 | 0.171 |
| LY411575 | −0.590 | −0.389 | −0.593 | −0.596 |
| LY411575 | −1.015 | −0.550 | −0.931 | −0.917 |
| Maraviroc | 0.350 | −0.294 | 0.274 | 0.262 |
| Maraviroc | 1.262 | 0.187 | 0.365 | 0.754 |
| Maraviroc | 0.034 | −0.030 | 0.225 | 0.028 |
| Maraviroc | 0.495 | 0.270 | 0.547 | 0.400 |
| Mdivi-1 | 0.511 | 0.026 | 0.715 | 0.520 |
| Mdivi-1 | 0.196 | −0.072 | 0.481 | 0.152 |
| Mdivi-1 | −0.310 | −0.356 | −0.093 | −0.196 |
| Mdivi-1 | −2.655 | −1.838 | −1.749 | −2.328 |
| Memantine HCl | 0.586 | 0.214 | 0.489 | 0.420 |
| Memantine HCl | 0.112 | 0.219 | 0.038 | 0.134 |
| Memantine HCl | 0.328 | −0.016 | 0.745 | 0.292 |
| Memantine HCl | −0.138 | 0.145 | −0.407 | −0.142 |
| Methotrexate | −1.029 | −1.489 | −0.745 | −0.943 |
| Methotrexate | −0.831 | −1.028 | −0.349 | −0.627 |
| Methotrexate | −1.143 | −1.982 | −1.130 | −0.866 |
| Methotrexate | −1.007 | −1.176 | −0.527 | −0.718 |
| Mirabegron | 0.958 | 0.101 | 1.039 | 0.863 |
| Mirabegron | −0.103 | −0.380 | −0.280 | −0.090 |
| Mirabegron | −0.317 | 0.068 | −0.346 | −0.363 |
| Mirabegron | −0.065 | −0.150 | 0.113 | −0.064 |
| MK-1775 | 0.424 | 0.536 | 0.317 | 0.440 |
| MK-1775 | −1.404 | −1.242 | −1.150 | −1.378 |
| MK-1775 | −1.345 | −1.697 | −1.545 | −1.357 |
| MK-1775 | −1.643 | −1.818 | −0.993 | −1.331 |
| MK-2206 2HCl | 0.460 | 0.242 | 0.230 | 0.403 |
| MK-2206 2HCl | 0.536 | −0.074 | −0.481 | 0.232 |
| MK-2206 2HCl | −1.546 | −0.453 | −1.586 | −1.686 |
| MK-2206 2HCl | −2.370 | −1.730 | −1.642 | −1.628 |
| MK-2866 (GTx-024) | 0.466 | 0.004 | 0.314 | 0.378 |
| MK-2866 (GTx-024) | 0.322 | −0.285 | 0.150 | 0.164 |
| MK-2866 (GTx-024) | 0.506 | −0.199 | 0.169 | 0.307 |
| MK-2866 (GTx-024) | −0.463 | −0.814 | −0.515 | −0.543 |
| MK-8245 | 0.449 | 0.453 | 0.168 | 0.442 |
| MK-8245 | 1.194 | −0.219 | 0.200 | 0.527 |
| MK-8245 | −0.399 | −0.054 | −0.322 | −0.357 |
| MK-8245 | −0.799 | −0.921 | −0.716 | −0.752 |
| ML130 (Nodinitib-1) | 0.093 | 0.170 | 0.247 | 0.083 |
| ML130 (Nodinitib-1) | 0.088 | 0.172 | 0.051 | 0.090 |
| ML130 (Nodinitib-1) | 0.426 | 0.398 | 0.487 | 0.490 |
| ML130 (Nodinitib-1) | −0.271 | 0.545 | 0.208 | −0.305 |
| ML133 HCl | 0.404 | −0.026 | 0.282 | 0.324 |
| ML133 HCl | −0.118 | −0.437 | −0.131 | −0.129 |
| ML133 HCl | 0.565 | −0.560 | 0.740 | 0.368 |
| ML133 HCl | −0.089 | −0.133 | −0.152 | −0.088 |
| ML161 | 0.113 | 0.278 | 0.117 | 0.096 |
| ML161 | −0.173 | 0.087 | −0.239 | −0.148 |
| ML161 | 0.158 | 0.229 | 0.354 | 0.152 |
| ML161 | −0.429 | −0.107 | −0.421 | −0.282 |
| ML347 | 0.608 | 0.464 | 1.159 | 0.546 |
| ML347 | 0.715 | 0.557 | 1.286 | 0.729 |
| ML347 | 0.467 | 0.403 | 0.509 | 0.450 |
| ML347 | −1.303 | −0.307 | −0.643 | −1.245 |
| MLN2238 | −2.478 | −2.218 | −1.766 | −1.588 |
| MLN2238 | −2.317 | −0.972 | −1.837 | −1.620 |
| MLN2238 | −2.280 | −1.669 | −1.787 | −1.733 |
| MLN2238 | −2.394 | −1.891 | −2.223 | −1.739 |
| MLN8054 | 0.274 | 0.080 | 0.618 | 0.241 |
| MLN8054 | 1.081 | 0.035 | 0.061 | 0.554 |
| MLN8054 | −0.562 | −0.959 | −0.713 | −0.486 |
| MLN8054 | −1.274 | −1.480 | −0.870 | −0.964 |
| MM-102 | −0.177 | −0.485 | −0.233 | −0.148 |
| MM-102 | −0.009 | −0.493 | −0.084 | −0.010 |
| MM-102 | −0.226 | −0.877 | 0.128 | −0.218 |
| MM-102 | −1.252 | −1.350 | −0.792 | −0.881 |
| MNS (3,4-Methylenedioxy- | 0.493 | −0.026 | 0.518 | 0.451 |
| CE≤-nitrostyrene, MDBN) | | | | |
| MNS (3,4-Methylenedioxy- | 0.648 | 0.148 | −0.082 | 0.331 |
| CE≤-nitrostyrene, MDBN) | | | | |
| MNS (3,4-Methylenedioxy- | 0.124 | 0.166 | 0.270 | 0.073 |
| CE≤-nitrostyrene, MDBN) | | | | |
| MNS (3,4-Methylenedioxy- | −1.157 | −0.338 | −0.670 | −0.676 |
| CE≤-nitrostyrene, MDBN) | | | | |
| Moclobemide (Ro 111163) | 0.953 | 0.257 | 0.302 | 0.495 |
| Moclobemide (Ro 111163) | 0.607 | 0.148 | 0.415 | 0.348 |
| Moclobemide (Ro 111163) | 0.078 | 0.227 | 0.007 | 0.056 |
| Moclobemide (Ro 111163) | 0.123 | 0.115 | −0.219 | 0.135 |
| Mozavaptan | 0.557 | 0.521 | 0.283 | 0.266 |
| Mozavaptan | 1.068 | 0.427 | −0.203 | 0.560 |
| Mozavaptan | 0.688 | 0.274 | 0.340 | 0.775 |
| Mozavaptan | −1.745 | −0.812 | −1.544 | −1.480 |
| MRS 2578 | 0.612 | −0.407 | 0.854 | 0.544 |
| MRS 2578 | 0.430 | 0.124 | 0.290 | 0.387 |
| MRS 2578 | 0.149 | −0.439 | 0.204 | 0.155 |
| MRS 2578 | 0.082 | −0.537 | 0.202 | 0.061 |
| Mubritinib (TAK 165) | −0.768 | −1.444 | −0.464 | −0.875 |
| Mubritinib (TAK 165) | −0.996 | −0.856 | −0.949 | −0.772 |
| Mubritinib (TAK 165) | −1.934 | −1.523 | −1.662 | −1.891 |
| Mubritinib (TAK 165) | −1.901 | −1.072 | −2.433 | −2.222 |
| Naftopidil | 0.097 | −0.131 | −0.179 | 0.061 |
| Naftopidil | −0.830 | −0.525 | −0.661 | −0.734 |
| Naftopidil | −0.625 | −0.523 | −0.167 | −0.735 |
| Naftopidil | −0.510 | −0.405 | −0.723 | −0.547 |
| Naltrexone HCl | 0.014 | 0.306 | −0.073 | 0.014 |
| Naltrexone HCl | 0.164 | 0.212 | −0.044 | 0.171 |
| Naltrexone HCl | 0.164 | 0.015 | 0.138 | 0.161 |
| Naltrexone HCl | 0.294 | 0.251 | 0.274 | 0.281 |
| Naproxen | 0.434 | −0.269 | 0.728 | 0.306 |
| Naproxen | 0.370 | −0.219 | 0.399 | 0.295 |
| Naproxen | −1.393 | −0.991 | −0.577 | −1.230 |
| Naproxen | −1.389 | −1.547 | −0.606 | −0.735 |
| NC | −2.210 | NA | NA | NA |
| NC | −2.111 | NA | NA | NA |
| NC | −2.058 | NA | NA | NA |
| NC | −2.069 | NA | NA | NA |
| Nebivolol | 1.254 | 1.057 | 1.220 | 0.751 |
| Nebivolol | 1.826 | 0.670 | 0.870 | 1.637 |
| Nebivolol | 0.446 | 1.162 | 0.692 | 0.432 |
| Nebivolol | 0.681 | 0.663 | 0.855 | 0.588 |
| Necrostatin-1 | 0.498 | 0.289 | 0.385 | 0.426 |
| Necrostatin-1 | 0.248 | 0.293 | 0.162 | 0.156 |
| Necrostatin-1 | 0.597 | 0.024 | 0.504 | 0.406 |
| Necrostatin-1 | 1.482 | 0.200 | 0.464 | 1.452 |
| NH125 | −0.738 | −0.591 | −0.290 | −0.718 |
| NH125 | −1.536 | −0.945 | −1.448 | −1.078 |
| NH125 | −2.312 | −2.391 | −1.465 | −1.537 |
| NH125 | −2.421 | −1.592 | −1.139 | −1.214 |
| Nilotinib (AMN-107) | 1.520 | 1.056 | 1.974 | 1.672 |
| Nilotinib (AMN-107) | 1.896 | 1.237 | 1.234 | 2.118 |
| Nilotinib (AMN-107) | 0.210 | 0.877 | 0.694 | 0.186 |
| Nilotinib (AMN-107) | −1.271 | −0.031 | −0.907 | −1.146 |
| Nilvadipine | 0.571 | 0.590 | 0.576 | 0.358 |
| Nilvadipine | 0.085 | 0.302 | 0.024 | 0.074 |
| Nilvadipine | 0.460 | 0.500 | 0.216 | 0.208 |
| Nilvadipine | −0.053 | 0.402 | 0.072 | −0.033 |
| NLG919 | −0.014 | −0.327 | −0.534 | −0.015 |
| NLG919 | −0.551 | −0.544 | −0.802 | −0.273 |
| NLG919 | 0.324 | −0.456 | −0.593 | 0.157 |
| NLG919 | −0.281 | −0.699 | −0.151 | −0.311 |
| NMS-873 | 0.447 | 0.480 | 0.321 | 0.430 |
| NMS-873 | −1.475 | −0.191 | −1.020 | −1.109 |
| NMS-873 | −2.218 | −1.328 | −1.284 | −1.257 |
| NMS-873 | −2.425 | −1.863 | −1.356 | −1.427 |
| NPS-2143 | 0.948 | 0.350 | 1.165 | 0.797 |
| NPS-2143 | −0.401 | −0.326 | −0.394 | −0.424 |
| NPS-2143 | −1.039 | −0.478 | −0.450 | −0.702 |
| NPS-2143 | −0.752 | −0.245 | −0.636 | −0.648 |
| NSC 23766 | 0.516 | 0.152 | 0.735 | 0.514 |
| NSC 23766 | 0.584 | 0.414 | 0.266 | 0.429 |
| NSC 23766 | 0.202 | 0.188 | 0.300 | 0.138 |
| NSC 23766 | 0.197 | 0.447 | 0.064 | 0.120 |
| NSC 319726 | 0.386 | 0.453 | 0.777 | 0.335 |
| NSC 319726 | −0.227 | −0.340 | −0.299 | −0.053 |
| NSC 319726 | −1.601 | −0.475 | −0.721 | −0.851 |
| NSC 319726 | −2.117 | −6.061 | −1.478 | −1.991 |
| NSC 405020 | 0.092 | −0.216 | 0.084 | 0.102 |
| NSC 405020 | 0.598 | −0.196 | −0.277 | 0.270 |
| NSC 405020 | 0.010 | −0.189 | 0.189 | 0.011 |
| NSC 405020 | −0.394 | −0.180 | −0.567 | −0.444 |
| NSC697923 | 0.234 | −0.106 | 0.411 | 0.127 |
| NSC697923 | 0.699 | −0.049 | 0.392 | 0.405 |
| NSC697923 | −0.993 | −0.379 | −0.709 | −1.093 |
| NSC697923 | −1.744 | −0.773 | −0.736 | −0.829 |
| NU7026 | 0.135 | 0.339 | 0.158 | 0.100 |
| NU7026 | 0.218 | 0.331 | 0.132 | 0.139 |
| NU7026 | −0.364 | 0.355 | −0.335 | −0.228 |
| NU7026 | −1.282 | −0.221 | −1.611 | −1.471 |
| NVP-ADW742 | 1.586 | 1.405 | 0.943 | 0.817 |
| NVP-ADW742 | 1.716 | 1.665 | 0.493 | 0.574 |
| NVP-ADW742 | −1.073 | 0.905 | −0.741 | −0.761 |
| NVP-ADW742 | −1.355 | −0.721 | −0.923 | −0.858 |
| OC000459 | 0.585 | 0.242 | 0.431 | 0.540 |
| OC000459 | 1.286 | −0.177 | 0.496 | 0.855 |
| OC000459 | 0.842 | 0.243 | 0.583 | 0.913 |
| OC000459 | 0.331 | 0.289 | 0.283 | 0.327 |
| Odanacatib (MK-0822) | 0.591 | 0.304 | 0.340 | 0.439 |
| Odanacatib (MK-0822) | 1.100 | 0.439 | 0.446 | 0.425 |
| Odanacatib (MK-0822) | 0.230 | −0.017 | 0.147 | 0.166 |
| Odanacatib (MK-0822) | −0.478 | 0.011 | −0.222 | −0.481 |
| OG-L002 | 0.148 | 0.242 | −0.001 | 0.157 |
| OG-L002 | 0.165 | 0.343 | −0.080 | 0.166 |
| OG-L002 | −0.005 | 0.469 | 0.206 | −0.006 |
| OG-L002 | −0.540 | 0.323 | −0.759 | −0.540 |
| Oligomycin A | −1.574 | −1.491 | −1.525 | −1.163 |
| Oligomycin A | −1.772 | −1.031 | −1.748 | −1.312 |
| Oligomycin A | −1.739 | −1.273 | −1.858 | −1.155 |
| Oligomycin A | −1.865 | −1.361 | −1.691 | −1.321 |
| Org 27569 | 1.124 | 0.621 | 0.660 | 0.680 |
| Org 27569 | 0.210 | 0.255 | 0.513 | 0.159 |
| Org 27569 | 0.380 | 1.026 | 0.681 | 0.288 |
| Org 27569 | 0.008 | 0.913 | 0.268 | 0.005 |
| OSI-420 | 0.380 | 0.190 | 0.581 | 0.314 |
| OSI-420 | −1.429 | −0.190 | −2.220 | −1.518 |
| OSI-420 | −2.462 | −2.365 | −2.158 | −1.850 |
| OSI-420 | −2.418 | −2.151 | −2.152 | −2.126 |
| OSI-906 (Linsitinib) | −0.169 | −1.173 | 0.076 | −0.125 |
| OSI-906 (Linsitinib) | −0.768 | −0.401 | −0.808 | −0.728 |
| OSI-906 (Linsitinib) | −1.862 | −0.793 | −1.204 | −1.236 |
| OSI-906 (Linsitinib) | −2.122 | −1.164 | −1.670 | −1.308 |
| OSU-03012 (AR-12) | 0.815 | 0.628 | 0.817 | 0.915 |
| OSU-03012 (AR-12) | 0.728 | 0.217 | 0.305 | 0.499 |
| OSU-03012 (AR-12) | 0.390 | 0.747 | 0.487 | 0.442 |
| OSU-03012 (AR-12) | −0.829 | 0.156 | −0.459 | −0.699 |
| OTX015 | −1.806 | −0.114 | −1.144 | −1.171 |
| OTX015 | −2.265 | −0.656 | −1.269 | −1.227 |
| OTX015 | −2.264 | −2.370 | −1.177 | −1.236 |
| OTX015 | −2.342 | −2.112 | −1.656 | −1.444 |
| Ouabain | 0.262 | 0.028 | −0.026 | 0.298 |
| Ouabain | 0.223 | 0.424 | 0.304 | 0.256 |
| Ouabain | 0.017 | 0.214 | 0.107 | 0.018 |
| Ouabain | 0.321 | −0.360 | 0.400 | 0.243 |
| Oxcarbazepine | 0.275 | −0.074 | 0.042 | 0.312 |
| Oxcarbazepine | 0.993 | −0.370 | −0.001 | 0.432 |
| Oxcarbazepine | 0.271 | −0.285 | 0.318 | 0.317 |
| Oxcarbazepine | −0.371 | −0.483 | −0.399 | −0.304 |
| Oxymetazoline HCl | 0.924 | 0.511 | 0.595 | 0.937 |
| Oxymetazoline HCl | 0.752 | 0.397 | 0.134 | 0.657 |
| Oxymetazoline HCl | −0.079 | −0.020 | 0.198 | −0.093 |
| Oxymetazoline HCl | −0.299 | 0.401 | 0.057 | −0.331 |
| Ozagrel | 0.941 | 0.171 | 0.491 | 0.447 |
| Ozagrel | 0.623 | 0.625 | 0.408 | 0.522 |
| Ozagrel | 0.670 | −0.218 | 0.609 | 0.605 |
| Ozagrel | 0.069 | 0.396 | −0.080 | 0.081 |
| Ozagrel HCl | 0.770 | 0.101 | 0.647 | 0.519 |
| Ozagrel HCl | 0.473 | 0.324 | 0.000 | 0.492 |
| Ozagrel HCl | 0.870 | 0.445 | 0.343 | 0.769 |
| Ozagrel HCl | 0.392 | 0.552 | −0.047 | 0.343 |
| P22077 | −1.176 | −0.537 | 0.171 | −0.596 |
| P22077 | 0.421 | 0.133 | −0.047 | 0.402 |
| P22077 | −0.045 | −0.475 | 0.194 | −0.041 |
| P22077 | −0.408 | −0.418 | −0.423 | −0.262 |
| PAC-1 | 0.827 | 0.295 | 1.045 | 0.672 |
| PAC-1 | 0.384 | 0.407 | 0.465 | 0.381 |
| PAC-1 | −0.041 | −0.864 | 0.703 | −0.035 |
| PAC-1 | 0.597 | 0.005 | 0.690 | 0.623 |
| Pacritinib (SB1518) | 0.429 | −0.016 | 0.716 | 0.353 |
| Pacritinib (SB1518) | −0.208 | 0.250 | −0.225 | −0.179 |
| Pacritinib (SB1518) | −1.964 | −0.660 | −1.158 | −1.473 |
| Pacritinib (SB1518) | −2.711 | −1.726 | −1.631 | −1.348 |
| Palbociclib (PD-0332991) HCl | 0.027 | 0.544 | −0.040 | 0.025 |
| Palbociclib (PD-0332991) HCl | −0.271 | −0.521 | −0.259 | −0.304 |
| Palbociclib (PD-0332991) HCl | −0.787 | −0.108 | −0.422 | −0.618 |
| Palbociclib (PD-0332991) HCl | −1.316 | −1.756 | −1.070 | −1.007 |
| Pancuronium dibromide | 1.380 | 0.706 | 0.900 | 0.873 |
| Pancuronium dibromide | 0.605 | 0.819 | 0.349 | 0.396 |
| Pancuronium dibromide | 0.638 | −0.032 | 0.967 | 0.747 |
| Pancuronium dibromide | 0.029 | 0.167 | 0.181 | 0.020 |
| Panobinostat (LBH589) | −0.223 | 0.855 | 0.013 | −0.153 |
| Panobinostat (LBH589) | −0.273 | 0.646 | −0.248 | −0.281 |
| Panobinostat (LBH589) | −1.845 | −1.460 | −1.224 | −1.586 |
| Panobinostat (LBH589) | −2.071 | −1.149 | −1.537 | −1.734 |
| PD0325901 | −1.372 | −0.549 | −1.135 | −1.292 |
| PD0325901 | −1.418 | −1.981 | −1.202 | −1.058 |
| PD0325901 | −1.466 | −0.944 | −1.003 | −1.236 |
| PD0325901 | −1.740 | −0.872 | −1.599 | −1.457 |
| PD128907 HCl | 0.752 | 1.178 | 0.756 | 0.814 |
| PD128907 HCl | 1.745 | 1.387 | 1.288 | 1.578 |
| PD128907 HCl | 0.917 | 0.908 | 0.907 | 0.989 |
| PD128907 HCl | 0.513 | 0.846 | 1.054 | 0.611 |
| PD184352 (CI-1040) | −0.126 | 0.608 | −0.194 | −0.147 |
| PD184352 (CI-1040) | −0.302 | 0.389 | −0.506 | −0.294 |
| PD184352 (CI-1040) | −1.152 | −0.433 | −1.193 | −1.194 |
| PD184352 (CI-1040) | −1.546 | −1.043 | −0.867 | −1.116 |
| PF-04217903 | 0.714 | 0.367 | 0.830 | 0.776 |
| PF-04217903 | 1.716 | 0.587 | 0.694 | 1.105 |
| PF-04217903 | 0.318 | −0.144 | 1.021 | 0.291 |
| PF-04217903 | 0.819 | 0.287 | 0.675 | 0.585 |
| PF-3845 | 0.173 | −0.111 | 0.588 | 0.121 |
| PF-3845 | −0.397 | 0.147 | −0.799 | −0.445 |
| PF-3845 | −0.208 | −0.366 | −0.153 | −0.211 |
| PF-3845 | −1.262 | −0.929 | −1.104 | −1.163 |
| PF-4708671 | 0.882 | 0.966 | 0.629 | 0.995 |
| PF-4708671 | 1.115 | 0.854 | 0.617 | 0.697 |
| PF-4708671 | 1.352 | 1.118 | 1.852 | 1.440 |
| PF-4708671 | −0.156 | −0.836 | 0.200 | −0.115 |
| PF-5274857 | 1.209 | −0.235 | 0.449 | 0.602 |
| PF-5274857 | −0.220 | −0.138 | −0.299 | −0.259 |
| PF-5274857 | 0.270 | 0.178 | 0.093 | 0.298 |
| PF-5274857 | 0.494 | −0.181 | 0.661 | 0.519 |
| PF-562271 | −0.568 | −0.397 | −0.676 | −0.623 |
| PF-562271 | −1.381 | −0.737 | −1.005 | −1.183 |
| PF-562271 | −2.145 | −2.039 | −1.117 | −1.404 |
| PF-562271 | −2.677 | −2.208 | −1.691 | −1.639 |
| PF-573228 | 0.739 | 0.253 | 0.100 | 0.669 |
| PF-573228 | 0.013 | −0.158 | −0.415 | 0.015 |
| PF-573228 | −1.128 | −1.295 | −0.178 | −0.542 |
| PF-573228 | −1.970 | −1.519 | −1.197 | −1.624 |
| PFI-1 (PF-6405761) | 0.397 | 0.422 | 0.510 | 0.364 |
| PFI-1 (PF-6405761) | 0.670 | 0.890 | 0.300 | 0.335 |
| PFI-1 (PF-6405761) | −0.759 | 0.678 | −0.805 | −0.701 |
| PFI-1 (PF-6405761) | −1.254 | −0.649 | −0.533 | −0.885 |
| PHA-665752 | 1.054 | 1.262 | 1.667 | 1.234 |
| PHA-665752 | 2.410 | 2.078 | 1.474 | 1.350 |
| PHA-665752 | 0.927 | 1.851 | 1.150 | 0.747 |
| PHA-665752 | −1.066 | 0.048 | −0.999 | −1.119 |
| PHA-793887 | 0.300 | 0.394 | 0.191 | 0.164 |
| PHA-793887 | 0.644 | 0.457 | 0.097 | 0.735 |
| PHA-793887 | −0.521 | −0.420 | −1.246 | −0.546 |
| PHA-793887 | −1.885 | −1.675 | −1.989 | −1.448 |
| Piceatannol | 0.609 | 0.688 | 0.477 | 0.325 |
| Piceatannol | 0.289 | −0.265 | 0.303 | 0.301 |
| Piceatannol | 0.102 | 0.133 | 0.124 | 0.051 |
| Piceatannol | −0.673 | −0.125 | −0.119 | −0.429 |
| Pifithrin-CE° | 0.494 | 0.645 | 0.571 | 0.359 |
| Pifithrin-CE° | 0.583 | 0.432 | 0.237 | 0.370 |
| Pifithrin-CE° | 1.176 | 0.445 | 1.398 | 1.335 |
| Pifithrin-CE° | 0.244 | 0.285 | 0.014 | 0.193 |
| Pimobendan | 0.837 | −0.053 | 0.311 | 0.518 |
| Pimobendan | 0.328 | 0.591 | 0.493 | 0.370 |
| Pimobendan | 1.256 | 0.508 | 0.650 | 0.690 |
| Pimobendan | −0.869 | 0.450 | −0.592 | −0.868 |
| PluriSIn #1 (NSC 14613) | 0.369 | −0.535 | 0.344 | 0.373 |
| PluriSIn #1 (NSC 14613) | −0.045 | −0.350 | 0.020 | −0.043 |
| PluriSIn #1 (NSC 14613) | −0.857 | −0.988 | −0.808 | −0.560 |
| PluriSIn #1 (NSC 14613) | −2.405 | −3.099 | −1.807 | −1.678 |
| PNU-120596 | 0.376 | −0.183 | 0.745 | 0.306 |
| PNU-120596 | 0.526 | 0.221 | −0.093 | 0.377 |
| PNU-120596 | 0.418 | 0.364 | 0.511 | 0.406 |
| PNU-120596 | −0.318 | 0.398 | −0.524 | −0.359 |
| Pomalidomide | −0.183 | 0.211 | −0.020 | −0.186 |
| Pomalidomide | −0.531 | 0.782 | −0.011 | −0.425 |
| Pomalidomide | −0.745 | −0.398 | −0.372 | −0.825 |
| Pomalidomide | −1.324 | −0.427 | −1.211 | −1.228 |
| PP2 | 0.377 | −0.439 | 0.262 | 0.322 |
| PP2 | 0.941 | 0.344 | 0.116 | 0.593 |
| PP2 | 0.733 | −0.356 | 1.065 | 0.569 |
| PP2 | 0.173 | −0.492 | −0.178 | 0.158 |
| PR-619 | 0.786 | 0.199 | 0.832 | 0.615 |
| PR-619 | 0.302 | 0.265 | 0.276 | 0.181 |
| PR-619 | 0.522 | 0.050 | 0.488 | 0.533 |
| PR-619 | −1.245 | −0.575 | −1.207 | −1.110 |
| Pralatrexate | −1.184 | −0.768 | −1.023 | −0.954 |
| Pralatrexate | −1.139 | −1.512 | −1.350 | −1.321 |
| Pralatrexate | −1.117 | −2.057 | −0.901 | −1.219 |
| Pralatrexate | −1.388 | −2.975 | −1.104 | −1.185 |
| Pramipexole | 0.323 | 0.318 | 0.402 | 0.290 |
| Pramipexole | 0.797 | 0.532 | 0.233 | 0.476 |
| Pramipexole | 0.267 | 0.076 | 0.249 | 0.240 |
| Pramipexole | 0.460 | 0.640 | 0.039 | 0.445 |
| Propranolol HCl | 1.116 | 0.227 | 0.414 | 0.758 |
| Propranolol HCl | 1.319 | 0.186 | 0.320 | 0.841 |
| Propranolol HCl | 0.591 | 0.308 | 1.047 | 0.641 |
| Propranolol HCl | 0.260 | 0.629 | 0.175 | 0.260 |
| PRT062607 (P505-15, BIIB057) HCl | −0.216 | −0.318 | 0.040 | −0.234 |
| PRT062607 (P505-15, BIIB057) HCl | 0.790 | 0.261 | 0.229 | 0.592 |
| PRT062607 (P505-15, BIIB057) HCl | −0.797 | −0.165 | −0.194 | −0.485 |
| PRT062607 (P505-15, BIIB057) HCl | −1.929 | −0.689 | −1.089 | −0.983 |
| PTC-209 | 0.047 | −0.141 | 0.273 | 0.040 |
| PTC-209 | 0.293 | −0.407 | −0.428 | 0.128 |
| PTC-209 | −1.871 | −2.629 | −0.950 | −1.020 |
| PTC-209 | −1.938 | −3.695 | −0.964 | −1.034 |
| PYR-41 | 1.247 | 0.688 | 1.040 | 0.746 |
| PYR-41 | 0.494 | 0.651 | 0.436 | 0.455 |
| PYR-41 | 0.314 | 0.340 | 0.317 | 0.191 |
| PYR-41 | 0.832 | 0.189 | 0.777 | 0.588 |
| Pyrimethamine | 0.271 | 0.023 | −0.044 | 0.308 |
| Pyrimethamine | −0.169 | −0.880 | −0.152 | −0.145 |
| Pyrimethamine | −1.047 | −1.536 | −1.299 | −1.012 |
| Pyrimethamine | −1.822 | −1.498 | −0.819 | −1.472 |
| Quizartinib (AC220) | 0.996 | 1.080 | 0.899 | 0.882 |
| Quizartinib (AC220) | 0.538 | 0.299 | 0.409 | 0.389 |
| Quizartinib (AC220) | −0.499 | −1.240 | −0.542 | −0.396 |
| Quizartinib (AC220) | −1.292 | −0.944 | −0.847 | −0.753 |
| Raltegravir (MK-0518) | 0.889 | 0.206 | 0.890 | 1.004 |
| Raltegravir (MK-0518) | 0.236 | 0.284 | 0.257 | 0.183 |
| Raltegravir (MK-0518) | 0.790 | 0.428 | 0.242 | 0.638 |
| Raltegravir (MK-0518) | 1.180 | 0.381 | 0.285 | 1.317 |
| Ramelteon | 0.196 | −0.086 | 0.445 | 0.156 |
| Ramelteon | 0.667 | 0.342 | 0.468 | 0.356 |
| Ramelteon | −0.059 | −0.531 | −0.125 | −0.064 |
| Ramelteon | 0.006 | 0.467 | 0.049 | 0.005 |
| Ranitidine | −0.131 | −0.062 | −0.143 | −0.085 |
| Ranitidine | 0.460 | 0.320 | 0.090 | 0.432 |
| Ranitidine | −0.256 | −0.242 | −0.380 | −0.235 |
| Ranitidine | −0.276 | −0.526 | −0.352 | −0.237 |
| Rasagiline Mesylate | 0.904 | 0.317 | 0.345 | 0.512 |
| Rasagiline Mesylate | 0.497 | 0.199 | 0.108 | 0.555 |
| Rasagiline Mesylate | 0.093 | 0.149 | 0.355 | 0.090 |
| Rasagiline Mesylate | 0.165 | 0.236 | −0.183 | 0.102 |
| Rebamipide | −0.089 | −0.724 | 0.290 | −0.074 |
| Rebamipide | 0.094 | −0.727 | −0.809 | 0.034 |
| Rebamipide | −0.686 | −0.579 | −0.688 | −0.440 |
| Rebamipide | −0.410 | −0.533 | −0.891 | −0.428 |
| RepSox | 0.956 | −0.147 | 1.290 | 0.680 |
| RepSox | 2.267 | −0.287 | 0.636 | 1.815 |
| RepSox | 1.105 | −0.676 | 0.665 | 0.933 |
| RepSox | −0.147 | −2.004 | −0.544 | −0.106 |
| Ridaforolimus (Deforolimus, MK-8669) | 0.320 | 0.897 | 0.049 | 0.370 |
| Ridaforolimus (Deforolimus, MK-8669) | 0.449 | 0.181 | 1.067 | 0.508 |
| Ridaforolimus (Deforolimus, MK-8669) | 0.125 | 0.435 | 0.534 | 0.121 |
| Ridaforolimus (Deforolimus, MK-8669) | 0.249 | 0.259 | 0.273 | 0.273 |
| Rigosertib (ON-01910) | −1.996 | −2.151 | −1.459 | −1.633 |
| Rigosertib (ON-01910) | −2.295 | −3.311 | −1.633 | −1.950 |
| Rigosertib (ON-01910) | −2.196 | −2.491 | −1.787 | −1.809 |
| Rigosertib (ON-01910) | −2.137 | −2.325 | −1.702 | −1.878 |
| Rimonabant | 0.574 | 0.487 | 0.836 | 0.485 |
| Rimonabant | −0.018 | 0.581 | −0.501 | −0.016 |
| Rimonabant | −0.340 | 0.481 | −0.236 | −0.189 |
| Rimonabant | −0.631 | 0.167 | −0.343 | −0.567 |
| Rivaroxaban | 0.877 | 0.069 | 0.836 | 0.847 |
| Rivaroxaban | −0.172 | 0.544 | −0.228 | −0.131 |
| Rivaroxaban | 0.406 | 0.113 | 1.166 | 0.421 |
| Rivaroxaban | −0.313 | 0.142 | −0.392 | −0.337 |
| Rizatriptan Benzoate | 1.158 | 0.455 | 0.335 | 0.676 |
| Rizatriptan Benzoate | 0.122 | 0.299 | 0.431 | 0.120 |
| Rizatriptan Benzoate | −0.055 | 0.386 | 0.267 | −0.057 |
| Rizatriptan Benzoate | 0.680 | −0.177 | −0.003 | 0.440 |
| RKI-1447 | −0.361 | −1.130 | −0.047 | −0.320 |
| RKI-1447 | −0.416 | −0.402 | −0.762 | −0.356 |
| RKI-1447 | −0.111 | −0.720 | −0.079 | −0.120 |
| RKI-1447 | −1.919 | −1.096 | −0.908 | −1.093 |
| Rolipram | 1.545 | 0.696 | 1.497 | 1.167 |
| Rolipram | 1.009 | 0.362 | 0.622 | 0.794 |
| Rolipram | 2.178 | 1.191 | 0.933 | 1.021 |
| Rolipram | 1.442 | 0.987 | 0.715 | 1.372 |
| Rotundine | 0.979 | 1.221 | 0.770 | 0.716 |
| Rotundine | 0.315 | 1.197 | 0.289 | 0.343 |
| Rotundine | 0.515 | 0.656 | 0.641 | 0.488 |
| Rotundine | 0.615 | 0.621 | 0.842 | 0.444 |
| Roxatidine Acetate HCl | 0.228 | −0.057 | 0.526 | 0.124 |
| Roxatidine Acetate HCl | 0.640 | 0.054 | −0.235 | 0.276 |
| Roxatidine Acetate HCl | −0.340 | −0.319 | −0.011 | −0.330 |
| Roxatidine Acetate HCl | −0.670 | −0.672 | −0.573 | −0.675 |
| Ruxolitinib (INCB018424) | 0.973 | 1.303 | 1.179 | 0.882 |
| Ruxolitinib (INCB018424) | 0.433 | 1.194 | 0.472 | 0.434 |
| Ruxolitinib (INCB018424) | 0.677 | 1.316 | 0.795 | 0.634 |
| Ruxolitinib (INCB018424) | 2.047 | 1.606 | 1.763 | 1.289 |
| S3I-201 | 0.216 | 0.962 | 0.509 | 0.216 |
| S3I-201 | 1.016 | 0.540 | 0.576 | 0.685 |
| S3I-201 | −0.023 | 0.186 | 0.248 | −0.020 |
| S3I-201 | −0.336 | −0.302 | −0.134 | −0.358 |
| Safinamide Mesylate | 1.950 | 0.574 | 1.381 | 1.182 |
| Safinamide Mesylate | 1.233 | 0.639 | 0.924 | 1.469 |
| Safinamide Mesylate | 1.523 | 0.668 | 1.152 | 1.483 |
| Safinamide Mesylate | 0.922 | 1.173 | 0.484 | 0.917 |
| Sal003 | 0.597 | 0.529 | 0.762 | 0.527 |
| Sal003 | 0.432 | 0.514 | 0.655 | 0.497 |
| Sal003 | 0.261 | 0.432 | 0.586 | 0.242 |
| Sal003 | −2.638 | −1.767 | −1.393 | −1.258 |
| SANT-1 | 0.723 | 0.807 | 0.328 | 0.502 |
| SANT-1 | 0.153 | 0.098 | 0.001 | 0.105 |
| SANT-1 | 0.650 | 0.311 | 0.779 | 0.419 |
| SANT-1 | −0.773 | −0.138 | −0.437 | −0.796 |
| SAR131675 | 0.957 | 0.488 | 0.452 | 0.917 |
| SAR131675 | 0.623 | −0.177 | 0.583 | 0.451 |
| SAR131675 | 0.171 | 0.158 | 0.158 | 0.107 |
| SAR131675 | 0.000 | 0.133 | −0.698 | 0.000 |
| SB203580 | 0.960 | 0.541 | 0.462 | 0.714 |
| SB203580 | 0.637 | 0.335 | 0.794 | 0.589 |
| SB203580 | 0.498 | 0.572 | 0.523 | 0.540 |
| SB203580 | 0.447 | 0.297 | 0.185 | 0.316 |
| SB408124 | 1.280 | 1.045 | 1.092 | 0.650 |
| SB408124 | 1.250 | 0.972 | 1.037 | 0.729 |
| SB408124 | 0.531 | 0.919 | 0.524 | 0.377 |
| SB408124 | −0.959 | 0.195 | −0.354 | −0.414 |
| SB415286 | 1.031 | 0.529 | 0.858 | 0.763 |
| SB415286 | −0.051 | −0.014 | 0.109 | −0.043 |
| SB415286 | −0.345 | −0.330 | 0.310 | −0.266 |
| SB415286 | −0.270 | 0.257 | −0.135 | −0.270 |
| SB431542 | 0.873 | 1.022 | 0.940 | 0.890 |
| SB431542 | 2.001 | 1.636 | 1.050 | 0.883 |
| SB431542 | 1.440 | 1.149 | 1.223 | 1.235 |
| SB431542 | 0.817 | 0.513 | 0.230 | 0.604 |
| SB705498 | 0.607 | 0.335 | 0.402 | 0.404 |
| SB705498 | 0.147 | −0.143 | −0.424 | 0.081 |
| SB705498 | −0.197 | −0.013 | −0.007 | −0.178 |
| SB705498 | −0.151 | −0.119 | −0.185 | −0.180 |
| SB742457 | −0.357 | −0.710 | 0.069 | −0.254 |
| SB742457 | −0.189 | −0.418 | −0.421 | −0.176 |
| SB742457 | −0.019 | −0.455 | 0.365 | −0.022 |
| SB742457 | −0.041 | −0.763 | 0.111 | −0.037 |
| SB743921 | −2.078 | −1.029 | −1.684 | −1.744 |
| SB743921 | −2.125 | −2.700 | −1.654 | −1.649 |
| SB743921 | −2.201 | −1.425 | −1.871 | −1.953 |
| SB743921 | −2.262 | −1.987 | −1.505 | −2.080 |
| SC-514 | 0.415 | 0.152 | 0.571 | 0.318 |
| SC-514 | −0.391 | 0.037 | −0.055 | −0.442 |
| SC-514 | 0.049 | 0.418 | 0.404 | 0.045 |
| SC-514 | 0.161 | 0.613 | 0.563 | 0.181 |
| SC144 | 0.635 | 0.525 | 0.629 | 0.670 |
| SC144 | 0.094 | 0.426 | −0.115 | 0.098 |
| SC144 | −0.846 | −0.595 | −0.147 | −0.540 |
| SC144 | −1.362 | −1.047 | −0.621 | −0.593 |
| Selumetinib (AZD6244) | 0.024 | 0.208 | 0.132 | 0.018 |
| Selumetinib (AZD6244) | −0.304 | −0.502 | −0.192 | −0.271 |
| Selumetinib (AZD6244) | −1.132 | −0.534 | −1.006 | −1.225 |
| Selumetinib (AZD6244) | −1.367 | −1.212 | −1.307 | −1.081 |
| Semagacestat (LY450139) | −0.088 | 0.435 | 0.161 | −0.092 |
| Semagacestat (LY450139) | −0.040 | −0.653 | −0.157 | −0.021 |
| Semagacestat (LY450139) | −0.524 | −0.362 | −0.702 | −0.589 |
| Semagacestat (LY450139) | −1.045 | −1.051 | −0.925 | −0.990 |
| Sertraline HCl | −0.310 | −0.203 | −0.168 | −0.185 |
| Sertraline HCl | −0.493 | 0.235 | −0.750 | −0.554 |
| Sertraline HCl | 0.148 | 0.233 | 0.409 | 0.158 |
| Sertraline HCl | −0.135 | 0.098 | −0.052 | −0.147 |
| SGC0946 | 0.937 | 0.185 | 0.786 | 0.770 |
| SGC0946 | −0.635 | −0.340 | −0.145 | −0.274 |
| SGC0946 | 1.422 | −0.180 | 1.576 | 0.896 |
| SGC0946 | 0.421 | −0.312 | 0.610 | 0.468 |
| SGI-1027 | 0.386 | 0.429 | 0.545 | 0.281 |
| SGI-1027 | 0.936 | 0.724 | 0.556 | 0.607 |
| SGI-1027 | 0.608 | 0.337 | 0.824 | 0.411 |
| SGI-1027 | 0.114 | 0.108 | −0.044 | 0.112 |
| SGI-1776free base | 1.031 | 0.492 | 0.963 | 1.190 |
| SGI-1776free base | 0.569 | 0.825 | 0.270 | 0.494 |
| SGI-1776free base | 0.074 | 0.709 | 0.096 | 0.075 |
| SGI-1776free base | −2.052 | −1.338 | −2.066 | −1.497 |
| Sirtinol | 0.500 | −0.510 | 0.835 | 0.550 |
| Sirtinol | 0.570 | 0.034 | 0.002 | 0.468 |
| Sirtinol | 0.183 | −0.410 | 0.508 | 0.192 |
| Sirtinol | 0.168 | −0.270 | 0.768 | 0.195 |
| Sitaxentan sodium | −0.112 | 0.323 | 0.476 | −0.122 |
| Sitaxentan sodium | −0.183 | −0.383 | −0.427 | −0.147 |
| Sitaxentan sodium | 0.881 | −0.221 | 0.288 | 0.428 |
| Sitaxentan sodium | −0.304 | −0.231 | −0.332 | −0.242 |
| SKI II | 0.581 | 1.101 | 0.511 | 0.590 |
| SKI II | 0.797 | 0.728 | 0.680 | 0.894 |
| SKI II | 0.392 | 0.624 | 0.746 | 0.437 |
| SKI II | −1.235 | −0.709 | −0.720 | −0.685 |
| SMI-4a | 0.232 | −0.263 | 0.083 | 0.092 |
| SMI-4a | −0.405 | −0.399 | −0.430 | −0.238 |
| SMI-4a | −0.271 | −0.426 | 0.176 | −0.172 |
| SMI-4a | −0.867 | −0.769 | −0.444 | −0.880 |
| SN-38 | −2.708 | −2.221 | −1.387 | −1.581 |
| SN-38 | −2.601 | −1.347 | −1.608 | −1.524 |
| SN-38 | −2.527 | −1.626 | −1.434 | −1.306 |
| SN-38 | −2.615 | −3.000 | −1.133 | −1.277 |
| SNS-032 (BMS-387032) | 0.455 | −0.268 | 0.304 | 0.468 |
| SNS-032 (BMS-387032) | −1.333 | −0.205 | −1.294 | −1.462 |
| SNS-032 (BMS-387032) | −1.949 | −1.418 | −1.741 | −1.293 |
| SNS-032 (BMS-387032) | −2.332 | −2.459 | −1.544 | −1.975 |
| SNS-314 Mesylate | −0.745 | −0.596 | −0.541 | −0.620 |
| SNS-314 Mesylate | −0.663 | −1.607 | −0.472 | −0.691 |
| SNS-314 Mesylate | −0.863 | −1.563 | −1.097 | −0.624 |
| SNS-314 Mesylate | −1.282 | −1.573 | −1.055 | −0.947 |
| Sodium 4-Aminosalicylate | 1.049 | 0.325 | 0.829 | 0.838 |
| Sodium 4-Aminosalicylate | 0.323 | 0.524 | 0.334 | 0.385 |
| Sodium 4-Aminosalicylate | 0.638 | 0.130 | 0.728 | 0.405 |
| Sodium 4-Aminosalicylate | 0.924 | 0.850 | 0.749 | 0.875 |
| Sorafenib | 0.805 | 0.089 | 0.351 | 0.537 |
| Sorafenib | 1.062 | 0.557 | 0.569 | 0.760 |
| Sorafenib | 0.094 | −0.275 | 0.303 | 0.089 |
| Sorafenib | −0.402 | 0.424 | −0.197 | −0.442 |
| Sotrastaurin | 0.630 | 0.027 | 0.605 | 0.450 |
| Sotrastaurin | −0.275 | −0.376 | −0.642 | −0.308 |
| Sotrastaurin | −0.551 | −0.677 | 0.058 | −0.587 |
| Sotrastaurin | −1.060 | −0.424 | −0.975 | −0.936 |
| SP600125 | 1.213 | 0.583 | 1.259 | 1.186 |
| SP600125 | 0.597 | 0.314 | 0.220 | 0.460 |
| SP600125 | 0.580 | 0.257 | 0.571 | 0.505 |
| SP600125 | 0.541 | −0.065 | 0.190 | 0.458 |
| SRPIN340 | 0.727 | 0.758 | 0.697 | 0.512 |
| SRPIN340 | 0.636 | 1.131 | 0.337 | 0.739 |
| SRPIN340 | 0.646 | 0.957 | 0.852 | 0.481 |
| SRPIN340 | 0.501 | 0.809 | 0.146 | 0.414 |
| SRT1720 | 1.231 | 1.380 | 1.510 | 1.250 |
| SRT1720 | 0.642 | 1.128 | 0.649 | 0.751 |
| SRT1720 | 0.327 | 1.177 | 0.610 | 0.318 |
| SRT1720 | −1.249 | −1.363 | −0.702 | −0.985 |
| SSR128129E | 0.555 | 0.321 | 0.613 | 0.517 |
| SSR128129E | 0.789 | 0.609 | 0.360 | 0.830 |
| SSR128129E | 0.151 | 0.438 | 0.306 | 0.141 |
| SSR128129E | −0.612 | −0.087 | −1.002 | −0.694 |
| Stattic | 0.973 | −0.157 | 1.288 | 0.910 |
| Stattic | 0.216 | −0.013 | 0.155 | 0.169 |
| Stattic | 0.532 | 0.107 | 0.822 | 0.532 |
| Stattic | −0.121 | −0.058 | −0.171 | −0.120 |
| Stavudine (d4T) | 0.867 | 0.331 | 0.589 | 0.890 |
| Stavudine (d4T) | 0.008 | 0.471 | 0.030 | 0.008 |
| Stavudine (d4T) | −0.018 | 0.272 | 0.084 | −0.019 |
| Stavudine (d4T) | −0.773 | −0.191 | −0.861 | −0.707 |
| STF-118804 | −1.660 | −0.171 | −0.797 | −1.002 |
| STF-118804 | −1.374 | −2.409 | −1.119 | −0.861 |
| STF-118804 | −1.753 | −2.411 | −0.788 | −0.973 |
| STF-118804 | −1.937 | −1.154 | −0.762 | −1.019 |
| SU11274 | 0.745 | 0.817 | 0.734 | 0.793 |
| SU11274 | 1.926 | 0.841 | 0.876 | 0.756 |
| SU11274 | 0.051 | 0.172 | 0.053 | 0.057 |
| SU11274 | −1.941 | −0.686 | −1.469 | −1.440 |
| Suvorexant (MK-4305) | 1.202 | 0.700 | 1.213 | 1.035 |
| Suvorexant (MK-4305) | 0.920 | 0.584 | 1.151 | 1.030 |
| Suvorexant (MK-4305) | 0.655 | 0.930 | 0.998 | 0.573 |
| Suvorexant (MK-4305) | 0.846 | 0.625 | 0.438 | 0.657 |
| T0070907 | −0.030 | 0.380 | 0.077 | −0.028 |
| T0070907 | −0.010 | 0.078 | −0.034 | −0.011 |
| T0070907 | 0.526 | 0.183 | 0.394 | 0.526 |
| T0070907 | −2.014 | −0.953 | −1.103 | −1.056 |
| T0901317 | 0.717 | 0.084 | 0.746 | 0.545 |
| T0901317 | 0.496 | 0.344 | 0.427 | 0.450 |
| T0901317 | 0.450 | 0.155 | 0.575 | 0.339 |
| T0901317 | −0.218 | 0.162 | −0.143 | −0.253 |
| Tadalafil | 0.588 | −0.014 | 0.405 | 0.390 |
| Tadalafil | 0.613 | 0.866 | 0.132 | 0.429 |
| Tadalafil | 1.444 | 1.116 | 0.760 | 1.263 |
| Tadalafil | 0.954 | 0.755 | 0.232 | 0.439 |
| TAE226 (NVP-TAE226) | −2.154 | −1.616 | −1.553 | −1.565 |
| TAE226 (NVP-TAE226) | −2.434 | −1.327 | −2.206 | −1.589 |
| TAE226 (NVP-TAE226) | −2.585 | −3.060 | −2.049 | −1.751 |
| TAE226 (NVP-TAE226) | −2.753 | −2.702 | −1.516 | −1.704 |
| TAK-700 (Orteronel) | 0.732 | −0.557 | 0.744 | 0.785 |
| TAK-700 (Orteronel) | 0.580 | −0.066 | 0.520 | 0.479 |
| TAK-700 (Orteronel) | 0.599 | −0.294 | 0.766 | 0.457 |
| TAK-700 (Orteronel) | 0.206 | 0.257 | −0.017 | 0.177 |
| TAK-875 | 1.369 | 0.566 | 0.685 | 1.528 |
| TAK-875 | 0.088 | −0.592 | 0.232 | 0.094 |
| TAK-875 | −0.201 | −0.383 | −0.322 | −0.209 |
| TAK-875 | −0.388 | −0.829 | −0.762 | −0.151 |
| Tandutinib (MLN518) | 0.937 | 1.861 | 0.639 | 0.753 |
| Tandutinib (MLN518) | 1.151 | 1.334 | 0.748 | 1.002 |
| Tandutinib (MLN518) | 0.600 | 1.342 | 0.660 | 0.432 |
| Tandutinib (MLN518) | 0.647 | 1.580 | 0.223 | 0.355 |
| Tariquidar | 0.260 | −0.208 | 0.307 | 0.183 |
| Tariquidar | 0.474 | 0.129 | 0.286 | 0.403 |
| Tariquidar | 0.380 | 0.699 | 0.275 | 0.439 |
| Tariquidar | 0.124 | 0.561 | 0.272 | 0.124 |
| TCID | 0.404 | −0.267 | 0.499 | 0.481 |
| TCID | 0.109 | −0.026 | −0.157 | 0.093 |
| TCID | −0.425 | −0.781 | 0.202 | −0.381 |
| TCID | −0.350 | −0.748 | −0.111 | −0.292 |
| TCS 359 | 0.637 | 0.525 | 0.358 | 0.443 |
| TCS 359 | 0.449 | 0.770 | 0.346 | 0.368 |
| TCS 359 | −0.041 | 0.153 | 0.129 | −0.036 |
| TCS 359 | 0.170 | −0.123 | −0.073 | 0.173 |
| Telmisartan | 0.905 | 0.396 | 1.254 | 0.774 |
| Telmisartan | 0.687 | 0.573 | 0.909 | 0.690 |
| Telmisartan | 0.405 | 0.236 | 0.463 | 0.344 |
| Telmisartan | −0.021 | 0.589 | −0.048 | −0.015 |
| Temsirolimus (CCI-779, NSC 683864) | 0.533 | 1.454 | 0.749 | 0.637 |
| Temsirolimus (CCI-779, NSC 683864) | 0.984 | 1.926 | 0.639 | 1.000 |
| Temsirolimus (CCI-779, NSC 683864) | 0.857 | 1.953 | 1.522 | 0.870 |
| Temsirolimus (CCI-779, NSC 683864) | 0.989 | 0.348 | 0.718 | 0.953 |
| Tenofovir | 0.292 | 0.054 | 0.392 | 0.282 |
| Tenofovir | −0.514 | −0.193 | −0.718 | −0.493 |
| Tenofovir | −1.103 | −0.413 | −0.859 | −1.028 |
| Tenofovir | −1.755 | −0.799 | −1.734 | −1.498 |
| Tenofovir Disoproxil Fumarate | 1.345 | 0.220 | 0.562 | 0.606 |
| Tenofovir Disoproxil Fumarate | 0.558 | −0.005 | −0.479 | 0.257 |
| Tenofovir Disoproxil Fumarate | −0.422 | −0.421 | −0.621 | −0.429 |
| Tenofovir Disoproxil Fumarate | −1.724 | −0.704 | −1.052 | −0.973 |
| Tenovin-6 | 0.179 | 0.158 | 0.622 | 0.180 |
| Tenovin-6 | −0.704 | −0.831 | −0.215 | −0.409 |
| Tenovin-6 | −2.123 | −1.422 | −0.720 | −1.087 |
| Tenovin-6 | −2.226 | −1.836 | −0.835 | −0.966 |
| TG100-115 | 0.469 | −0.532 | 0.750 | 0.525 |
| TG100-115 | 0.208 | 0.179 | −0.250 | 0.243 |
| TG100-115 | −0.120 | −0.034 | 0.029 | −0.125 |
| TG100-115 | −0.644 | −0.416 | −0.160 | −0.763 |
| Thiazovivin | 0.919 | 0.464 | 0.709 | 0.710 |
| Thiazovivin | 0.385 | 0.416 | 0.683 | 0.323 |
| Thiazovivin | 0.412 | 0.692 | 0.173 | 0.286 |
| Thiazovivin | 0.239 | 0.647 | −0.609 | 0.196 |
| Ticagrelor | 0.272 | −0.146 | 0.546 | 0.148 |
| Ticagrelor | 0.003 | −0.055 | −0.155 | 0.001 |
| Ticagrelor | 0.117 | −0.320 | 0.456 | 0.076 |
| Ticagrelor | 0.141 | −0.310 | 0.083 | 0.082 |
| Ticlopidine HCl | 0.337 | −0.428 | 0.376 | 0.395 |
| Ticlopidine HCl | 0.465 | −0.110 | 0.095 | 0.541 |
| Ticlopidine HCl | −0.139 | −0.743 | −0.016 | −0.099 |
| Ticlopidine HCl | 0.494 | −0.020 | −0.269 | 0.340 |
| Tie2 kinase inhibitor | 0.116 | 0.013 | 0.087 | 0.122 |
| Tie2 kinase inhibitor | −0.139 | 0.259 | 0.023 | −0.156 |
| Tie2 kinase inhibitor | 0.155 | −0.680 | 0.465 | 0.173 |
| Tie2 kinase inhibitor | −0.629 | −0.583 | −0.180 | −0.451 |
| Tioxolone | 0.961 | 0.641 | 0.508 | 0.472 |
| Tioxolone | −0.002 | 0.273 | 0.094 | −0.002 |
| Tioxolone | 0.150 | 0.151 | 0.497 | 0.152 |
| Tioxolone | 0.178 | 1.024 | 0.229 | 0.193 |
| Tofacitinib (CP-690550, Tasocitinib) | 0.139 | 0.467 | −0.131 | 0.131 |
| Tofacitinib (CP-690550, Tasocitinib) | 0.067 | 0.764 | −0.102 | 0.052 |
| Tofacitinib (CP-690550, Tasocitinib) | 0.160 | 0.412 | −0.058 | 0.179 |
| Tofacitinib (CP-690550, Tasocitinib) | 0.062 | 0.828 | −0.026 | 0.053 |
| Tolazoline HCl | 1.095 | 0.840 | 1.006 | 1.017 |
| Tolazoline HCl | 0.824 | 0.520 | 0.414 | 0.552 |
| Tolazoline HCl | 0.444 | −0.135 | 0.526 | 0.293 |
| Tolazoline HCl | −0.409 | −0.379 | −0.339 | −0.436 |
| Tolfenamic Acid | 0.763 | 0.507 | 0.255 | 0.844 |
| Tolfenamic Acid | 0.336 | −0.010 | 0.186 | 0.328 |
| Tolfenamic Acid | −0.146 | −0.546 | 0.308 | −0.148 |
| Tolfenamic Acid | −0.411 | −0.294 | −0.304 | −0.401 |
| Tolvaptan | 0.557 | 0.343 | 0.483 | 0.516 |
| Tolvaptan | 0.201 | −0.026 | −0.407 | 0.114 |
| Tolvaptan | −0.313 | −0.263 | −0.167 | −0.352 |
| Tolvaptan | −0.968 | −0.217 | −0.811 | −1.007 |
| Torcetrapib | 2.070 | 0.706 | 0.543 | 2.223 |
| Torcetrapib | 0.289 | 0.154 | −0.488 | 0.129 |
| Torcetrapib | −0.634 | −0.445 | −0.821 | −0.451 |
| Torcetrapib | −0.361 | −0.540 | −0.105 | −0.374 |
| Toremifene Citrate | 0.506 | 0.729 | 0.509 | 0.605 |
| Toremifene Citrate | 1.483 | 0.169 | 0.343 | 0.641 |
| Toremifene Citrate | −0.219 | −0.901 | 0.103 | −0.107 |
| Toremifene Citrate | −1.827 | −1.492 | −1.329 | −1.113 |
| Tosedostat (CHR2797) | −0.177 | −0.311 | −0.013 | −0.103 |
| Tosedostat (CHR2797) | −0.864 | 0.179 | −1.074 | −0.908 |
| Tosedostat (CHR2797) | −0.828 | −0.620 | −0.384 | −0.713 |
| Tosedostat (CHR2797) | −1.783 | −1.278 | −1.101 | −1.362 |
| TPCA-1 | 0.265 | −0.206 | 0.383 | 0.243 |
| TPCA-1 | −0.190 | −0.012 | −0.310 | −0.192 |
| TPCA-1 | −0.347 | −0.017 | −0.592 | −0.409 |
| TPCA-1 | −1.900 | −1.872 | −1.143 | −1.661 |
| Tranylcypromine (2-PCPA) HCl | 0.321 | −0.100 | 0.217 | 0.297 |
| Tranylcypromine (2-PCPA) HCl | −0.242 | 0.229 | −0.508 | −0.209 |
| Tranylcypromine (2-PCPA) HCl | 0.230 | 0.110 | 0.376 | 0.253 |
| Tranylcypromine (2-PCPA) HCl | −0.619 | −0.357 | −0.296 | −0.699 |
| Trelagliptin | 0.130 | 0.475 | 0.063 | 0.129 |
| Trelagliptin | 0.378 | −0.235 | −0.162 | 0.179 |
| Trelagliptin | −0.143 | 0.132 | −0.249 | −0.128 |
| Trelagliptin | −0.705 | 0.371 | −0.227 | −0.345 |
| Triamterene | 0.310 | −0.046 | 0.992 | 0.293 |
| Triamterene | 0.331 | −0.277 | 0.241 | 0.318 |
| Triamterene | −0.419 | −0.251 | −0.357 | −0.353 |
| Triamterene | −1.873 | −1.665 | −1.728 | −1.829 |
| Trichostatin A (TSA) | −0.801 | 0.492 | −0.443 | −0.642 |
| Trichostatin A (TSA) | −1.643 | −2.373 | −1.699 | −1.231 |
| Trichostatin A (TSA) | −1.357 | −1.064 | −1.182 | −1.084 |
| Trichostatin A (TSA) | −1.304 | −0.830 | −1.236 | −1.023 |
| Trimebutine | −0.002 | 0.025 | −0.121 | −0.002 |
| Trimebutine | 0.010 | −0.448 | −0.180 | 0.009 |
| Trimebutine | −1.793 | −3.799 | −1.294 | −1.482 |
| Trimebutine | −2.150 | −0.992 | −1.346 | −1.348 |
| Tropicamide | −0.239 | 0.206 | −0.243 | −0.149 |
| Tropicamide | −0.182 | −0.188 | 0.336 | −0.184 |
| Tropicamide | 0.466 | 0.543 | 0.106 | 0.515 |
| Tropicamide | 0.029 | 0.119 | 0.018 | 0.030 |
| Trospium chloride | 0.767 | 0.222 | 1.702 | 0.737 |
| Trospium chloride | 0.758 | −0.087 | 0.987 | 0.801 |
| Trospium chloride | −0.052 | 0.656 | −0.020 | −0.055 |
| Trospium chloride | 0.541 | 0.512 | 0.991 | 0.648 |
| TWS119 | 1.338 | 0.819 | 0.892 | 1.336 |
| TWS119 | 0.916 | 1.045 | 0.291 | 0.715 |
| TWS119 | 0.284 | 0.257 | 0.221 | 0.257 |
| TWS119 | −1.831 | −2.366 | −1.448 | −1.525 |
| Tyrphostin AG 879 | −0.539 | −0.849 | 0.054 | −0.553 |
| Tyrphostin AG 879 | −0.543 | −0.589 | −0.452 | −0.428 |
| Tyrphostin AG 879 | −0.333 | −0.779 | −0.693 | −0.335 |
| Tyrphostin AG 879 | −0.795 | −0.641 | −0.970 | −0.760 |
| U-104 | 0.718 | −0.202 | 0.525 | 0.508 |
| U-104 | 1.239 | 0.245 | 0.106 | 1.131 |
| U-104 | −0.108 | −0.523 | 0.382 | −0.094 |
| U-104 | −0.149 | −0.066 | −0.056 | −0.143 |
| U0126-EtOH | 0.735 | −0.248 | 0.524 | 0.474 |
| U0126-EtOH | 0.484 | −0.641 | 0.094 | 0.184 |
| U0126-EtOH | −0.457 | −0.558 | −0.320 | −0.393 |
| U0126-EtOH | −1.066 | −1.075 | −0.988 | −0.988 |
| UNC2250 | 0.915 | 0.942 | 0.742 | 0.666 |
| UNC2250 | 0.733 | 1.037 | 0.358 | 0.443 |
| UNC2250 | 0.866 | 0.366 | 0.585 | 0.816 |
| UNC2250 | −1.323 | −0.141 | −0.823 | −0.902 |
| UNC669 | 0.301 | −0.255 | 0.318 | 0.189 |
| UNC669 | −0.187 | 0.306 | −0.045 | −0.190 |
| UNC669 | 0.160 | −0.250 | 0.327 | 0.127 |
| UNC669 | 0.011 | 0.260 | 0.204 | 0.009 |
| URB597 | 0.915 | 0.791 | 0.822 | 0.938 |
| URB597 | 0.135 | 0.152 | −0.006 | 0.140 |
| URB597 | 0.108 | 0.449 | 0.254 | 0.087 |
| URB597 | −0.282 | −0.071 | −0.126 | −0.303 |
| Vandetanib (ZD6474) | −0.896 | −0.944 | −0.578 | −0.621 |
| Vandetanib (ZD6474) | −0.350 | −0.723 | −0.488 | −0.194 |
| Vandetanib (ZD6474) | −2.480 | −1.917 | −1.696 | −1.585 |
| Vandetanib (ZD6474) | −3.192 | −1.213 | −1.490 | −1.611 |
| Varespladib (LY315920) | 1.770 | 1.496 | 1.531 | 1.297 |
| Varespladib (LY315920) | 1.013 | 1.239 | 0.751 | 1.045 |
| Varespladib (LY315920) | 1.136 | 0.400 | 1.209 | 0.717 |
| Varespladib (LY315920) | 0.327 | 0.876 | 0.294 | 0.166 |
| VE-821 | 0.841 | 0.404 | 0.725 | 0.574 |
| VE-821 | 0.573 | 0.393 | 0.649 | 0.665 |
| VE-821 | −0.023 | −0.395 | 0.448 | −0.024 |
| VE-821 | −2.775 | −2.063 | −1.944 | −1.947 |
| VE-822 | 0.670 | −0.336 | 0.460 | 0.376 |
| VE-822 | −0.414 | −0.378 | −0.321 | −0.296 |
| VE-822 | −1.950 | −1.420 | −1.216 | −1.243 |
| VE-822 | −2.073 | −1.556 | −1.027 | −1.051 |
| Veliparib (ABT-888) | 0.682 | 0.412 | 0.536 | 0.413 |
| Veliparib (ABT-888) | 1.157 | 0.142 | 1.398 | 1.304 |
| Veliparib (ABT-888) | −0.100 | −0.071 | 0.171 | −0.084 |
| Veliparib (ABT-888) | −0.882 | −0.697 | −0.507 | −0.776 |
| Vemurafenib (PLX4032, RG7204) | 0.779 | 0.346 | 0.702 | 0.673 |
| Vemurafenib (PLX4032, RG7204) | 0.969 | −0.111 | 0.394 | 0.464 |
| Vemurafenib (PLX4032, RG7204) | 0.357 | −0.007 | 0.305 | 0.271 |
| Vemurafenib (PLX4032, RG7204) | 0.143 | 0.042 | 0.149 | 0.090 |
| Vildagliptin (LAF-237) | 0.642 | 0.398 | 0.877 | 0.391 |
| Vildagliptin (LAF-237) | 0.731 | 0.410 | 0.735 | 0.344 |
| Vildagliptin (LAF-237) | 0.245 | 0.369 | 0.728 | 0.143 |
| Vildagliptin (LAF-237) | 0.081 | 0.754 | 0.717 | 0.071 |
| Voriconazole | 1.163 | 0.687 | 0.908 | 1.355 |
| Voriconazole | 0.774 | 0.910 | 0.724 | 0.732 |
| Voriconazole | 0.628 | 0.656 | 0.577 | 0.596 |
| Voriconazole | 0.309 | 1.038 | 0.176 | 0.343 |
| VU 0357121 | 0.984 | 0.503 | 0.493 | 0.783 |
| VU 0357121 | 0.217 | 0.477 | −0.100 | 0.248 |
| VU 0357121 | 0.048 | 0.039 | 0.239 | 0.039 |
| VU 0357121 | −0.219 | −0.191 | −0.277 | −0.255 |
| VU 0364439 | 0.696 | 0.051 | 0.723 | 0.598 |
| VU 0364439 | 0.801 | −0.215 | 0.094 | 0.952 |
| VU 0364439 | −0.281 | −0.796 | 0.070 | −0.290 |
| VU 0364439 | −0.246 | 0.023 | −0.482 | −0.203 |
| VU 0364770 | 0.533 | 0.046 | 0.550 | 0.311 |
| VU 0364770 | 0.809 | 0.185 | 0.531 | 0.718 |
| VU 0364770 | 0.899 | 0.310 | 0.982 | 1.021 |
| VU 0364770 | 0.427 | −0.235 | 0.481 | 0.306 |
| VX-680 (Tozasertib, MK-0457) | −0.501 | −0.367 | −0.263 | −0.259 |
| VX-680 (Tozasertib, MK-0457) | −1.586 | −1.525 | −0.962 | −1.043 |
| VX-680 (Tozasertib, MK-0457) | −1.991 | −1.701 | −0.927 | −1.256 |
| VX-680 (Tozasertib, MK-0457) | −2.208 | −1.755 | −1.396 | −1.694 |
| VX-745 | 0.999 | 0.615 | 0.954 | 1.176 |
| VX-745 | 0.347 | 0.517 | 0.031 | 0.339 |
| VX-745 | 0.376 | 0.065 | 0.192 | 0.362 |
| VX-745 | 0.244 | 0.072 | −0.043 | 0.202 |
| VX-765 | 0.587 | 0.535 | 0.429 | 0.497 |
| VX-765 | 0.206 | 0.773 | 0.374 | 0.218 |
| VX-765 | 0.392 | 0.618 | 0.434 | 0.408 |
| VX-765 | 1.125 | 0.601 | 0.215 | 0.684 |
| VX-809 (Lumacaftor) | 1.408 | 0.434 | 0.652 | 0.729 |
| VX-809 (Lumacaftor) | −0.175 | −0.203 | −0.316 | −0.179 |
| VX-809 (Lumacaftor) | −0.470 | −0.313 | −0.303 | −0.530 |
| VX-809 (Lumacaftor) | −0.755 | 0.069 | −0.830 | −0.732 |
| Wnt-C59 (C59) | −1.566 | −1.424 | −0.833 | −0.837 |
| Wnt-C59 (C59) | −1.820 | −1.171 | −0.815 | −0.836 |
| Wnt-C59 (C59) | −2.022 | −1.060 | −1.194 | −1.178 |
| Wnt-C59 (C59) | −1.944 | −0.888 | −0.933 | −0.916 |
| WZ4002 | 0.740 | 0.990 | 0.730 | 0.639 |
| WZ4002 | 0.294 | 0.122 | 0.203 | 0.256 |
| WZ4002 | −0.707 | 0.506 | −0.762 | −0.787 |
| WZ4002 | −2.062 | −0.706 | −1.488 | −1.515 |
| WZ4003 | 0.759 | 1.008 | 0.469 | 0.826 |
| WZ4003 | −0.219 | 0.418 | −0.365 | −0.201 |
| WZ4003 | −0.229 | 0.212 | −0.154 | −0.232 |
| WZ4003 | −0.919 | −0.033 | −0.623 | −0.720 |
| WZ811 | 1.913 | 0.330 | 0.934 | 1.746 |
| WZ811 | 0.048 | −0.070 | 0.135 | 0.037 |
| WZ811 | 0.479 | 0.171 | 0.862 | 0.484 |
| WZ811 | 0.124 | 0.160 | 0.050 | 0.137 |
| XAV-939 | 0.475 | −0.340 | 0.262 | 0.381 |
| XAV-939 | 0.550 | −0.095 | 0.138 | 0.500 |
| XAV-939 | −0.442 | −0.983 | −0.292 | −0.326 |
| XAV-939 | −1.495 | −1.620 | −0.990 | −1.003 |
| XL335 | 0.601 | 0.326 | 0.975 | 0.467 |
| XL335 | −0.708 | −0.925 | −0.407 | −0.378 |
| XL335 | 0.290 | −0.807 | −0.390 | 0.106 |
| XL335 | −1.167 | −0.452 | −1.224 | −1.273 |
| YM155 (Sepantronium Bromide) | 1.114 | 0.486 | 0.962 | 1.204 |
| YM155 (Sepantronium Bromide) | −0.250 | −0.144 | −0.290 | −0.217 |
| YM155 (Sepantronium Bromide) | −0.883 | −1.517 | −0.813 | −0.795 |
| YM155 (Sepantronium Bromide) | −1.360 | −1.277 | −0.762 | −0.804 |
| YO-01027 | 0.681 | 0.552 | 0.292 | 0.448 |
| YO-01027 | −0.372 | −0.420 | 0.289 | −0.285 |
| YO-01027 | −0.133 | −0.460 | −0.492 | −0.104 |
| YO-01027 | −1.191 | −0.816 | −1.458 | −0.742 |
| ZCL278 | −0.693 | −0.765 | −0.543 | −0.430 |
| ZCL278 | 0.501 | 0.045 | −0.321 | 0.220 |
| ZCL278 | −0.513 | −0.479 | 0.101 | −0.542 |
| ZCL278 | −0.518 | −0.173 | 0.071 | −0.281 |
| Zebularine | 0.597 | 0.113 | 0.212 | 0.425 |
| Zebularine | 0.501 | −0.001 | 0.088 | 0.569 |
| Zebularine | 0.329 | −0.247 | 0.343 | 0.241 |
| Zebularine | 0.658 | −0.390 | 0.467 | 0.506 |
| Zibotentan (ZD4054) | 1.084 | 0.337 | 0.836 | 1.198 |
| Zibotentan (ZD4054) | 0.844 | −0.027 | 0.549 | 0.977 |
| Zibotentan (ZD4054) | −0.248 | −0.745 | −0.014 | −0.143 |
| Zibotentan (ZD4054) | 0.321 | −0.004 | 0.998 | 0.302 |
| ZM 306416 | 0.041 | −0.237 | 0.605 | 0.041 |
| ZM 306416 | −1.863 | −1.459 | −1.467 | −1.629 |
| ZM 306416 | −2.609 | −2.213 | −1.851 | −2.107 |
| ZM 306416 | −2.789 | −3.172 | −1.770 | −2.141 |
| ZM 447439 | 1.105 | 1.545 | 1.035 | 1.280 |
| ZM 447439 | 0.697 | 1.105 | 0.820 | 0.709 |
| ZM 447439 | 0.477 | 0.458 | 0.355 | 0.342 |
| ZM 447439 | −0.851 | −0.361 | −0.706 | −0.584 |
| Zosuquidar (LY335979) 3HCl | 0.800 | 0.038 | 0.912 | 0.753 |
| Zosuquidar (LY335979) 3HCl | 0.819 | 0.621 | 0.542 | 0.901 |
| Zosuquidar (LY335979) 3HCl | 0.832 | 0.699 | 0.828 | 0.686 |
| Zosuquidar (LY335979) 3HCl | −1.296 | −0.394 | −1.535 | −1.349 |

TABLE 5B
Secondary Screen

| Treatment | Dose (uM) | Dose.FC.CTG | Dose.FC.LYZ.NS | Dose.FC.LYZ.S | Z.CTG | Z.LYZ.NS |
|---|---|---|---|---|---|---|
| Bosutinib (SKI-606) | 0.02 | 0.216 | 0.133 | 0.103 | 1.944 | 1.061 |
| Bosutinib (SKI-606) | 0.04 | 0.139 | −0.067 | −0.086 | 1.254 | −0.535 |
| Bosutinib (SKI-606) | 0.08 | 0.082 | −0.070 | −0.072 | 0.735 | −0.562 |
| Bosutinib (SKI-606) | 0.16 | 0.128 | 0.068 | 0.007 | 1.152 | 0.545 |
| CCT128930 | 0.02 | −0.117 | −0.241 | −0.319 | −1.054 | −1.927 |
| CCT128930 | 0.04 | 0.122 | −0.020 | −0.014 | 1.101 | −0.164 |
| CCT128930 | 0.08 | 0.166 | 0.031 | 0.005 | 1.494 | 0.248 |
| CCT128930 | 0.16 | −0.088 | −0.160 | −0.217 | −0.797 | −1.281 |
| Dapagliflozin | 0.02 | 0.085 | 0.022 | −0.019 | 0.770 | 0.173 |
| Dapagliflozin | 0.04 | 0.174 | 0.150 | 0.069 | 1.569 | 1.197 |
| Dapagliflozin | 0.08 | 0.149 | 0.058 | 0.069 | 1.346 | 0.462 |
| Dapagliflozin | 0.16 | 0.035 | −0.060 | −0.082 | 0.311 | −0.479 |
| Finasteride | 0.02 | 0.066 | 0.056 | −0.019 | 0.596 | 0.445 |
| Finasteride | 0.04 | −0.180 | −0.189 | −0.260 | −1.623 | −1.510 |
| Finasteride | 0.08 | −0.011 | −0.191 | −0.242 | −0.099 | −1.522 |
| Finasteride | 0.16 | −0.065 | −0.203 | −0.253 | −0.588 | −1.617 |
| KPT-330 | 0.02 | 0.126 | −0.005 | −0.004 | 1.135 | −0.038 |
| KPT-330 | 0.04 | 0.029 | −0.040 | −0.077 | 0.263 | −0.318 |
| KPT-330 | 0.08 | 0.141 | 0.142 | 0.134 | 1.273 | 1.132 |
| KPT-330 | 0.16 | 0.120 | 0.244 | 0.215 | 1.080 | 1.947 |
| Nilotinib (AMM-107) | 0.10 | 0.092 | 0.092 | 0.044 | 0.830 | 0.734 |
| Nilotinib (AMN-107) | 0.20 | 0.122 | 0.161 | 0.117 | 1.098 | 1.283 |
| Nilotinib (AMN-107) | 0.40 | 0.141 | 0.101 | −0.007 | 1.275 | 0.805 |
| Nilotinib (AMN-107) | 0.80 | 0.233 | 0.165 | 0.143 | 2.104 | 1.317 |
| PF-4708671 | 0.50 | −0.049 | 0.005 | −0.040 | −0.443 | 0.039 |
| PF-4708671 | 1.00 | −0.039 | −0.200 | −0.245 | −0.354 | −1.600 |
| PF-4708671 | 2.00 | −0.076 | −0.221 | −0.272 | −0.684 | −1.764 |
| PF-4708671 | 4.00 | 0.067 | 0.015 | −0.042 | 0.601 | 0.120 |
| PHA-665752 | 0.10 | 0.100 | 0.210 | 0.133 | 0.898 | 1.675 |
| PHA-665752 | 0.20 | −0.032 | −0.027 | −0.089 | −0.288 | −0.219 |
| PHA-665752 | 0.40 | 0.303 | 0.099 | 0.076 | 2.735 | 0.789 |
| PHA-665752 | 0.80 | 0.185 | 0.090 | 0.048 | 1.664 | 0.722 |
| Rolipram | 0.50 | 0.053 | −0.043 | −0.082 | 0.477 | −0.342 |
| Rolipram | 1.00 | 0.133 | 0.152 | 0.104 | 1.198 | 1.213 |
| Rolipram | 2.00 | 0.202 | 0.099 | 0.030 | 1.819 | 0.793 |
| Rolipram | 4.00 | 0.065 | −0.051 | −0.071 | 0.583 | −0.407 |
| Ruxolitinib | 2.50 | −0.014 | −0.157 | −0.182 | −0.128 | −1.251 |
| (INCB018424) | | | | | | |
| Ruxolitinib | 5.00 | −0.224 | −0.138 | −0.217 | −2.016 | −1.098 |
| (INCB018424) | | | | | | |
| Ruxolitinib | 10.00 | −0.153 | −0.084 | −0.168 | −1.380 | −0.671 |
| (INCB018424) | | | | | | |
| Ruxolitinib | 20.00 | 0.166 | 0.065 | 0.027 | 1.496 | 0.522 |
| (INCB018424) | | | | | | |
| Safinamide Mesylate | 0.50 | −0.014 | −0.156 | −0.213 | −0.127 | −1.244 |
| Safinamide Mesylate | 1.00 | −0.082 | −0.231 | −0.294 | −0.743 | −1.847 |
| Safinamide Mesylate | 2.00 | −0.011 | −0.046 | −0.099 | −0.098 | −0.368 |
| Safinamide Mesylate | 4.00 | −0.002 | −0.034 | −0.086 | −0.022 | −0.272 |
| SB431542 | 0.06 | 0.160 | 0.089 | 0.067 | 1.439 | 0.709 |
| SB431542 | 0.12 | 0.120 | 0.116 | 0.072 | 1.078 | 0.927 |
| SB431542 | 0.24 | 0.023 | 0.105 | 0.027 | 0.206 | 0.838 |
| SB431542 | 0.48 | 0.083 | −0.016 | −0.050 | 0.752 | −0.128 |
| Varespladib | 0.02 | 0.027 | −0.091 | −0.138 | 0.244 | −0.723 |
| (LY315920) | | | | | | |
| Varespladib | 0.04 | 0.113 | 0.015 | −0.044 | 1.019 | 0.123 |
| (LY315920) | | | | | | |
| Varespladib | 0.08 | 0.207 | 0.090 | 0.065 | 1.862 | 0.718 |
| (LY315920) | | | | | | |
| Varespladib | 0.16 | 0.148 | 0.156 | 0.102 | 1.337 | 1.244 |
| (LY315920) | | | | | | |

| Treatment | Dose (uM) | Z.LYZ.S | repSSMD.CTG | repSSMD.LYZ.NS | repSSMD.LYZ.S |
|---|---|---|---|---|---|
| Bosutinib (SKI-606) | 0.02 | 0.788 | 1.251 | 1.620 | 1.147 |
| Bosutinib (SKI-606) | 0.04 | −0.663 | 0.876 | −0.666 | −0.977 |
| Bosutinib (SKI-606) | 0.08 | −0.554 | 0.584 | −0.903 | −0.892 |
| Bosutinib (SKI-606) | 0.16 | 0.053 | 0.830 | 0.711 | 0.089 |
| CCT128930 | 0.02 | −2.445 | −0.787 | −2.551 | −3.825 |
| CCT128930 | 0.04 | −0.108 | 0.922 | −0.204 | −0.157 |
| CCT128930 | 0.08 | 0.041 | 1.047 | 0.366 | 0.069 |
| CCT128930 | 0.16 | −1.661 | −0.528 | −1.532 | −2.302 |
| Dapagliflozin | 0.02 | −0.144 | 0.643 | 0.231 | −0.225 |
| Dapagliflozin | 0.04 | 0.528 | 0.979 | 1.267 | 0.751 |
| Dapagliflozin | 0.08 | 0.530 | 0.777 | 0.537 | 0.879 |
| Dapagliflozin | 0.16 | −0.626 | 0.253 | −0.753 | −0.929 |
| Finasteride | 0.02 | −0.147 | 0.358 | 0.362 | −0.197 |
| Finasteride | 0.04 | −1.996 | −0.994 | −2.570 | −3.825 |
| Finasteride | 0.08 | −1.859 | −0.083 | −2.177 | −2.574 |
| Finasteride | 0.16 | −1.942 | −0.549 | −2.738 | −3.144 |
| KPT-330 | 0.02 | −0.028 | 0.959 | −0.050 | −0.044 |
| KPT-330 | 0.04 | −0.587 | 0.179 | −0.506 | −0.955 |
| KPT-330 | 0.08 | 1.031 | 0.916 | 1.621 | 1.678 |
| KPT-330 | 0.16 | 1.646 | 0.779 | 2.691 | 2.337 |
| Nilotinib (AMM-107) | 0.10 | 0.339 | 0.571 | 1.309 | 0.652 |
| Nilotinib (AMN-107) | 0.20 | 0.897 | 0.777 | 1.683 | 1.324 |
| Nilotinib (AMN-107) | 0.40 | −0.053 | 0.902 | 1.064 | −0.055 |
| Nilotinib (AMN-107) | 0.80 | 1.099 | 1.535 | 1.063 | 0.953 |
| PF-4708671 | 0.50 | −0.305 | −0.324 | 0.058 | −0.496 |
| PF-4708671 | 1.00 | −1.879 | −0.284 | −1.909 | −2.451 |
| PF-4708671 | 2.00 | −2.088 | −0.558 | −2.529 | −3.604 |
| PF-4708671 | 4.00 | −0.320 | 0.414 | 0.171 | −0.503 |
| PHA-665752 | 0.10 | 1.019 | 0.755 | 2.400 | 1.586 |
| PHA-665752 | 0.20 | −0.680 | −0.244 | −0.350 | −1.201 |
| PHA-665752 | 0.40 | 0.585 | 1.415 | 0.942 | 0.906 |
| PHA-665752 | 0.80 | 0.365 | 1.023 | 1.142 | 0.542 |
| Rolipram | 0.50 | −0.631 | 0.368 | −0.478 | −0.936 |
| Rolipram | 1.00 | 0.801 | 0.832 | 1.715 | 1.107 |
| Rolipram | 2.00 | 0.227 | 1.420 | 0.950 | 0.169 |
| Rolipram | 4.00 | −0.543 | 0.447 | −0.543 | −0.736 |
| Ruxolitinib | 2.50 | −1.400 | −0.090 | −1.701 | −1.806 |
| (INCB018424) | | | | | |
| Ruxolitinib | 5.00 | −1.667 | −1.168 | −1.569 | −2.281 |
| (INCB018424) | | | | | |
| Ruxolitinib | 10.00 | −1.289 | −1.030 | −1.103 | −2.212 |
| (INCB018424) | | | | | |
| Ruxolitinib | 20.00 | 0.208 | 0.952 | 0.930 | 0.382 |
| (INCB018424) | | | | | |
| Safinamide Mesylate | 0.50 | −1.637 | −0.118 | −1.484 | −2.413 |
| Safinamide Mesylate | 1.00 | −2.259 | −0.547 | −2.985 | −3.571 |
| Safinamide Mesylate | 2.00 | −0.757 | −0.060 | −0.556 | −1.243 |
| Safinamide Mesylate | 4.00 | −0.658 | −0.013 | −0.431 | −0.942 |
| SB431542 | 0.06 | 0.514 | 0.654 | 1.073 | 0.855 |
| SB431542 | 0.12 | 0.553 | 0.566 | 1.560 | 0.985 |
| SB431542 | 0.24 | 0.211 | 0.141 | 0.830 | 0.301 |
| SB431542 | 0.48 | −0.387 | 0.527 | −0.149 | −0.449 |
| Varespladib | 0.02 | −1.057 | 0.187 | −0.763 | −1.652 |
| (LY315920) | | | | | |
| Varespladib | 0.04 | −0.339 | 0.518 | 0.213 | −0.641 |
| (LY315920) | | | | | |
| Varespladib | 0.08 | 0.497 | 1.114 | 1.251 | 0.838 |
| (LY315920) | | | | | |
| Varespladib | 0.16 | 0.784 | 0.853 | 1.675 | 1.156 |
| (LY315920) | | | | | |

The results of primary and secondary screening reflect a mixture of potential effects arising from small molecule treatment which may result in increases in total LYZ secretion. This includes contributions from enhanced Paneth cell differentiation, altered Paneth cell activity and changes in total cell number concurrent with differentiation. To better inform how the 6 compounds identified in the screen increased total secreted LYZ, and to isolate only those which enhance Paneth differentiation robustly, Applicants next utilized flow cytometry to measure the changes in Paneth cell representation within the treated organoids. As another measure to ensure that Applicants do not select for compounds which manifest their behavior only in specific in vitro settings, Applicants performed the analyses in the conventional 3-D culture method to control for 2.5-D culture system-specific effects. Single live cells were selected by several gating strategies and Paneth cells were identified as lysozyme-high, CD24-mid, side scatter-high (SSC-high) (FIG. 27E). The 6 hit compounds were provided at the most potent dose from 2.5-D screening, with organoids in ENR+CD media for 6 days with media change every other day. Only one compound, KPT-330 the most potent compound in validation screening, significantly enhanced the mature Paneth cell population within organoids, suggesting KPT-330 induces PC differentiation (FIG. 23E). To evaluate whether the culture media supplements C and D may alter the effects of the 6 hits, Applicants also performed lysozyme assay in the canonical ENR culture condition in 3-D (because Paneth cells exist in an immature state within ENR, Applicants were unable to robustly quantify Paneth cell number via flow cytometry). This again shows that only KPT-330 is enhancing Paneth cell-specific activity in the conventional organoid culture condition, and led us to focus solely on KPT-330 and its potential mechanism of Xpo1 inhibition going forward (FIG. 27F).

### Validating Xpo1 as Molecular Target Enhancing Paneth Cell Differentiation

Applicants next sought to confirm the predicted on-target activity of KPT-330 and demonstrate dose-dependency of treatment in enhancing Paneth cell differentiation. KPT-330 is a first-in-class orally-administered FDA-approved drug against multiple myeloma, targeting a nuclear transporter, XPO1 (also known as CRM-1).

Administration of KPT-330 below 160 nM for 6 days (in primary screening higher concentrations proved toxic) showed enhanced Paneth cell activity in a dose-dependent manner, with 160 nM of KPT-330 as the most effective dose among tested concentrations, as evidenced by the LYZ secretion levels in both basal and Cch-stimulated conditions (FIG. 23F). To confirm that KPT-330 is acting via the inhibition of Xpo1, Applicants used two additional Xpo1 inhibitors: KPT-8602, a second-generation compound based on KPT-330, and leptomycin B, a canonical inhibitor. Flow cytometry analyses revealed both KPT-8602 and leptomycin B increased the proportion of PC in the organoids (FIG. 23G). Additionally, LYZ secretion assays with the additional Xpo1 inhibitors showed similar Paneth cell-enrichments both in conventional (ENR) and Paneth-differentiation (ENR+CD) culture conditions (FIG. 27G, 27H). Applicants also utilized Western blotting as an alternative method to assess the abundance of lysozyme within organoids for indirectly measuring Paneth cell enrichment using a different antibody than used in flow cytometry. LYZ expression levels per unit weight were enhanced by three XPO1 inhibitors (FIG. 271), consistent with the results of LYZ secretion assays and flow cytometry analyses.

KPT-330 is a type of small molecule known as a selective inhibitor of nuclear export (SINE), these molecules act by suppressing the Xpo1-regulated nuclear export of multiple proteins and mRNAs from the nucleus to the cytoplasm, including genes involved in stem cell maintenance and differentiation as well as inflammatory stress response (Sendino et al., 2018). Proteins shuttled by the transporter Xpo1 are marked with a nuclear export signal (NES). Additionally, Xpo1 is known to regulate cell cycle through Xpo1's export-independent role in the regulation of mitosis (Forbes et al., 2015). Based on this evidence, Applicants hypothesized that Xpo1 inhibition was providing for enhanced Paneth cell differentiation by directing ISCs to modulate their differentiation trajectories through alterations in either developmental signaling within the nucleus and/or interfering with cell cycle.

Longitudinal scRNA-Seq of Differentiation Reveals Dynamic Population Shifts with Xpo1 Inhibition Resulting in Paneth Cell Enrichment

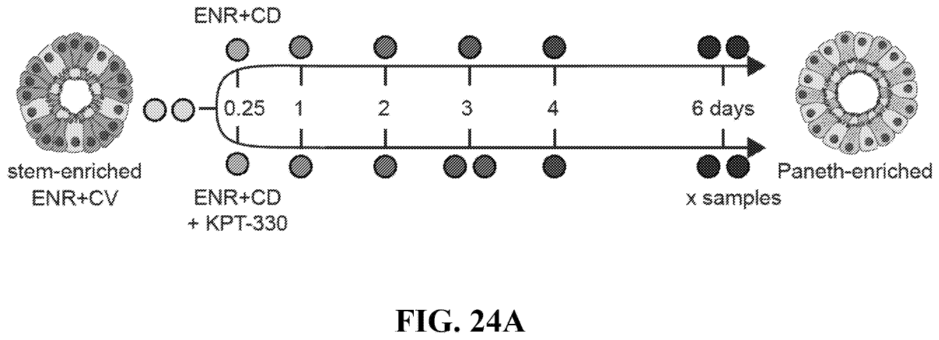

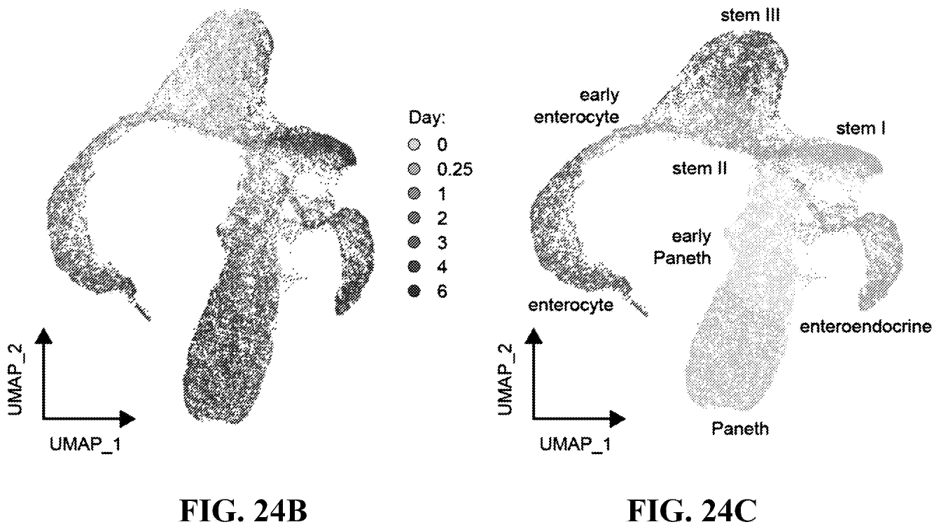

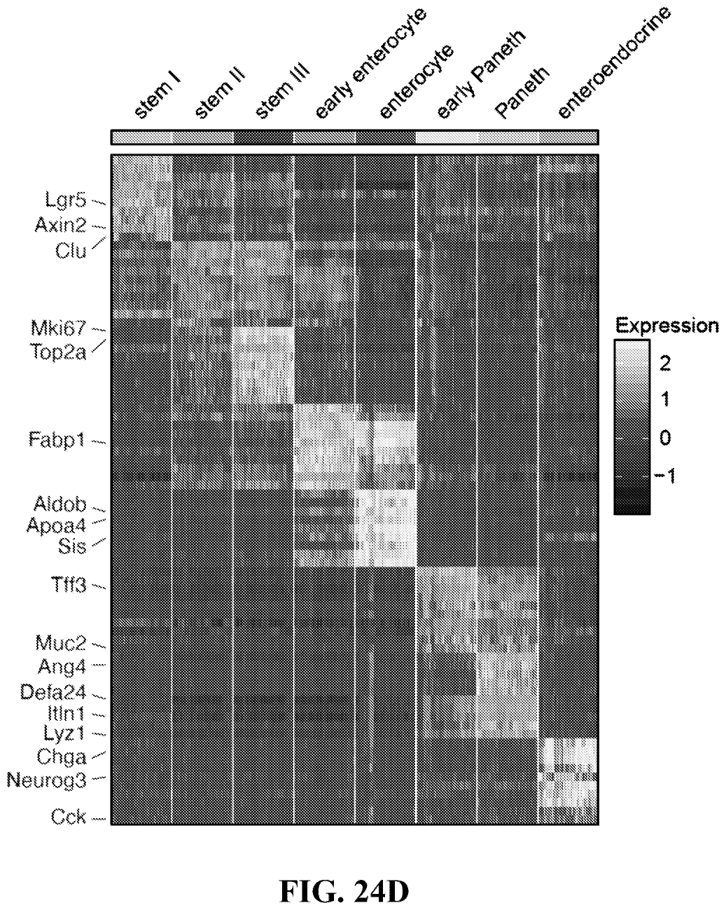

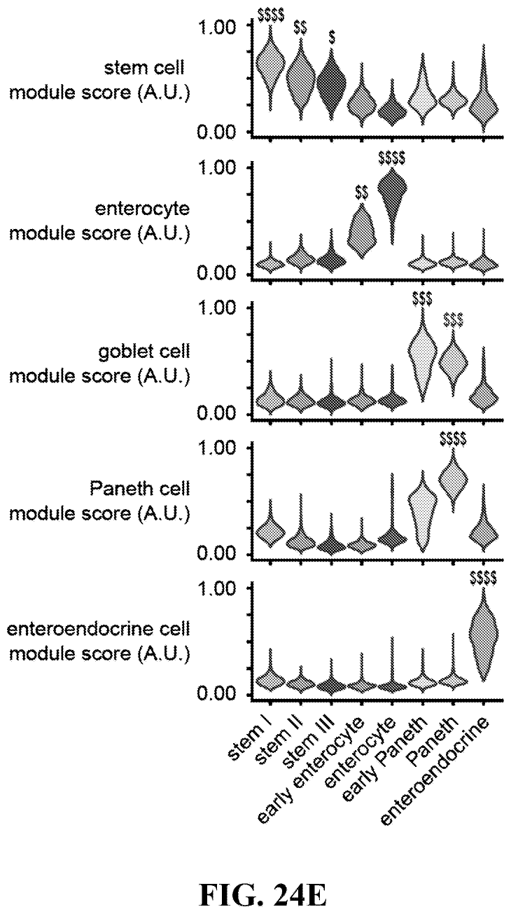

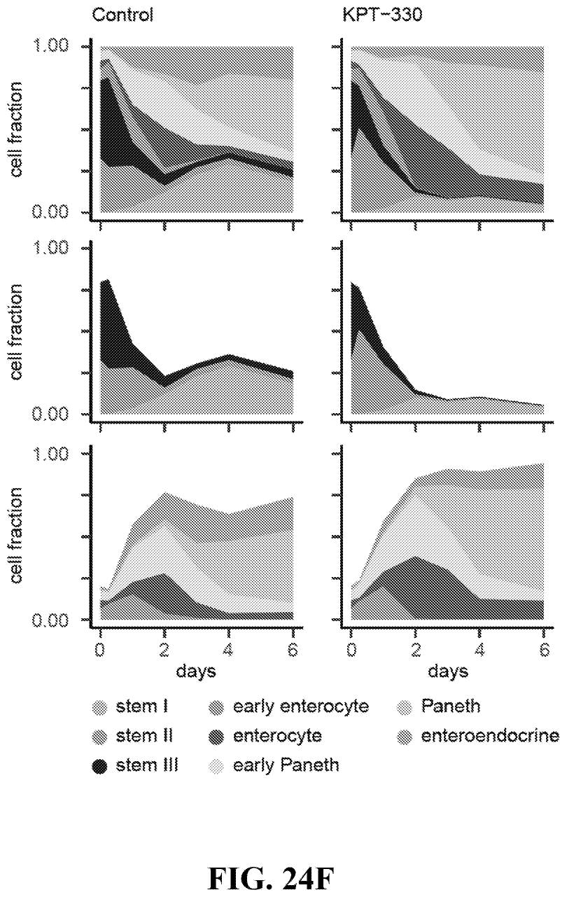

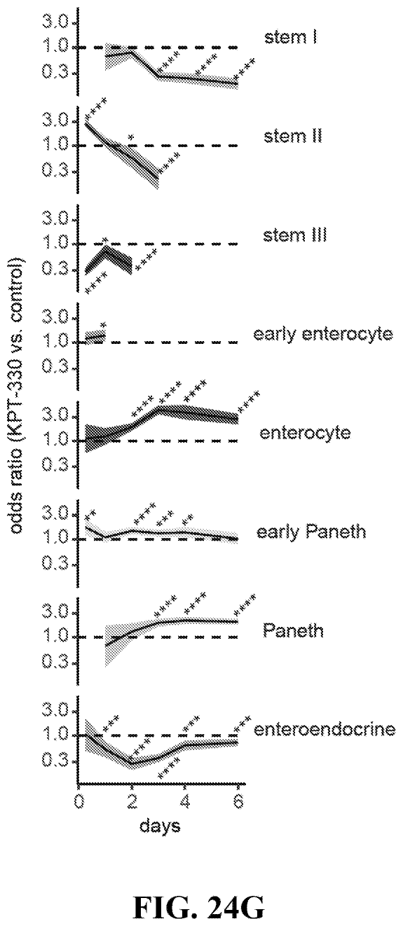

To test the hypothesis that KPT-330 drives Paneth differentiation by altering ISC behavior, Applicants utilized single-cell RNA-sequencing (scRNA-seq) via the recently updated Seq-Well microwell technology (Hughes et al., 2019) to perform a longitudinal comparison between untreated and KPT-330 treated organoids over the same 6-day differentiation, with a particular emphasis on early timepoints (FIG. 24A).

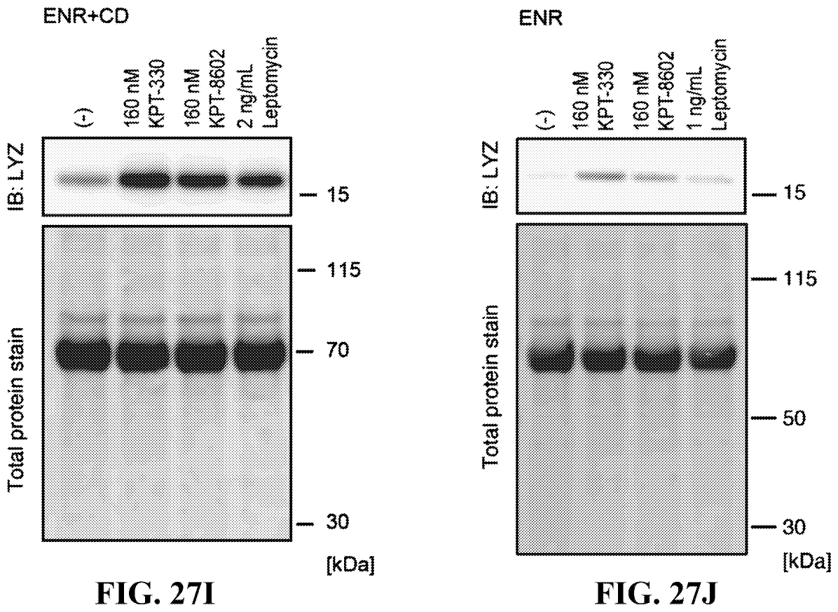

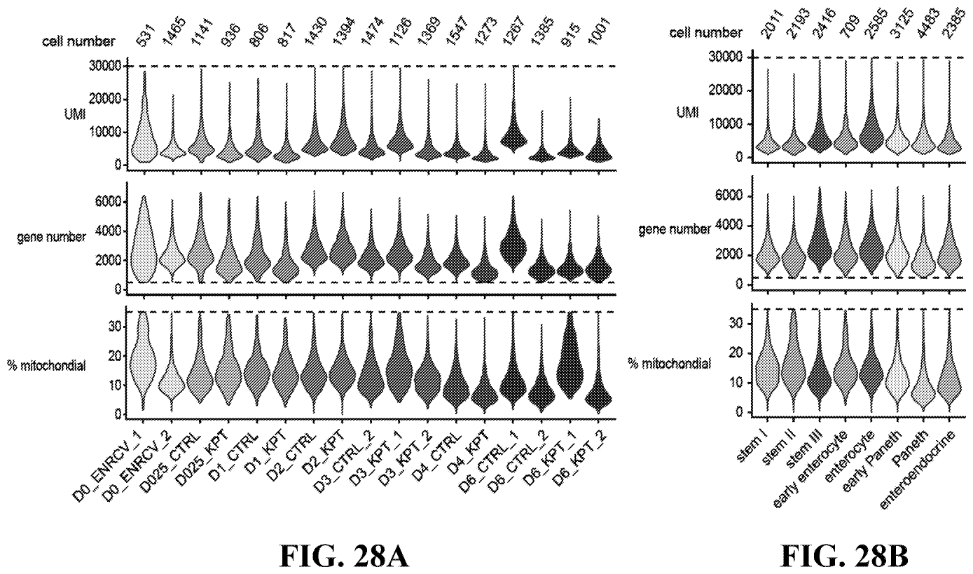

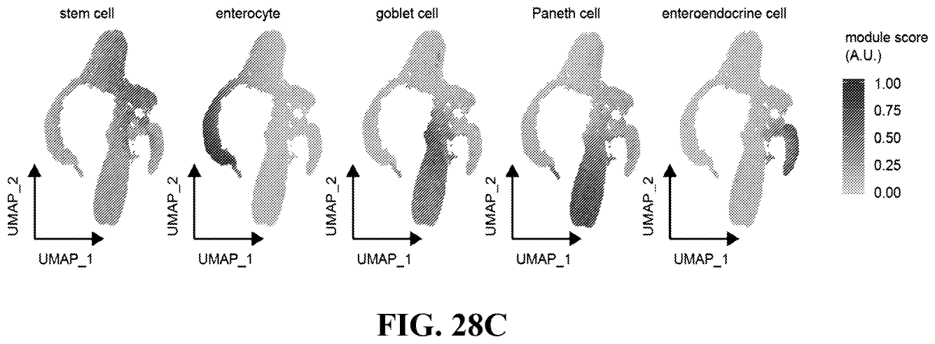

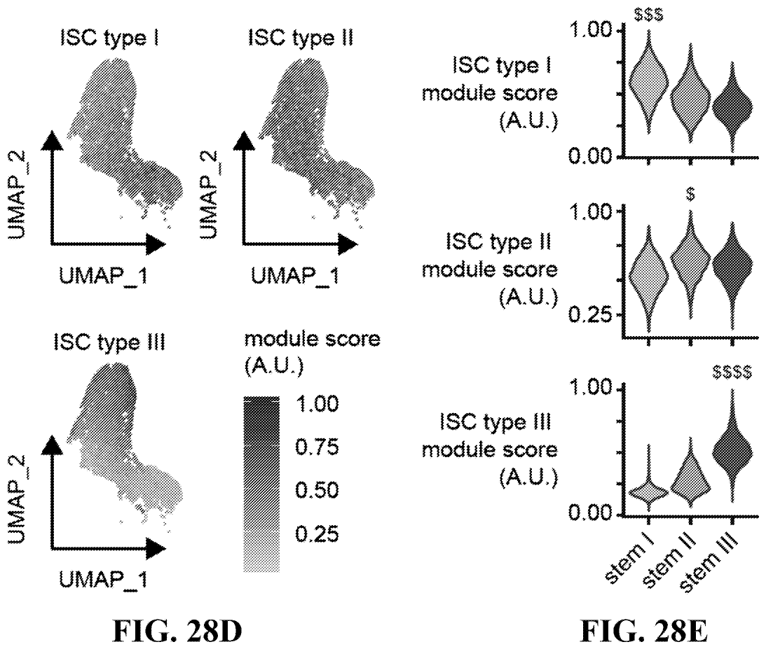

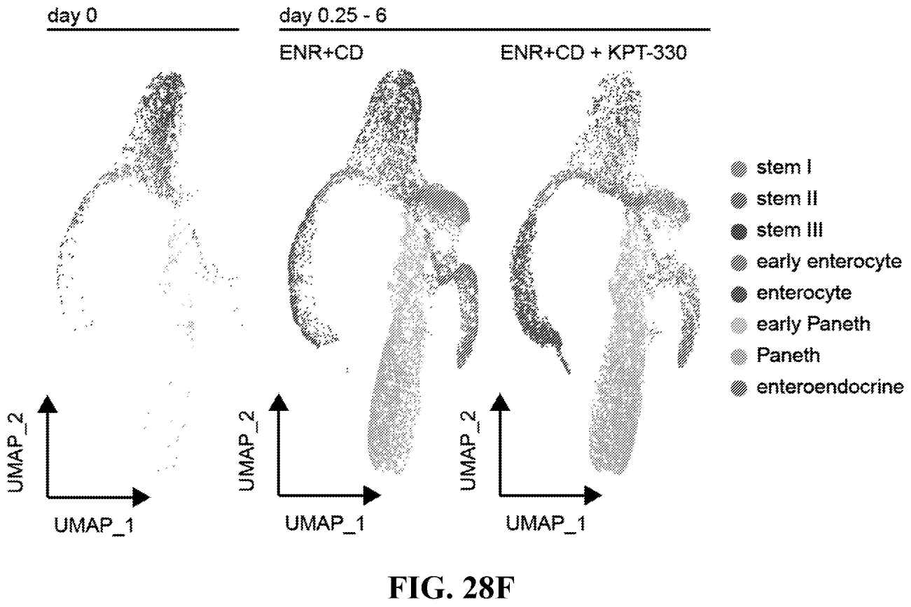

Applicants collected 18 samples corresponding to pre-differentiation ENR+CV organoids (n=2) and both ENR+CD and ENR+CD+KPT-330 (160 nM) at 6 hours (0.25 days, n=1), 1 day (n=1), 2 days (n=1), 3 days (n=2), 4 days (n=1), and 6 days (n=2). For time points beyond 2 days, media was refreshed every other day. Following the Seq-Well protocol Applicants followed a standard library preparation, sequencing, and alignment process (see Methods). Due to a technical failure in membrane sealing during Seq-Well, one sample (day 3 ENR+CD) was not included in analysis. Prior to analysis, cell-by-gene digital expression matrices were pre-processed to remove cellular barcodes with less than 500 unique genes, greater than 35% of unique molecular identifiers (UMIs) corresponding to mitochondrial genes, low outliers in standardized house-keeping gene expression (Tirosh et al., 2016), barcodes with greater than 30,000 UMIs, and cellular doublets identified through manual inspection and use of the DoubletFinder algorithm (McGinnis et al., 2019). The resulting dataset consists of 19,877 cells spanning the 17 samples collected. UMI, percent mitochondrial, and detected gene distributions are similar across samples, with likely differences due to variations in library preparation and sequencing depth (FIG. 28A). To better control for potential batch effects that may arise from differences in library preparation, dimensional reduction and clustering was performed following normalization with regularized negative bionomical regression as implemented in Seurat V3 via SCTransform (Hafemeister and Satija, 2019).

Unsupervised UMAP reduction of the complete dataset nicely reveals the time-course structure along with branches suggestive of distinct lineages arising over the course of differentiation (FIG. 24B). Louvain clustering separated the data into 8 clusters, which Applicants manually annotated (FIG. 24C) as mature epithelial and stem cells. Importantly, Applicants observed that each cluster possessed highly similar quality metrics (FIG. 28B), while being enriched for expression of canonical markers of multiple intestinal epithelial cell types (FIG. 24D). To contextualize the cellular identity of the 8 clusters, Applicants used lineage-defining gene sets from a murine small intestinal scRNA-seq atlas (Haber et al., 2017) to score each cell relative to all others using the strategy of Tirosh et al., 2016 (FIG. 28C). In addition to showing that the UMAP branches correspond to lineages along trajectories of differentiation over real time from a stem-like pool, scores also correspond well with unique clusters of the 8 identified (FIG. 24E). Lineage module scoring combined with the expression of select lineage-defining genes allowed Applicants to classify the 8 clusters as 3 stem-like, 2 enterocyte, 2 Paneth, and enteroendocrine, aligning with the expectation that ENR+CD differentiation should enrich for secretory epithelium, principally Paneth and to a lesser extent enteroendocrine. To better contextualize the 3 stem-like clusters, and assess potential physiological relevance, Applicants again performed module scoring over gene sets identified to correspond to known ISC subsets in vivo (Biton et al., 2018) (FIG. 28D). Applicants see clear alignment with the type III and slight enrichment for type I ISCs per the nomenclature of Biton et al., additionally there is slight enrichment for a distinct type II (FIG. 28E) though it may also be that this population is a transient intermediate between stem populations, sharing markers with the other two (FIG. 24D). Accordingly, Applicants adopted the naming scheme of Biton et al. to describe the three ISC populations, type I appearing enriched for canonical markers of ISCs, including LGR5, type III being most distinguished by the high expression of many genes involved in cell cycle, and type II appearing as a transitory or intermediate population between I and III.

Having annotated the complete dataset, Applicants next sought to explore what factors distinguish the organoids treated with KPT-330 versus control. Importantly, in the combined dataset Applicants see that across all three conditions, day 0 ENR+CV, and day 0.25-6 ENR+CD and ENR+CD+KPT-330 there are not unique cell populations, but rather shifts in the abundance of cell types with KPT-330 treatment (FIG. 28F). In fact, looking over the time course, Applicants see clear changes in the relative abundance of different cell populations between KPT-330 treated and control. Both conditions begin with over 75% of cells as either stem II or stem III, but by day 0.25 Applicants see a relative shift from mostly stem III to mostly stem II (from rapidly cycling to transitory stem). Further, by day 2 and extending through day 6 Applicants see the emergence of stem I, accounting for approximately 25% of the cells in the control condition, but a much smaller proportion in KPT-330 treated organoids. Looking at the differentiating populations Applicants see the rapid emergence of early enterocytes and early Paneth cells at day 1, with the continued differentiation to enterocytes and eventual disappearance of enterocytes by day 4. Early Paneth appears to crest with enterocytes followed by a transition to Paneth cells continuing to day 6. The major distinguishing factors between KPT-330 treated and control in the differentiating populations is the relative early enrichment for enterocytes, later enrichment for Paneth cells, and suppression of the non-cycling stem I population (FIG. 24E). To better quantify the differences in representation between KPT-330 treated and control over time, Applicants constructed a 2×2 contingency table of each individual cell type relative to all others at each timepoint where that cluster accounted for at least 1% of cells in both KPT-330 and control samples. Applicants present the relative enrichment or depletion of a cell population with KPT-330 treatment over time as the odds ratio (OR) with a corresponding 95% confidence interval. This again shows the relative depletion of stem I (and stem II & III) and enteroendocrine cells over time along with the corresponding enrichment of enterocytes and Paneth cells (FIG. 24G). This agrees to a large extent with the flow cytometry observations of a 2-fold increase in mature Paneth cells at day 6 of differentiation with KPT-330 (the day 6 Paneth cell OR from scRNA-seq being ˜2), while also showing the unexpected early enrichment of enterocytes and longer-term depletion of a subset of stem cells, stem I.

Xpo1 Inhibition Drives Cycling ‘stem II/III’ ISCs into a Pro-Differentiation State Via Stress Response and Suppression of Mitogen Signaling

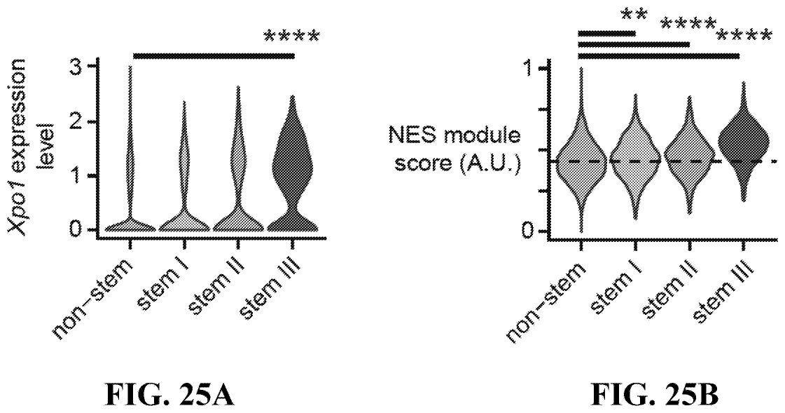

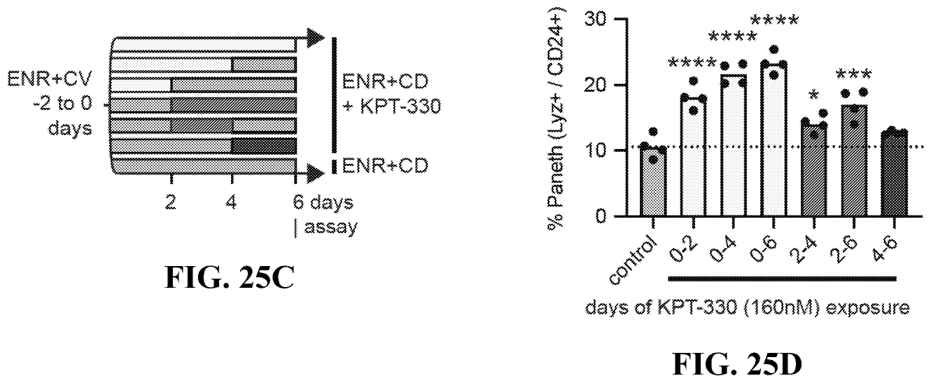

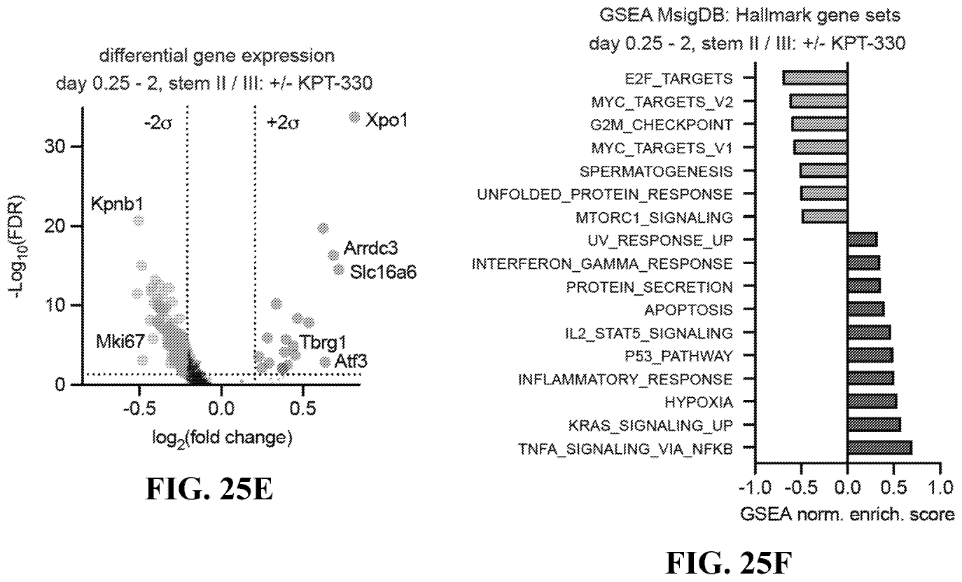

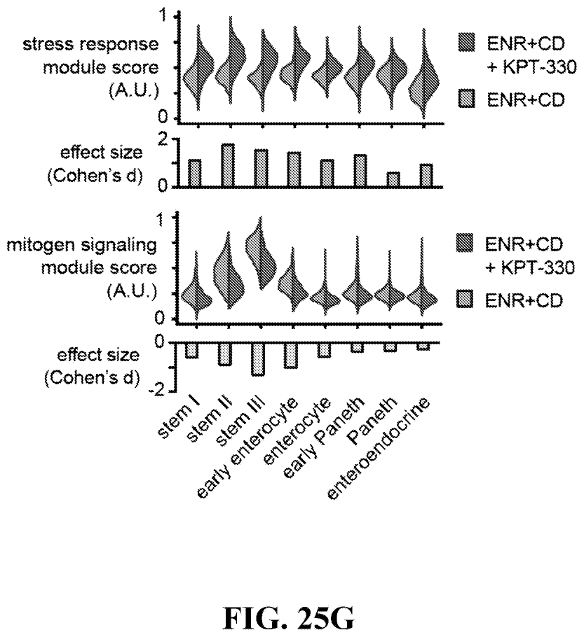

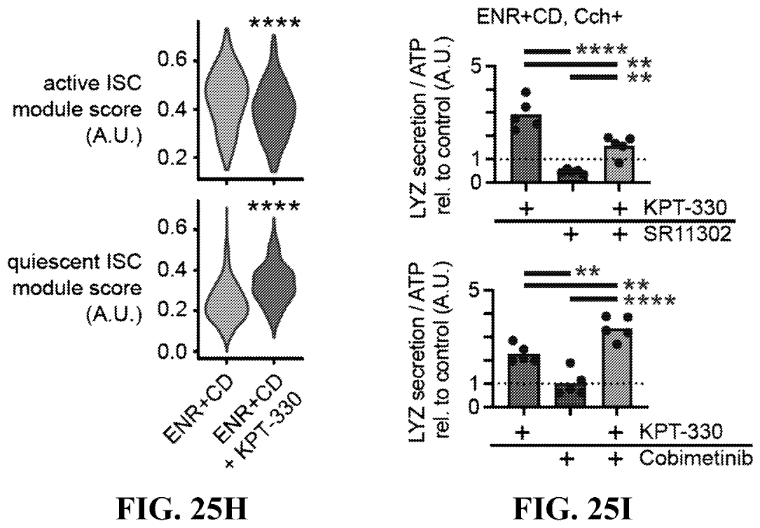

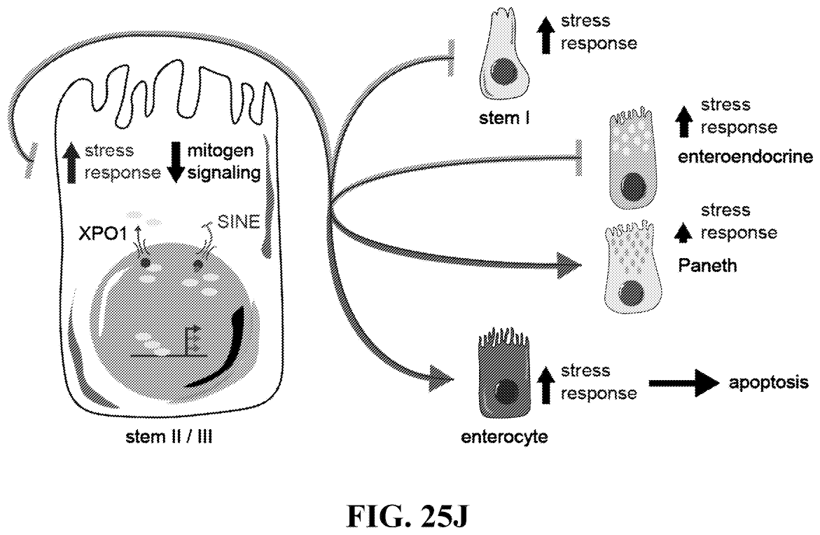

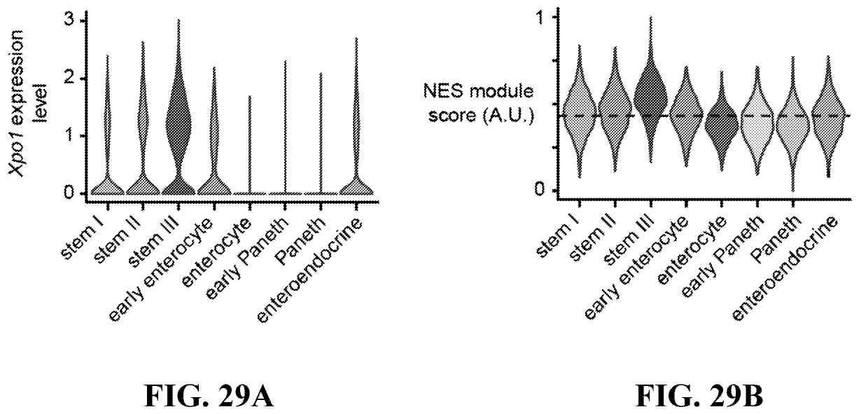

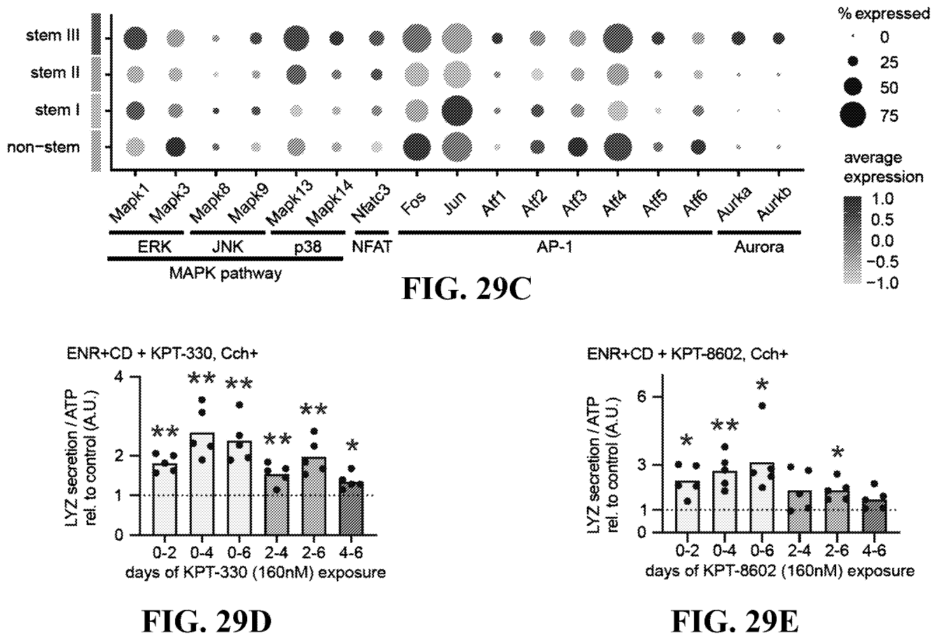

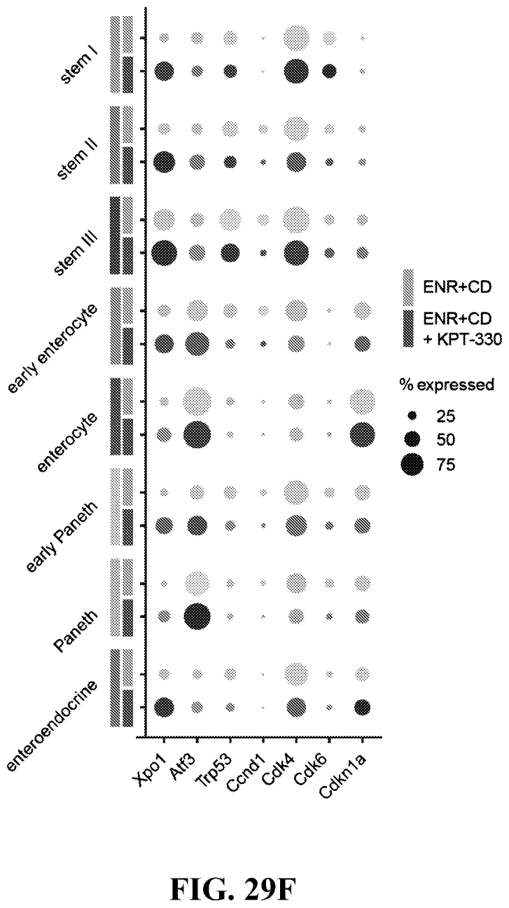

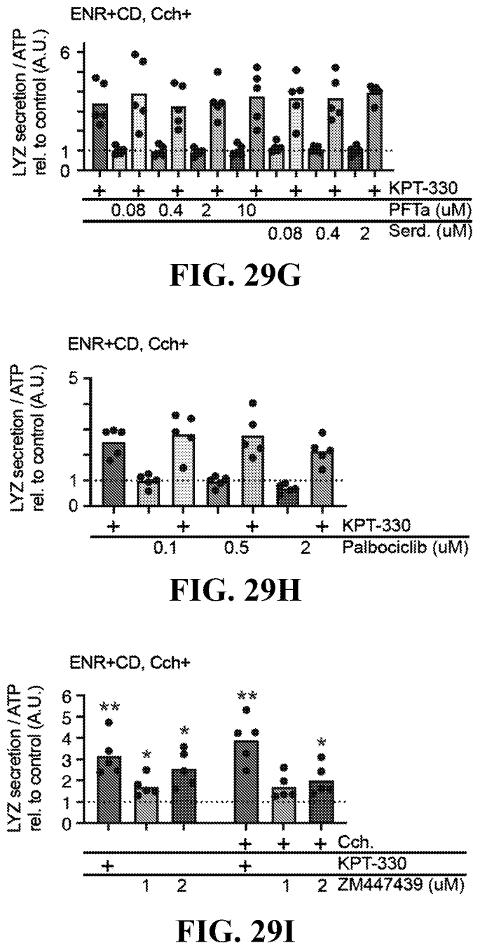

The observation of compositional changes over the course of differentiation are consistent with Xpo1 inhibition acting as a pro-differentiation agent within the stem II/III populations most abundant at the beginning of differentiation. In fact, Applicants see that in non-treated organoids, the expression of Xpo1 is significantly enriched in the actively cycling stem III population (FIG. 25A & FIG. 29A). Applicants also observe that the expression of genes known to contain a NES, which are required for the nuclear efflux via Xpo1 and therefore moderated by Xpo1 inhibition, are enriched in the stem cell populations, most significantly in stem III (FIG. 25B & FIG. 29B) (Fu et al., 2013). More specifically, Applicants know that Xpo1 is an important mediator of nuclear signaling processes including the mitogen-activated protein kinase (MAPK) pathway, NFAT, AP-1, and Aurora kinase activity during cell division (Sendino et al., 2018; Sun et al., 2016). Applicants observe the expression of many key mediators in these pathways within the stem populations, and see particular stem III-enrichment in members of MAPK (Mapk1, Mapk9, Mapk13, Mapk14), NFAT (Nfatc3), AP-1 (Atf1), and Aurora kinases (Aurka, Aurkb) (FIG. 29C).

To further establish whether the stem II/III population is the principal cellular target of Xpo1 inhibition, Applicants leveraged the dynamic nature of the system and exposed differentiating organoids to KPT-330, over varied timespans. Because the abundance of stem, differentiating, and mature populations change through this course, by inhibiting Xpo1 over every continuous 2, 4, and 6-day interval in the 6-day differentiation and measuring final abundance and function of mature Paneth cells at the end of differentiation, Applicants can infer the relative effect of Xpo1 inhibition on each cell type (FIG. 25C). Applicants see that of all 2-day KPT-330 treatments, day 0-2 results in the greatest enrichment in mature Paneth cells, with longer durations of exposure following day 2 providing additional, albeit lesser enrichment. Further Applicants see that day 2-4 produces moderate enrichment, while day 4-6 is no different than untreated (by flow cytometry) or minorly enriched (by LYZ secretion assay) (FIG. 25D & FIG. 29D). Using an additional SINE, KPT-8602, Applicants observe similar enrichment behavior as KPT-330 (FIG. 29E). This data supports that Xpo1 inhibition is principally altering stem II/III differentiation—the largest effects of Xpo1 inhibition are concurrent with periods in the differentiation course where stem II/III populations are most abundant. However, this data also suggests that the effect of Xpo1 inhibition may not be entirely stem-dependent, given the lesser, but significant increases in Paneth cell number and function with later treatment.

To better understand the pleiotropic effects of Xpo1 inhibition which may mediate differentiation within stem II/III, Applicants examined the differentially expressed genes between KPT-330 treated and untreated stem II/III populations in the earliest stages of differentiation when they are most abundant (day 0.25-2). Both the most significantly enriched (Xpo1) and depleted (Kpnb1—a nuclear importin) genes suggest that these cells are significantly impacted by KPT-330 treatment and are enacting changes in expression to reestablish homeostasis of nuclear cargo transit (FIG. 25E & Table 3A). Notable genes with significantly increased expression include Arrdc3 (known to regulate proliferative processes), Slc16a6 (a principal transporter of ketone bodies—recently shown to be instructional in ISC fate decisions), Tbgr1 (a growth inhibitor), and Atf3 (regulates stress response in ISCs) (Cheng et al., 2019; Draheim et al., 2010; Jadhav and Zhang, 2017; Zhou et al., 2017a). Genes down-regulated by KPT-330 treatment appear related to proliferation and cell cycle, notably the marker Mki67. Notably, of these responses to KPT-330 treatment within stem II/III, Xpo1, Atf3, Trp53 (p53), Ccnd1, Cdk4/6, and Cdkn1a (p21) expression are increased across all cell types (at all times), but with significant differences in the fraction of cells in each which express each gene (FIG. 29F). This suggests that there are both stem II/III specific responses and pan-epithelial responses to Xpo1 inhibition.

To better contextualize the transcriptional response to KPT-330 treatment in stem II/III cells, Applicants performed gene set enrichment analyses (GSEA) using the v7 molecular signatures database (MSigDB) hallmark collection which represent specific well-defined biological states or processes across systems (Liberzon et al., 2015; Subramanian et al., 2005). Significant gene sets with FDR<0.05 reveal two major programs differentially enriched following KPT-330 treatment, with enrichment or depletion quantified through the GSEA normalized enrichment score (a quantification of the degree to which a gene set is over-represented at either extreme of the full ranked list of differentially expressed genes) (FIG. 25F & Table 3B). KPT-330 treatment suppresses programs downstream of mitogen-driven signaling, notable targets of E2F, and MYC, as well as genes involved in cell cycle (G2M checkpoint), while up-regulating programs broadly resembling a complex stress response (NFkB signaling, hypoxia, inflammatory response). Compellingly, these responses are in strong agreement with the known effects of Xpo1 inhibition in the context of malignancy.

Applicants next sought to examine whether either of these responses, as embodied by the significant differentially expressed genes in stem II/III (day 0.25-2), may be pan-epithelial or restricted to the actively cycling stem II/III populations. Interestingly, Applicants see that the stress response module is substantially increased across all cell populations during differentiation, with the greatest effects in the stem II/III and early mature cell populations, and lowest effect in the mature Paneth cells (FIG. 25G). Conversely, Applicants see that the mitogen signaling module is selectively decreased in the stem II/III and early enterocyte populations relative to all others. This selectivity is likely due to the fact that the majority of mitogen signaling occurs within the proliferative stem II/III populations, and is closer to a floor in the mature populations. Combined Applicants see that the SINE-induced stress response appears to be a pan-epithelial response, while the modulation of mitogen signaling is restricted to the actively cycling stem cells. Recent work on mitogen and stress response control of re-entry into cell cycle may provide important context on the necessity of overlap of these two responses (Yang et al., 2017). Specifically, mother cells will transmit P53 protein and Ccnd1 transcripts to daughter cells, which, based on the abundance of transmitted signal with either immediately re-enter cell cycle, or commit to a quiescent state.

Transitions between quiescence and proliferation within the ISC niche have important roles in tissue homeostasis and regeneration. Quiescent pools of crypt-residing or adjacent cells serve as reserve populations which upon injury-dependent depletion of cycling stem cells will reestablish cycling progenitors and maintain homeostatic tissue regeneration (Ayyaz et al., 2019; Yousefi et al., 2017). Further, Applicants know that a transient quiescent intestinal stem cell state facilitates secretory enteroendocrine cell differentiation (Basak et al., 2017). To explore if similar phenomenon is occurring following Xpo1 inhibition, Applicants mapped the gene modules identified by Basak et al. of active and quiescent ISCs onto the early (day 0.25-2) stem II/III cells, where Applicants do in fact see a transition into quiescence with KPT-330 treatment (FIG. 25H). Combined with the observation that Xpo1 inhibition blocks the emergence of the non-cycling stem I population, the data suggest a model wherein SINE-induced stress response and disruption of mitogen signaling instruct proliferative progenitors to exit cell cycle and differentiate preferentially towards the Paneth and enterocyte lineages, while limiting the accumulation of ‘reserve’ non-cycling stem I cells and enteroendocrine cells.

Applicants sought to clarify this conceptual model with the use of additional small molecule inhibitors known to modulate discrete components of the hypothesized differentiation process. Applicants began by treating organoids along the ENR+CD differentiation course with a small molecule inhibitor of AP-1, SR11302, to test whether AP-1 is critical to the SINE-induced stress response, both alone and in combination with KPT-330. Applicants observe that when added with KPT-330, SR1302 significantly decreases functional LYZ secretion at the end of the 6-day differentiation, however when added alone, SR11302 also decreases functional LYZ secretion (FIG. 25I). This suggests that AP-1 signaling is an important mediator of Paneth differentiation from ISCs, though is only suggestive and not conclusive that Xpo1 inhibition acts on AP-1 to mediate its pro-differentiation effect. Applicants next tested whether P53 is an important downstream mediator by repeating the above assay with two known P53 inhibitors, pifithrin-α (PFTa) and serdemetan (serd.). Across a wide dose-range neither P53 inhibitor altered Paneth cell differentiation either alone or in combination with KPT-330, suggesting that the KPT-330 stress response is not dependent on P53 signaling (FIG. 29G). With the same assay Applicants began to probe the mitogen signaling response by adding the MEK inhibitor, cobimetinib (as shown by Basak et al. to induce the quiescent ISC population), in combination with KPT-330. Interestingly, cobimetinib alone did not significantly alter Paneth cell differentiation, however, when used in combination with KPT-330 proved to significantly enhance Paneth cell differentiation as measured by functional LYZ secretion (FIG. 25I). Applicants next sought to test whether regulation of cell cycle via mitogen signaling may be an important downstream mediator following Xpo1 inhibition. Inhibition of Cdk4/6 with palbociclib alone and in combination with KPT-330 did not alter Paneth cell differentiation (FIG. 29I), however inhibition of aurora kinase b with ZM447439 did significantly increase Paneth cell differentiation (notably ZM447439 was also a lower-effect size hit of the primary screen) (FIG. 29I). Combined, these experiments suggest that the SINE-induced stress response may be mediated by AP-1 and not P53, while suppression of mitogen signaling is not dependent on ERK, but is further enhanced by ERK inhibition, and that the non-exported related action of Xpo1 during cell cycle (which interacts with aurora kinase) may further contribute to the observed pro-differentiation effect.

In total, the analyses suggest that Xpo1 inhibition drives Paneth cell enrichment via the induction of a pan-epithelial stress response and suppression of mitogen signaling within the cycling ISC population (stem II/III). This response results in the cycling stem population becoming transiently quiescent, thereby favoring differentiation towards the Paneth and enterocyte lineages (the latter being a short-lived population relative to the former) over a more balanced transition to the mature lineages and the quiescent stem pool (stem I) (FIG. 25J).

### Low Dose Oral Xpo1 Administration In Vivo Induces Selective Expansion of the Paneth Cell Compartment

Based on the understanding of Xpo1 inhibition in stem-enriched organoids, Applicants hypothesized that SINE compounds may selectively enrich the epithelium for Paneth cells in vivo. The findings in organoids suggest that SINE treatment is independent of the niche cues of Wnt and Notch, acts specifically on cycling stem cells, which are abundant in the epithelial crypts, and while Xpo1 inhibition may enrich for both Paneth cells and enterocytes, by virtue of the relatively long Paneth cell lifespan (Ireland et al., 2005), Applicants would expect a longer-term accumulation of Paneth cells in vivo relative to enterocytes. Additionally, because the organoid data suggests Xpo1 inhibition does not expand the stem cell pool, but rather rebalances patterns of differentiation, Applicants may expect an increase in Paneth cell number following SINE treatment in vivo to be both restricted to the spatial constraints of non-hypertrophic crypts, and proportional to the total abundance of cycling progenitors, suggesting that the total increase in Paneth cell number may be modest, and require a particularly sensitive method of quantification.

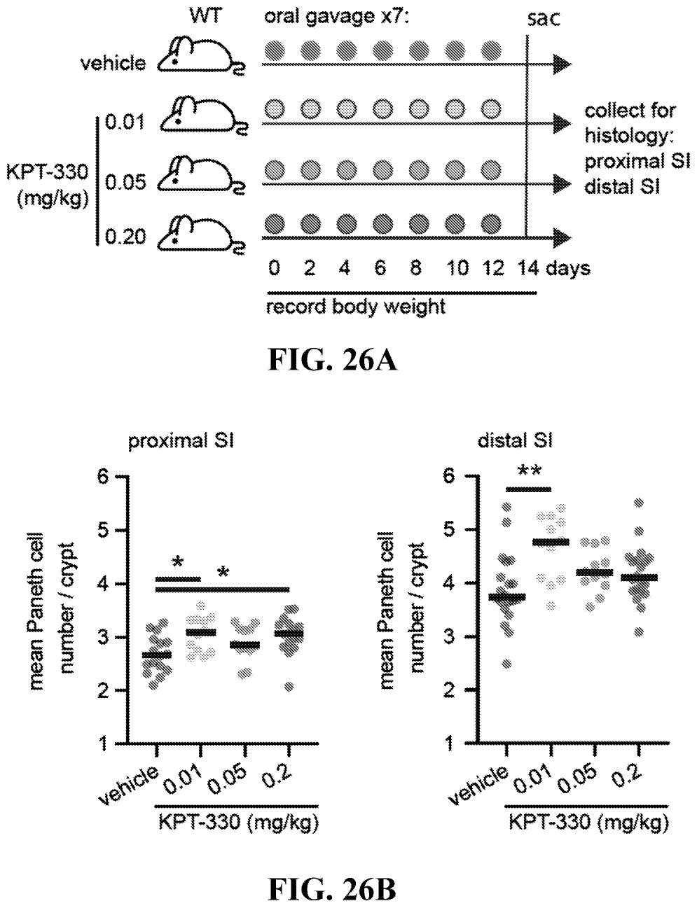

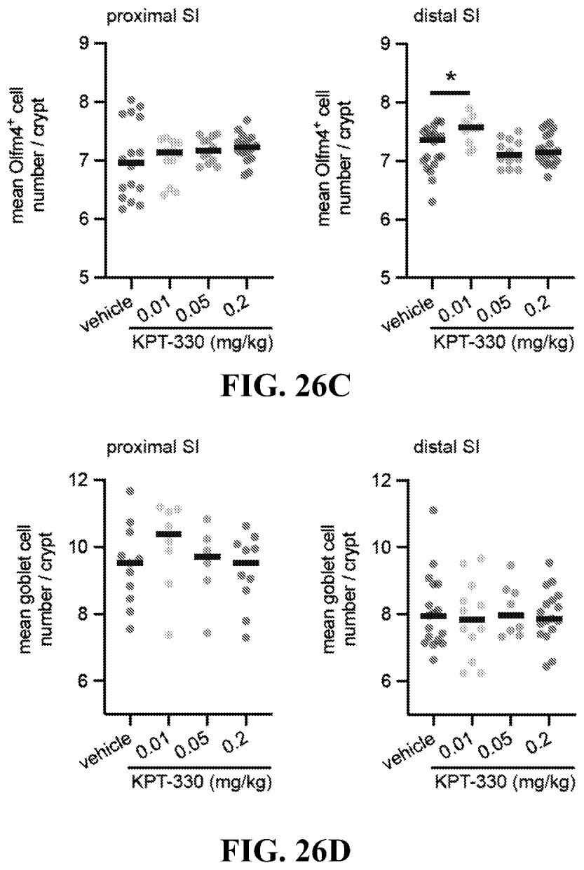

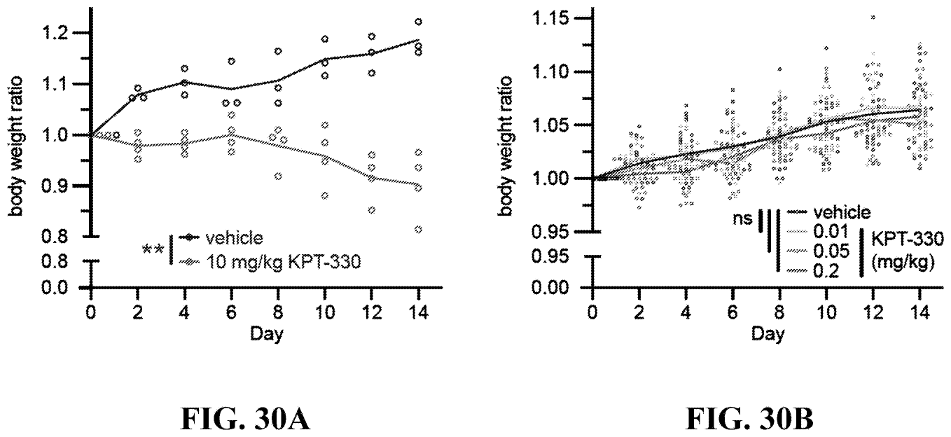

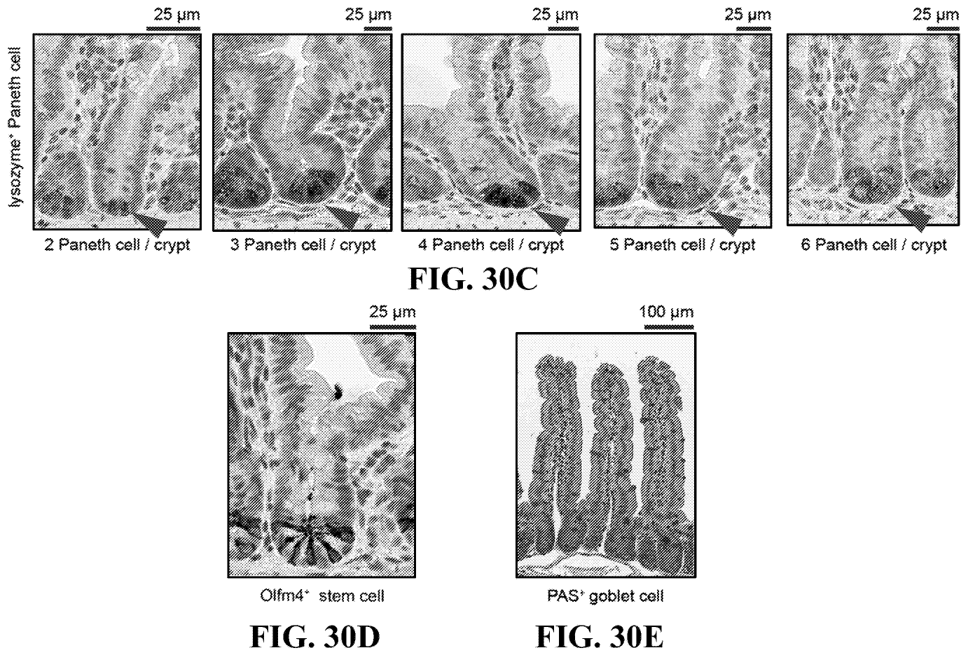

Following a similar protocol as previously reported for SINE treatment in the context of cancer (Arango et al., 2017; Azmi et al., 2013; Hing et al., 2016; Zheng et al., 2014), KPT-330 was administered at a dose 10 mg/kg via oral gavage every other day over a two-week span in C57BL/6 wild-type mice, and body weight was monitored for any clear toxicity. Within the treatment group Applicants observed significant weight loss (FIG. 30A) indicative of toxicity. Given animal weight loss on the standard chemotherapeutic dosage regimen, and additional evidence that sustained dosage of SINEs adversely impacts T cell populations (Tyler et al., 2017), Applicants sought to explore dosing regimens well below 10 mg/kg, to see if a pro-Paneth phenotype may exist below potential toxicities. Accordingly, Applicants repeated the two-week study with oral gavage of KPT-330 every other day at doses corresponding to 50-fold (0.2 mg/kg), 200-fold (0.05 mg/kg), and 1,000-fold (0.01 mg/kg) below the 10 mg/kg dose conventionally used in the setting of cancer. Applicants tracked animal weight every other day, and at day 14 collected the proximal and distal thirds of the small intestine for histological quantification of Paneth, stem, and goblet populations (FIG. 26A). In this lower-dose regime, Applicants observe no significant changes in animal weight at any dose, suggesting Applicants are outside the range of gross toxicity (FIG. 30B). Samples were prepared for histology by the ‘swiss-roll’ technique, and following staining, were blinded and randomized before manual counting of well-preserved features. Paneth cells were counted within well preserved crypts, with at minimum 30 crypts quantified per animal (representative images FIG. 30C) and then averaged to get mean Paneth cells per crypt in distal and proximal thirds of the small intestine. Compellingly, within this lower dose regime Applicants observed significant increases in Paneth abundance in both the proximal and distal small intestine at doses of 0.01 mg/kg, and proximally at 0.2 mg/kg. Applicants additionally quantified the abundance of Olfm4+ stem cells as well as PAS+ goblet cells within the same animals to ascertain whether the effect of SINE treatment was restricted to the Paneth cell compartment (representative images FIG. 30D,E). Interestingly, Applicants observe a significant increase in Olfm4+ stem cells within the distal SI at doses of 0.01 mg/kg corresponding to the group with the greatest increase in Paneth cells (FIG. 26C), suggesting a potential expansion of the stem cell niche commensurate with increased Paneth cell abundance. Applicants did not overserve any significant changes in the developmentally-related goblet cell population (FIG. 26D). In total, this data suggests the SINE-treatment may be a meaningful approach to specifically increase Paneth cell abundance in vivo, and further validates the framework for using models of organoid differentiation for small molecule screening.

### DISCUSSION

Paneth cells of the small intestine are involved in a broad range of activities including maintenance of the small intestinal epithelial barrier, shaping the gut microbiota, and communicating with the immune system. With the Paneth cell differentiation model Applicants have advanced a scalable platform to probe for drivers of Paneth cell differentiation from ISCs. Building upon previously established small molecule-driven enrichment and differentiation of LGR5+ISCs into secretory and absorptive progeny of the intestinal epithelium, Applicants set forth to characterize the secretory cells derived from WNT activation and Notch inhibition with a goal of advancing a Paneth-cell enriched culture. By assessing ISC-enriched organoid differentiation, Applicants see greatly increased markers of Paneth cells, after the described conditions.

The present invention provides motivation for the delivery of low doses of small molecules that inhibit nuclear export directly to the crypts. Additionally, there is motivation to use methods of delivery, such that low doses are delivered to the crypts for sustained periods. Applicants can test for the ideal window of measurement as PCs are long-lived. Applicants hypothesize that if PCs are measured after 2+ weeks there will be further accumulation. Applicants hypothesize that barrier function can be increased if SINEs (pro-differentiation) are combined with agents to increase the stem cell pool, such as CHIR or VPA. The results provide for the pleiotropic nature of Xpo1 inhibition, as Xpo1 inhibition was previously used as a chemotherapeutic agent at high doses.

The cells described herein show rapid transcriptional maturity and are morphologically similar to in vivo cells. The cell enrichment described herein is far superior to existing models. The organoid model enables specific investigation of the dynamics of single cell types revealing signals that would otherwise be obscured in vivo.

### Methods

Mice. Proximal small intestine was isolated from wild-type C57BL/6 mice of both sexes, aged between one and six months in all experiments.

Crypt isolation and culture. Small intestinal crypts were isolated as previously described²³. Briefly, the small intestine was harvested, opened longitudinally, and washed with ice-cold Dulbecco's Phosphate Buffer Saline without calcium chloride and magnesium chloride (PBS0) (Sigma-Aldrich) to clear the luminal contents. The tissue was cut into 2-4 mm pieces with scissors and washed repeatedly by gently pipetting the fragments using a 10-ml pipette until the supernatant was clear. Fragments were rocked on ice with crypt isolation buffer (2 mM EDTA in PBS0; Life Technologies) for 30 min. After isolation buffer was removed, fragments were washed with cold PBS0 by pipetting up and down to release the crypts. Crypt-containing fractions were combined, passed through a 70-μm cell strainer (BD Bioscience), and centrifuged at 300 rcf for 5 min. The cell pellet was resuspended in basal culture medium (2 mM GlutaMAX (Thermo Fisher Scientific) and 10 mM HEPES (Life Technologies) in Advanced DMEM/F12 (Invitrogen)) and centrifuged at 200 rcf for 2 min to remove single cells. Crypts were then cultured in a Matrigel culture system (described below) in small intestinal crypt medium (100× N2 supplement (Life Technologies), 100× B27 supplement (Life Technologies), 500× N-acetyl-L-cysteine (Sigma-Aldrich) in basal culture medium) supplemented with differentiation factors at 37° C. with 5% CO₂. Pen/strep (100×) was added for the first four days of culture post-isolation only.

Small intestinal crypts were cultured as previously described. Briefly, crypts were resuspended in basal culture medium at a 1:1 ratio with Corning™ Matrigel™ Membrane Matrix—GFR (Fisher Scientific) and plated at the center of each well of 24-well plates. Following Matrigel polymerization, 500 μl crypt culture medium (ENR+CV) containing growth factors EGF (50 ng/ml, Life Technologies), Noggin (100 ng/ml, PeproTech) and R-spondin 1 (500 ng/ml, PeproTech) and small molecules CHIR99021 (3 μM, LC Laboratories or Selleckchem) and valproic acid (1 mM, Sigma-Aldrich) was added to each well. ROCK inhibitor Y-27632 (Y, 10 μM, R&D Systems) was added for the first two days of ISC culture only. Cell culture medium was changed every other day. After 4 days of culture, crypt organoids were expanded as and enriched for ISCs under the ENR+CV condition. Expanding ISCs were passaged every 6 days in the ENR+CV condition.

Organoid culture, differentiation, and passaging. After 4 days of culture under ENR+CV condition, ISCs were differentiated to PCs. Briefly, ISC culture gel and medium were homogenized via mechanical disruption and centrifuged at 300 g for 3 min at 4° C. Supernatant was removed and the pellet resuspended in basal culture medium repeatedly until the cloudy Matrigel was almost gone. On the last repeat, pellet was resuspended in basal culture medium, the number of organoids counted, and centrifuged at 300 g for 3 min at 4° C. The cell pellet was resuspended in basal culture medium at a 1:1 ratio with Matrigel and plated at the center of each well of 24-well plates (˜250 organoids/well). Following Matrigel polymerization, 500 μl crypt culture medium (ENR+CD) containing growth factors EGF (50 ng/ml), Noggin (100 ng/ml) and R-spondin 1 (500 ng/ml) and small molecules CHIR99021 and DAPT (10 μM, Sigma-Aldrich) was added to each well. Cell culture medium was changed every other day.

High-throughput screening. For 384-well plate high-throughput screening, ISC-enriched organoids were passaged and split to single cells with TyrpLE (Thermo Fisher Scientific) and cultured for 2-3 days in ENR+CVY prior to transfer to a “2.5D” 384-well plate culture system. To prepare for “2.5D” plating, cell-laden Matrigel and media were homogenized via mechanical disruption and centrifuged at 300 g for 3 min at 4° C. Supernatant was removed and the pellet washed and spun in basal culture medium repeatedly until the cloudy Matrigel above the cell pellet was gone. On the final wash, pellet was resuspended in basal culture medium, the number of organoids counted, and the cell pellet was resuspended in ENR+CD medium at ˜7 clusters/μL. 384-well plates were first filled with 10 μL of 70% Matrigel (30% basal media) coating in each well using a Tecan Evo 150 Liquid Handling Deck, and allowed to gel at 37° C. for 5 minutes. Then L of cell-laden media was plated at the center of each well of 384-well plates with the liquid handler, and the plates were spun down at 100 g for 2 minutes to embed organoids on the Matrigel surface. Compound libraries were pinned into prepped cell plates using 50 nL pins into 30 μL media/well. Cells were cultured at 37° C. with 5% CO₂ for six days in ENR+CD medium supplemented with the tested compounds with a media change at three days. On day six, lysozyme secretion and cell viability were assessed using Lysozyme Assay Kit (EnzChek) and CellTiter-Glo 3D Cell Viability Assay (Promega), respectively, according to the manufacturers' protocols. Briefly, screen plates were washed 3× with FluoroBrite basal media (2 mM GlutaMAX and 10 mM HEPES in FluoroBrite DMEM (Thermo Fisher Scientific)) using a BioTek 406 plate washer with 10 min incubations followed by a 1 min centrifugation at 200 g to settle media between washes. After removal of the third wash, 30 μL of non-stimulated FluoroBrite basal media was added to each screen well using a Tecan Evo 150 Liquid Handling Deck from a non-stimulated treatment master plate, and plates were incubated for 30 min at 37° C. After 30 minutes, the top 15 L of media from each well of the screen plate was transferred to a non-stimulated LYZ assay plate containing 15 μL of 20× DQ LYZ assay working solution using a Tecan Evo 150 Liquid Handling Deck. The non-stimulated LYZ assay plate was covered, shaken for 10 min, incubated for 50 min at 37° C., then fluorescence measured (shake 10 s; 494 mm/518 nm) using a Tecan M1000 Plate Reader. After the media transfer to the non-stimulated LYZ assay plate, the remaining media was removed from the screen plate and 30 μL of Stimulated FluoroBrite basal media (supplemented with 10 μM CCh) was added to each screen well using a Tecan Evo 150 Liquid Handling Deck from a stimulated treatment master plate, and plates were incubated for 30 min at 37° C. After 30 minutes, the top 15 μL of media from each well of the screen plate was transferred to a stimulated LYZ assay plate containing 15 μL of 20× DQ LYZ assay working solution using a Tecan Evo 150 Liquid Handling Deck. The stimulated LYZ assay plate was covered, shaken for 10 min, incubated for 50 min at 37° C., then fluorescence measured (shake 10 s; 494 mm/518 nm) using a Tecan M1000 Plate Reader. Finally, 8 μL of CTG 3D was added to each well of the screen plate, which was shaken for 30 min at room temperature, then luminescence read (shake 10 s; integration time 0.5-1 s) to measure ATP.

Primary screens were performed using the Target Selective Inhibitor Library (Selleck Chem). Assays were performed in triplicate using four compound concentrations (0.08, 0.4, 2, and 10 μM).

Screen Analysis. A custom R script and pipeline was used for analysis of all screen results. Results (excel or .csv files) were converted into a data frame containing raw assay measurements corresponding to metadata for plate position, treatments, doses, cell type, and stimulation. Raw values were log₁₀ transformed, then a LOESS normalization was applied to each plate and assay to remove systematic error and column/row/edge effects using the formula (Mpindi et al., 2015):

\hat{x}ᵢⱼ=xᵢⱼ−(loess.fitᵢⱼ−median(loess.fitᵢⱼ)) (1)

where \hat{x}ᵢⱼ is the loess fit result, xᵢⱼ is the log₁₀ transformed value at row i and column j, and loess. fitᵢⱼ is the value from loess smoothed data at row i and column j calculated using R loess function with span 1.

Following LOESS normalization, a plate-wise fold change (FC) calculation was performed on each well to normalize plates across the experiment. This was calculated by subtracting the median of the plate (as control) from the LOESS normalized values:

FCᵢⱼ=\hat{x}ᵢⱼ−median(\hat{x}ᵢⱼ) (2)

Replicate strictly standardized mean difference (SSMD) was used to determine the statistical effect size of each treatment in each assay (treatment and dose grouped by replicate, n=3) relative to the plate using the formula for the robust uniformly minimal variance unbiased estimate (UMVUE) (Zhang, 2011):

$$SSMD = \frac{Γ ( \frac{n - 1}{2} )}{Γ ( \frac{n - 2}{2} )} \sqrt{\frac{2}{n - 1}} \frac{\overline{{d}_{i}}}{\sqrt{{w}_{i} {s}_{i}^{2} + {w}_{0} {s}_{0}^{2}}} \qquad ( 3 )$$

where dᵢ and sᵢ are respectively the sample mean and standard deviation of dᵢⱼs where dᵢⱼ is the FC for the ith treatment on the jth plate. Γ(·) is a gamma function. s₀² is an adjustment factor equal to the median of all sᵢ²s to provide a more stable estimate of variance. wᵢ and w₀ are weights equal to 0.5 with the constraint of wᵢ+w₀=1. n is the replicate number.

Mean FC (the arithmetic mean of all samples grouped by treatment and dose across replicates) was used to determine the z-score for each treatment and dose with the formula:

$$z = \frac{meanFC}{{SD}_{pop}} \qquad ( 4 )$$

$$where {SD}_{pop} is the standard deviation of all mean - FC ’ s .$$

All calculated statistics were combined in one finalized data table and exported as a .csv file for hit identification. A primary screen “hit” was defined as having SSMDs for both LYZ assays greater than the optimal critical value (β_{α}_{₁}=0.997) and being in the top 10% of a normal distribution of FC values for both assays with a z-score cutoff >1.282. β_{α}_{₁} was determined by minimizing the false positive (FPL) and false negative (FNL) levels for up-regulation SSMD-based decisions by solving for the intersection of the formulas (Zhang, 2011):

$${F}_{t ( n - 1 , \sqrt{n} {β}_{2} )} ( \frac{{β}_{{α}_{1}}}{k} ) = 1 - FPL \qquad ( 5 )$$

$$and FNL = {F}_{t ( n - 1 , \sqrt{n} {β}_{1} )} ( \frac{{β}_{{α}_{1}}}{k} ) \qquad ( 6 )$$

$$where k = \sqrt{\frac{1}{n}} \qquad ( 7 )$$

where Fₜ(n−1, \sqrt{nβ})(·) is the cumulative distribution function of non-central t-distribution t(n−1, \sqrt{nβ}) and n is the number of replicates, β₂ is a SSMD bound for FPL of 0.25 (at least very weak effect), and β₁ is a SSMD bound for FNL of 3 (at least strong effect).

Hit treatments were thus selected to have a well-powered statistical effect size as well as a strong biological effect size. Optimal dose per hit treatment was determined by SSMD for both LYZ assays.

Secondary lysozyme secretion assay screen. Confirmatory secondary screening with primary hits was performed using the above 384-well plate method. The screen was conducted with 4-plate replicates with a base media of ENR+CD. Media was supplemented with compound at day 0 and day 3 (n=8 well replicates per dose) at four different doses: two-fold above, two-fold below, and four-fold below the optimal final dose for each respective treatment. Additionally, each plate carried a large number of ENR+DMSO or ENR+CD+DMSO (vehicle) control wells (n=100 for ATP, and n=25 for LYZ.NS and LYZ.S) for robust normalization. ATP, non-stimulated lysozyme activity and CCh-stimulated lysozyme activity was again measured and the collected data was again processed in a custom R-script, per primary screen with slight modification. Values were log₁₀ transformed, and a plate-wise FC was calculated for each well based on the median value of ENR+CD+DMSO (vehicle) control wells to normalize plate to plate variability. The following formula was used:

FCᵢⱼ=xᵢⱼ−median(x_{POS}) (8)

Where xᵢⱼ is the log₁₀ transformed value at row i and column j, and x_{POS} are the values of the positive control wells. For the ATP assay, all vehicle-only wells were used as the control. For the LYZ.NS assay, non-stimulated vehicle only wells were used. For the LYZ.S assay, vehicle only wells that were non-stimulated in the LYZ.NS assay then stimulated in the LYZ.S were used.

Once normalized, the replicate SSMD was calculated using formula (3) to quantify statistical effect size with 8 replicate differences taken relative to the respective plate ENR+DMSO or ENR+CD+DMSO median value. A primary hit was considered validated when SSMDs for both LYZ assays was greater than the optimal critical value (β_{α}_{₁}) of 0.889. β_{α}_{₁} was determined using formula (5) with an FPL error of 0.05 for a more stringent cut off, FNL was not considered. Optimal doses were chosen for treatments with multiple validated doses by taking the most potent (highest mean fold change relative to ENR+CD control) dose in both LYZ assays.

Lysozyme secretion assay. ISC-enriched organoids in 3D Matrigel culture were passaged to a 48- or 96-well plate and cultured with ENR or ENR+CD media containing DMSO or each drug for 6 days. DMSO- or drug-containing media were changed every other day. On day 6, cells were washed with basal media twice and treated with basal media with or without 10 μM carbamoylcholine chloride for 3 h in a CO₂ incubator at 37° C. A part of the conditioned media was collected and used for lysozyme assay (Thermo, E-22013) following the manufacturer's instruction. The fluorescence was measured using excitation/emission of 485/530 nm. CellTiter-Glo 3D Reagent (Promega, G9681) was added afterward, and the cell culture plate was incubated on an orbital shaker at RT for 30 min to induce cell lysis and to stabilize the luminescent signal. The solution was replaced to a 96-well white microplate, and luminescent signals were measured by a microplate reader (infinite M200, Tecan). The standard curve was prepared by diluting recombinant ATP (Promega, P1132). For both assays, a polynomial cubic curve was fitted to a set of standard data, and each sample value was calculated on the Microsoft Excel.

Flow cytometry. ISC-enriched organoids in 3D Matrigel culture were passaged to a 48-well plate and induced differentiation for 6 days by ENR+CD media containing DMSO or each drug indicated in the figures. DMSO- or drug-containing media were changed every other day. On day 6, cells were washed twice with basal media, then harvested from Matrigel by the mechanical disruption in TrypLE Express (Thermo, #12605010) to remove Matrigel and dissociate organoids to single cells. After vigorous pipetting and incubation at 37° C. for 15 min, the cell solution was diluted twice with basal media and centrifuged at 300 ref for 3 min. The cell pellet was resuspended in FACS buffer (PBS containing 2% FBS) and replaced into a 96-well Clear Round Bottom Ultra-Low Attachment Microplate (Corning, #7007). The cell solution was centrifuged again at 300 rcf for 3 min at 4° C. to pellet the cells. Cells were stained with Zombie-violet dye (BioLegend, #423113) at 100× for viability staining for 20 min at RT in the dark. After centrifugation for 3 min at 300 rcf, cells were fixed in fixation buffer (FACS buffer containing 1% formaldehyde (Thermo, #28906)) for 15 min on ice in the dark. Cells were centrifuged again for 3 min at 300 rcf and blocked with staining buffer (FACS buffer containing 0.5% Tween20 (Sigma, P2287)) for 15 min at RT in the dark. Pelleted cells by the centrifugation for 3 min at 300 rcf are stained with FITC-conjugated anti-lysozyme antibody (Dako, F0372) and APC-conjugated anti-CD24 antibody (Biolegend, #138505) at 100× for 45 min at RT in the dark. The cell pellet was washed once with FACS buffer, resuspended in FACS buffer, and filtered through 5 mL test tube with cell strainer snap cap (Corning, #352235). Flow cytometry was performed using an LSR Fortessa (BD; Koch Institute Flow Cytometry Core at MIT). Flow cytometry data were analyzed using FlowJo X v10.6.1 software.

Western blotting. Organoid-containing gel was homogenized in basal medium and centrifuged at 300 rcf for 3 min. Organoid pellet was lysed with ice-cold Pierce IP Lysis Buffer (Thermo Fisher Scientific, #87787) containing Halt Protease Inhibitor Cocktail, EDTA-Free (Thermo Fisher Scientific, #87785) and incubated on ice for 20 min. The lysate was centrifuged at 17,000 rcf for 10 min, and the supernatant was combined with NuPAGE LDS Sample Buffer (Thermo Fisher Scientific, NP0007). Protein concentration was determined by Pierce 660 nm Protein Assay (Thermo Fisher Scientific, #22660) and normalized to the lowest concentration among each sample set. Samples were incubated at 70° C. for 10 minutes and resolved by SDS-PAGE using NuPAGE 4-12% Bis-Tris Protein Gels (Thermo Fisher Scientific) followed by electroblotting onto Immun-Blot PVDF Membrane (Biorad, 1620174) using Criterion Blotter with Plate Electrodes (Biorad, #1704070). The membranes were blocked with 2% Blotting-Grade Blocker (Biorad, 1706404) in TBS-T (25 mM Tris-HCl, 140 mM NaCl, 3 mM Potassium Chloride and 0.1% Tween 20) and then probed with appropriate antibodies, diluted in TBS-T containing 2% BSA (Sigma, A7906) and 0.05% sodium azide (Sigma, #71289). The primary antibody against lysozyme was purchased from Abcam (ab108508). HRP-linked anti-rabbit IgG antibodies were purchased from Cell Signaling Technology (#7074). Chemiluminescent signals were detected by LAS4000 (GE Healthcare) using Amersham ECL Select Western Blotting Detection Reagent (GE Healthcare, #45-000-999), and total protein signals were obtained by Odyssey Imaging System (LI-COR Biosciences) using REVERT Total Protein Stain Kit (LI-COR Biosciences, #926-11010).

Animal study. 8-10 weeks old wild type C57BL/6NCrl male mice (#027) were purchased from Charles River. Mice were housed under 12 h light/dark cycle and provided food and water ad libitum. 0.01, 0.05, 0.2 or 10 mg/kg of KPT-330 were injected orally using a disposable gavage needle (Cadence Science, #9921) at 10 μL/g weight. KPT-330 was dissolved in DMSO initially and further diluted in sterile PBS containing Pluronic F-68 Non-ionic Surfactant (Gibco, #24040032) and Polyvinylpyrrolidone (PVP, Alfa Aesar, A14315, average M.W. 58,000); the final concentration of DMSO is 2%, Pluronic is 0.5%, and PVP is 0.5%. KPT-330 was administered every other day for two weeks, 7 injections in total (days 0, 2, 4, 6, 8, 10, 12), and mice were sacrificed at day 14. All animal studies are approved by the Committee on Animal Care (CAC) at Massachusetts Institute of Technology.

Histology. The small intestine (SI) was collected from mice and divided into three parts. Only proximal and distal SI were kept in PBS, and medial SI was discarded. Each SI was opened longitudinally and washed in PBS. SI was rolled using the Swiss-rolling technique and incubated in 10% Neutral Buffered Formalin (VWR, 10790-714) for 24 h at RT. Fixed tissues were embedded in paraffin, and 4 μm sections were mounted on slides. For immunohistochemistry, slides were deparaffinized, antigen retrieved using heat-induced epitope retrieval at 97° C. for 20 min using citrate buffer pH 6, and probed with appropriate antibodies followed by DAB staining. For McManus Periodic Acid Schiff (PAS) reaction, slides were deparaffinized, oxidized in periodic acid, and stained with Schiff reagent (Poly Scientific, s272) followed by counterstaining with Harris Hematoxylin. Slides were scanned by Aperio Slide Scanner (Leica) and cells were counted on Aperio eSlide Manager. Slides were randomized before counting, and all cell types were counted in all well-preserved crypts along the longitudinal crypt-villus axis.

Single-cell RNA-sequencing. A single-cell suspension was obtained from organoids cultured under conditions for the differentiation time course as described above. Applicants utilized the Seq-Well platform for massively parallel scRNA-seq to capture transcriptomes of single cells on barcoded mRNA capture beads. Full methods on implementation of this platform are available in Hughes, et al. (2019). Highly Efficient, Massively-Parallel Single-Cell RNA-Seq Reveals Cellular States and Molecular Features of Human Skin Pathology. BioRxiv 689273. In brief, 20,000 cells from one organoid condition were loaded onto one array containing 100,000 barcoded mRNA capture beads. The loaded arrays containing cells and beads were then sealed using a polycarbonate membrane with a pore size of 0.01 μm, which allows for exchange of buffers but retains biological molecules confined within each microwell. Subsequent exchange of buffers allows for cell lysis, transcript hybridization, and bead recovery before performing reverse transcription en masse. Following reverse transcription and exonuclease treatment to remove excess primers, PCR amplification was carried out using KAPA HiFi PCR Mastermix with 2,000 beads per 50 μL reaction volume. Libraries were then pooled and purified using Agencourt AMPure XP beads (Beckman Coulter, A63881) by a 0.6×SPRI followed by a 0.8×SPRI and quantified using Qubit hsDNA Assay (Thermo Fisher). Libraries were constructed using the Nextera Tagmentation method on a total of 800 pg of pooled cDNA library per sample. Tagmented and amplified sequences were purified at a 0.6×SPRI ratio yielding library sizes with an average distribution of 650-750 base pairs in length as determined using the Agilent hsD1000 Screen Tape System (Agilent Genomics). Arrays were sequenced with an Illumina NovaSeq System. The read structure was paired end with Read 1 starting from a custom read 1 primer containing 20 bases with a 12 bp cell barcode and 8 bp unique molecular identifier (UMI) and Read 2 being 50 bases containing transcript information.

Single-cell RNA-sequencing computational pipelines and analysis. Read alignment was performed as in (Macosko et al., 2015). Briefly, for each sequencing run, raw sequencing data was converted to demultiplexed FASTQ files using bcl2fastq2 based on Nextera N700 indices corresponding to individual samples/arrays. Reads were then aligned to mm10 genome using the Drop-Seq tools v2.3 on the Tera portal maintained by the Broad Institute using standard settings. Individual reads were tagged according to the 12-bp barcode sequencing and the 8-bp UMI contained in Read 1 of each fragment. Following alignment, reads were binned onto 12-bp cell barcodes and collapsed by their 8-bp UMI. Digital gene expression matrices (e.g. cell by gene tables) for each sample were obtained from quality filtered and mapped reads and UMI-collapsed data, are deposited in GSE100274, and were utilized as input into Seurat v3 for further analysis.

To analyze ENR+CV, ENR, and ENR+CD organoids together, Applicants merged UMI matrices across all genes detected in any condition and generated a matrix retaining all cells with at least 1000 UMI detected. This table was then utilized to setup the Seurat object in which any cell with at least 400 unique genes was retained and any gene expressed in at least 5 cells was retained. The object was initiated with log-normalization, scaling, and centering set to True. Before performing dimensionality reduction, data was subset to include cells with less than 8,000 UMI, and a list of 1,676 most variable genes was generated by including genes with an average normalized and scaled expression value greater than 0.14 and with a dispersion (variance/mean) greater than 0.4. The total number of ENR+CV, ENR, and ENR+CD cells included in the analysis was 985, 2,544, and 2,382, respectively with quality metrics for nGene, nUMI, and percentage of ribosomal and mitochondrial genes reported in FIG. 30. Applicants then performed principal component analysis over the list of variable genes. For both clustering and t-stochastic neighbor embedding (tSNE), Applicants utilized the first 12 principal components based on the elbow method, as upon visual inspection of genes contained within, each contributed to important biological processes of intestinal cells. Applicants used FindClusters with a resolution of 1.35 and 1000 iterations of tSNE to identify 14 clusters across the 3 input samples. To identify genes which defined each cluster, Applicants performed a ROC test implemented in Seurat with a threshold set to an AUC of 0.60.

Transcriptional Scoring. To determine the fractional contribution to a cell's transcriptome of a gene list, Applicants summed the total log(scaled UMI+1) expression values for genes within a list of interest and divided by the total amount of scaled UMI detected in that cell giving a proportion of a cell's transcriptome dedicated to producing those genes. From the proteomic screen, Applicants took a list of upregulated proteins (249) or downregulated proteins (212) that were detected within the single-cell RNA-sequencing data. To determine the relationship to in vivo Paneth cells and EECs, Applicants took reference data from two Seq-Well experiments run on epithelial cells dissociated from the ileal region of the small intestine of two C57BL/6J mice run in separate experiments. Ileum was first rinsed in 30 mL of ice cold PBS and allowed to settle. The segment was then sliced with scissors and transferred to 10 mL epithelial cell solution (HBSS Ca/Mg-Free 10 mM EDTA, 100 U/mL penicillin, 100 μg/mL streptomycin, 10 mM HEPES, 2% FCS (ThermoFisher)) freshly supplemented with 200 μL of 0.5 M EDTA. The epithelial separation from the underlying Lamina propria was performed for 15 minutes at 37° C. in a rotisserie rack with end-over-end rotation. The tube was then removed and placed on ice immediately for 10 minutes before shaking vigorously 15 times. Visual macroscopic inspection of the tube at this point should yield visible epithelial sheets, and microscopic examination confirms the presence of single-layer sheets and crypt-villus structures. The epithelial fraction was spun down at 400 g for 7 minutes and resuspended in 1 mL of epithelial cell solution before transferring to a 1.5 mL Eppendorf tube to minimize time spent centrifuging. Cells were spun down at 800 g for 2 minutes and resuspended in TrypLE Express for 5 minutes in a 37° C. bath followed by gentle trituration with a P1000 pipette. Cells were spun down at 800 g for 2 minutes and resuspended in ACK lysis buffer (ThermoFisher) for 3 minutes on ice to remove red blood cells and dying cells. Cells were spun down at 800 g for 2 minutes and resuspended in 1 mL of epithelial cell solution and placed on ice for 3 minutes before triturating with a P1000 pipette and filtering into a new Eppendorf through a 40 μm cell strainer (Falcon/VWR). Cells were spun down at 800 g for 2 minutes and then resuspended in 200 μL of epithelial cell solution and placed on ice for counting. Single-cell RNA-seq data was then generated as described in (Single-cell RNA-sequencing and Single-cell RNA-sequencing computational pipelines and analysis) sections of methods. To generate Paneth and EEC signatures, Applicants ran unbiased SNN-graph based clustering, performed a ROC test, identified the two mature Paneth and EEC clusters, and report all genes with an AUC above 0.60, and use all genes with an AUC above 0.65 for scoring, within each cluster (gene lists in Table 6) representing any gene with enrichment in Paneth and EE cells. These lists capture genes which are enriched in Paneth (Lyz-high) and EE (Chga-high) cells and separate them from the rest of the cells present in intestinal epithelium. For pathway analysis, Applicants inspected curated gene lists deposited in the GSEA platform and used KEGG-derived Wnt and Reactome-derived Notch and Respiratory Electron Transport Chain signatures (Table 3B). In vivo transcription factors for PCs and EECs were determined by matching the PC and EEC signature gene sets with transcription factors from the Riken Transcription Factor Database (TFdb—genome.gsc.riken.jp/TFdb/), and then including only those TFs which were robustly identified in the proteome dataset.

TABLE 6
Marker genes from organoid differentiation time course single-cell RNA-seq,
as determined by Wilcoxon differential expression testing of cluster versus
rest. The list of genes was obtained using the following significance cut-
offs: false discovery rate (FDR) <0.05, Log2 fold-change >0.5.

| stem I | stem II | stem III | early enterocyte | enterocyte |
|---|---|---|---|---|
| Nkd1 | Cps1 | Mki67 | Dmbt1 | Cyp3a11 |
| Prox1 | Clca4 | Top2a | Krt19 | Cyp2c29 |
| Ifitm3 | Ncl | Tubb5 | Gstm3 | Aldob |
| Smoc2 | Ppp1r1b | Smc4 | Ces2a | Apoa4 |
| Hmgcs2 | Rplp1 | Cenpe | Fabp1 | Cyp2b10 |
| Lgr5 | Rps26 | Ccna2 | Akr1c19 | Sis |
| Cfi | Rpl32 | Kif11 | St3gal4 | Apoa1 |
| Ifitm1 | Hsp90ab1 | Cps1 | Phgr1 | Fabp1 |
| Axin2 | mt-Rnr1 | Tpx2 | Lgals4 | Prap1 |
| Vim | Rpl13 | Cenpf | Pycard | Gsta1 |
| Nav1 | Rps19 | Rrm1 | Coro2a | Gstm3 |
| Tnfrsf19 | Gpx2 | Smc2 | Akr1b8 | Aldh1a1 |
| Cachd1 | Tkt | Cdca3 | Ccl25 | Ces2a |
| Pdgfa | Npm1 | Ncapd2 | Cyp2c66 | S100g |
| Rnf43 | Hook1 | Cdca8 | Mttp | Mttp |
| Rgmb | Gas5 | Nucks1 | Ces2e | Adh1 |
| Pls3 | Rpl14 | Ube2c | Gsta4 | Cyp2d26 |
| Tns3 | Rps18 | Tmpo | Cbr3 | Alpi |
| Slc12a2 | Rps20 | D17H6S56E-5 | Cdh17 | Gstm1 |
| Gtf2i | Mybbp1a | Ckap2l | Gsta1 | Fabp2 |
| Sox4 | Rps24 | Ranbp1 | Tm4sf5 | Khk |
| Ogt | Shmt1 | Ncl | Lgals2 | Apoc2 |
| Utrn | Rps21 | Incenp | Gstm1 | Dak |
| Lgr4 | Pdgfa | Clca4 | Hsd17b2 | Cyp4f14 |
| Malat1 | Anp32b | Anp32b | Abcb1a | Cyp2c66 |
| Gif | Cdca7 | Racgap1 | Mgat4a | Reg1 |
| Gpld1 | Rps10 | Kif20b | Mgst2 | Fth1 |
| Lmo7 | Kcnq1 | Kif15 | Ugdh | 2210404007Rik |
| Soat1 | Mcm6 | Casc5 | Aoc1 | Cyp3a13 |
| Txnip | 1500012F01Rik | Plk1 | Mt1 | Slc5a1 |
| Ppap2b | Hmgn1 | Lig1 | Mt2 | Ces2e |
| Adcy1 | Mcm3 | Birc5 | Ces1f | Cyp3a25 |
| Kif12 | Cluh | Dnmt1 | Mgst3 | Cyb5 |
| Sema3c | Amica1 | Dek | Adh1 | Papss2 |
| Tead2 | Gm10800 | Ect2 | Rgn | Ephx1 |
| Vwa2 | Sdc4 | Ptma | Fzd5 | Ace2 |
| Robo1 | Cftr | Uhrf1 | Acot1 | 2010106E10Rik |
| Cdo1 | Reg3g | Cdca2 | Slc39a5 | Adh6a |
| Marcksl1 | | Tuba1b | Cyb5 | Scd1 |
| Nedd4 | | Ccdc34 | Myo1a | Reg3b |
| Slc14a1 | | Kif23 | Ces2g | Maoa |
| Myh9 | | Cdk1 | Cyp2c55 | Gpd1 |
| D930015E06Rik | | Anln | Ckmt1 | Creb3l3 |
| Lamb2 | | Ncapg | 2200002D01Rik | Apob |
| Rev3l | | Hook1 | Gpx1 | Ccl25 |
| Kcnq1 | | Ncaph | Slc26a3 | Fmo5 |
| Cers4 | | Bub1b | Cbr1 | Cyp2j6 |
| Prom1 | | Kpnb1 | Ugt2b34 | Fam213a |
| Ets2 | | Cbx5 | Scd1 | Cyp2c55 |
| Zfp618 | | 2810417H13Rik | Fabp2 | Tm4sf20 |
| Sp5 | | Mcm5 | S100g | Pigr |
| Ttc3 | | Kif4 | Enpep | Oat |
| Flna | | Atad2 | Dbi | Pklr |
| Sorbs2 | | Tacc3 | Cyb5b | 4931406C07Rik |
| Zfp503 | | Hmmr | Sult1d1 | Ugdh |
| Arhgef26 | | Hjurp | Isx | Cyp2c65 |
| Ldb1 | | Ssrp1 | Dgat1 | Rbp2 |
| Cdk4 | | Shmt1 | Pigr | Acsl5 |
| Irx5 | | Ppp1r1b | Gsta3 | Ces1f |
| Id3 | | Aspm | Alpi | Gsta4 |
| Ttr | | Rangap1 | Pcsk5 | Al747448 |
| Ctnnb1 | | Tubb4b | Arg2 | Slc2a5 |
| Rbms3 | | Prc1 | Nlrp6 | Mt1 |
| Itm2b | | Pbk | Cyp2c65 | Ugt2b34 |
| Cpn1 | | Mcm6 | Ano6 | Enpep |
| Cers6 | | Lbr | Lrp1 | Guca2b |
| Pkm | | Knstrn | Mal2 | Clic5 |
| Ptprf | | Lmnb1 | Pgd | Areg |
| Zmynd8 | | Cluh | Atp5b | Akr1c19 |
| Thra | | Topbp1 | Otc | Mme |
| Psap | | Cenpa | Plcb3 | Akp3 |
| Adcy6 | | Kif20a | Ugt2b5 | Mgst2 |
| Tmc4 | | Anp32e | Npc1l1 | Mgam |
| D4Wsu53e | | Tcof1 | Klf5 | Mep1b |
| Ddx17 | | Nusap1 | Bpnt1 | Arg2 |
| Aplp2 | | Arl6ip1 | Aldoa | Retsat |
| Igfbp4 | | Oxct1 | Rbp2 | Krt20 |
| Irf2bp2 | | Hnrnpab | Abcc3 | Ugt2b5 |
| Fam13a | | Mcm7 | Ugt1a7c | Gm766 |
| Mex3a | | Rrm2 | Rplp1 | Aoc1 |
| Ehf | | Sae1 | Chchd10 | Sepp1 |
| Phldb2 | | Rad21 | Mdh2 | Cryl1 |
| Dsp | | Usp1 | Cds1 | Cyb5r3 |
| Npc2 | | Mcm3 | Acaa2 | Ckmt1 |
| Sorcs2 | | Serbp1 | Aadac | Mogat2 |
| Rbm5 | | Pa2g4 | Ccnd2 | Cideb |
| Dtx4 | | Ipo5 | Bche | Ggt1 |
| Cyr61 | | Ckap5 | Ppp1r14d | Exoc3l4 |
| Lphn2 | | Gpx2 | Uqcr11 | Tm4sf5 |
| Sdc4 | | Cdc20 | Serpinb1a | 43892 |
| Peg3 | | Dut | Uqcrq | Fbp2 |
| Arl4c | | Hist1h1b | Serpinb6a | Hsd17b11 |
| Sfrs18 | | Npm1 | Akr1c13 | Slc11a2 |
| Clu | | Ybx1 | Hadha | Ugt2a3 |
| Prrc2b | | Ybx3 | Ckb | Reg3a |
| Atrx | | Nasp | Idh1 | Bche |
| Pbx1 | | Ckb | Tkt | Dbi |
| Chd6 | | Melk | Ugt1a1 | Mgst1 |
| Tcf12 | | Mybbp1a | Hkdc1 | Tm6sf2 |
| Plekhg3 | | Hells | Txn1 | Gna11 |
| Klhl24 | | Hspd1 | Cluh | H2-Q2 |
| Mfge8 | | Tyms | Cyp2j6 | Galm |
| Zfp266 | | C330027C09Rik | Hsd17b11 | Ano6 |
| Zfp292 | | Set | Prpsap1 | Reep6 |
| Cdk6 | | Hspa9 | Hsd17b13 | Acox1 |
| Grn | | Nhp2 | Lypd8 | Ephx2 |
| Echdc2 | | Smc1a | Cpt1a | Mt2 |
| 9330162012Rik | | Ndc1 | Anxa13 | Cyp4v3 |
| Ddx58 | | Tkt | Aldh9a1 | Akr1b7 |
| Nfib | | Nop58 | Sema4g | Slc2a2 |
| Hdac11 | | C1qbp | Mgst1 | St3gal4 |
| ATF7 | | Cit | Atpif1 | Gpi1 |
| Ifitm2 | | Whsc1 | Nomol | Sult1b1 |
| Jarid2 | | Pcsk9 | Vdr | Lct |
| Wdr6 | | Phb2 | Ppp1r1b | Hadh |
| Itpr2 | | Banf1 | 2010001M06Rik | Idh1 |
| Tia1 | | Fgfbp1 | Gna11 | Sult1d1 |
| Zfp704 | | Srsf1 | Gsr | Npc1l1 |
| Ago1 | | Spag5 | Hnf4g | Btnl1 |
| Gaa | | Arhgap11a | Elovl6 | 2200002D01Rik |
| Mpp3 | | Mcm10 | Mvp | Itfg3 |
| Lpp | | Hmgb2 | Acadvl | Slc40a1 |
| Itga6 | | Tcp1 | Ldha | Cyb5b |
| Man2b1 | | Ccnb2 | Etfb | Slc39a5 |
| Bex1 | | Nup133 | Anxa2 | Abcc2 |
| Rec8 | | Ezh2 | Nfe2l2 | Cndp2 |
| Anpep | | Dctpp1 | Itfg3 | Cbr1 |
| Fnbp1l | | Add3 | Cd38 | Eps8l3 |
| Pld3 | | Dlgap5 | Tmem54 | Cgref1 |
| Lrig1 | | Vars | Ahcyl2 | Pgrmc1 |
| Mecom | | Ncapg2 | Ifi27l2b | Abcg5 |
| Hspa4l | | Clspn | Net1 | Gstm4 |
| Tbx3 | | Uqcr10 | Hadh | Ndrg1 |
| Clca4 | | Foxm1 | Hnf4a | Chp2 |
| Sox9 | | Nap1l1 | Cox7b | G6pc |
| Zfp703 | | Ddx21 | Rpl13 | Mpp1 |
| Lbh | | Tomm40 | Mtch2 | Acaa2 |
| Cxadr | | Acat1 | Por | Cdhr2 |
| Krt23 | | Apex1 | Lad1 | Gsto1 |
| | | Ckap2 | Cox6b1 | Entpd5 |
| | | Hnrnpa2b1 | Egln3 | Plac8 |
| | | Dkc1 | Uqcr10 | Atp1b1 |
| | | Supt16 | Fam162a | Slc9a3rl |
| | | Pcna | Eps8l3 | Sar1b |
| | | Naa50 | Reep6 | Bco2 |
| | | Spc24 | Cox6c | Cdr2 |
| | | Mrpl12 | Slc35g1 | Abcg8 |
| | | Cftr | Acadl | Cda |
| | | Hnrnpu | Hbegf | Ldha |
| | | Park7 | Sdhb | Dhrs1 |
| | | Rpa1 | Ccdc88c | Abcb1a |
| | | Dbf4 | Cldn2 | Mdh2 |
| | | Myb | Ech1 | Srxn1 |
| | | Hirip3 | Abcc2 | Spink3 |
| | | Aurka | Myo1d | Mtch2 |
| | | Esco2 | Pfkp | Me1 |
| | | Chaf1a | Pank3 | Arl4a |
| | | Mcm4 | Ndrg1 | Dgat1 |
| | | Rfc5 | | Rdh7 |
| | | 2700094K13Rik | | Pbld2 |
| | | Psat1 | | Acp5 |
| | | Tomm70a | | Lpcat3 |
| | | Mcm2 | | Gsta3 |
| | | Ppm1g | | Fbln1 |
| | | Nudc | | Dgat2 |
| | | Bzw2 | | Hsd17b4 |
| | | Snx5 | | Hnf4g |
| | | Cyc1 | | Cat |
| | | Rbbp7 | | Anxa2 |
| | | Rplp1 | | Por |
| | | Gart | | Bpnt1 |
| | | Prmt5 | | Ehhadh |
| | | Rpl13 | | Sord |
| | | Timm50 | | Anpep |
| | | Nup85 | | Ms4a10 |
| | | Eftud2 | | Myo1a |
| | | Espl1 | | Acadl |
| | | Fads1 | | Slc35g1 |
| | | Noc2l | | Acaa1b |
| | | Brca2 | | B2m |
| | | Cct6a | | Hsd17b13 |
| | | Fen1 | | Scin |
| | | Sgol1 | | Slc6a19 |
| | | Prim1 | | Xdh |
| | | Gmnn | | Mdh1 |
| | | E2f8 | | Sec23a |
| | | Nek2 | | Cbr3 |
| | | Dnajc9 | | Txn1 |
| | | Mthfd1 | | Pcsk5 |
| | | Dtymk | | Pls1 |
| | | Hnf4a | | Coro2a |
| | | Rpl14 | | Cldn15 |
| | | Aqp4 | | Hdhd3 |
| | | Bub1 | | Dusp5 |
| | | Rps2 | | Tmem236 |
| | | Spc25 | | Gpt |
| | | Pole | | Tmbim6 |
| | | Kif22 | | Rnd2 |
| | | Iqgap3 | | Nqo1 |
| | | Asf1b | | Atp2b1 |
| | | Rps26 | | Slc25a45 |
| | | Mis18bp1 | | Crip1 |
| | | Mbnl1 | | Vdr |
| | | Cep55 | | Slc26a6 |
| | | Kcnq1 | | Abcc3 |
| | | Cct2 | | 2010002M12Rik |
| | | Fkbp4 | | Ces2c |
| | | Rpl8 | | 2010003K11Rik |
| | | Atp5b | | Cdh17 |
| | | Cotl1 | | Cdhr5 |
| | | Nsun2 | | H2-Q10 |
| | | Nop56 | | Ifi30 |
| | | Lyar | | Slc6a20a |
| | | Hspa4 | | Slc27a2 |
| | | Lsm4 | | Hkdc1 |
| | | Fus | | Ugt1a1 |
| | | Shmt2 | | Slc26a3 |
| | | Srsf2 | | Slc3a1 |
| | | Lonp1 | | H2-T3 |
| | | Nfib | | Lrrc19 |
| | | Nolc1 | | Crot |
| | | H1f0 | | Ank |
| | | Hmgn1 | | Plk3 |
| | | Hist1h1e | | Phyh |
| | | Gsr | | Lpgat1 |
| | | Tagln2 | | Slc20a1 |
| | | Atp5k | | Atp1a1 |
| | | Rsl1d1 | | Akr1c13 |
| | | Stip1 | | Slc27a4 |
| | | Uqcrq | | Mgst3 |
| | | Plcb3 | | Gda |
| | | Reg3g | | Gls |
| | | Picalm | | Cds1 |
| | | | | Atp5g3 |
| | | | | Lgals3 |
| | | | | Lgals4 |
| | | | | Adipor2 |
| | | | | Fam213b |
| | | | | Ak2 |
| | | | | Abcd3 |
| | | | | Slc30a10 |
| | | | | Mgat4a |
| | | | | Iqgap2 |
| | | | | Slc46a1 |
| | | | | Gm8909 |
| | | | | Slc37a4 |
| | | | | Apoc3 |
| | | | | Slc13a1 |
| | | | | Naprt1 |
| | | | | Sod1 |
| | | | | Vil1 |
| | | | | Ucp2 |
| | | | | Neurl3 |
| | | | | Gramd3 |
| | | | | Ppa1 |
| | | | | Gclm |
| | | | | Slc7a9 |
| | | | | Prpsap1 |
| | | | | 2010001M06Rik |
| | | | | Ctsb |
| | | | | Cldn23 |
| | | | | Akr1c14 |
| | | | | Kcnk5 |
| | | | | Treh |
| | | | | Prdx1 |
| | | | | Tmem205 |
| | | | | Fahd1 |
| | | | | Elovl6 |
| | | | | Pgd |
| | | | | Cyp2c68 |
| | | | | Gm10768 |
| | | | | Leap2 |
| | | | | Agpat2 |
| | | | | Ppp1r14d |
| | | | | Isx |
| | | | | Prdx5 |
| | | | | Myo5b |
| | | | | Zfp36 |
| | | | | Hbegf |
| | | | | Sdhd |
| | | | | Clec2h |
| | | | | Serpinb6a |
| | | | | Slc25a25 |
| | | | | Ier5 |
| | | | | Gipc2 |
| | | | | Krt19 |
| | | | | Gng12 |
| | | | | Acad11 |
| | | | | Rragd |
| | | | | Otc |
| | | | | mt-Nd2 |
| | | | | Lypd8 |
| | | | | Gss |
| | | | | Faah |
| | | | | Slc25a15 |
| | | | | Osgin1 |
| | | | | Sdhb |
| | | | | Ctsz |
| | | | | Cdkn2b |
| | | | | H2-K1 |
| | | | | Alas1 |
| | | | | Lipa |
| | | | | Crat |
| | | | | Slc37a2 |
| | | | | Ech1 |
| | | | | Chchd10 |
| | | | | Tmem54 |
| | | | | Aldh2 |
| | | | | Oit1 |
| | | | | Ckb |
| | | | | Gde1 |
| | | | | Lad1 |
| | | | | Akr1b8 |
| | | | | Tcn2 |
| | | | | Efna1 |
| | | | | Cobl |
| | | | | Stk17b |
| | | | | Cldn3 |
| | | | | Msra |
| | | | | Naip1 |
| | | | | Pank3 |
| | | | | Ces1d |
| | | | | Mvp |
| | | | | Slc16a10 |
| | | | | Aco2 |
| | | | | mt-Nd1 |
| | | | | Pepd |
| | | | | Vat1 |
| | | | | Phgr1 |
| | | | | Calml4 |
| | | | | Aprt |
| | | | | Slc39a14 |
| | | | | Myh14 |
| | | | | Igsf23 |
| | | | | Entpd7 |
| | | | | Abhd6 |
| | | | | Cystm1 |
| | | | | Espn |
| | | | | A1cf |
| | | | | Itpk1 |
| | | | | Ogdh |
| | | | | Pard6b |
| | | | | Acadvl |
| | | | | Cth |
| | | | | Aldh1a7 |
| | | | | Dstn |
| | | | | Pnrc1 |
| | | | | Ugt1a7c |
| | | | | Gstm6 |
| | | | | Txndc17 |
| | | | | Pgm2 |
| | | | | Decr1 |
| | | | | Mall |
| | | | | Gpd2 |
| | | | | Trim31 |
| | | | | Sfn |
| | | | | 2210407C18Rik |
| | | | | Ms4a8a |
| | | | | Esrra |
| | | | | Cyp2d34 |
| | | | | Sqstm1 |
| | | | | Uqcrfs1 |
| | | | | Acbd4 |
| | | | | Dhrs11 |
| | | | | Ahcyl2 |
| | | | | Ptgr1 |
| | | | | Misp |
| | | | | Edf1 |
| | | | | Pfkp |
| | | | | Nfe2l2 |
| | | | | Hadha |
| | | | | Btnl2 |
| | | | | Asah2 |
| | | | | Nlrp6 |
| | | | | Egln3 |
| | | | | Prdx6 |
| | | | | Muc3 |
| | | | | Ace |
| | | | | Aldh3a2 |
| | | | | Aadac |
| | | | | C77080 |
| | | | | Pxmp4 |
| | | | | Heph |
| | | | | Ankrd9 |
| | | | | Emp1 |
| | | | | Tfg |
| | | | | Aldoc |
| | | | | Fzd5 |
| | | | | Plin3 |
| | | | | Dpyd |
| | | | | Cox7b |
| | | | | Car9 |
| | | | | Abcg2 |
| | | | | Prlr |
| | | | | Rnf186 |
| | | | | Mocs2 |
| | | | | Ppara |
| | | | | Sdha |
| | | | | Pla2g12b |
| | | | | Rab30 |
| | | | | Rhoc |
| | | | | Cyp2d10 |
| | | | | Gpr128 |
| | | | | Slc25a1 |
| | | | | Dhrs4 |
| | | | | Tob1 |
| | | | | Net1 |
| | | | | Pmaip1 |
| | | | | Nr1i3 |
| | | | | Prkg2 |
| | | | | Cox6a1 |
| | | | | Plekhb2 |
| | | | | Mylk |
| | | | | Glod5 |
| | | | | Anks4b |
| | | | | Tmem45b |
| | | | | Hagh |
| | | | | Fuca1 |
| | | | | Cdkn1a |
| | | | | Tmem120a |
| | | | | Cd302 |
| | | | | Cpt1a |
| | | | | Ociad2 |
| | | | | Serpinb1a |
| | | | | Sdhc |
| | | | | Smdt1 |
| | | | | Ifi35 |
| | | | | Slc26a2 |
| | | | | Rab6a |
| | | | | Slc30a1 |
| | | | | Uqcrc1 |
| | | | | Clec2e |
| | | | | Hsd17b2 |
| | | | | Shpk |
| | | | | Rnf103 |
| | | | | Synj2bp |
| | | | | Mep1a |
| | | | | Ppap2a |
| | | | | Cox5a |
| | | | | Cox4i1 |
| | | | | Adtrp |
| | | | | Tgoln1 |
| | | | | Slc16a6 |
| | | | | Ifi27l2b |

| early Paneth | Paneth | enteroendocrine |
|---|---|---|
| Fcgbp | Gm15284 | Chgb |
| Agr2 | Ang4 | Chga |
| Tff3 | Gm14851 | Cpe |
| Spink4 | Defa21 | Scg2 |
| Ccl9 | Defa22 | Neurog3 |
| Guca2a | Defa24 | Ptprn2 |
| Rnase4 | Defa17 | Aplp1 |
| Selm | Itln1 | Neurod1 |
| Rnase1 | Nupr1 | Ghrl |
| Muc2 | Lyz1 | Map1b |
| Ang | AY761184 | Vwa5b2 |
| Ern2 | Clps | Pla2g2f |
| Pdia5 | Pnliprp2 | Cplx2 |
| Creb3l1 | Defa3 | Peg3 |
| Spdef | Mmp7 | Gadd45a |
| Fxyd3 | Defa26 | Fev |
| Mmp7 | Thbs1 | Ets1 |
| Txndc5 | Gm15315 | Pcsk1 |
| Galnt7 | Guca2a | Cacna1a |
| Tmed3 | Spink4 | Cnot6l |
| Rap1gap | Atf3 | Sct |
| Pdia6 | Acta1 | Pam |
| Kit | Gm15293 | Syt13 |
| Bhlha15 | Tff3 | Trp53i11 |
| Cd24a | Defa-rs1 | Olfm1 |
| 5330417C22Rik | Habp2 | Igfbp2 |
| Atp2a3 | Herpud1 | Rab3c |
| Rab15 | Cldn4 | Miat |
| Slc12a8 | Tspan1 | Hepacam2 |
| Muc13 | Sytl2 | Cadps |
| Klf4 | Muc2 | Insm1 |
| Gcnt3 | Serp1 | Lrp11 |
| Gfpt1 | Nucb2 | St18 |
| Qsox1 | mmu-mir-6236 | Prnp |
| Galnt12 | Lars2 | Mtch1 |
| Fam46c | Bhlha15 | Rph3al |
| Spint2 | Qsox1 | Rundc3a |
| Calr | Gm10104 | Nefm |
| Sytl2 | Tanc2 | Fgd2 |
| Asph | Rnase1 | Dst |
| Pdia3 | Fam46c | Rfx6 |
| Pdia4 | Egr1 | Pak3 |
| Habp2 | Dnajc3 | Itpr1 |
| Dnajc10 | 5330417C22Rik | Cacna2d1 |
| Manf | Gm15292 | Myt1 |
| Ssr2 | Lrrc26 | Scg3 |
| Tmprss2 | Slc12a8 | Nkx2-2 |
| Tpd52 | Fos | Map3k15 |
| Fkbp11 | Tmed6 | Runx1t1 |
| Dnajc3 | Klf4 | Tecpr1 |
| Hspa5 | Hspa5 | Resp18 |
| Nucb2 | Mlph | Hk2 |
| Hsp90b1 | Chac1 | Sepp1 |
| Hdlbp | Agr2 | Igsf8 |
| Atoh1 | P4hb | Gpr112 |
| Ssr3 | Cd24a | Hap1 |
| Serp1 | Gm15564 | Ddc |
| Tm9sf3 | Mia3 | Kcnj3 |
| Clps | D10Bwg1379e | Scgn |
| Sec61a1 | Muc13 | Rimbp2 |
| Lyz1 | Krt18 | Tox3 |
| Krt18 | Eif2ak3 | Malat1 |
| Edem2 | Ern2 | Tbc1d9 |
| Ssr4 | Rnase4 | Lect2 |
| Sec11c | Smim14 | Camk2b |
| Hyou1 | Slc1a4 | Chd7 |
| Hid1 | Ssr3 | Marcksl1 |
| H13 | Spdef | Ceacam10 |
| Wars | Tram1 | Nisch |
| Dll4 | Spcs3 | Hmgn3 |
| Lman1 | Pycr1 | Gfra3 |
| | Rab3d | Setbp1 |
| | Tspan13 | Phldb2 |
| | Galnt6 | Rap1gap2 |
| | Atp2a3 | Maged1 |
| | Asph | Prkar1a |
| | Rrbp1 | Kcnb2 |
| | Ssr4 | Nudt4 |
| | Hdlbp | Cd177 |
| | Fkbp11 | Slc18a1 |
| | Rap1gap | Nfasc |
| | Selm | Stxbp5l |
| | Cdhr5 | Stxbp1 |
| | Ccl9 | Mical2 |
| | Hyou1 | Nbea |
| | Cyp2e1 | KCTD12 |
| | Fosb | Rbp4 |
| | Pla2g1b | Dopey2 |
| | Klf6 | Phip |
| | Btg2 | Pkdcc |
| | Ppp1r15a | Bex2 |
| | | Pcsk1n |
| | | Prox1 |
| | | Rev3l |
| | | Marcks |
| | | Tuba1a |
| | | Acly |
| | | Epb4.2 |
| | | Bace1 |
| | | Neto2 |
| | | Phf16 |
| | | Gm609 |
| | | Map4k4 |
| | | Ids |
| | | Pcdhga9 |
| | | Pde1c |
| | | Sbk1 |
| | | Celf3 |
| | | Jhdm1d |
| | | Pnmal2 |
| | | Pax4 |
| | | Ngfrap1 |
| | | Trim35 |
| | | Tmed8 |
| | | Sez6l2 |
| | | Scg5 |
| | | Wdr6 |
| | | Gnao1 |
| | | Fam183b |
| | | Ina |
| | | Neurod2 |
| | | Gnai1 |
| | | Scn3a |
| | | Arhgef2 |
| | | Tpst2 |
| | | Cdkn1c |
| | | Rims2 |
| | | Tmem86a |
| | | Dennd2a |
| | | Pbx1 |
| | | Anxa6 |
| | | Pax6 |
| | | Bambi |
| | | Disp2 |
| | | Dbn1 |
| | | Kcnh6 |
| | | 2510009E07Rik |
| | | Cdh2 |
| | | Rcor2 |
| | | Cep170 |
| | | Gdf11 |
| | | Unc13a |
| | | Gng4 |
| | | Ctsf |
| | | Tubb3 |
| | | Cacna1h |
| | | Gramd1a |
| | | Mdm1 |
| | | Fbxl16 |
| | | Kif1a |
| | | D630036G22Rik |
| | | Pim2 |
| | | Rtn2 |
| | | Syp |
| | | Pnmal1 |
| | | Snap25 |
| | | Gck |
| | | 6430573F11Rik |
| | | Fat4 |
| | | Ret |
| | | Dner |
| | | Ffar2 |
| | | Dact1 |
| | | Gadd45g |
| | | Neb |
| | | Gch1 |
| | | Tpm4 |
| | | Ephb3 |
| | | Itm2c |
| | | Chd4 |
| | | Hoxb8 |
| | | Tm4sf4 |
| | | Rogdi |
| | | Gse1 |
| | | Fgd6 |
| | | Rfx3 |
| | | Msx1 |
| | | Eml5 |
| | | Gnptg |
| | | Ldb1 |
| | | Akap9 |
| | | Phc2 |
| | | Tle1 |
| | | Dnmt3a |
| | | Kdm1a |
| | | Slc25a4 |
| | | Tmem176b |
| | | Kmt2a |
| | | Mast2 |
| | | Tshz1 |
| | | Gnai2 |
| | | Wnt3 |
| | | Atp6v1b2 |
| | | Serpinb1a |
| | | Phf21b |
| | | Kif12 |
| | | D19Ertd737e |
| | | Amotl2 |
| | | Cmip |
| | | Mycbp2 |
| | | Ddx6 |
| | | Rap1b |
| | | Neo1 |
| | | Krt7 |
| | | Abcc4 |
| | | Ttll7 |
| | | Cxxc5 |
| | | E330009J07Rik |
| | | Atxn10 |
| | | Fryl |
| | | Trp53bp1 |
| | | Slc36a1 |
| | | Cck |
| | | Abca7 |
| | | Rbfox2 |
| | | Gdi1 |
| | | Hspa4l |
| | | Txnip |
| | | Dach1 |
| | | Rnf32 |
| | | Mlxipl |
| | | Cst3 |
| | | Sptan1 |
| | | C530008M17Rik |
| | | Sox4 |
| | | Rasd1 |
| | | Tbc1d8 |
| | | Rbms3 |
| | | Cyp4b1 |
| | | Ssh2 |
| | | Ptov1 |
| | | Camk2n1 |
| | | Dpysl2 |
| | | Smpd3 |
| | | 2010107G23Rik |
| | | Ddx17 |
| | | Btg1 |
| | | 5330417C22Rik |
| | | Gng12 |
| | | Fam115a |
| | | Cd81 |
| | | Myo1e |
| | | Ccnl2 |
| | | Ttc3 |
| | | Cbfa2t2 |
| | | Dpp4 |
| | | Zmynd11 |
| | | Msi2 |
| | | Tmem184c |
| | | Zcchc11 |
| | | Vezf1 |
| | | Hes6 |
| | | Cbfa2t3 |
| | | Lcorl |
| | | Fxyd3 |
| | | Dip2b |
| | | Fam135a |
| | | Chn2 |
| | | Tspan12 |
| | | Mpzl1 |
| | | Aim1 |
| | | Chd3 |
| | | Hopx |
| | | Madd |
| | | Ssbp3 |
| | | Golim4 |
| | | Smad1 |
| | | Cyth2 |
| | | Serinc1 |
| | | Parm1 |
| | | Slc43a2 |
| | | Htatsf1 |
| | | Smarcd2 |
| | | Ubl3 |
| | | Atp6v0b |
| | | Ankib1 |
| | | Ica1 |
| | | Paxip1 |
| | | Mtss1 |
| | | Ncald |
| | | Rgcc |
| | | Ttr |
| | | Rhob |
| | | Parp1 |
| | | Lgals3bp |
| | | Cdkn1a |

Quantification and statistical analysis. In each experiment, multiple mice models were analyzed as biological replicates: n=3 mice for data reported in FIG. 23; n=5 for data reported in FIG. 23E; n=8 single-well replicates randomly selected rom 5 donor mice for data in FIG. 23F; n=13 co-culture well replicates randomly selected from 4 donor mice for data reported in FIG. 23G; n=2 mice (2 technical replicates each) for data reported in FIG. 24; n=1 C57BL/6 mouse and 1 [in vivo genotype] mouse for data reported in FIG. 25; n=3 mice for data reported in FIG. 26. Graphs show mean±SEM, unless otherwise noted. Unpaired 2-tail t-test and 2-way ANOVA-multiple comparison were used to assess statistical significance. * indicates p<0.05, ** p<0.01*** p<0.001, and **** p<0.0001.

### REFERENCES

Arango, N. P., Yuca, E., Zhao, M., Evans, K. W., Scott, S., Kim, C., Gonzalez-Angulo, A. M., Janku, F., Ueno, N. T., Tripathy, D., et al. (2017). Selinexor (KPT-330) demonstrates antitumor efficacy in preclinical models of triple-negative breast cancer. Breast Cancer Res. 19, 93.
Ayyaz, A., Kumar, S., Sangiorgi, B., Ghoshal, B., Gosio, J., Ouladan, S., Fink, M., Barutcu, S., Trcka, D., Shen, J., et al. (2019). Single-cell transcriptomes of the regenerating intestine reveal a revival stem cell. Nature 569, 121-125.
Azmi, A. S., Aboukameel, A., Bao, B., Sarkar, F. H., Philip, P. A., Kauffman, M., Shacham, S., and Mohammad, R. M. (2013). Selective Inhibitors of Nuclear Export Block Pancreatic Cancer Cell Proliferation and Reduce Tumor Growth in Mice. Gastroenterology 144, 447-456.
Barker, N., van Es, J. H., Kuipers, J., Kujala, P., van den Born, M., Cozijnsen, M., Haegebarth, A., Korving, J., Begthel, H., Peters, P. J., et al. (2007). Identification of stem cells in small intestine and colon by marker gene Lgr5. Nature 449, 1003-1007.
Basak, O., Beumer, J., Wiebrands, K., Seno, H., van Oudenaarden, A., and Clevers, H. (2017). Induced Quiescence of Lgr5+ Stem Cells in Intestinal Organoids Enables Differentiation of Hormone-Producing Enteroendocrine Cells. Cell Stem Cell 20, 177-190.e4.
Beyaz, S., Mana, M. D., Roper, J., Kedrin, D., Saadatpour, A., Hong, S.-J., Bauer-Rowe, K. E., Xifaras, M E., Akkad, A., Arias, E., et al. (2016). High-fat diet enhances sternness and tumorigenicity of intestinal progenitors. Nature 531, 53-58.
Bhatia, S. N., and Ingber, D. E. (2014). Microfluidic organs-on-chips. Nat. Biotechnol. 32, 760-772.
Biton, M., Haber, A. L., Rogel, N., Burgin, G., Beyaz, S., Schnell, A., Ashenberg, O., Su, C. W., Smillie, C., Shekhar, K., et al. (2018). T Helper Cell Cytokines Modulate Intestinal Stem Cell Renewal and Differentiation. Cell 1-14.
Cheng, C. W., Biton, M., Haber, A. L., Gunduz, N., Eng, G., Gaynor, L. T., Tripathi, S., Calibasi-Kocal, G., Rickelt, S., Butty, V. L., et al. (2019). Ketone Body Signaling Mediates Intestinal Stem Cell Homeostasis and Adaptation to Diet. Cell 178, 1115-1131.e15.
Clevers, H. (2016). Modeling Development and Disease with Organoids. Cell 165, 1586-1597.
Dionne, S., Laberge, S., Deslandres, C., and Seidman, E. G. (2003). Modulation of cytokine release from colonic explants by bacterial antigens in inflammatory bowel disease. Clin. Exp. Immunol. 133, 108-114.
Draheim, K. M., Chen, H. B., Tao, Q., Moore, N., Roche, M., and Lyle, S. (2010). ARRDC3 suppresses breast cancer progression by negatively regulating integrin B4. Oncogene 29, 5032-5047.
Eriguchi, Y., Takashima, S., Oka, H., Shimoji, S., Nakamura, K., Uryu, H., Shimoda, S., Iwasaki, H., Shimono, N., Ayabe, T., et al. (2012). Graft-versus-host disease disrupts intestinal microbial ecology by inhibiting Paneth cell production of α-defensins. Blood 120, 223-231.
Forbes, D. J., Travesa, A., Nord, M. S., and Bernis, C. (2015). Nuclear transport factors: Global regulation of mitosis. Curr. Opin. Cell Biol. 35, 78-90.
Fre, S., Huyghe, M., Mourikis, P., Robine, S., Louvard, D., and Artavanis-Tsakonas, S. (2005). Notch signals control the fate of immature progenitor cells in the intestine. Nature 435, 964-968.
Fu, S. C., Huang, H. C., Horton, P., and Juan, H. F. (2013). ValidNESs: A database of validated leucine-rich nuclear export signals. Nucleic Acids Res. 41, 338-343.
Gassler, N. (2017). Paneth cells in intestinal physiology and pathophysiology. World J. Gastrointest. Pathophysiol. 8, 150-160.
Haber, A. L., Biton, M., Rogel, N., Herbst, R. H., Shekhar, K., Smillie, C., Burgin, G., Delorey, T. M., Howitt, M. R., Katz, Y., et al. (2017). A single-cell survey of the small intestinal epithelium. Nature.
Hafemeister, C., and Satija, R. (2019). Normalization and variance stabilization of single-cell RNA-seq data using regularized negative binomial regression. Genome Biol. 20, 296.
Han, T., Schatoff, E. M., Murphy, C., Zafra, M. P., Wilkinson, J. E., Elemento, O., and Dow, L. E. (2017). R-Spondin chromosome rearrangements drive Wnt-dependent tumour initiation and maintenance in the intestine. Nat. Commun. 8, 15945.
Hayase, E., Hashimoto, D., Nakamura, K., Noizat, C., Ogasawara, R., Takahashi, S., Ohigashi, H., Yokoi, Y., Sugimoto, R., Matsuoka, S., et al. (2017). R-Spondin1 expands Paneth cells and prevents dysbiosis induced by graft-versus-host disease. J. Exp. Med. 214, 3507-3518.
Hing, Z. A., Fung, H. Y. J., Ranganathan, P., Mitchell, S., El-Gamal, D., Woyach, J. A., Williams, K., Goettl, V. M., Smith, J., Yu, X., et al. (2016). Next-generation XPO1 inhibitor shows improved efficacy and in vivo tolerability in hematological malignancies. Leukemia 30, 2364-2372.
Hughes, T. K., Wadsworth, M. H., Gierahn, T. M., Do, T., Weiss, D., Andrade, P. R., Ma, F., Silva, B. J. de A., Shao, S., Tsoi, L. C., et al. (2019). Highly Efficient, Massively-Parallel Single-Cell RNA-Seq Reveals Cellular States and Molecular Features of Human Skin Pathology. BioRxiv 689273.
Ireland, H., Houghton, C., Howard, L., and Winton, D. J. (2005). Cellular inheritance of a Cre-activated reporter gene to determine paneth cell longevity in the murine small intestine. Dev. Dyn. 233, 1332-1336.
Jadhav, K., and Zhang, Y. (2017). Activating transcription factor 3 in immune response and metabolic regulation. Liver Res. 1, 96-102.
Jensen, J., Pedersen, E. E., Galante, P., Hald, J., Heller, R. S., Ishibashi, M., Kageyama, R., Guillemot, F., Serup, P., and Madsen, O. D. (2000). Control of endodermal endocrine development by Hes-1. Nat. Genet. 24, 36-44.
Khor, B., Gardet, A., and Xavier, R. J. (2011). Genetics and pathogenesis of inflammatory bowel disease. Nature 474, 307-317.
Kim, K. A., Kakitani, M., Zhao, J., Oshima, T., Tang, T., Binnerts, M., Liu, Y., Boyle, B., Park, E., Emtage, P., et al. (2005). Mitogenic influence of human R-spondin1 on the intestinal epithelium. Science 309, 1256-1259.
Langhans, S. A. (2018). Three-Dimensional in Vitro Cell Culture Models in Drug Discovery and Drug Repositioning. Front. Pharmacol. 9, 1-14.
Liberzon, A., Birger, C., Thorvaldsdóttir, H., Ghandi, M., Mesirov, J. P., and Tamayo, P. (2015). The Molecular Signatures Database Hallmark Gene Set Collection. Cell Syst. 1, 417-425.
Liu, T. C., Gurram, B., Baldridge, M. T., Head, R., Lam, V., Luo, C., Cao, Y., Simpson, P., Hayward, M., Holtz, M. L., et al. (2016). Paneth cell defects in Crohn's disease patients promote dysbiosis. JCI Insight 1, e86907.
Macosko, E. Z., Basu, A., Satija, R., Nemesh, J., Shekhar, K., Goldman, M., Tirosh, I., Bialas, A. R., Kamitaki, N., Martersteck, E. M., et al. (2015). Highly parallel genome-wide expression profiling of individual cells using nanoliter droplets. Cell 161, 1202-1214.
McElroy, S. J., Underwood, M. A., and Sherman, M. P. (2013). Paneth cells and necrotizing enterocolitis: a novel hypothesis for disease pathogenesis. Neonatology 103, 10-20.
McGinnis, C. S., Murrow, L. M., and Gartner, Z. J. (2019). DoubletFinder: Doublet Detection in Single-Cell RNA Sequencing Data Using Artificial Nearest Neighbors. Cell Syst. 8, 329-337.e4.
McGuckin, M. a, Eri, R., Simms, L. a, Florin, T. H. J., and Radford-Smith, G. (2009). Intestinal barrier dysfunction in inflammatory bowel diseases. Inflamm. Bowel Dis. 15, 100-113.
Mead, B. E., Ordovas-Montanes, J., Braun, A. P., Levy, L. E., Bhargava, P., Szucs, M. J., Ammendolia, D. A., MacMullan, M. A., Yin, X., Hughes, T. K., et al. (2018). Harnessing single-cell genomics to improve the physiological fidelity of organoid-derived cell types. BMC Biol. 16, 62.
von Moltke, J., Ji, M., Liang, H., and Locksley, R. M. (2016). Tuft-cell-derived IL-25 regulates an intestinal ILC2-epithelial response circuit. Nature 529, 221-225.
Mpindi, J. P., Swapnil, P., Dmitrii, B., Jani, S., Saeed, K., Wennerberg, K., Aittokallio, T., Östling, P., and Kallioniemi, O. (2015). Impact of normalization methods on high-throughput screening data with high hit rates and drug testing with dose-response data. Bioinformatics 31, 3815-3821.
Naik, S., Larsen, S. B., Gomez, N. C., Alaverdyan, K., Sendoel, A., Yuan, S., Polak, L., Kulukian, A., Chai, S., and Fuchs, E. (2017). Inflammatory memory sensitizes skin epithelial stem cells to tissue damage. Nature 550, 475-480.
Okubo, T., and Hogan, B. L. M. (2004). Hyperactive Wnt signaling changes the developmental potential of embryonic lung endoderm. J. Biol. 3, 11.
Ordovas-Montanes, J., Dwyer, D. F., Nyquist, S. K., Buchheit, K. M., Vukovic, M., Deb, C., Wadsworth, M. H., Hughes, T. K., Kazer, S. W., Yoshimoto, E., et al. (2018). Allergic inflammatory memory in human respiratory epithelial progenitor cells. Nature 560, 649-654.
Pinto, D., Gregorieff, A., Begthel, H., and Clevers, H. (2003). Canonical Wnt signals are essential for homeostasis of the intestinal epithelium. Genes Dev. 17, 1709-1713.
Ranga, A., Gjorevski, N., and Lutolf, M. P. (2014). Drug discovery through stem cell-based organoid models. Adv. Drug Deliv. Rev. 69-70C, 19-28.
Sansom, O. J, Reed, K. R., Hayes, A. J., Ireland, H., Brinkmann, H., Newton, I. P., Batlle, E., Simon-Assmann, P., Clevers, H., Nathke, I. S., et al. (2004). Loss of Apc in vivo immediately perturbs Wnt signaling, differentiation, and migration. Genes Dev. 18, 1385-1390.
Sato, T., Vries, R. G., Snippert, H. J., van de Wetering, M., Barker, N., Stange, D. E., van Es, J. H., Abo, A., Kujala, P., Peters, P. J., et al. (2009). Single Lgr5 stem cells build crypt-villus structures in vitro without a mesenchymal niche. Nature 459, 262-265.
Sendino, M., Omaetxebarria, M. J., and Rodriguez, J. A. (2018). Hitting a moving target: inhibition of the nuclear export receptor XPO1/CRM1 as a therapeutic approach in cancer. Cancer Drug Resist.
Sherman, M. P., Bennett, S. H., Hwang, F. F. Y., Sherman, J., and Bevins, C. L. (2005). Paneth cells and antibacterial host defense in neonatal small intestine. Infect. Immun. 73, 6143-6146.
Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: A knowledge-based approach for interpreting genome-wide expression profiles. Proc. Natl. Acad. Sci. 102, 15545-15550.
Sun, Q., Chen, X., Zhou, Q., Burstein, E., Yang, S., and Jia, D. (2016). Inhibiting cancer cell hallmark features through nuclear export inhibition. Signal Transduct. Target. Ther. 1, 34-36.
Tanner, S. M., Berryhill, T. F., Ellenburg, J. L., Jilling, T., Cleveland, D. S., Lorenz, R. G.,
and Martin, C. A. (2015). Pathogenesis of necrotizing enterocolitis: modeling the innate immune response. Am. J. Pathol. 185, 4-16.
Tetteh, P. W., Basak, O., Farin, H. F., Wiebrands, K., Kretzschmar, K., Begthel, H., van den Born, M., Korving, J., de Sauvage, F., van Es, J. H., et al. (2016). Replacement of Lost Lgr5-Positive Stem Cells through Plasticity of Their Enterocyte-Lineage Daughters. Cell Stem Cell 18, 203-213.
Tirosh, I., Izar, B., Prakadan, S. M., Wadsworth, M. H., Treacy, D., Trombetta, J. J., Rotem, A., Rodman, C., Lian, C., Murphy, G., et al. (2016). Dissecting the multicellular ecosystem of metastatic melanoma by single-cell RNA-seq. Science 352, 189-196.
Tyler, P. M., Servos, M. M., de Vries, R. C., Klebanov, B., Kashyap, T., Sacham, S., Landesman, Y., Dougan, M., and Dougan, S. K. (2017). Clinical Dosing Regimen of Selinexor Maintains Normal Immune Homeostasis and T-cell Effector Function in Mice: Implications for Combination with Immunotherapy. Mol. Cancer Ther. 16, 428-439.
VanDussen, K. L., and Samuelson, L. C. (2010). Mouse atonal homolog 1 directs intestinal progenitors to secretory cell rather than absorptive cell fate. Dev. Biol. 346, 215-223.
VanDussen, K. L., Carulli, A. J., Keeley, T. M., Patel, S. R., Puthoff, B. J., Magness, S T., Tran, I T., Maillard, I., Siebel, C., Kolterud, A., et al. (2012). Notch signaling modulates proliferation and differentiation of intestinal crypt base columnar stem cells. Development 139, 488-497.
White, J. R., Gong, H., Pope, B., Schlievert, P., and McElroy, S. J. (2017). Paneth-cell-disruption-induced necrotizing enterocolitis in mice requires live bacteria and occurs independently of TLR4 signaling. Dis. Model. Mech. 10, 727-736.
Wu, A., Yu, B., Zhang, K., Xu, Z., Wu, D., He, J., Luo, J., Luo, Y., Yu, J., Zheng, P., et al. (2020). Transmissible gastroenteritis virus targets Paneth cells to inhibit the self-renewal and differentiation of Lgr5 intestinal stem cells via Notch signaling. Cell Death Dis. 11, 40.
Xavier, R. J., and Podolsky, D. K. (2007). Unravelling the pathogenesis of inflammatory bowel disease. Nature 448, 427-434.
Yan, K. S., Gevaert, O., Zheng, G. X. Y., Anchang, B., Probert, C. S., Larkin, K. A., Davies, P. S., Cheng, Z., Kaddis, J. S., Han, A., et al. (2017). Intestinal Enteroendocrine Lineage Cells Possess Homeostatic and Injury-Inducible Stem Cell Activity. Cell Stem Cell 21, 78-90.e6.
Yang, H. W., Chung, M., Kudo, T., and Meyer, T. (2017). Competing memories of mitogen and p53 signalling control cell-cycle entry. Nature 549, 404-408.
Yin, X., Farin, H. F., van Es, J. H., Clevers, H., Langer, R., and Karp, J. M. (2014). Niche-independent high-purity cultures of Lgr5+ intestinal stem cells and their progeny. Nat. Methods 11, 106-112.
Yousefi, M., Li, L., and Lengner, C. J. (2017). Hierarchy and Plasticity in the Intestinal Stem Cell Compartment. Trends Cell Biol. 27, 753-764.
Zhang, X. D. (2011). Hit Selection in Genome-Scale RNAi Screens with Replicates. In Optimal High-Throughput Screening, (Cambridge: Cambridge University Press), pp. 83-108.
Zheng, Y., Gery, S., Sun, H., Shacham, S., Kauffman, M., and Koeffler, H. P. (2014). KPT-330 inhibitor of XPO1-mediated nuclear export has anti-proliferative activity in hepatocellular carcinoma. Cancer Chemother. Pharmacol. 74, 487-495.
Zhou, J., Edgar, B. A., and Boutros, M. (2017a). ATF3 acts as a rheostat to control JNK signalling during intestinal regeneration. Nat. Commun. 8, 1-15.
Zhou, X., Geng, L., Wang, D., Yi, H., Talmon, G., and Wang, J. (2017b). R-Spondin1/LGR5 Activates TGFβ Signaling and Suppresses Colon Cancer Metastasis. Cancer Res. 77, 6589-6602.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of differentiating a stem cell or a stem cell enriched population of cells, comprising:
contacting the population of cells with one or more cell cycle inhibitors; and/or
contacting the population of cells with one or more agents capable of increasing expression or activity of one or more genes or gene products selected from the group consisting of:
Atf3; or
Atf3, Klf6, Hbegf, Ubc, Muc13; or
Klf6, Atf3, Hbegf, Ubc, Muc13, 2210404007Rik, Pigr, Ndrg1, S1c16a6, Btg2, Thbs1, Cldn4, Edn1, Smim6, Arrdc3, Abcg8, Tmem171, Prr15, Rnase4, Itln1, Mmp7, Gm15284, Defa17, Lyz1, Tff3, Rps26, Rpl13, Hmgn1, Sox4, Malat1, S1c12a2, Atp1a1, Tmprss2, Gls, Mt2, Mt1, mmu-mir-6236 and Gm26924; or
upregulated genes in Table 3A; or
upregulated pathways in Table 3B, and/or
contacting the population of cells with one or more agents capable of decreasing expression or activity of one or more genes or gene products selected from the group consisting of:
Topbp1, Rpa1, Mcm7, Atad2, Lig1, Whsc1, Dut1, Urf1, Mcm3, Mcm5, Mcm4, Hells, Mcm6, Mcm2, Pcna, Tgm2, Crip2, Fen1, Ccnd1, Dtl, Cdt1, Ptma, Hook1, Tubb5, Mki67, Ranbp1 and Kpnb1; or
Itpr3, Cbr1, Kmt2a, Hsph1, Gm2697, Bex1, Cad, Rpph1, Gclc, Cbr3, Pfkp, Ugdh, Elovl6, Slc25a4, Galk1, Wars, Slc38a1, Parp1, Mrpl12, Gsr, Gnl3, Ybx3, Ckb, Isynal, Nop56, Nolc1, Reep6, Ccnd2, Srm, Gpx2, Add3, Oxct1, Nhp2, Bzw2, Prmt5, Rangap1, H1f0, Sae1, C1qbp, Tpi1, Hmgcs1, Smoc2, Axin2, Cdk4, Reg3 g, Igfbp4, Rgcc, Maged1, Tcof1, Dctpp1, Nasp, Gart, Rif1, Paics, Hjurp, Ezh2, Rrm1, Dnmt1, Ipo5, Fads1, Topbp1, Rpa1, Mcm7, Atad2, Lig1, Whsc1, Dut1, Urf1, Mcm3, Mcm5, Mcm4, Hells, Mcm6, Mcm2, Pcna, Tgm2, Crip2, Fen1, Ccnd1, Dtl, Cdt1, Ptma, Hook1, Tubb5, Mki67, Ranbp1 and Kpnb1; or
downregulated genes in Table 3A; or
downregulated pathways in Table 3B, and/or
contacting the population of cells with one or more agents capable of inducing a quiescent stem cell signature.

2. The method of claim 1, wherein the one or more agents capable of inducing a quiescent stem cell signature comprise a map kinase inhibitor.

3. The method of claim 2, wherein the map kinase inhibitor is cobimetinib.

4. The method of any of claims 1 to 3, wherein the one or more agents comprises a small molecule, genetic modifying agent, nucleic acid molecule encoding a gene product, protein, or any combination thereof.

5. The method of claim 4, wherein the genetic modifying agent comprises a CRISPR system, RNAi system, a zinc finger nuclease system, a TALE system, or a meganuclease.

6. The method of claim 5, wherein the CRISPR system is a Class 1 or Class 2 CRISPR system.

7. The method of claim 6, wherein the Class 2 system comprises a Type II Cas polypeptide.

8. The method of claim 7, wherein the Type II Cas is a Cas9.

9. The method of claim 6, wherein the Class 2 system comprises a Type V Cas polypeptide.

10. The method of claim 9, wherein the Type V Cas is Cas12a, Cas12b, Cas12c, Cas12d (CasY), Cas12e (CasX), or Cas14.

11. The method of claim 6, wherein the Class 2 system comprises a Type VI Cas polypeptide.

12. The method of claim 11, wherein the Type VI Cas is Cas13a, Cas13b, Cas13c or Cas13d.

13. The method of any of claims 6 to 12, wherein the CRISPR system comprises a dCas fused or otherwise linked to a nucleotide deaminase.

14. The method of claim 13, wherein the nucleotide deaminase is a cytidine deaminase or an adenosine deaminase.

15. The method of claim 6, wherein the CRISPR system is a prime editing system.

16. The method of any one of claims 1 to 15, further comprising contacting the population of cells with a Wnt agonist.

17. The method of claim 16, wherein the Wnt agonist is CHIR99021.

18. The method of any one of claims 1 to 17, further comprising contacting the population of cells with a Notch inhibitor.

19. The method of claim 18, wherein the Notch inhibitor is DAPT.

20. The method of any one of claims 1 to 19, wherein the stem cells are leucine-rich repeat-containing G-protein coupled receptor 5-positive (LGR5+) cells.

21. The method of claim 20, wherein the LGR5+ cells are LGR5+ intestinal stem cells (ISC), LGR5+ cochlear progenitors (LCP), LGR5+ stem cells of the respiratory epithelium, or LGR5+ stem cells of the skin.

22. The method of any one of claims 1 to 21, wherein the population of cells is an in vivo population of cells.

23. The method of any one of claims 1 to 21, wherein the population of cells is an in vitro organoid model system.

24. The method of claim 23, further comprising obtaining intestinal cells from a subject.

25. The method of claim 23, further comprising obtaining inner ear cells from a subject.

26. The method of any of claims 1 to 25, wherein secretory cells are increased in the population of cells.

27. The method of claim 26, wherein the secretory cells comprise Paneth cells.

28. The method of claim 27, wherein the Paneth cells express one or more genes or gene products selected from the group consisting of human lysozyme (LYZ), a human alpha defensin (DEFA), human matrix metalloproteinase-7 (MMP-7), and cluster of differentiation 24 (CD24).

29. The method of claim 28, wherein the human alpha defensin is human alpha defensin 5 (DEFA5) or human alpha defensin 6 (DEFA6).

30. The method of any of claims 1 to 25, wherein sensory cells are increased in the population of cells.

31. The method of claim 30, wherein the sensory cells comprise inner ear hair cells.

32. The method of any of claims 23 to 31, wherein the method comprises:
a) culturing an LGR5+ enriched population of cells in a hydrogel matrix in the presence of EGF, Noggin, R-spondin 1, CHIR99021 and valproic acid (ENR+CV);
b) culturing the ENR+CV cells in the presence of EGF, Noggin, R-spondin 1, CHIR99021 and DAPT (ENR+CD); and
c) contacting the cells according to claim 1.

33. The method of any of claims 1 to 32, wherein the method further comprises introducing differentiated cells to a subject.

34. The method of any of claims 1 to 33, wherein the method is for treating a condition or disease by increasing Paneth cells in a subject in need thereof.

35. The method of claim 34, wherein the condition or disease is selected from the group consisting of graft-versus-host disease (GVHD), inflammatory bowel disease (IBD), Crohn's disease, necrotizing enterocolitis, microbial dysbiosis, impaired intestinal epithelial barrier function, obesity, intestinal inflammation, allergy, respiratory inflammation, asthma, and psoriasis.

36. The method of claim 34 or 35, wherein the method comprises targeted administration of the one or more agents or cells to the intestine of the subject.

37. The method of any of claims 1 to 33, wherein the method is for treating hearing loss by increasing hair cells in the inner ear of a subject in need thereof.

38. The method of claim 37, wherein the method comprises targeted administration of the one or more agents or cells to the inner ear of the subject.

39. A population of cells produced by the method of any of claims 1 to 32.

40. A method of treating a condition or disease by increasing Paneth cells in a subject in need thereof comprising systemically administering an inhibitor of nuclear export to the subject in a dosage less than or equal to 0.2 mg/kg.

41. The method of claim 40, wherein the dosage is between 0.01 to 0.2 mg/kg.

42. The method of claim 40, wherein the dosage is less than or equal to 0.01 mg/kg.

43. The method of any of claims 40 to 42, wherein the inhibitor is administered orally.

44. The method of any of claims 40 to 42, wherein the inhibitor is administered by injection.

45. The method of any of claims 40 to 44, wherein the inhibitor of nuclear export inhibits XPO1.

46. The method of claim 45, wherein the inhibitor is KPT-330.

47. The method of claim 45, wherein the inhibitor is KPT-8602.

48. The method of claim 45, wherein the inhibitor is Leptomycin B.

49. The method of any of claims 40 to 48, wherein the condition or disease is selected from the group consisting of inflammatory bowel disease (IBD), Crohn's disease, intestinal inflammation, graft-versus-host disease (GVHD), necrotizing enterocolitis, microbial dysbiosis, impaired intestinal epithelial barrier function, obesity, allergy, respiratory inflammation, asthma, and psoriasis.

50. A method of detecting intestinal cell types comprising detecting in a sample comprising intestinal cells the expression of one or markers selected from Table 6 for one or more cell types selected from the group consisting of stem I cells, stem II cells, stem III cells, early enterocytes, enterocytes, early Paneth cells, Paneth cells and enteroendocrine cells.

51. The method of claim 50, wherein the fraction of each cell type is determined.

52. A method of identifying an agent capable of differentiating a stem cell or a stem cell enriched population of cells, comprising:
a) applying a candidate agent to an intestinal organoid model; and
b) detecting one or more intestinal cell types according to claim 50, whereby a shift in cell types indicates that the agent is capable of differentiating a stem cell or a stem cell enriched population of cells.

53. A method of differentiating a stem cell or a stem cell enriched population of cells comprising contacting the population of cells with one or more inhibitors of aurora kinase b.

54. The method of claim 53, wherein the inhibitor is ZM447439.
